SEQUENCE LISTING (290 sequences)

SEQ ID NO: 1 - RNA, 82 nt, Artificial Sequence
  Synthetic polynucleotide
guuuuagagcuagaaauagcaaguuaaaauaaaggcuaguccguuaucaacuugaaaaaguggcaccgagucggugcuuuuu

SEQ ID NO: 2 - RNA, 93 nt, S. pyogenes
guuuaagagcuaugcuggaaagccacggugaaaaaguucaacuauugccugaucggaauaaauuugaacgauacgacagucggugcuuuuuuu

SEQ ID NO: 3 - RNA, 82 nt, S. pyogenes
guuuaagagcuagaaauagcaaguuuaaauaaggcuaguccguuaucaacuugaaaaaguggcaccgagucggugcuuuuuu

SEQ ID NO: 4 - RNA, 97 nt, S. thermophilus
guuuuuguacucucaagauucaauaaucuugcagaagcuacaaagauaaggcuucaugccgaaaucaacacccugucauuuuauggcaggguguuuu

SEQ ID NO: 5 - RNA, 97 nt, S. thermophilus
guuuuagagcuguguuguuuguuaaaacaacacagcgaguuaaaauaaggcuuaguccguacucaacuugaaaagguggcaccgauucgguguuuuu

SEQ ID NO: 6 - RNA, 74 nt, C. jejuni
aagaaauuuaaaaagggacuaaaauaaagaguuugcgggacucugcgggguuacaauccccuaaaaccgcuuuu

SEQ ID NO: 7 - RNA, 95 nt, F. novicida
aucuaaaauuauaaauguaccaaauaauuaaugcucuguaaucauuuaaaaguauuuugaacggaccucuguuugacacgucugaauaacuaaaa

SEQ ID NO: 8 - RNA, 109 nt, S. thermophilus2
uguaagggacgccuuacacaguuacuuaaaucuugcagaagcuacaaagauaaggcuucaugccgaaaucaacacccugucauuuuauggcaggguguuuucguuauuu

SEQ ID NO: 9 - RNA, 110 nt, M. mobile
uguauuucgaaauacagauguacaguuaagaauacauaagaaugauacaucacuaaaaaaaggcuuuaugccguaacuacuacuuauuuucaaaauaaguaguuuuuuuu

SEQ ID NO: 10 - RNA, 92 nt, L. innocua
auuguuaguauucaaaauaacauagcaaguuaaaauaaggcuuuguccguuaucaacuuuuaauuaaguagcgcuguuucggcgcuuuuuuu

SEQ ID NO: 11 - RNA, 89 nt, S. pyogenes
guuggaaccauucaaaacagcauagcaaguuaaaauaaggcuaguccguuaucaacuugaaaaaguggcaccgagucggugcuuuuuuu

SEQ ID NO: 12 - RNA, 107 nt, S. mutans
guuggaaucauucgaaacaacacagcaaguuaaaauaaggcagugauuuuuaauccaguccguacacaacuugaaaaagugcgcaccgauucggugcuuuuuuauuu

SEQ ID NO: 13 - RNA, 96 nt, S. thermophilus
uugugguuugaaaccauucgaaacaacacagcgaguuaaaauaaggcuuaguccguacucaacuugaaaagguggcaccgauucgguguuuuuuuu

SEQ ID NO: 14 - RNA, 102 nt, N. meningitidis
acauauugucgcacugcgaaaugagaaccguugcuacaauaaggccgucugaaaagaugugccgcaacgcucugccccuuaaagcuucugcuuuaaggggca

SEQ ID NO: 15 - RNA, 110 nt, P. multocida
gcauauuguugcacugcgaaaugagagacguugcuacaauaaggcuucugaaaagaaugaccguaacgcucugccccuugugauucuuaauugcaaggggcaucguuuuu

SEQ ID NO: 16 - RNA, 93 nt, S. pyogenes; identical to SEQ ID NO: 2

SEQ ID NO: 17 - DNA, 80 bp, Artificial Sequence
  Synthetic polynucleotide
gggttagagctagaaatagcaagttaacctaaggctagtccgttatcaacttgaaaaagtggcaccgagtcggtgctttt

SEQ ID NO: 18 - protein, 1368 aa, S. pyogenes [sequence omitted]

SEQ ID NO: 19 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 20 - protein, 1368 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1368 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 23 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 24 - protein, 80 aa, Artificial Sequence
  Synthetic polypeptide
GGGTTAGAGCTAGAAATAGCAAGTTAACCTAAGGCTAGTCCGTTATCAACTTGAAAAAGTGGCACCGAGTCGGTGCTTTT

SEQ ID NO: 25 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 26 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 27 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 28 - protein, 1300 aa, Artificial Sequence
  Synthetic polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 29 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 30 - protein, 575 aa, Homo sapiens
MDPRGILKAFPKRQKIHADASSKVLAKIPRREEGEEAEEWLSSLRAHVVRTGIGRARAELFEKQIVQHGGQLCPAQGPGVTHIVVDEGMDYERALRLLRLPQLPPGAQLVKSAWLSLCLQERRLVDVAGFSIFIPSRYLDHPQPSKAEQDASIPPGTHEALLQTALSPPPPPTRPVSPPQKAKEAPNTQAQPISDDEASDGEETQVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVSQEENGQQQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 31 - protein, 120 aa, Artificial Sequence
  Synthetic polypeptide
  MISC_FEATURE: May be absent
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 32 - protein, 150 aa, Artificial Sequence
  Synthetic polypeptide
  MISC_FEATURE (6)..(150): May be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 33 - protein, 150 aa, Artificial Sequence
  Synthetic polypeptide
  MISC_FEATURE (6)..(150): May be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 34 - protein, 16 aa, Artificial Sequence
  Synthetic polypeptide
SGSETPGTSESATPES

SEQ ID NO: 35 - protein, 16 aa, Artificial Sequence
  Synthetic polypeptide
SGSETPGTSESATPES

SEQ ID NO: 36 - protein, 4 aa, Artificial Sequence
  Synthetic polypeptide
AGVF

SEQ ID NO: 37 - protein, 5 aa, Artificial Sequence
  Synthetic polypeptide
ALALA

SEQ ID NO: 38 - protein, 15 aa, Artificial Sequence
  Synthetic polypeptide
VPFLLEPDNINGKTC

SEQ ID NO: 39 - protein, 12 aa, Artificial Sequence
  Synthetic polypeptide
GSAGSAAGSGEF

SEQ ID NO: 40 - protein, 12 aa, Artificial Sequence
  Synthetic polypeptide
SIVAQLSRPDPA

SEQ ID NO: 41 - protein, 10 aa, Artificial Sequence
  Synthetic polypeptide
MKIIEQLPSA

SEQ ID NO: 42 - protein, 10 aa, Artificial Sequence
  Synthetic polypeptide
VRHKLKRVGS

SEQ ID NO: 43 - protein, 12 aa, Artificial Sequence
  Synthetic polypeptide
GHGTGSTGSGSS

SEQ ID NO: 44 - protein, 7 aa, Artificial Sequence
  Synthetic polypeptide
MSRPDPA

SEQ ID NO: 45 - protein, 12 aa, Artificial Sequence
  Synthetic polypeptide
GSAGSAAGSGEF

SEQ ID NO: 46 - protein, 12 aa, Artificial Sequence
  Synthetic polypeptide
SGSETPGTSESA

SEQ ID NO: 47 - protein, 21 aa, Artificial Sequence
  Synthetic polypeptide
SGSETPGTSESATPEGGSGGS

SEQ ID NO: 48 - protein, 4 aa, Artificial Sequence
  Synthetic polypeptide
GGSM

SEQ ID NO: 49 - protein, 198 aa, Homo sapiens [sequence omitted]

SEQ ID NO: 50 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 51 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 52 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 53 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 54 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 55 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 56 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 57 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 58 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 59 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 60 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 61 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 62 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 63 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 64 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 65 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 66 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 67 - protein, 229 aa, Rattus norvegicus [sequence omitted]

SEQ ID NO: 68 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 69 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 70 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 71 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 72 - DNA, 74 bp, Artificial Sequence
  Synthetic polynucleotide
aattgtgagcggataacaattgacattgtgagcggataacaagatactgagcacatcagcaggacgcactgacc

SEQ ID NO: 73 - DNA, 55 bp, Artificial Sequence
  Synthetic polynucleotide
tccctatcagtgatagagaaaagaattcaaaagatctaaagaggagaaaggatct

SEQ ID NO: 74 - DNA, 112 bp, E. coli
acattgattatttgcacggcgtcacactttgctatgccatagcatttttatccataagattagcggatcctacctgacgctttttatcgcaactctctactgtttctccata

SEQ ID NO: 75 - DNA, 190 bp, Artificial Sequence
  Synthetic polynucleotide
gacaggagaagaattgagacaggagaagaattgagacaggagaagaattgagacaggagaagaattgagacaggagaagaattgagattggtggggggctataaaagggggtgggggcgttcgtcctcactctagatctgcgatctaagtaagcttggcattccggtactgttggtaaagccaccatggc

SEQ ID NO: 76 - DNA, 122 bp, Artificial Sequence
  Synthetic polynucleotide
gacaggagaagaattgagattggtggggggctataaaagggggtgggggcgttcgtcctcactctagatctgcgatctaagtaagcttggcattccggtactgttggtaaagccaccatggc

SEQ ID NO: 77 - DNA, 318 bp, Artificial Sequence
  Synthetic polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 78 - DNA, 5133 bp, Artificial Sequence
  Synthetic polynucleotide
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaa

SEQ ID NO: 79 - DNA, 5649 bp, Artificial Sequence
  Synthetic polynucleotide
atggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgatggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtaccggcggtggaggctcagcagaatacgtacgagctctgtttgacttcaatgggaatgacgaggaggatctcccctttaagaagggcgatattctccgcatcagagataagcccgaagaacaatggtggaatgccgaggatagcgaagggaaaaggggcatgattctggtgccatatgtggagaaatattccggtgactacaaagaccatgatggggattacaaagaccacgacatcgactacaaagacgacgacgataaatcagggatgacagacgccgagtacgtgcgcattcatgagaaactggatatttacaccttcaagaagcagttcttcaacaacaagaaatctgtgtcacaccgctgctacgtgctgtttgagttgaagcgaaggggcgaaagaagggcttgcttttggggctatgccgtcaacaagccccaaagtggcaccgagagaggaatacacgctgagatattcagtatccgaaaggtggaagagtatcttcgggataatcctgggcagtttacgatcaactggtattccagctggagtccttgcgctgattgtgccgagaaaattctggaatggtataatcaggaacttcggggaaacgggcacacattgaaaatctgggcctgcaagctgtactacgagaagaatgcccggaaccagataggactctggaatctgagggacaatggtgtaggcctgaacgtgatggtttccgagcactatcagtgttgtcggaagattttcatccaaagctctcataaccagctcaatgaaaaccgctggttggagaaaacactgaaacgtgcggagaagtggagatccgagctgagcatcatgatccaggtcaagattctgcataccactaagtctccagccgttggtcccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctatctggtggttctcccaagaagaagaggaaagtcgaggatccaaagaagaaaaggaaggttgaagaccccaagaaaaagaggaaggtggatgggatcggctcaggcagcaacggcggtggaggttcagacgctttggacgatttcgatctcgatatgctcggttctgacgccctggatgatttcgatctggatatgctcggcagcgacgctctcgacgatttcgacctcgacatgctcgggtcagatgccttggatgattttgacctggatatgctc

SEQ ID NO: 80 - DNA, 57 bp, Artificial Sequence
  Synthetic polynucleotide
  misc_feature (34)..(38): n is a, c, g, or t
acactctttccctacacgacgctcttccgatctnnnnntgctgcccgacaaccacta

SEQ ID NO: 81 - DNA, 60 bp, Artificial Sequence
  Synthetic polynucleotide
  misc_feature (32)..(36): n is a, c, g, or t
cggcattcctgctgaaccgctcttccgatctnnnnntgaacaaccaccacttcaagtggg

SEQ ID NO: 82 - DNA, 60 bp, Artificial Sequence
  Synthetic polynucleotide
  misc_feature (33)..(37): n is a, c, g, or t
cactctttccctacacgacgctcttccgatctnnnnnggacagcagagatccagtttggt

SEQ ID NO: 83 - DNA, 60 bp, Artificial Sequence
  Synthetic polynucleotide
  misc_feature (31)..(35): n is a, c, g, or t
ggcattcctgctgaaccgctcttccgatctnnnnntcgcagatctagagtgaggacgaac

SEQ ID NO: 84 - DNA, 62 bp, Artificial Sequence
  Synthetic polynucleotide
  misc_feature (34)..(38): n is a, c, g, or t
acactctttccctacacgacgctcttccgatctnnnnnttttatcgcaactctctactgttt

SEQ ID NO: 85 - DNA, 60 bp, Artificial Sequence
  Synthetic polynucleotide
  misc_feature (31)..(35): n is a, c, g, or t
ggcattcctgctgaaccgctcttccgatctnnnnnttcaagttgataacggactagcctt

SEQ ID NO: 86 - DNA, 80 bp, Artificial Sequence; identical to SEQ ID NO: 17
  Synthetic polynucleotide

SEQ ID NO: 87 - DNA, 80 bp, Artificial Sequence
  Synthetic polynucleotide
gttttagagctagaaatagcaagttaaaataaggctagtccgttatcaacttgaaaaagtggcaccgagtcggtgctttt

SEQ ID NO: 88 - DNA, 80 bp, Artificial Sequence; identical to SEQ ID NO: 17
  Synthetic polynucleotide

SEQ ID NO: 89 - DNA, 80 bp, Artificial Sequence; identical to SEQ ID NO: 87
  Synthetic polynucleotide

SEQ ID NO: 90 - DNA, 24 bp, Artificial Sequence
  Synthetic polynucleotide
cccccccccctatgtacatacagt

SEQ ID NO: 91 - DNA, 19 bp, Artificial Sequence
  Synthetic polynucleotide
taagtcggagtactgtcct

SEQ ID NO: 92 - DNA, 20 bp, Artificial Sequence
  Synthetic polynucleotide
taagtcggagtactgttcct

SEQ ID NO: 93 - DNA, 23 bp, Artificial Sequence
  Synthetic polynucleotide
taagtcggagtactgttagacct

SEQ ID NO: 94 - DNA, 23 bp, Artificial Sequence
  Synthetic polynucleotide
taagtcggagtactgttagagct

SEQ ID NO: 95 - DNA, 26 bp, Artificial Sequence
  Synthetic polynucleotide
taagtcggagtactgttagagctcct

SEQ ID NO: 96 - DNA, 31 bp, Artificial Sequence
  Synthetic polynucleotide
taagtcggagtactgttagagctagagtact

SEQ ID NO: 97 - DNA, 33 bp, Artificial Sequence
  Synthetic polynucleotide
taagtcggagtactgttagagctagaaatagct

SEQ ID NO: 98 - DNA, 34 bp, Artificial Sequence
  Synthetic polynucleotide
taagtcggagtactgttagagctagaaatagcct

SEQ ID NO: 99 - DNA, 50 bp, Artificial Sequence
  Synthetic polynucleotide
taagtcggagtactgttagagctagaaatagctagctattagagctagct

SEQ ID NO: 100 - DNA, 54 bp, Artificial Sequence
  Synthetic polynucleotide
taagtcggagtactgttagagctagaaatagcagaaatagctagaaatagtact

SEQ ID NO: 101 - DNA, 55 bp, Artificial Sequence
  Synthetic polynucleotide
taagtcggagtactgttagagctagaaatagcaagttaacctaacttgctaacct

SEQ ID NO: 102 - DNA, 68 bp, Artificial Sequence
  Synthetic polynucleotide
taagtcggagtactgttagagctagaaatagctattagctaatagctattagttagcaagtactaact

SEQ ID NO: 103 - DNA, 24 bp, Artificial Sequence
  Synthetic polynucleotide
cacccccccctatgtacatacagt

SEQ ID NO: 104 - DNA, 24 bp, Artificial Sequence
  Synthetic polynucleotide
cacccacccctatgtacatacagt

SEQ ID NO: 105 - DNA, 24 bp, Artificial Sequence
  Synthetic polynucleotide
cacccatccctatgtacatacagt

SEQ ID NO: 106 - DNA, 29 bp, Artificial Sequence
  Synthetic polynucleotide
gggcccccccccctatgtacatacagtgg

SEQ ID NO: 107 - DNA, 29 bp, Artificial Sequence
  Synthetic polynucleotide
  misc_feature (27)..(27): n is a, c, g, or t
gggcccccccccctatgtacatacagngg

SEQ ID NO: 108 - DNA, 29 bp, Artificial Sequence
  Synthetic polynucleotide
gggcccccccccctatgtacatacagcgg

SEQ ID NO: 109 - DNA, 27 bp, Artificial Sequence
  Synthetic polynucleotide
gggcccccccctatgtacatacagtgg

SEQ ID NO: 110 - DNA, 29 bp, Artificial Sequence
  Synthetic polynucleotide
gggtccccccccctatgtacatacagtgg

SEQ ID NO: 111 - DNA, 29 bp, Artificial Sequence
  Synthetic polynucleotide
gggtcccccccccctatgtacatacagtg

SEQ ID NO: 112 - DNA, 29 bp, Artificial Sequence
  Synthetic polynucleotide
gggccccccccctatgtacatacagtggg

SEQ ID NO: 113 - DNA, 29 bp, Artificial Sequence
  Synthetic polynucleotide
gggccccccccccttatgtacatacagtg

SEQ ID NO: 114 - DNA, 29 bp, Artificial Sequence
  Synthetic polynucleotide
gggcccccccccccctatgtacatacagt

SEQ ID NO: 115 - DNA, 29 bp, Artificial Sequence
  Synthetic polynucleotide
ggcccccccccccctatgtacatacagtg

SEQ ID NO: 116 - DNA, 29 bp, Artificial Sequence
  Synthetic polynucleotide
gggcccccccccccatgtacatacagtgg

SEQ ID NO: 117 - DNA, 29 bp, Artificial Sequence
  Synthetic polynucleotide
ggcccccccccctatgtacatacagtggg

SEQ ID NO: 118 - DNA, 29 bp, Artificial Sequence
  Synthetic polynucleotide
ggaccccccccccctatgtacatacagtg

SEQ ID NO: 119 - DNA, 29 bp, Artificial Sequence
  Synthetic polynucleotide
ggacccccccccctatgtacatacagtgg

SEQ ID NO: 120 - DNA, 29 bp, Artificial Sequence
  Synthetic polynucleotide
gggccccccccccccctatgtacatacag

SEQ ID NO: 121 - DNA, 29 bp, Artificial Sequence
  Synthetic polynucleotide
gggccccccccccatgtacatacagtggg

SEQ ID NO: 122 - DNA, 29 bp, Artificial Sequence
  Synthetic polynucleotide
  misc_feature (28)..(28): n is a, c, g, or t
gggccccccccccctatgtacatacagng

SEQ ID NO: 123 - DNA, 29 bp, Artificial Sequence
  Synthetic polynucleotide
gggccccccccccctatgtacatacaggg

SEQ ID NO: 124 - DNA, 29 bp, Artificial Sequence
  Synthetic polynucleotide
gggccccccccccctatgtacatacagtg

SEQ ID NO: 125 - DNA, 29 bp, Artificial Sequence
  Synthetic polynucleotide
ggccccccccccctatgtacatacagtgg

SEQ ID NO: 126 - DNA, 29 bp, Artificial Sequence
  Synthetic polynucleotide
gggccccccccccctatgtacatacagtt

SEQ ID NO: 127 - DNA, 29 bp, Artificial Sequence
  Synthetic polynucleotide
gggccccccctccctatgtacatacagtg

SEQ ID NO: 128 - DNA, 29 bp, Artificial Sequence
  Synthetic polynucleotide
gggcccccccttcctatgtacatacagtg

SEQ ID NO: 129 - DNA, 29 bp, Artificial Sequence
  Synthetic polynucleotide
gggcccccccttctatgtacatacagtgg

SEQ ID NO: 130 - DNA, 29 bp, Artificial Sequence
  Synthetic polynucleotide
gggccccccctcctatgtacatacagtgg

SEQ ID NO: 131 - DNA, 29 bp, Artificial Sequence
  Synthetic polynucleotide
gggccccccttcctatgtacatacagtgg

SEQ ID NO: 132 - DNA, 29 bp, Artificial Sequence
  Synthetic polynucleotide
gggccccccccctcctatgtacatacagt

SEQ ID NO: 133 - DNA, 29 bp, Artificial Sequence
  Synthetic polynucleotide
gggccccccccttctatgtacatacagtg

SEQ ID NO: 134 - DNA, 29 bp, Artificial Sequence
  Synthetic polynucleotide
ggggccccccccctatgtacatacagtgg

SEQ ID NO: 135 - DNA, 29 bp, Artificial Sequence
  Synthetic polynucleotide
gggccccccccctctatgtacatacagtg

SEQ ID NO: 136 - DNA, 29 bp, Artificial Sequence
  Synthetic polynucleotide
gggcccccccccttatgtacatacagtgg

SEQ ID NO: 137 - DNA, 29 bp, Artificial Sequence
  Synthetic polynucleotide
gggccccccccctcttatgtacatacagt

SEQ ID NO: 138 - DNA, 29 bp, Artificial Sequence
  Synthetic polynucleotide
gggccccccccctatgtacatacagtggg

SEQ ID NO: 139 - DNA, 29 bp, Artificial Sequence
  Synthetic polynucleotide
gggcccccctcccctatgtacatacagtg

SEQ ID NO: 140 - DNA, 29 bp, Artificial Sequence
  Synthetic polynucleotide
gggcccccctccctatgtacatacagtgg

SEQ ID NO: 141 - DNA, 29 bp, Artificial Sequence
  Synthetic polynucleotide
gggccccccgcccctatgtacatacagtg

SEQ ID NO: 142 - DNA, 29 bp, Artificial Sequence
  Synthetic polynucleotide
gggccccccgccctatgtacatacagtgg

SEQ ID NO: 143 - DNA, 29 bp, Artificial Sequence
  Synthetic polynucleotide
gggccccccaccctatgtacatacagtgg

SEQ ID NO: 144 - DNA, 29 bp, Artificial Sequence
  Synthetic polynucleotide
gggccccccacccctatgtacatacagtg

SEQ ID NO: 145 - DNA, 29 bp, Artificial Sequence
  Synthetic polynucleotide
gggcccccccctcctatgtacatacagtg

SEQ ID NO: 146 - DNA, 29 bp, Artificial Sequence
  Synthetic polynucleotide
gggcccccccctccctatgtacatacagt

SEQ ID NO: 147 - DNA, 29 bp, Artificial Sequence
  Synthetic polynucleotide
gggccccccccgcctatgtacatacagtg

SEQ ID NO: 148 - DNA, 29 bp, Artificial Sequence
  Synthetic polynucleotide
  misc_feature (27)..(27): n is a, c, g, or t
gggcccccccccctatgtacatacagngg

SEQ ID NO: 149 - DNA, 29 bp, Artificial Sequence
  Synthetic polynucleotide
gggcccccccccctatgtacatacagtgg

SEQ ID NO: 150 - DNA, 29 bp, Artificial Sequence
  Synthetic polynucleotide
gggccacccccccctatgtacatacagtg

SEQ ID NO: 151 - DNA, 29 bp, Artificial Sequence
  Synthetic polynucleotide
gggccccccccccctatgtacatacagtg

SEQ ID NO: 152 - DNA, 29 bp, Artificial Sequence
  Synthetic polynucleotide
gggcccccccgcctatgtacatacagtgg

SEQ ID NO: 153 - DNA, 29 bp, Artificial Sequence
  Synthetic polynucleotide
gggcccccccccgcctatgtacatacagt

SEQ ID NO: 154 - DNA, 29 bp, Artificial Sequence
  Synthetic polynucleotide
gggcccccccaccctatgtacatacagtg

SEQ ID NO: 155 - DNA, 29 bp, Artificial Sequence
  Synthetic polynucleotide
ggggcccccccccctatgtacatacagtg

SEQ ID NO: 156 - DNA, 29 bp, Artificial Sequence
  Synthetic polynucleotide
gggccccccccccctatgtacatacagtg

SEQ ID NO: 157 - DNA, 29 bp, Artificial Sequence
  Synthetic polynucleotide
ggccccccccccctatgtacatacagtgg

SEQ ID NO: 158 - DNA, 29 bp, Artificial Sequence
  Synthetic polynucleotide
gggccccccccccctatgtacatacagtt

SEQ ID NO: 159 - DNA, 29 bp, Artificial Sequence
  Synthetic polynucleotide
gggccccccccccctatgtgcatacagtg

SEQ ID NO: 160 - DNA, 29 bp, Artificial Sequence
  Synthetic polynucleotide
  misc_feature (28)..(28): n is a, c, g, or t
gggccccccccccctatgtacatacagng

SEQ ID NO: 161 - DNA, 29 bp, Artificial Sequence
  Synthetic polynucleotide
gggccccccccccctatgtacatacaggg

SEQ ID NO: 162 - DNA, 29 bp, Artificial Sequence
  Synthetic polynucleotide
gggccccccccccccctatgtacatacag

SEQ ID NO: 163 - DNA, 28 bp, Artificial Sequence
  Synthetic polynucleotide
gggcccccccctatgtacatacagtggg

SEQ ID NO: 164 - DNA, 29 bp, Artificial Sequence
  Synthetic polynucleotide
ggacccccccccctatgtacatacagtgg

SEQ ID NO: 165 - DNA, 29 bp, Artificial Sequence
  Synthetic polynucleotide
gggtcccccccccctatgtacatacagtg

SEQ ID NO: 166 - DNA, 29 bp, Artificial Sequence
  Synthetic polynucleotide
gggtccccccccctatgtacatacagtgg

SEQ ID NO: 167 - DNA, 29 bp, Artificial Sequence
  Synthetic polynucleotide
gggccccctccccctatgtacatacagtg

SEQ ID NO: 168 - DNA, 29 bp, Artificial Sequence
  Synthetic polynucleotide
gggccccctcccctatgtacatacagtgg

SEQ ID NO: 169 - DNA, 29 bp, Artificial Sequence
  Synthetic polynucleotide
gggcccccgccccctatgtacatacagtg

SEQ ID NO: 170 - DNA, 29 bp, Artificial Sequence
  Synthetic polynucleotide
gggcccccaccccctatgtacatacagtg

SEQ ID NO: 171 - DNA, 29 bp, Artificial Sequence
  Synthetic polynucleotide
gggcccccccccccctatgtacatacagt

SEQ ID NO: 172 - DNA, 29 bp, Artificial Sequence
  Synthetic polynucleotide
ggcccccccccccctatgtacatacagtg

SEQ ID NO: 173 - DNA, 29 bp, Artificial Sequence
  Synthetic polynucleotide
ggcccccccccctatgtacatacagtggg

SEQ ID NO: 174 - DNA, 29 bp, Artificial Sequence
  Synthetic polynucleotide
gggccccccccccttatgtacatacagtg

SEQ ID NO: 175 - DNA, 29 bp, Artificial Sequence
  Synthetic polynucleotide
gggcccccccccctctatgtacatacagt

SEQ ID NO: 176 - DNA, 21 bp, Artificial Sequence
  Synthetic polynucleotide
ccccccccccccccccccccc

SEQ ID NO: 177 - protein, 7 aa, Artificial Sequence
  Synthetic polypeptide
PPPPPPP

SEQ ID NO: 178 - DNA, 21 bp, Artificial Sequence
  Synthetic polynucleotide
ttttttttccccccccccccc

SEQ ID NO: 179 - protein, 7 aa, Artificial Sequence
  Synthetic polypeptide
FFFPPPP

SEQ ID NO: 180 - DNA, 21 bp, Artificial Sequence
  Synthetic polynucleotide
ttttttttttccccccccccc

SEQ ID NO: 181 - protein, 7 aa, Artificial Sequence
  Synthetic polypeptide
FFFSPPP

SEQ ID NO: 182 - DNA, 21 bp, Artificial Sequence
  Synthetic polynucleotide
tttttttttttttcccccccc

SEQ ID NO: 183 - protein, 7 aa, Artificial Sequence
  Synthetic polypeptide
FFFFSPP

SEQ ID NO: 184 - DNA, 21 bp, Artificial Sequence
  Synthetic polynucleotide
ttttttttttttttccccccc

SEQ ID NO: 185 - protein, 7 aa, Artificial Sequence
  Synthetic polypeptide
FFFFFPP

SEQ ID NO: 186 - DNA, 21 bp, Artificial Sequence
  Synthetic polynucleotide
tttttttttttttttcccccc

SEQ ID NO: 187 - protein, 7 aa, Artificial Sequence
  Synthetic polypeptide
FFFFFPP

SEQ ID NO: 188 - DNA, 21 bp, Artificial Sequence
  Synthetic polynucleotide
ttttttttttttttttttttt

SEQ ID NO: 189 - protein, 7 aa, Artificial Sequence
  Synthetic polypeptide
FFFFFFF

SEQ ID NO: 190 - DNA, 21 bp, Artificial Sequence
  Synthetic polynucleotide
cccctttttcccccccccccc

SEQ ID NO: 191 - protein, 7 aa, Artificial Sequence
  Synthetic polypeptide
PLFPPPP

SEQ ID NO: 192 - DNA, 21 bp, Artificial Sequence
  Synthetic polynucleotide
cccttttttcccccccccccc

SEQ ID NO: 193 - protein, 7 aa, Artificial Sequence
  Synthetic polypeptide
PFFPPPP

SEQ ID NO: 194 - DNA, 21 bp, Artificial Sequence
  Synthetic polynucleotide
ttttttattgccccccccccc

SEQ ID NO: 195 - protein, 7 aa, Artificial Sequence
  Synthetic polypeptide
FFLAPPP

SEQ ID NO: 196 - DNA, 21 bp, Artificial Sequence
  Synthetic polynucleotide
tgttgagttcccccccaccca

SEQ ID NO: 197 - protein, 6 aa, Artificial Sequence
  Synthetic polypeptide
CVPPHP

SEQ ID NO: 198 - DNA, 21 bp, Artificial Sequence
  Synthetic polynucleotide
cacccctttttccccacccac

SEQ ID NO: 199 - protein, 7 aa, Artificial Sequence
  Synthetic polypeptide
HPFFPTH

SEQ ID NO: 200 - DNA, 21 bp, Artificial Sequence
  Synthetic polynucleotide
cccccttttttcccccccccc

SEQ ID NO: 201 - protein, 7 aa, Artificial Sequence
  Synthetic polypeptide
PPFFPPP

SEQ ID NO: 202 - DNA, 21 bp, Artificial Sequence
  Synthetic polynucleotide
gtttttttttttccccccccc

SEQ ID NO: 203 - protein, 7 aa, Artificial Sequence
  Synthetic polypeptide
VFFFPPP

SEQ ID NO: 204 - DNA, 21 bp, Artificial Sequence
  Synthetic polynucleotide
tttttttttaccccccccccc

SEQ ID NO: 205 - protein, 7 aa, Artificial Sequence
  Synthetic polypeptide
FFFTPPP

SEQ ID NO: 206 - DNA, 21 bp, Artificial Sequence
  Synthetic polynucleotide
ttcccacagacacccacctgc

SEQ ID NO: 207 - protein, 7 aa, Artificial Sequence
  Synthetic polypeptide
FPQTPTC

SEQ ID NO: 208 - DNA, 21 bp, Artificial Sequence
  Synthetic polynucleotide
tcccaagaccatcacctctca

SEQ ID NO: 209 - protein, 7 aa, Artificial Sequence
  Synthetic polypeptide
SQDHHLS

SEQ ID NO: 210 - DNA, 21 bp, Artificial Sequence
  Synthetic polynucleotide
ttttttttttattccgaggag

SEQ ID NO: 211 - protein, 7 aa, Artificial Sequence
  Synthetic polypeptide
FFFYSKK

SEQ ID NO: 212 - DNA, 23 bp, Artificial Sequence
  Synthetic polynucleotide
ccccccccccccccccccccccc

SEQ ID NO: 213 - DNA, 22 bp, Artificial Sequence
  Synthetic polynucleotide
ttttttttttcccccccccccc

SEQ ID NO: 214 - DNA, 31 bp, Artificial Sequence
  Synthetic polynucleotide
ggtgaaccccccctatgtacatacagttgga

SEQ ID NO: 215 - DNA, 31 bp, Artificial Sequence; identical to SEQ ID NO: 214
  Synthetic polynucleotide

SEQ ID NO: 216 - DNA, 31 bp, Artificial Sequence
  Synthetic polynucleotide
ggtgaaccctttttatgtacatacagttgga

SEQ ID NO: 217 - DNA, 23 bp, Artificial Sequence
  Synthetic polynucleotide
tcaacatataggcctgatttaga

SEQ ID NO: 218 - DNA, 23 bp, Artificial Sequence
  Synthetic polynucleotide
tcaacatataaatttgatttaga

SEQ ID NO: 219 - DNA, 23 bp, Artificial Sequence
  Synthetic polynucleotide
tcaacatataggcctggtttaga

SEQ ID NO: 220 - DNA, 23 bp, Artificial Sequence
  Synthetic polynucleotide
tcaacatataggtttggtttaga

SEQ ID NO: 221 - DNA, 23 bp, Artificial Sequence
  Synthetic polynucleotide
tcaatttataggtttggtttaga

SEQ ID NO: 222 - DNA, 14 bp, Artificial Sequence
  Synthetic polynucleotide
tgcgggtgggttta

SEQ ID NO: 223 - DNA, 23 bp, Artificial Sequence
  Synthetic polynucleotide
tcaacatataggcctgatttaga

SEQ ID NO: 224 - DNA, 23 bp, Artificial Sequence
  Synthetic polynucleotide
tcaacatataaatttgatttaga

SEQ ID NO: 225 - DNA, 23 bp, Artificial Sequence
  Synthetic polynucleotide
tcaacatataggcctgatttaga

SEQ ID NO: 226 - DNA, 23 bp, Artificial Sequence
  Synthetic polynucleotide
tcaacatataggtttgatttaga

SEQ ID NO: 227 - DNA, 23 bp, Artificial Sequence
  Synthetic polynucleotide
tcaacatataggtttgatttaga

SEQ ID NO: 228 - DNA, 23 bp, Artificial Sequence
  Synthetic polynucleotide
tcaacatataggcctgatttaga

SEQ ID NO: 229 - DNA, 23 bp, Artificial Sequence
  Synthetic polynucleotide
tcaacatataggtttgatttaga

SEQ ID NO: 230 - DNA, 27 bp, Artificial Sequence
  Synthetic polynucleotide
tcaacatataggcctgatttagagggg

SEQ ID NO: 231 - DNA, 25 bp, Artificial Sequence
  Synthetic polynucleotide
tcaacatataggcctgatttagaaa

SEQ ID NO: 232 - DNA, 25 bp, Artificial Sequence
  Synthetic polynucleotide
tcaacatataggtttgatttagaaa

SEQ ID NO: 233 - DNA, 23 bp, Artificial Sequence
  Synthetic polynucleotide
tcaacatataggcctgatttaga

SEQ ID NO: 234 - DNA, 23 bp, Artificial Sequence
  Synthetic polynucleotide
tcaacatataggtttgatttaga

SEQ ID NO: 235 - DNA, 23 bp, Artificial Sequence
  Synthetic polynucleotide
tcaacatataggtttgatttaga

SEQ ID NO: 236 - DNA, 48 bp, Artificial Sequence
  Synthetic polynucleotide
aaatcatctcaccagggtcatctcaccagggtcatctcaccagggtca

SEQ ID NO: 237 - DNA, 48 bp, Artificial Sequence
  Synthetic polynucleotide
aaatcatctcaccaaaatcatctcaccaaaatcatctcaccagggtca

SEQ ID NO: 238 - DNA, 48 bp, Artificial Sequence
  Synthetic polynucleotide
aaatcatctcaccaaaatcatctcaccaaaatcatctcaccaaaatca

SEQ ID NO: 239 - DNA, 67 bp, Artificial Sequence
  Synthetic polynucleotide
tggtgagatgagactggtgagatgagactggtgagatgagactggtgagatgagattggacgcgtaa

SEQ ID NO: 240 - DNA, 20 bp, Artificial Sequence
  Synthetic polynucleotide
tgagactggtgagatgagat

SEQ ID NO: 241 - DNA, 22 bp, Artificial Sequence
  Synthetic polynucleotide
tgaccatagccggtagaccagg

SEQ ID NO: 242 - DNA, 22 bp, Artificial Sequence
  Synthetic polynucleotide
tgaccatagccggtagattagg

SEQ ID NO: 243 - DNA, 22 bp, Artificial Sequence
  Synthetic polynucleotide
tgaccatagttggtagattagg

SEQ ID NO: 244 - DNA, 22 bp, Artificial Sequence
  Synthetic polynucleotide
tgattatagttggtagattagg

SEQ ID NO: 245 - DNA, 22 bp, Artificial Sequence
  Synthetic polynucleotide
tcaccatcgccggtagataagg

SEQ ID NO: 246 - DNA, 22 bp, Artificial Sequence
  Synthetic polynucleotide
tcatcatcgtcggtagataagg

SEQ ID NO: 247 - DNA, 22 bp, Artificial Sequence
  Synthetic polynucleotide
ttatcattgttggtagataagg

SEQ ID NO: 248 - DNA, 22 bp, Artificial Sequence
  Synthetic polynucleotide
ttattattgttggtagataagg

SEQ ID NO: 249 - DNA, 23 bp, Artificial Sequence
  Synthetic polynucleotide
ttaggccatttaatcgccattgg

SEQ ID NO: 250 - DNA, 23 bp, Artificial Sequence
  Synthetic polynucleotide
ttaggttatttaatcgccattgg

SEQ ID NO: 251 - DNA, 38 bp, Artificial Sequence
  Synthetic polynucleotide
cccaatggcgattaaatggcctaaatagagcctatggg

SEQ ID NO: 252 - DNA, 38 bp, Artificial Sequence
  Synthetic polynucleotide
cccaatggcgattaaatggtttaaatagagcctatggg

SEQ ID NO: 253 - DNA, 38 bp, Artificial Sequence
  Synthetic polynucleotide
cccaatggcgattaaataacctaaatagagcctatggg

SEQ ID NO: 254 - DNA, 38 bp, Artificial Sequence
  Synthetic polynucleotide
cccaatggcgattaaataatttaaatagagcctatggg

SEQ ID NO: 255 - DNA, 39 bp, Artificial Sequence
  Synthetic polynucleotide
ccccagtagtgcaaatgggcccaagttttagagctagga

SEQ ID NO: 256 - DNA, 39 bp, Artificial Sequence
  Synthetic polynucleotide
ccccagtagtgcaaataaatttaagttttagagctagga

SEQ ID NO: 257 - DNA, 38 bp, Artificial Sequence
  Synthetic polynucleotide
aggtaggacgggtggaggacccaggggtaaaggagagg

SEQ ID NO: 258 - DNA, 38 bp, Artificial Sequence
  Synthetic polynucleotide
aggtaggacgggtggaggatttaggggtaaaggagagg

SEQ ID NO: 259 - DNA, 38 bp, Artificial Sequence
  Synthetic polynucleotide
aggtaggatgggtggaggatttaggggtaaaggagagg

SEQ ID NO: 260 - DNA, 34 bp, Artificial Sequence
  Synthetic polynucleotide
gataggacccaatggcgattaaatggcctaaata

SEQ ID NO: 261 - DNA, 34 bp, Artificial Sequence
  Synthetic polynucleotide
gataggatttaatggcgattaaatggcctaaata

SEQ ID NO: 262 - DNA, 34 bp, Artificial Sequence
  Synthetic polynucleotide
gataggatttaatggcgattaaataacctaaata

SEQ ID NO: 263 - DNA, 34 bp, Artificial Sequence
  Synthetic polynucleotide
gataggacccaatggcgattaaataacctaaata

SEQ ID NO: 264 - DNA, 34 bp, Artificial Sequence; identical to SEQ ID NO: 262
  Synthetic polynucleotide

SEQ ID NO: 265 - DNA, 34 bp, Artificial Sequence
  Synthetic polynucleotide
cctggataggacccaatggcgattaaatggccta

SEQ ID NO: 266 - DNA, 34 bp, Artificial Sequence
  Synthetic polynucleotide
cctggataggacccaataacaattaaatggccta

SEQ ID NO: 267 - DNA, 34 bp, Artificial Sequence
  Synthetic polynucleotide
cctggataggacccaataacaattaaataaccta

SEQ ID NO: 268 - DNA, 34 bp, Artificial Sequence
  Synthetic polynucleotide
cctggataggacccaatggcgattaaataaccta

SEQ ID NO: 269 - DNA, 34 bp, Artificial Sequence; identical to SEQ ID NO: 267
  Synthetic polynucleotide

SEQ ID NO: 270 - DNA, 60 bp, Artificial Sequence
  Synthetic polynucleotide
tgaaccggacggagagccagggaggagagccagggaggagagccagggaggagagttggg

SEQ ID NO: 271 - DNA, 60 bp, Artificial Sequence
  Synthetic polynucleotide
tgaaccggacggagagccagggaggagagccagggaggagagttagggaggagagttggg

SEQ ID NO: 272 - DNA, 60 bp, Artificial Sequence
  Synthetic polynucleotide
tgaaccggacggagagccagggaggagagttagggaggagagttagggaggagagttggg

SEQ ID NO: 273 - DNA, 60 bp, Artificial Sequence
  Synthetic polynucleotide
tgaaccggacggagagttagggaggagagttagggaggagagttagggaggagagttggg

SEQ ID NO: 274 - DNA, 60 bp, Artificial Sequence
  Synthetic polynucleotide
tgaattggacggagagttagggaggagagttagggaggagagttagggaggagagttggg

SEQ ID NO: 275 - DNA, 43 bp, Artificial Sequence
  Synthetic polynucleotide
cccccacacccccgacccccacccacccccccgcccccaaccc

SEQ ID NO: 276 - DNA, 38 bp, Artificial Sequence; identical to SEQ ID NO: 251
  Synthetic polynucleotide

SEQ ID NO: 277 - DNA, 38 bp, Artificial Sequence; identical to SEQ ID NO: 251
  Synthetic polynucleotide

SEQ ID NO: 278 - DNA, 38 bp, Artificial Sequence; identical to SEQ ID NO: 254
  Synthetic polynucleotide

SEQ ID NO: 279 - DNA, 25 bp, Artificial Sequence
  Synthetic polynucleotide
cccaatggcgattaaatggcctaaa

SEQ ID NO: 280 - DNA, 25 bp, Artificial Sequence
  Synthetic polynucleotide
cccaatggcgattaaataacctaaa

SEQ ID NO: 281 - DNA, 55 bp, Artificial Sequence
  Synthetic polynucleotide
cccgatcaaattacctgggccgttttagagctaggatcaaattacctaaacctgg

SEQ ID NO: 282 - DNA, 55 bp, Artificial Sequence
  Synthetic polynucleotide
cccgatcaaattacctaaaccgttttagagctaggatcaaattacctaaacctgg

SEQ ID NO: 283 - DNA, 55 bp, Artificial Sequence; identical to SEQ ID NO: 282
  Synthetic polynucleotide

SEQ ID NO: 284 - DNA, 69 bp, Artificial Sequence
  Synthetic polynucleotide
tatgcagcaacgagacgtcacggcagacggcaaacgactgtcctggataggtcacgttggtgtagatgg

SEQ ID NO: 285 - DNA, 64 bp, Artificial Sequence
  Synthetic polynucleotide
tatgtagcaacgagacgtcacggcagatggtaaacgactgtcctggataggttggtgtagatgg

SEQ ID NO: 286 - DNA, 23 bp, Artificial Sequence
  Synthetic polynucleotide
ccgaggagaggtaccatgtctaa

SEQ ID NO: 287 - DNA, 23 bp, Artificial Sequence
  Synthetic polynucleotide
ccgaggagaggtaccatatctaa

SEQ ID NO: 288 - DNA, 23 bp, Artificial Sequence
  Synthetic polynucleotide
gacaggagaagaattgagattgg

SEQ ID NO: 289 - DNA, 23 bp, Artificial Sequence
  Synthetic polynucleotide
gadaggagaagaattgagattgg

SEQ ID NO: 290 - DNA, 59 bp, Artificial Sequence
  Synthetic polynucleotide
gcaccccaggcccccccctttatgcttccggctcgccccccgtgtggaattgtgagcgg

(19) United States
(12) Patent Application Publication
(10) Pub. No.: US 2020/0063127 A1
(43) Pub. Date: Feb. 27, 2020

(54) DNA WRITERS, MOLECULAR RECORDERS AND USES THEREOF

(71) Applicant: Massachusetts Institute of Technology, Cambridge, MA (US)

(72) Inventors: Timothy Kuan-Ta Lu, Cambridge, MA (US); Fahim Farzadfard, Boston, MA (US)

(73) Assignee: Massachusetts Institute of Technology, Cambridge, MA (US)

(21) Appl. No.: 16/485,822

(22) PCT Filed: Feb. 14, 2018
(86) PCT No.: PCT/US2018/018173

§ 371 Date: Aug. 14, 2019

Related U.S. Application Data
(60) Provisional application No. 62/597,376, filed on Dec. 11, 2017.
(60) Provisional application No. 62/520,206, filed on Jun. 15, 2017.
(60) Provisional application No. 62/459,485, filed on Feb. 15, 2017.

(51) Int. Cl. C12N 15/11; C12N 9/22; C12N 15/62; C12N 9/78

(52) U.S. Cl. CPC C12N 15/111; C12N 9/22; C12N 15/62; C12N 2310/20; C12Y 305/04005; C12Y 305/04004; C12N 2800/80; C12N 9/78

(57) ABSTRACT

Provided herein are compositions, systems, and methods for continuous and accumulative modification of a target site.

### RELATED APPLICATIONS

This application is a national stage filing under 35 U.S.C. § 371 of International Patent Application Serial No. PCT/US2018/018173, filed Feb. 14, 2018, which claims priority under 35 U.S.C. § 119(e) to U.S. provisional application No. 62/459,485, filed Feb. 15, 2017, and to U.S. provisional application No. 62/520,206, filed Jun. 15, 2017, and to U.S. provisional application No. 62/597,376, filed Dec. 11, 2017, the contents of each of which is incorporated herein by reference in its entirety.

### FEDERALLY SPONSORED RESEARCH

This invention was made with Government support under Grant No. N00014-13-1-0424 awarded by the Office of Naval Research, Grant No. P50 GM098792 awarded by the National Institutes of Health, and Grant No. CCF-1521925 awarded by the National Science Foundation. The Government has certain rights in the invention.

### REFERENCE TO SEQUENCE LISTING SUBMITTED ELECTRONICALLY

The Sequence Listing named M065670403US03-SEQ-ZJG having a size of 247 kb is incorporated herein by reference in its entirety.

### BACKGROUND

Many molecular events and interactions in biological systems are transient, and thus hard to study in their natural contexts. Some molecules are capable of converting these transient signals into long-lasting records, ideally in a continuous fashion, for later retrieval. By looking at the recorded information, one can deduce information about the original transient signal, such as the dynamics of the signal or the chronology of molecular events.

### SUMMARY

Provided herein, in some aspects are DNA writers that enable manipulation (mutation) of DNA of living cells in a dynamic, targeted, and autonomous fashion, with nucleotide resolution and in response to cues of interest. DNA provides an ideal medium for biological memory because it is replicated at high fidelity within cells, is compatible with living cells, and is present ubiquitously in biological systems. These DNA writers offer unprecedented capacities to record transient biological information and signaling dynamics into long-lasting DNA memory (molecular recorders), perform memory and logic operations (DOMINO (DNA-based Ordered Memory and Iteration Network Operating System) platform), and engineer biomolecules and cellular phenotypes (DRIVE (Directed and Recurring In Vivo Evolution) platform).

DNA-based molecular recorders, for example, convert transient signals into long lasting DNA memory at much higher rates relative to natural mutation rates. These molecular recorder systems can artificially elevate mutation rates within targeted genomic segments and write the targeted mutations (memory states) into DNA. The molecular recorder function, as provided herein, can be operationally linked to events of interest through a “controller” (e.g., a regulatory element, such as promoter, or other transient event, such as neural pulses or protein-protein interaction events) to record the dynamics of the controller activity. Alternatively, the molecular recorders can be used as “hypermutation” devices that continuously diversifies a target sequence, for example, at each cell generation, without necessarily being linked to a specific cellular cue. Thus, the diversified sequence can be used to infer the chronological order of the events and evolutionary (or developmental) history of cells over time (lineage tracing).

Current molecular recording technologies, by contrast, such as “molecular clocks,” rely solely on mutation accumulation and can only be used in instances where mutations accumulate at a significantly high levels. Natural mutation rates, however, are very low, thus current molecular recording technologies are limited to evolutionary timescales and cannot be used to record events occurring during shorter timescales, such as during developmental events (e.g., formation of multicellular organisms from single cells). These existing systems, limited in duration and scale, can have an adverse impact on a living cell.

The molecular recorder systems of the present disclosure can be generalized, scaled, and used to continuously and autonomously write new information into targeted DNA memory registers in a step-wise fashion without inducing adverse impacts to a living cell. The compositions, systems, and methods provided herein enable long-term continuous and accumulative molecular modification of a nucleic acid target site via conservative and step-wise DNA editing schemes that, for example, can be used for lineage tracing applications. These systems are useful for a wide range of areas, including biotechnology, biological research, and biomedicine.

Thus, some aspects of the present disclosure provide a cell comprising (a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM), (b) a RNA-guided endonuclease, and (c) an enzyme that catalyzes the addition of nucleotides to the 3′ end of a nucleic acid.

Other aspects of the present disclosure provide a method comprising maintaining a cell that comprises (a) a RNA-guided endonuclease, (b) an enzyme that catalyzes the addition of nucleotides to the 3′ end of a nucleic acid, and (c) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM), under conditions that result in the addition of random nucleotides to the SDS.

Still other aspects of the present disclosure provide a kit comprising (a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM), (b) an RNA-guided endonuclease or an engineered nucleic acid encoding an RNA-guided endonuclease, and (c) a terminal deoxynucleotidyl transferase (TdT) or an engineered nucleic acid encoding a TdT.

Yet other aspects of the present disclosure provide a cell engineered to include an array of repetitive deoxycytosine nucleotides (dC)-rich (dC-rich) DNA sequences that include deoxycytosine nucleotides integrated into a locus of the genome of the cell and comprising (a) an engineered nucleic acid comprising a promoter operably linked to a nucleotide sequence encoding a guide ribonucleic acid (gRNA) that targets the array of repetitive dC-rich DNA sequences, and (b) a fusion protein comprising a catalytically-inactive Cas9 fused to cytidine deaminase. “Cytosine deaminase” and “cytidine deaminase” may be used interchangeable herein.

Some aspects of the present disclosure provide a method comprising maintaining a cell engineered to include an array of repetitive deoxycytosine nucleotides (dC)-rich DNA sequences that include deoxycytosine nucleotides (dC) integrated into a locus of the genome of the cell and comprising (a) an engineered nucleic acid comprising a promoter operably linked to a nucleotide sequence encoding a guide ribonucleic acid (gRNA) targets the array of repetitive dC-rich DNA sequences, and (b) a fusion protein comprising a catalytically-inactive Cas9 fused to cytidine deaminase, under conditions that result in targeted mutations in the array of repetitive DNA sequences at dC positions.

Further aspects of the present disclosure provide a kit comprising (a) an engineered nucleic acid comprising an array of repetitive deoxycytosine nucleotides (dC)-rich DNA sequences, (b) an engineered nucleic acid comprising a promoter operably linked to a nucleotide sequence encoding a guide ribonucleic acid (gRNA) that targets the array of repetitive dC-rich DNA sequences, and (c) a fusion protein comprising a catalytically-inactive Cas9 fused to cytidine deaminase, or a nucleic acid encoding a fusion protein comprising a catalytically-inactive Cas9 fused to cytidine deaminase.

Other aspects of the present disclosure provide a cell comprising (a) an engineered nucleic acid comprising a promoter operably linked to a nucleotide sequence encoding a self-targeting guide ribonucleic acid (stgRNA) that comprises a C-rich specificity determining sequence (SDS) and a protospacer adjacent motif (PAM), and (b) a fusion protein comprising a catalytically-inactive Cas9 fused to cytidine deaminase.

Still other aspects of the present disclosure provide a method comprising maintaining a cell that comprises (a) an engineered nucleic acid comprising a promoter operably linked to a nucleotide sequence encoding a self-targeting guide ribonucleic acid (stgRNA) that comprises a C-rich specificity determining sequence (SDS) and a protospacer adjacent motif (PAM), and (b) a fusion protein comprising a catalytically-inactive Cas9 fused to cytidine deaminase, under conditions that result in targeted mutations in the stgRNA.

Some aspects of the present disclosure provide a kit comprising (a) an engineered nucleic acid comprising a promoter operably linked to a nucleotide sequence encoding a self-targeting guide ribonucleic acid (stgRNA) that comprises a C-rich specificity determining sequence (SDS) having and a protospacer adjacent motif (PAM), and (b) a fusion protein comprising a catalytically-inactive Cas9 fused to cytidine deaminase.

Further aspects of the present disclosure provide a method comprising maintaining a cell that comprises (a) a nucleic acid comprising a regulatory element operably linked to a target sequence, (b) an engineered nucleic acid comprising an inducible promoter operably linked to a nucleotide sequence encoding a guide ribonucleic acid (gRNA) that comprises a specificity determining sequence (SDS) that targets the regulatory sequence, and (c) a fusion protein comprising a catalytically-inactive Cas9 fused to an epigenetic effector, under conditions that result in an accumulation of targeted epigenetic changes in the vicinity of the target sequence.

Further still, aspects of the present disclosure provide in vivo diversification methods, comprising: (a) introducing into a cell (i) an engineered nucleic acid encoding a biomolecule that has at least one variable region, (ii) an engineered nucleic acid encoding a guide ribonucleic acid (gRNA) that targets the at least one variable region, and (iii) an engineered nucleic acid encoding a fusion protein comprising a catalytically-inactive Cas9 fused to a mutator domain or a Cas9 nickase fused to a mutator domain (i.e., base editor enzyme); and (b) maintaining the cell under conditions that results in diversification of the at least one variable region to produce diversified biomolecules.

Also provided, in some aspects, are cells comprising: (a) a first inducible promoter operably linked to a nucleic acid encoding a first input gRNA that targets a first SDS region of an output gRNA; (b) a second inducible promoter operably linked to a nucleic acid encoding a second input gRNA that targets a second SDS region of the output gRNA; (c) a third promoter operably linked to a nucleic acid encoding the output gRNA; (d) a fourth promoter operably linked to a nucleic acid encoding a fusion protein comprising a catalytically-inactive Cas9 fused to a mutator domain or a Cas9 nickase fused to a mutator domain; and (e) a target nucleic acid, wherein the output gRNA targets the target nucleic only following transcription of the first and second input gRNAs and binding of the first and second input gRNAs to the output gRNA.

### BRIEF DESCRIPTION OF THE DRAWINGS

The accompanying drawings are not intended to be drawn to scale. For purposes of clarity, not every component may be labeled in every drawing.

FIG. 1 depicts an example of a molecular recorder system. In this system, referred to as “mammalian SCRIBE” (Synthetic Cellular Recorders Integrating Biological Events) a self-targeting guide RNA (stgRNA) locus is continuously and autonomously cleaved in the present of Cas9. The double-stranded DNA (dsDNA) breaks introduced to the stgRNA locus are repaired by the error-prone non-homologous end joining (NHEJ) repair mechanism, which result in mutated stgRNAs (indel formation) that undergo additional rounds of cleavage and error-prone repair.

FIG. 2 depicts an example of a molecular recorder system of the present disclosure, referred to as “ramSCRIBE” (random additive memory SCRIBE). This system comprises a stgRNA that accumulates random barcodes in the presence of Cas9 and Terminal Deoxynucleotidyl Transferase (TdT), for example. A stgRNA locus is continuously and autonomously cleaved by Cas9, and random nucleotides are added to the dsDNA breaks by TdT, which can then be repaired by NHEJ. During this process, random barcodes are sequentially added to the stgRNA locus at the dsDNA break site, resulting in an increase in the length of the stgRNA specificity determining sequence (SDS).

FIG. 3 depicts yet another example of a molecular recorder system of the present disclosure, referred to as “ENGRAM” (ENGineered Random Accumulative Memory). This system comprises a catalytically-inactive Cas9 (dCas9) or a Cas9 nickase (nCas9) fused to a cytidine deaminase targeted to an array of repetitive DNA sequences by a complementary guide RNA. The deaminase domain introduces targeted mutations into the DNA array at dC positions. Uracil DNA Glycosylase Inhibitor (ugi) peptide (which inhibits repair of deaminated cytidines in DNA, can be fused to d/nCas9 to increase targeted mutation rate. The system avoids dsDNA breaks, thus avoiding shortening/lengthening of the sgRNA locus.

FIG. 4 depicts another example of a molecular recorder system of the present disclosure, referred to as “ENGRAmSCRIBE.” This system comprises a stgRNA locus that continuously and autonomously directs a dCas9 (or nCas9)-cytidine deaminase fusion protein to a stgRNA locus, enabling continuous diversification of the stgRNA locus, while avoiding dsDNA breaks or shortening/lengthening of the stgRNA locus.

FIG. 5 depicts yet another example of a molecular recorder system of the present disclosure, referred to as “epiSCRIBE” (epigenetic SCRIBE). This system comprises a dCas9 fused to an epigenetic effector domain targeted to a regulatory element (e.g. a promoter or an enhancer) by a complementary guide RNA. The epigenetic effector domain introduces targeted epigenetic changes into the vicinity of the target sequence. The accumulation of these changes results in the activation or repression of the targeted regulatory element, which can be read out by functional assays or sequencing.

FIGS. 6A-6C shows the lengthening of the stgRNA locus by the ramSCRIBE system. A modified stgRNA locus was PCR amplified and analyzed by T7 Endonuclease assay (FIG. 6A). Insertion of nucleotides at the dsDNA break site was favored when TdT was expressed along with Cas9 (FIG. 6B). A trace of random barcodes sequentially added to the stgRNA locus was detected in cells expressing the ramSCRIBE system via high throughput sequencing (FIG. 6C). Starting from the wild-type sequence, random nucleotides (highlighted) were sequentially added to a Cas9 cleavage site by TdT and NHEJ repair machinery. Individual barcodes (shaded in FIG. 6C) were called based on the available reads. Barcode calling and resolution of individual barcodes may be modified by increasing the sequencing depth.

FIG. 7 shows mutations introduced by an ENGRAM system into an integrated genomic locus.

FIGS. 8A-8B show accumulated mutations introduced by an ENGRAmSCRIBE system at a stgRNA locus. The modified stgRNA locus was PCR amplified and analyzed by T7 Endonuclease assay or high throughput sequencing. Mutations were detected in cells expressing stgRNA and nCas9_PmCDA1. T7 endo cleavage products were not detected in cells expressing gRNA (FIG. 8A). A trace of random mutations accumulated in the poly C region was detected in the stgRNA locus for cells expressing (C)10 TATGTACATACAGT stgRNA (SEQ ID NO: 78) (FIG. 8B).

FIGS. 9A-9C show evolutionary trees reconstituted from sequencing data obtained from cells expressing stgRNA and PGAL1_dCas9 (negative control, FIG. 9A), PGAL1_dCas9_PmCDA1 (FIG. 9B), or PGAL1_nCas9_PmCDA1 (FIG. 9C).

FIGS. 10A-10C show examples of targeted in vivo diversity generation in protein scaffolds using the “DRIVE” (Directed and Recurring In Vivo Evolution) platform of the present disclosure. FIG. 10A shows that a dCas9/cytidine deaminase fusion can be targeted by guide RNA (gRNA) to specific regions of a protein, RNA or DNA scaffold (e.g. an antibody) to generate a library of variants in vivo. FIG. 10B shows an example of targeting a 21 base pair poly-C region of a protein for in vivo diversity generation using a dCas9/cytidine deaminase fusion. A Sanger chromatogram shows successful diversification of the poly-C target with mainly dC to dT mutations. FIG. 10C shows representative variants identified by high-throughput sequencing of the sample subjected to the diversification scheme shown in FIG. 10B.

FIGS. 11A-11C show examples of in vivo diversification of biomolecule scaffolds using DRIVE. FIG. 11A shows an example of continuous diversity generation and screening of a biomolecule. FIG. 11B shows an examples of a self-targeting stgRNA that can be encoded downstream of a scaffold of interest to build a continuous fast-evolvable system. FIG. 11C shows an example of how individual gRNAs can be transformed into a population of bacteria, which can be then used a diversity generator population.

FIG. 12 shows an alignment of the sequence of T7 tail fiber with tail fibers from some of the relative phages that could infect other bacteria. The colored bars represent variable positions that can be targeted for diversification by DRIVE.

FIGS. 13A-13B show examples of continuous phage host range engineering using DRIVE. FIG. 13A shows an example of how targeted diversity can be introduced into bacteriophage tail fiber (and/or other segments of a phage genome that are connected to its host specificity). FIG. 13B shows that instead of using a single-diversity generator host, individual gRNAs can be transformed into a population of bacteria which can then be used as a diversity generator population.

FIGS. 14A-14C show examples of systems endowed with a synthetic Lamarckian evolution capacity. FIG. 14A shows an example of DNA writing and diversity generation by Cas9-mutators coupled to external inputs to build organisms and gene networks with the ability to undergo Lamarckian evolution. FIG. 14B shows that phages harboring a site specific mutator circuit can use the DRIVE system to increase the evolution of their tail fiber when adapting to new hose. FIG. 14C shows another example, whereby cells can be engineered to diversify key residues in their surface receptors (e.g. those are essential for binding to surfaces), and adapt to new niches much faster than is possible with Darwinian evolution.

FIG. 15 shows how a pooled gRNA library targeting ORFs and regulatory elements are transformed into cell populations, enabling the production of gene knockout, as well as up-regulation and down-regulation of gene expression.

FIG. 16 shows an example of activating silent gene clusters in natural isolates or recalcitrant bacteria.

FIG. 17, left panel, shows a schematic design of the tested DNA writing system. FIG. 17, right panel, shows Sanger sequencing results for purified plasmids and the gRNA target in each sample.

FIG. 18A shows an example of combinatorial two-input AND gate built by DOMINOS logic. FIG. 18B shows an example of sequential two-input AND gate built by DOMINOS logic. FIG. 18C shows an example of sequential two-input DOMINO logic AND gate built in E. coli. Starting from a non-functional state, the output gRNA is modified by sequential addition of IPTG and aTc to media, thus changing the sequence of the output gRNA to a functional state that could bind to a predesigned sequence (in this case GFP).

FIG. 19 shows examples of two-input DOMINO logic gates.

FIG. 20A shows a synthetic circuit that can link a given input to gene expression and reinforce expression of a reporter in the presence of a desired input. FIG. 20B shows an example of a circuit that “forgets” an existing reinforced expression. FIG. 20C shows the generation of gRNA operator arrays by stepwise editing of a DNA sequence in vivo using DNA writers.

FIG. 21A shows a three input sequential AND-gate. FIG. 21B shows an example of a timer/integrator device.

FIG. 22A shows an example of a complex sequential circuit that uses genomic DNA as a memory tape to achieve a state-dependent genetic program. FIG. 22B, left panel, shows a schematic representative of a Turing machine, which is a hypothetical computing machine that can perform computation by modifying symbols on an infinite memory tape in using a read/write head, based on a predefined set of rules and input variables. FIG. 22B, right panel, shows that to build a biological Turing machine, the genomic DNA of living cells can be used as a form of memory tape, where A, C, G and T are the symbols on this tape.

FIGS. 23A-23E show incorporating memory and logic in living cells by DOMINO. FIG. 23A shows a schematic representation of DOMINO operators. DOMINO operators are enabled by a DNA read-write head that performs efficient and precise manipulation of genomic DNA with single-nucleotide resolution. In this device, nCas9 (READ module), along with cytidine deaminase (CDA, WRITE module) and a uracil DNA glycosylase (ugi, WRITE enhancer) are addressed to a desired genomic loci using gRNA with a complementary seed region (READ address). Localization of the CDA write module to the target results in the deamination of cytidine (dC) residues in target in the vicinity of 5′-end of the gRNA (WRITE address) and their conversion to dU residues, which are then preferentially repaired by the cellular machinery to dT (or dG to dA mutation if the negative strand of DNA is targeted by gRNA). By placing the DNA read-write module and the gRNA under the control of inducible signals, DNA writing for DOMINO operators can be tuned and controlled by external cues. Here, the basic DOMINO operator was schematized as an AND gate since it requires the expression of both the DNA read-write head (i.e., CDA-nCas9-ugi controlled by the “operational signal”) as well as the gRNA (regulated by “Input 1”) with a downstream feedback delay operator (to illustrate the unidirectional and memory aspect of the operator). DOMINO operators can be layered to a wide variety of memory and logic functions. Bold nucleotides on the target show the location of NGG PAM sequence. Targeted nucleotides are underlined. FIG. 23B shows combinatorial AND gate enabled by DOMINO where the output is ON only when both inputs have been present. Induction of the circuit with either of the two inducers (IPTG or Ara), results in editing of the target and transition to an intermediate state (states S1 or S2, respectively). Induction of the circuit with both gRNAs results in generation of the doubly edited DNA sequence (state S3), which is designated as ON state. FIG. 23C shows dynamics of allele frequencies obtained by Illumina High-Throughput Sequencing (HTS) for the circuit shown in FIG. 23B. E. coli cells were exposed to different inducer combinations for four days with serial dilution after each 24 hours. Error bars indicate standard deviation of three biological replicates. FIG. 23D shows position-specific mutant allele frequencies for the last time point (96 hours) of the experiment shown in FIG. 23C estimated from Sanger sequencing analysis by Sequalizer (see Materials and Methods). This data demonstrates the expected outcomes of AND gate behavior at the population level. The x-axis shows dC to dT or dG to dA mutations in the specified positions. For example, the G18A mutation means a dG to dA mutation in position 18 of the target sequence. Small boxes along the x-axis show the induction patterns and duration of induction used in each experiment. For example, the induction pattern of the last sample set ([IA][IA][IA][IA]) means that the samples were induced with aTc+IPTG+Ara for four days with dilutions every 24 hours. Error bars indicate standard deviation of three biological replicates. FIG. 23E shows that the output of DOMINO operators, which is in the form of mutations in DNA, can be converted to a gRNA, by flanking the target DNA sequence with a desired promoter and gRNA handle. This allows DOMINO operators to be linked to other DOMINO operators or host regulatory networks. To demonstrate this concept, a combinatorial DOMINO AND gate was designed with a target sequence flanked by a constitutive promoter and a modified gRNA handle. The modified gRNA handle harbored a dA to dG mutation in a position that was not essential for gRNA function (27). This modification (shown by an asterisk) was required to generate an NGG PAM motif for binding of one of the input gRNAs. Upon induction by both inducers, the input gRNAs can edit the Specificity-Determining Sequence (SDS) of the output gRNA. The doubly edited output gRNA can then bind to the GFP ORF and repress it via CRISPRi in E. coli. In this example, AND logic is realized on the target DNA register (i.e., the output gRNA) while NAND logic is achieved on the output GFP reporter. Error bars indicate standard deviation for three biological replicates.

FIGS. 24A-24E show building sequential logic by DOMINO operators. FIG. 24A shows sequential AND gate encoded with DOMINO operators. The output of a DOMINO operator was used as an input for another operator, which in turn mutates a non-canonical start codon (ACG) within the GFP ORF into a canonical (efficient) start codon (ATG), thus increasing GFP signal. The second gRNA (induced by Ara) can bind to and enact the start-codon mutation only after the first gRNA (induced by IPTG) has edited its target. FIG. 24B shows a GFP signal measured by flow cytometry for the circuit shown in FIG. 24A. Only when IPTG AND THEN Ara are applied, the sequential logic is satisfied, thus resulting in increased GFP signal. Error bars indicate standard deviation of three biological replicates. FIG. 24C shows position-specific mutation frequency obtained from Sequalizer analysis for the experiment shown in FIG. 24A. Consistent with GFP data, the highest frequency of ACG to ATG conversion (blue bars) was achieved when the samples were induced with IPTG AND THEN Ara. Error bars indicate standard deviation for three biological replicates. FIG. 24D shows a two-input/two-output race-detecting circuit. Two gRNAs were designed so that editing by one gRNA destroys the PAM domain for the other gRNA, thus inhibiting its binding. Sequential expression of each gRNA resulted in an output corresponding to the output of the first gRNA, independent of whether the second gRNA was expressed or not. Error bars indicate standard deviation for three biological replicates. FIG. 24E shows another example of sequential DOMINO logic, where sequential induction of cells with IPTG AND THEN Ara results in the sequential transition between two modified states (states S1 and S3, respectively). However, induction of cells with the reverse order (Ara AND THEN IPTG) only results in a one-step transition to state S2. Error bars indicate standard deviation for three biological replicates.

FIGS. 25A-25C show incorporating propagation delay and temporal logic into living cells. FIG. 25A shows time-dependent logic and tunable propagation delay can be programmed by DOMINO operator cascades. DOMINO operators possess an inherent propagation delay (the time required for transition from a non-modified state to modified state) that can be modulated in an analog fashion (stronger induction results in a shorter delay). Multiple DOMINO operators can be placed sequentially in an array to build longer delays and then coupled with other logic operators to build temporal logic. A series of overlapping repeats were constructed to serve as gRNA binding sites. Once expressed, the first gRNA (IPTG-inducible, pink) can bind to the downstream repeat, but not to the other instances of the repeats due to presence of dC residues in these repeats that form mismatches with the gRNA READ address. Upon binding the downstream repeat, the DNA read-write head can mutate these dC residues to dT in the immediately adjacent upstream repeat, thus creating a new binding site for this gRNA. In turn, this event recruits the read-write head once again and makes the third repeat available for binding. The second gRNA, which is under control of Ara, is only able to bind to and edit its target when the third copy of the repeat is edited by the first gRNA, thus encoding time-dependent sequential logic. FIG. 25B shows that E. coli cells harboring the circuit shown in FIG. 25A were exposed to different concentrations of the first inducer (IPTG) for 4 days with serial dilution after each day, followed by a one-day exposure to the second inducer (Ara). The propagation of the signal as manifested by sequential mutations in the repeat array was monitored by analyzing Sanger chromatograms with Sequalizer. Transitions between states occurred in a time- and IPTG-dosage dependent fashion, and only cells exposed to higher concentrations of IPTG (0.1 mM and 0.01 mM) accumulated mutations to the level that enabled a response to the second inducer (Ara) by the last day of experiment. FIG. 25C shows transitions between the memory states for samples shown in FIG. 25B assessed by HTS. Error bars indicates standard deviation for three biological replicates.

FIGS. 26A-26F show associative learning and online DNA-state reporting circuits in human cells. FIG. 26A shows that because DOMINO operators are CRISPR-Cas9-based, they can be functionalized with transcriptional and epigenetic modules to implement gene regulation integrated with computing and memory. As an example, the read-write head was functionalized with a transcriptional activator (VP64) and was used to sequentially edit and activate multiple operator sites that were arrayed in overlapping repeats (composed of four copies WT unmutated repeats (Op) followed by a downstream mutated repeat (Op*)) upstream of a minimal promoter (4xOp_1xOp*_GFP). At the presence of Op*-specific gRNA (gRNA(Op*)), this system allows for sequential conversion of Op sites to Op* and binding of the transactivator to the progressively mutated operator sites in the promoter, which in turn results in GFP signal increases. Therefore, cells harboring this circuit manifest sequential and permanent transitions between DNA states and increases in GFP in response to increased gRNA expression over time. Thus, the circuit can be considered as an example of associative learning. FIG. 26B shows that HEK 293T cells were transfected with the circuit shown in FIG. 26A via a two-step lentiviral delivery protocol and were grown with serial passaging every three days as indicated. At the end of each passage, GFP signal was assessed by microscopy and DNA memory state was assessed by HTS. FIG. 26C shows the average number of GFP-positive cells in different samples harboring either the Op*-specific gRNA (gRNA(Op*)) or a non-specific gRNA (gRNA(NS)) and either 4xOp_1xOp*_GFP or 1xOp*_GFP as reporter. The number of GFP-positive cells harboring 4xOp_1xOp*_GFP and gRNA(Op*) increased over time. In contrast, the number of GFP-positive cells in cultures harboring gRNA(NS) or 1xOp*_GFP and gRNA(Op*) did not change and remained at background levels. FIG. 26D shows a histogram of signal intensities for GFP-positive cells shown in FIG. 26C. Over time, the intensity of GFP-positive cells increased in samples harboring 4xOp_1xOP*_GFP and gRNA(Op*) gradually increased, reflected as a shift to the right in the histograms, indicating multi-stage GFP activation in these cells. The signal intensities in cells harboring gRNA(NS) or those that had 1xOp*_GFP and gRNA(Op*) remained at the background level. FIG. 26E shows dynamics of the frequency of the WT unmodified allele (state S0) in cultures harboring 4xOp_1xOp*_GFP and gRNA(Op*) assessed by HTS. The frequency of the unedited allele decreased linearly over time, indicating that the DNA writing circuit can be used as an analog recorder for the input gRNA. FIG. 26F shows dynamics of mutant allele frequencies (memory states S1 through S5) for the same samples as FIG. 26E, shown as time-series data and histograms. Consistent with the GFP data, the first four memory states (S1 through S4) started to accumulate sequentially (state S1, then state S2, then S3 and then S4) until they reached a plateau. Moreover, memory state S5, which corresponds to the highest GFP expression state, increased steadily over time, as was expected from the terminal product of the DNA memory circuit.

FIGS. 27A-27D show high-capacity, continuous, and long-term ENGRAM recorders for memorizing analog signals and chronicling molecular events. FIG. 27A shows a schematic representation of the ENGRAM high-capacity molecular recording system. A self-targeting gRNA (stgRNA) with a 43-bp C-rich SDS was placed under the control of a desired input. Once expressed, the stgRNA directs the DNA read-write head to its own locus, resulting in dC to dT (and with lower frequency to dG and dA) mutations that accumulate in the stgRNA locus as a function of duration and magnitude of signal controlling the gRNA expression. In this design, transitions between memory states are pseudo-random but accumulative, and always occur from a lower memory state (i.e., lower degree of mutations, S(n)) to a higher memory state (i.e., higher degree of mutations, S(n+i)). FIG. 27B shows that E. coli cells with the circuit shown in FIG. 27A were induced with aTc and different concentrations of Ara as indicated, and grown for 36 hours with dilution every 12 hours. Samples were taken at different time points throughout the experiment and assessed for allele frequencies by HTS. Frequency of mutants in the population increased continuously in a time- and Ara dosage-dependent manner, demonstrating that the recorder can continuously record analog information of an incoming signal. FIG. 27C shows unidirectional and pseudo-random mutations that accumulate in the specific positions (i.e., dC residues) within an stgRNA memory register can be considered as non-disruptive and probabilistic transitions between memory states. These mutations (i.e., memory states) can be used to trace back mutation trajectories and cellular lineages. FIG. 27D shows an example of a high-resolution cellular lineage generated from the samples shown in FIG. 27B (36 hour induction, aTc+0.2% Ara). Positions with the same sequence as the WT stgRNA allele are indicated by dots.

FIGS. 28A-28C show using Sequalizer to estimate position-specific mutant frequencies from Sanger chromatograms. FIG. 28A shows sequalizer analysis comparing two instances of WT unmutated (i.e., Ref samples) sequences (top) and a WT unmutated (Ref) sequence vs. Test sample containing a mixture of mutated and unmutated sequences (bottom). The y-axis shows differences between normalized Sanger chromatograms for the samples being compared (Ref #1 vs. Ref #2 or Ref vs. Test). Peaks in these plots indicate differences in the normalized chromatograms and thus mutations in corresponding positions. For example, the peak marked by a black arrow in the bottom plot indicates mutations of dG at position 18 in the Ref to dA in the Test sample. The numbers above target positions (i.e., positions 18-21), show the estimated mutant frequency in that position based on the Sequalizer algorithm, which takes into account the height of Sanger chromatograms in a given position to normalize the calculated difference values. FIG. 28B shows standard curves obtained by analyzing samples containing known mutant ratios by Sequalizer. Two plasmids encoding the pure WT and mutant sequences (as indicated) were mixed at different molar ratios. The mixtures were Sanger-sequenced and the obtained chromatograms were analyzed by Sequalizer. The estimated mutant frequencies at the four target positions were plotted against the known (i.e., experimentally mixed) mutant ratios. Error bars indicate standard deviation for six independent replicates. FIG. 28C shows the position-specific mutant frequencies measured by Sequalizer vs. HTS at four target positions for samples from the experiment described in FIG. 23B.

FIGS. 29A-29E show examples of additional circuits built using DOMINO operators. FIG. 29A shows a schematic representation and truth table for a combinatorial DOMINO OR gate. FIG. 29B shows Sequalizer results for the circuit shown in FIG. 29A shows that E. coli cells were induced for four days using the indicated patterns and position-specific mutant frequencies were assessed by Sequalizer analysis of Sanger chromatograms. Error bars indicate standard deviation for three biological replicates. FIG. 29C shows sequential AND gate built by a cascade of gRNAs, where the first (IPTG-inducible) gRNA edits and activates a downstream gRNA, which can then edit a downstream target. As demonstrated in this example, gRNA outputs of a DOMINO cascade can be independently regulated by using inducible promoters, such as an Ara-inducible promoter. This offers greater flexibility compared to using mutations as DOMINO outputs (e.g., designs shown in FIGS. 24A-24E and 25A-25C). FIG. 29D shows dynamics of allele frequencies (i.e., memory states) for the circuit shown in FIG. 29C assessed by HTS (top) and Sequalizer (bottom). Error bars indicate standard deviation for three biological replicates. FIG. 29E shows a multiplexer circuit, where the presence of three input gRNAs is converted to cis-encoded mutations in the target DNA locus (lacZ gene in E. coli). The circuit can be used to convert multiplexed transcriptional signals from various loci across a genome into DNA memory within a confined region. The multiplexed and DNA-encoded signals can then be analyzed and demultiplexed by HTS or Sanger sequencing to reveal information about the signals. The plots on the right show the Sequalizer output plots for cells containing no gRNA (top) and those containing three constitutively-expressed input gRNAs (bottom). Mutations in gRNA target sites are reflected as peaks in the bottom Sequalizer plot. This circuit is an example of a DOMINO circuit with more than two inputs, which can be readily extended to additional inputs for in vivo memory applications and storing information (spatial, temporal, or artificial) across a genome.

FIG. 30 shows regulation of gene expression by manipulating functional elements by DOMINO. Conditional conversion of a canonical, efficient initiation codon (ATG) to ATA (which is a non-efficient initiation codon) by an Ara-inducible DOMINO operator was used to down-regulate GFP expression in E. coli. Over time, the number of GFP-positive cells decreased and the frequency of mutants increased in induced samples while these quantities minimally changed in non-induced samples. For GFP measurements, samples were grown for six hours in LB with no inducers before flow cytometry to ensure removal of any repression (i.e., CRISPRi) effect enacted by bound CDA-nCas9-ugi. Error bars indicate standard deviation of three biological replicates.

FIGS. 31A-31B show dynamics of allele frequencies (memory states) for the race-detecting circuit shown in FIG. 24D (FIG. 31A) and the sequential logic circuit shown in FIG. 24E (FIG. 31B). In each subplot, the dominant allele in the last time point has been used to determine the memory state. Error bars indicate standard deviation for three biological replicates.

FIGS. 32A-32B show using DOMINO delay elements to temporally control the conversion of cryptic start codons into canonical start codons in three ORFs. FIG. 32A shows the schematic representation of the time-dependent codon conversion experiment. Three different ORFs with non-canonical (ACG) start codons and different number of delay elements (i.e., overlapping repeats) in their N-termini were placed in a synthetic operon. A gRNA was designed so that it could bind to the 3′-distal repeat element in each array. Sequential recruitment and editing of the repeat elements by this gRNA led to progressive mutation accumulation within the repeat elements toward the 5′-end and eventually editing of the upstream ACG codons to ATG. In this circuit, due to the presence of different number of delay elements in each array, different delay times and thus temporal regulation is achieved. The time required for start codon conversion for ORF 1 (t1) is expected to be longer than the time required for ORF 2 (t2) which itself is expected to be longer than the time required for the conversion in ORF 3 (t3). FIG. 32B shows that the E. coli cells harboring the indicated circuit in FIG. 32A were induced and then mutation accumulation in the arrays was monitored by Sanger sequencing and Sequalizer over time. Upon induction of the circuit, time-dependent accumulation of mutations was observed in all the three repeat arrays. The position corresponding to the start codon (shown by red arrow) in the third ORF, which possessed only two repeats in its N-terminus array, was the first that accumulated significant levels of mutations. This was followed by the second ORF, which contained four delay elements and thus experienced a longer delay compared to ORF 3. The first ORF, which possessed six repeats and was thus subject to the longest delay, was the last ORF in which mutations in the position corresponding to the cryptic start codon were accumulated. On the other hand, in non-induced cells, only low levels of mutations accumulated in the downstream repeat of each array and only at the later time points of the experiment, likely due to the background activity of the promoters. Nevertheless, no mutations were detected in positions corresponding to cryptic start codons in non-induced cells.

FIGS. 33A-33B show representative microscopy images and additional data for the experiment shown in FIG. 26A-26F. FIG. 33A shows representative microscopy images for cells harboring the 4xOp_1xOp*_GFP reporter and the Op*-specific gRNA (gRNA(Op*)) or a non-specific gRNA (gRNA(NS)). FIG. 33B shows dynamics of allele frequencies (memory states) for cells harboring the 4xOp_1xOp*_GFP reporter and gRNA(NS) (negative control). FIG. 33C shows dynamics of allele frequencies (memory states) for cells harboring the 1xOp*_GFP reporter and gRNA(Op*). The mutable dC residue within the gRNA target site was mutated with a constant rate into dT and constant but lower rates into dG and dA, reflecting the promiscuous repair of deaminated cytidine lesions in mammalian cells. The linear decrease in dC allele frequency, as well as the linear increases in dT, dG, and dA allele frequencies, can be used as an analog readout of gRNA expression duration or intensity.

FIG. 34 shows Pearson correlation between frequencies of modified alleles in different samples (obtained from the experiment described in FIG. 27B), plotted against the ratios of WT (S0) allele frequencies in the corresponding samples. Samples with similar frequencies of the WT allele (x-axis value close to 0) showed high correlation between their frequencies of mutant alleles as well, independent of their input histories. This was true even for samples that were induced for a long time with a low concentration of the input (Ara) compared with those that were induced for a short time with a high concentration of the input. This suggests that transitions between states are independent of input histories, and depends on the allele frequencies in the current state.

FIGS. 35A-35F show continuous synthetic Lamarckian evolution of cellular phenotypes enabled by coupling de novo diversity generation with continuous selection by DRIVE. FIG. 35A shows that continuous de novo targeted diversity generation can be coupled with a selective pressure (or screening) to allow optimizing phenotype of interest without concomitant increase in the global mutation rate. FIG. 35B shows that to achieve a large dynamic span in fitness, P_{lac} promoter of E. coli was weakened, which controls fitness (i.e., growth rate) of cells at the presence of lactose as the sole carbon source, by introducing 6-bp poly-dC into −35 and −10 regulatory boxes of this promoter to make a mutant P_{lac} promoter (P_{lac}(mut)). Complementary gRNAs targeting these two regulatory regions were then introduced to endow cells with the ability to site-specifically increase their de-novo mutation rate. FIG. 35C shows that cells harboring the DNA writer with or without the P_{lac}-targeting gRNAs were grown either in selective media (containing lactose as the sole carbon source) or non-selective media (containing glucose as the sole carbon source) for three successive grow and dilutions cycles. The growth rate of cells in lactose, as well as activity of P_{lac} promoter was monitored throughout the experiment. FIG. 35D shows the average population growth rate of parallel cultures with or without P_{lac}-targeting gRNAs in lactose. FIG. 35E shows P_{lac} activity activity for parallel cultures with or without P_{lac}-targeting gRNAs grown in lactose. FIG. 35F shows the sequence logo of position weight matrixes for the parental strain, as well as cells with or without P_{lac}-targeting gRNAs grown in either glucose or lactose are shown (top panel). Jensen-Shannon divergence for pair-wise comparison of these samples are shown in the bottom panel. For each subplot, positions that harbor different nucleotide distributions are indicated by the letters corresponding to each nucleotide. The letter in the upper section of each subplot correspond to the nucleotides over-represented in the sample in the corresponding column, while the letter in the lower section corresponds to the sample in the corresponding row. Comparing the mutant distribution in cells harboring P_{lac}-targeting gRNAs that were grown in the selective media (lactose) and non-selective media (glucose, reveals adaptive mutations (marked by red arrows) in the vicinity of gRNA target sites on the P_{lac}).

### DETAILED DESCRIPTION

The present disclosure provides several molecular recorder systems that may be used in living cells to convert transient signals into a form of memory that can be used, for example, to record cellular events of interest, to trace the cell lineage and/or to diversify a target sequence of interest.

Also provided herein is a platform referred to as “DRIVE” (Directed and Recurring In Vivo Evolution), which implements tools of the present disclosure (e.g., DNA writers and molecular recorder components) for in vivo targeted diversification of DNA-encoded sequences in living cells.

Further provided herein is a platform referred to as “DOMINO” (DNA-based Ordered Memory and Iteration Network Operating System), which is a highly transformative platform for building compact and scalable logic and memory operations in living cells and enables control of cellular phenotypes by executing unidirectional cascades of DNA writing events.

### Molecular Recorder Systems

Each of the molecule recorder systems provided herein include a ribonucleic acid (RNA)-guided endonuclease, a guide RNA (gRNA) that targets the RNA-guided nuclease to a target sequence, an enzyme that introduces mutations (barcodes) to the target site, and an additional molecule that functions to modify nucleic acid (e.g., terminal deoxynucleotidyl transferase (TdT), cytidine deaminase, or an epigenetic effector). Each of the foregoing components are described below.

As indicated above, the molecular recorder systems of the present disclosure artificially elevate mutation rates within targeted genomic segments and write the targeted mutations (memory states) into DNA. Thus, in some embodiments, the rate at which mutations are introduced into a target sequence may be 0.1 to 100 time, or 0.1 to 10 times, higher than a control mutation rate. For example, the rate at which mutations are introduced into a target sequence may be 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 50, or 100 times higher than a control mutation rate.

The control mutation rate may be a natural mutation rate, for example, the rate of mutation in a cell in its natural environment. The control mutation rate alternatively may be the rate of mutation introduced into a target site using another molecular recording technology (e.g., a molecular clock). Controls may be determined based on the particular applications for which the molecular recorders of the present disclosure are used.

ramSCRIBE Molecular Recorder System

The ramSCRIBE (random additive memory Synthetic Cellular Recorders Integrating Biological Events) system as provided herein includes a stgRNA that accumulates random barcodes in the presence of Cas9 nuclease and terminal deoxynucleotidyl transferase (TdT) (FIG. 2). The stgRNA locus is continuously cleaved by Cas9 and random nucleotides are added to the dsDNA breaks by TdT, which can then be repaired by NHEJ. The rate of nucleotides insertions is increased by the presence of TdT, compares to deletions at the dsDNA break sites. As a result, the rate of stgRNA shortening is reduced, the duration of recording is extended, and memory capacity is enhanced. During this process, random barcodes are added to the stgRNA locus at the break site in a step-wise manner, resulting in sequentially increase in the length of the stgRNA's specificity determining sequence (SDS). The sequential addition of the barcodes by TdT enables the recording of new events while preserving the previous barcodes, thus enabling tracing of the chronicle of molecular (indel formation) events unambiguously. For example, cellular lineage can be tracked by tracking the random barcodes that accumulate in the stgRNA locus.

Some aspects of the present disclosure provide cells comprising a ramSCRIBE system. The “generation of random additive memory” refers to the sequential addition (or subtraction) of random nucleotides at a target site, wherein a double-stranded DNA break is introduced by an RNA-guided nuclease (e.g., a Cas9 nuclease). Accordingly, in some embodiments, the cells in which random additive memory is generated comprises an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM), a RNA-guided endonuclease (e.g., Cas9 or Cpf1), and an enzyme that catalyzes the addition of nucleotides to the end of a nucleic acid.

Enzymes that catalyzes the addition of nucleotides to the end of a nucleic acid are known to those skilled in the art. In some embodiments, the enzyme is a DNA polymerase from the X-family of DNA polymerases. In some embodiments, the enzyme is a terminal deoxynucleotidyl transferase (TdT), a polymerase λ, or a polymerase μ. TdT is a specialized DNA polymerase expressed in immature, pre-B, pre-T lymphoid cells, and acute lymphoblastic leukemia/lymphoma cells. TdT adds N-nucleotides to the V, D, and J exons of the TCR and BCR genes during antibody gene recombination, enabling the phenomenon of junctional diversity. In humans, terminal transferase is encoded by the DNTT gene (e.g., as described in Motea et al., Biochim Biophys Acta. 2010 May; 1804(5): 1151-1166, incorporated herein by reference). Example amino acid sequence of TdT and polymerase are provided in Table 4.

Other examples of enzymes that catalyzes the addition of nucleotides to the end of a nucleic acid (including dsDNA breaks) include, but are not limited to, abiK RT (Wang, C. et al., Nucleic Acids Res. 2011 Sep. 1; 39(17):7620-9, incorporated herein by reference) and LigD (Aniukwu, J. et al., Genes Dev. 2008 Feb. 15; 22(4): 512-527, incorporated herein by reference). In some embodiments both LigD and Ku are used to catalyzes the addition of nucleotides to the end of a nucleic acid (Della, M. et al., Science. 2004 Oct. 2; 306(5696):683-5, incorporated herein by reference).

As an alternative to enzymes that catalyze the addition of nucleotides to the end of a nucleic acid (or to dsDNA breaks), enzymes that can recess DNA ends may be used in similar manner. For example, rather than using sequential addition of nucleotides to form a barcodes, sequential deletions (removal of) nucleotides may be used. Due to shortening guide RNAs, however, the recording capacity may be exhausted after multiple reactions. Examples of DNA end processing enzymes that can be used for sequential deletions include, but are not limited to, TREX2 and Artemis (Certo, T. et al., Nat Methods. 2012 October; 9(10): 973-975, incorporated herein by reference).

An enzyme that catalyzes the addition of nucleotides to the end of a nucleic acid DNA (e.g., TdT) may be expressed either separately or as a fusion to a RNA-guided endonuclease (e.g., Cas9). A fusion increases the local concentration of the corresponding DNA-end processing enzyme in the dsDNA break site, thus increasing the end processing activity. At the same time, this limits off-target activity of these enzymes on dsDNA breaks that naturally occurs, thus reducing unwanted effects.

Thus, fusion proteins are also contemplated herein. Methods of making a fusion protein are known to those skilled in the art. In some embodiments, the enzyme that adds random nucleotides to dsDNA breaks (e.g., TdT) may be fused to the N-terminus of the RNA-guided endonuclease (e.g., Cas9 or Cpf1). In some embodiments, the enzyme that adds random nucleotides to dsDNA breaks (e.g., TdT) may be fused to the C-terminus of the RNA-guided endonuclease (e.g., Cas9 or Cpf1).

Linkers may be used to fuse two protein partners to form a fusion protein. A “linker” is a chemical group or a molecule linking two molecules or moieties, e.g., two domains of a fusion protein, such as, for example, a nuclease-inactive Cas9 domain and a nucleic acid editing domain (e.g., a deaminase domain). Typically, the linker is positioned between (flanked by) two groups, molecules, domains, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer (e.g. a non-natural polymer, non-peptidic polymer), or chemical moiety. In some embodiments, the linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

Various linker lengths and flexibilities between the protein domains can be used (e.g., ranging from very flexible linkers of the form (GGGS)n (SEQ ID NO: 31), (GGGGS)ₙ (SEQ ID NO: 32), (GGS)ₙ, and (G)ₙ to more rigid linkers of the form (EAAAK)ₙ (SEQ ID NO: 33), SGSETPGTSESATPES (SEQ ID NO: 34) (see, e.g., Guilinger et, al., Nat. Biotechnol. 2014; 32(6): 577-82; the entire contents are incorporated herein by reference), (XP)ₙ, or a combination of any of these, wherein X is any amino acid and n is independently an integer between 1 and 30, in order to achieve the optimal length for deaminase activity for the specific application. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or if more than one linker or more than one linker motif is present, any combination thereof. In some embodiments, the linker comprises a (GGS)ₙ motif, wherein n is 1, 2, 3, 4, 5, 6, 7, 8, 9, 10, 11, 12, 13, 14 or 15. In some embodiments, the linker comprises a (GGS)ₙ motif, wherein n is 1, 3, or 7. In some embodiments, the linker comprises the amino acid sequence SGSETPGTSESATPES (SEQ ID NO: 35), also referred to as the XTEN linker. In some embodiments, the linker comprises an amino acid sequence chosen from the group including, but not limited to, AGVF (SEQ ID NO: 36), GFLG, FK, AL, ALAL, or ALALA (SEQ ID NO: 37). In some embodiments, suitable linker motifs and configurations include those described in Chen et al., Fusion protein linkers: property, design and functionality. Adv Drug Deliv Rev. 2013; 65(10):1357-69, which is incorporated herein by reference. In some embodiments, the linker may comprise any of the following amino acid sequences: VPFLLEPDNINGKTC (SEQ ID NO: 38), GSAGSAAGSGEF (SEQ ID NO: 39), SIVAQLSRPDPA (SEQ ID NO: 40), MKIIEQLPSA (SEQ ID NO: 41), VRHKLKRVGS (SEQ ID NO: 42), GHGTGSTGSGSS (SEQ ID NO: 43), MSRPDPA (SEQ ID NO: 44), GSAGSAAGSGEF (SEQ ID NO: 45), SGSETPGTSESA (SEQ ID NO: 46), SGSETPGTSESATPEGGSGGS (SEQ ID NO: 47), or GGSM (SEQ ID NO: 48). Additional suitable linker sequences will be apparent to those of skill in the art based on the instant disclosure.

The fusion protein (e.g., TdT-Cas9 fusion protein) described herein functions in the same manner as when the two fusion partners are in individual form. For example, the fusion protein is able to be directed to the target site by the stgRNA, wherein the Cas9 domain of the fusion protein introduces a dsDNA break and the TdT domain of the fusion protein adds random nucleotides to the dsDNA break.

### ENGRAM Molecular Recorder System

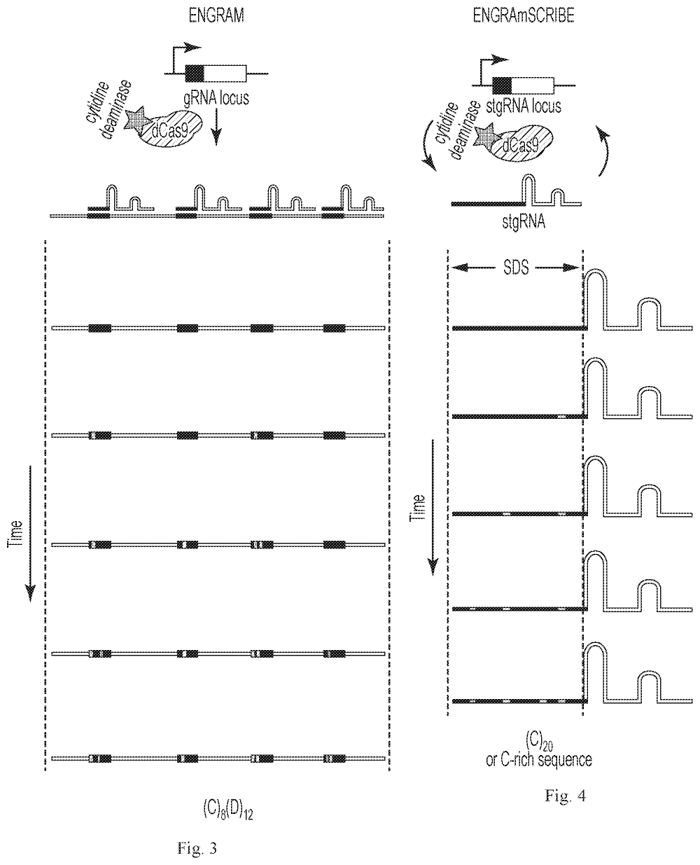

The ENGRAM (engineered random accumulative memory) system as provided herein is a minimally disruptive molecular recorder system that bypasses the need for dsDNA breaks, thus avoiding cellular toxicity and stgRNA shortening. The ENGRAM system does not rely on stochastic deletion-based mutations for editing a target DNA sequence, but instead introduces localized point mutations into the target sites in a step-wise fashion. The ENGRAM system includes a nuclease-inactive Cas9 (dCas9) or a Cas9 nickase (nCas9) fused to a DNA editing enzyme (e.g., a cytidine deaminase). The ENGRAM system may be targeted to an array of repetitive DNA sequences by a complementary guide RNA (FIG. 3). The deaminase domain introduces targeted mutations into the DNA array at dC positions. Newly-introduced mutations by the ENGRAM system do not rewrite the previous mutations (i.e., memory states), enabling tracing of the chronicle of events (e.g., cell lineage tracing). The accumulation of these mutations in the DNA array can be read out by sequencing. The SDS sequence is designed so that the seed sequence (e.g., 12 bp seed sequence) that is required for binding of dCas9 is not C-rich (e.g. C₈D₁₂). Thus only the residues that are non-essential for binding are mutated.

Since the ENGRAM system avoids dsDNA breaks, which could cause chromosomal rearrangement if multiple breaks occur simultaneously in the same cell, multiple memory units can operate orthogonally within a cell (i.e., highly scalable). Furthermore, the memory capacity of the ENGRAM system, which depends on the number of dC residues in the gRNA target sites, can be expanded by increasing the number of dC residues in the target sites. This can be achieved by incorporating arrays of C-rich gRNA target sites in the cells (or using naturally occurring repeats) or using multiple gRNAs that target different neighboring sequences within cells. Nonetheless, mutations within the first 12 bps of the gRNA target, closer to PAM, may abolish Cas9 binding, thus, in some embodiments, this region does not comprise dC residues.

Some aspects of the present disclosure provide cells comprising an ENGRAM systems. The “engineered random accumulative memory” refers to point mutations within a target site generated by an enzyme capable of converting one base to another without dsDNA break (e.g., a cytidine deaminase that converts a cytosine to a thymine). Accordingly, in some embodiments, the cell comprises an engineered nucleic acid comprising a promoter operably linked to a nucleotide sequence encoding a guide ribonucleic acid (gRNA) that targets the array of repetitive dC-rich DNA sequences, and a fusion protein comprising a RNA-guided DNA binding domain (e.g., dCas9, nCas9, or dCpf1) fused to cytidine deaminase (e.g., APOBEC1).

A “deaminase” refers to an enzyme that catalyzes the removal of an amine group from a molecule, or deamination, for example through hydrolysis. In some embodiments, the deaminase is a cytidine deaminase, catalyzing the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T, as described in Komor et al (Nature, Programmable editing of a target base in genomic DNA without double-stranded DNA cleavage, 533, 420-424 (2016), which is incorporated herein by reference). In some embodiments, the deaminase is a cytidine deaminase, catalyzing and promoting the conversion of cytosine to uracil (e.g., in RNA) or thymine (e.g., in DNA). In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variants do not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A “cytidine deaminase” refers to an enzyme that catalyzes the chemical reaction “cytosine+H₂O⇄uracil+NH₃” or “5-methyl-cytosine+H₂O⇄thymine+NH₃.” As it may be apparent from the reaction formula, such chemical reactions result in a C to U/T nucleobase change. In the context of a gene, such nucleotide change, or mutation, may in turn lead to an amino acid change in the protein, which may affect the protein's function, e.g., loss-of-function or gain-of-function. Subsequent DNA repair mechanisms ensure that uracil bases in DNA are replaced by T, as described in Komor et al. (Nature, 533, 420-424 (2016), which is incorporated herein by reference).

One example of a suitable class of cytidine deaminases is the apolipoprotein B mRNA-editing complex (APOBEC) family of cytidine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytidine deaminases all require a Zn²⁺-coordinating motif (His-X-Glu-X₂₃₋₂₆-Pro-Cys-X₂₋₄-Cys; SEQ ID NO: 72) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytidine deaminase is the activation-induced cytidine deaminase (AID), which is responsible for the maturation of antibodies by converting cytosines in ssDNA to uracils in a transcription-dependent, strand-biased fashion.

Methods of introducing point mutations using a fusion protein comprising a DNA binding domain (e.g., dCas9 or nCas9) fused to cytidine deaminase (e.g., APOBEC1) are known in the art (e.g., as described in Komor et al., Nature, 533, 420-424 (2016), incorporated herein by reference). Amino acid sequences of non-limiting, exemplary cytidine deaminases that may be used in accordance with the present disclosure are provided in Table 5.

One skilled in the art is familiar with methods of making fusion proteins. Any linker sequences known in the art and described herein may be used in the RNA-guided DNA binding domain-cytidine deaminase fusion proteins described herein. In some embodiments, the RNA-guided DNA binding domain is fused to the N-terminus of the cytidine deaminase. In some embodiments, the RNA-guided DNA binding domain is fused to the C-terminus of the cytidine deaminase.

In some embodiments, the target site for the RNA guided DNA binding domain-cytidine deaminase fusion protein is a nucleotide sequence that is rich in deoxycytosine nucleotides (dC-rich). Being “dC-rich” means at least 20% of the target site sequence is deoxycytosine. For example, a “dC-rich” DNA sequence contains at least 20%, at least 30%, at least 40%, at least 50%, at least 60%, at least 70%, at least 80%, at least 90%, at least 95%, at least 99%, or more deoxycytosine. In some embodiments, a “dC-rich” DNA sequence contains 20%, 25%, 30%, 35%, 40%, 45%, 50%, 55%, 60%, 65%, 70%, 75%, 80%, 85%, 90%, 95%, 99%, or 100% of deoxycytosine. A dC-rich DNA sequence may be 5-100 nucleotides long. For example, a dC-rich DNA sequence may be 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 amino acids long. In some embodiments, a dC-rich DNA sequence may be 5, 10, 15, 20, 25, 30, 35, 40, 45, 50, 55, 60, 65, 70, 75, 80, 85, 90, 95, 100 nucleotides long.

In some embodiments, the target site is a naturally occurring dC-rich DNA sequence, e.g., in the genome of the cell. In some embodiments, the target site is an engineered site that is integrated into the genome of the cell. In some embodiments, the engineered target site includes an array of repetitive dC-rich DNA sequences. An “array of repetitive dC-rich DNA sequences” refers to a series of dC-rich DNA sequences linked together to form an “array.” Each array may include more than one (e.g., 2, 3, 4, 5, 6, 7, 8, 9, 10, or more) repeat of dC-rich (e.g., containing at least 20%, at least 30%, at least 40%, at least 50%, at least 60%, at least 70%, at least 80%, at least 90%, at least 95%, at least 99%, or more deoxycytosine) DNA sequences. Linker nucleotide sequences may be present between each repeat. One skilled in the art is familiar with nucleotide sequences that may be used as linkers. The linker sequences may be designed to not contain any deoxycytosine.

The array of repetitive dC-rich DNA sequence may be integrated into a genomic site of the cell via any known methods in the art. For example, the integration may be mediated by site-specific recombination, ZFN or TALEN-mediated genome editing, or CRISPR/Cas9 mediated genome editing. One skilled in the art is familiar with these techniques.

### ENGRAmSCRIBE Molecular Recorder System

The ENGRAmSCRIBE platform combines features of mSCRIBE and ENGRAM. ENGRAmSCRIBE offers a long-term, compact, scalable and minimally disruptive DNA molecular recorder design in living cells. The ENGRAmSCRIBE systems includes a stgRNA locus that continuously directs dCas9 (or nCas9) fused to a cytidine deaminase to the stgRNA locus (FIG. 4), enabling continuous diversification of the stgRNA locus, while avoiding dsDNA breaks and shortening/lengthening of the stgRNA locus. As a result, mutations are continuously accumulated in the stgRNA locus as a function of stgRNA and d/nCas9-writer activity and expression, and can thus be used as a very compact memory register. Using stgRNA would allow to incorporate dC residues in the first 12 bp of the gRNA, thus expanding the memory capacity of the system. Thus, this platform enables to combine self-targeted writing into specific loci (thus achieving compact encoding with extended recording capacity) without needing to induce DNA double-strand breaks (thus avoiding cellular toxicity and extending the time-span of information that can be recorded). ENGRAmSCRIBE does not rely on stochastic deletion-based mutations to record information, thus enabling the chronicle of events to be deduced from the memory registers more easily. Similar to ENGRAM, the ENGRAmSCRIBE system offers a highly scalable design as multiple memory units that can operate orthogonally within the cell.

Provided herein are cells comprising the ENGRAmSCRIBE system. The SDS of the stgRNA in the ENGRAmSCRIBE system is cytosine rich (C-rich), providing substrate bases for the cytidine deaminase.

In some embodiments, repetitive sequences are inserted into the genome of a host cell, while in other embodiments, endogenous repetitive sequences are used. For example, DNA repeats in MUC1, MUC4 or telomeres of human genome may be targeted.

Non-repetitive sequences can also be used as a target (e.g. one guide RNA targeting one target site, or multiple guide RNAs targeting multiple target site). Having multiple target sites (e.g., either in repetitive form or in non-repetitive form targeted by multiple gRNAs) increases the recording capacity of the system, although a single target site is sufficient for recording.

The cytidine deaminase modules incorporated in the ENGRAM and ENGRAmSCRIBE introduce mutations into dC positions, resulting in a DNA lesion that is preferentially repaired as dT, although dG and dA are also generated at lower frequency. In ENGRAmSCRIBE, C-rich stgRNAs are used as starting memory loci, so that T, A, or G mutations will accumulate over time as a function of the duration and magnitude of stgRNA expression or d/nCas9-writer activity. For example, a stgRNA memory register with a 20-bp poly C specificity-determining sequence (SDS) would allow one to record up to 420˜1 trillion different memory states. Furthermore, the memory capacity of the system can be extended by increasing the range of mutations that can be written into DNA by using multiple different enzymes that can catalyze nucleotide changes (DNA writer modules). Unlike double-strand DNA breaks that are repaired by the error-prone non-homologous DNA end joining (NHEJ) repair pathway, the mutations that are introduced by cytidine deaminases are typically non-disruptive and do not introduce deletions. As a result, the chronicle of events (i.e., previous states) remain intact after each writing step, thus enabling faithfully tracking of event histories by sequencing the memory units. Furthermore, a standard curve for the average number of accumulated mutations observed per unit of time (or signal magnitude) can be obtained, which can then be used as a way to calibrate the system and measure the duration and/or magnitude values of signals. Since the system avoids double-strand DNA breaks, multiple orthogonal stgRNA memory registers can be safely used in parallel, thus allowing multiplexed recording of multiple signals directly in the genome of living cells. For example, different memory registers can be used to record different signals, or to simultaneously track cellular cues along with lineage history.

Introducing nicks into the DNA strand opposite to the deaminated base of DNA can enhance the incorporation of mutations into the sites of the deaminated bases. Thus, instead of dCas9, nCas9 can be fused to cytidine deaminases to enhance DNA writing efficiency (7). The editing efficiency of cytidine deaminases can be improved by fusing the uracil DNA glycosylase inhibitor (UGI) protein to the d/nCas9-cytidine deaminase fusion (8). Alternatively, the genes responsible for the repair of deaminated cytidine can be knocked down using CRISPR interference. In addition to cytidine deaminases, other types of base editors, such as adenosine deaminases (ADA) and/or proteins that cause mutator phenotypes such as MAGI (3-methyladenine DNA glycosylase), can be used (9).

### EpiSCRIBE Molecular Recorder System

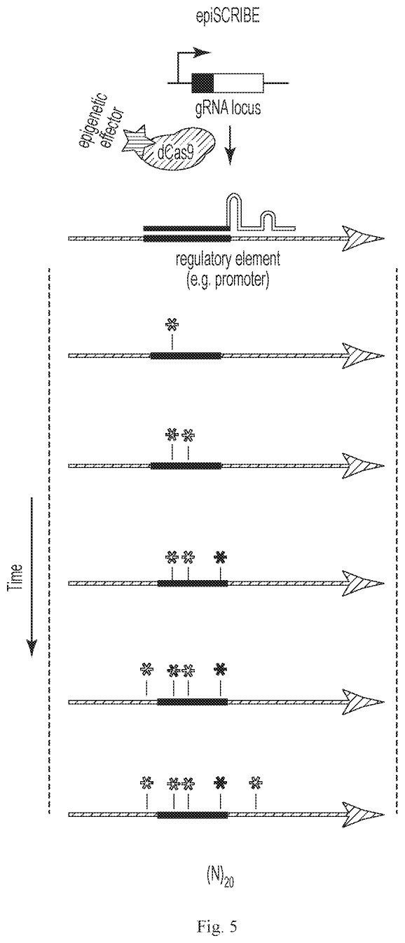

The epiSCRIBE (accumulative epigenetic modifications) system includes a dCas9 fused to an epigenetic effector domain targeted to a regulatory element (e.g. a promoter or an enhancer) by a complementary guide RNA (FIG. 5). The epigenetic effector domain introduces targeted epigenetic changes into the vicinity of the target sequence. The accumulation of these changes results in the activation or repression of the targeted regulatory element, which can be read out by functional assays or sequencing, and could be used as a way to trace cellular history. Unlike the other molecular recorder systems, this memory is stored in the epigenetic state of the DNA, avoiding the introduction of mutations in the target sequence.

Some aspects of the present disclosure provide cells comprising an epiSCRIBE systems. An “epigenetic modification” refers to a modification (e.g., addition or removal of a chemical group such as a methyl group or an acetyl group) to a genetic material (e.g., DNA) without substantially changing the sequence of the DNA. Non-limiting examples of an epigenetic modification includes DNA methylation, DNA demethylation, DNA hydroxymethylation, histone methylation, histone acetylation, histone phosphorylation, histone ubiquitination, histone citrullination, mRNA editing. An epigenetic modification influences (e.g., activates or suppresses) the expression or a genetic material (e.g., a gene). As used herein, an epigenetic modification encompasses modifications made to histones. A “histone” is a highly alkaline protein found in eukaryotic cell nuclei that package and order the DNA into structural units called nucleosomes. A histone modification is a covalent post-translational modification (PTM) to histone proteins which includes methylation, phosphorylation, acetylation, ubiquitination, and sumoylation. The PTMs made to histones can impact gene expression by altering chromatin structure or recruiting histone modifiers.

Accordingly, in some embodiments, the cell comprises an engineered nucleic acid comprising a nucleic acid comprising a regulatory element operably linked to a target sequence, a promoter operably linked to a nucleotide sequence encoding a guide ribonucleic acid (gRNA), and a fusion protein comprising a RNA-guided DNA binding domain (e.g., dCas9, nCas9, or dCpf1) fused to an epigenetic effector. An “epigenetic effector” refers to a protein that exerts an effect on the epigenetic states of a target site. Non-limiting examples of epigenetic effectors include any of the following classes of proteins: proteins acting as histones, histone variants or protamines; proteins performing post-translational modifications of histones or recognizing such modifications (histone modification ‘writers,’ ‘erasers’ or ‘readers’); proteins changing the general structure of chromatin (performing chromatin remodeling), including proteins that move, eject or restructure nucleosomes (ATP-dependent chromatin remodelers); proteins that incorporate histone variants into the nucleosomes; proteins assisting histone folding and assembly; proteins acting upon modifications of DNA or RNA in such a way that it affects gene expression, but not through RNA processing; and protein cofactors forming complexes with epigenetic factors, where complex formation is important for the activity (e.g., as described in Medvedeva et al., The Journal of Biological Databases and Curation, 2015).

One skilled in the art is familiar with methods of making fusion proteins. Any linker sequences known in the art and described herein may be used in the RNA-guided DNA binding domain-epigenetic effector fusion proteins described herein. In some embodiments, the RNA-guided DNA binding domain is fused to the N-terminus of the epigenetic effector. In some embodiments, the RNA-guided DNA binding domain is fused to the C-terminus of the epigenetic effector.

In some embodiments, the target sequence in the epiSCRIBE system is operably linked to a regulatory element. A “regulatory element” as used herein refers to a nucleotide sequence that regulates the expression of a gene (e.g., a gene downstream of the regulator element). Non-limiting examples of regulatory elements include promoters, transcriptional enhancers or suppressors. The regulatory element may be natural or synthetic.

RNA-guided DNA binding domain-epigenetic effector fusion protein is targeted by the gRNA to the target sequence, wherein the epigenetic effector introduces epigenetic modifications to the regulatory element in the vicinity of the target sequence, leading to activation of repression of a downstream gene (e.g., a gene encoding a detectable protein). Non-limiting examples of a detectable protein that may be used in the epiSCRIBE system include fluorescent proteins (e.g., eGFP, eYFP, eCFP, mKate2, mCherry, mPlum, mGrape2, mRaspberry, mGrape1, mStrawberry, mTangerine, mBanana, and mHoneydew), fluorescent RNAs (e.g., Spinach and Broccoli, as described in Paige et al., Science Vol. 333, Issue 6042, pp. 642-646, 2011, incorporated herein by reference), and enzyme that hydrolyzes an substrate to produce a detectable signal (e.g., a chemiluminescent signal). Such enzymes include, without limitation, beta-galactosidase (encoded by LacZ), horseradish peroxidase, or luciferase.

In some embodiments, a stgRNA is used in the epiSCRIBE system, enabling continuous generation of epigenetic modifications in the stgRNA locus.

### Directed and Recurring In Vivo Evolution—DRIVE

DRIVE enables the efficiently introduction of targeted mutations into sequences of interest on plasmid or genomic DNA, for example, in both prokaryotes and eukaryotes, independent of a host background. The DRIVE platform can be used to generate large libraries of protein, RNA and DNA variants in vivo, bypassing the bottlenecks associated with in vitro diversity generation methods. The DRIVE platform can readily replace the in vitro diversity generation steps in the established protein engineering systems such as phage display and yeast display, increasing the library diversity tremendously, while reducing the cost and labor required for building those libraries. Furthermore, because diversity generation is performed in vivo, this platform can be readily coupled with a continuous selection and screening setup. As such, these steps can be iterated automatically for many cycles, in some embodiments, without the need for human interruption, greatly facilitating and streamlining the evolutionary process. The DRIVE platform is useful, for example, in evolutionary engineering of genomically-encoded biomolecule scaffolds (e.g., therapeutic proteins such as antibodies as well as DNA and RNA aptamers), broadening phage host range, as well as many other biomedical and biotechnological applications described below. Furthermore, diversity generation can be linked to internal and external cellular cues, enabling a plethora of novel applications for engineering cellular phenotypes.

Exemplary features of DRIVE include, but are not limited to:
a tunable, reprogrammable, directed and continuous in vivo diversity generation strategy, which enables the production of a much larger and more diverse library relative to those produced by costly in vitro DNA synthesis methods (e.g., phage display and yeast display);
coupling to continuous selection and screening schemes, thus greatly facilitating and streamlining the evolutionary process;
targeting to produce libraries of variants of proteins, DNA and RNA scaffold of interest such as antibodies, synthetic and natural protein binding domains, RNA- and DNA-zymes and aptamers, as well as other applications such as broadening phage host range (e.g., by diversification of phage tail fibers);
interfacing with a host regulatory circuits, enabling control of the degree and timing of diversity generation;
building cells and gene circuits that can undergo accelerated evolution in response to internal and environmental cues (such as small molecule inducers); and
CRISPR-based, which renders DRIVE functional across different organisms, unlike current in vivo diversity generation technologies that are bound to a few organisms.

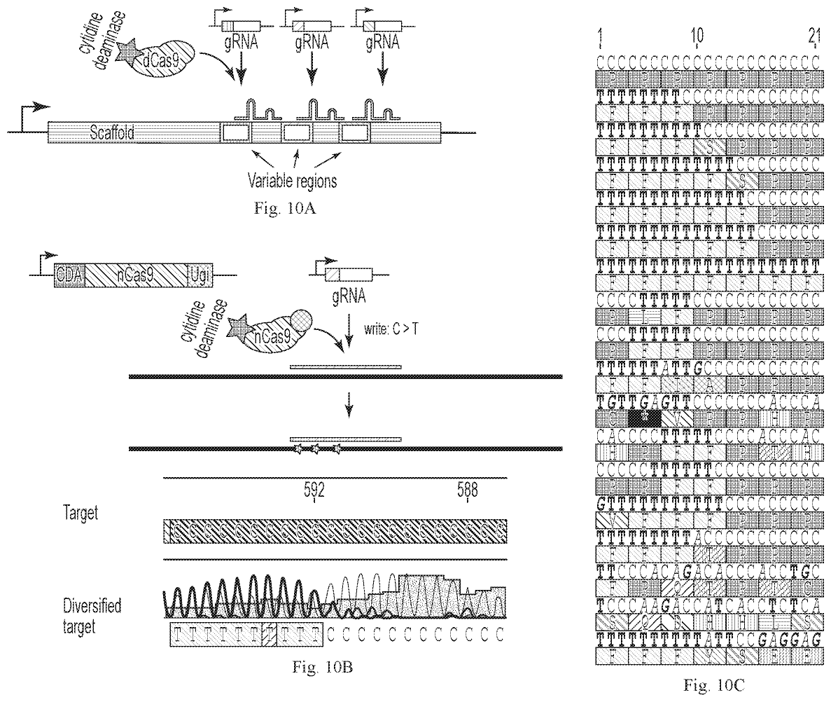

In order to generate targeted diversity in vivo without elevating the global mutation rate, the DRIVE platform uses d/nCas9 fused to a mutator domain/protein. For example, d/nCas9 fused to cytidine deaminases and/or Uracil DNA Glycosylase Inhibitor (ugi) can be used to mutate dC to dT, and with lower frequency dC to dG and dC to dA mutations. By expressing a complementary gRNA, the mutator protein can be direct to a desired target site (see, e.g., FIG. 10A). gRNA and mutator protein expression can be placed under the control of inducible promoters, for example, enabling the coupling of a desired signal to targeted diversity generation. The editing window can be tuned, for example, by changing the size of R-loop between the Specificity Determining Sequence (SDS) of gRNA and its target (e.g. by modifying SDS length) and by using different linker between Cas9 and cytidine deaminase. In addition to, or as alternative to, cytidine deaminase, other mutator domains may be used to generate other mutation spectrums and a more diversified library of variants. For example, adenine deaminases can be used to deaminate dA residues and generate dA to dG mutations. An ideal mutator for evolutionary engineering should be able to produce all the possible transition and transversion mutations in desired locations without elevating mutation rate. Mutator domains (i.e., base editor enzymes) such DNA glycosylases (e.g., alkA, alkB, Mag1 and AAG) can remove the glycosidic bond between the sugar and nitrogen base of damaged (and to some extent undamaged) bases of DNA and produce an apurinic/apyrimidinic (AP) site. The AP site is a non-coding residue and can then be filled by an error prone polymerase, leading to a random base substitution in that site, and the production of all the possible transition and transversion mutations in that site. Other domains such as reactive generator (ROS) proteins can also be used as mutator modules. Table 6 lists non-limiting examples of mutator domains that can be fused to dCas9 and/or nCas to generate various mutation spectrums. Depending on the application, different (or combinations of) mutator proteins with different mutation spectrums can be used.

TABLE 6
Exemplary Mutator Domains (also referred to herein as based editor enzymes).

| Mutator domain | Mutated residues | Type of mutations | outcome |
|---|---|---|---|
| Cytidine deaminase | dC | dC to dU | Mostly dC to dT |
| (e.g. APOBEC1, | | | mutations. Also |
| PmCDA) | | | generates dC to dA |
| | | | and dC to dG |
| | | | mutations with lower |
| | | | frequency |
| Adenine deaminase | dA | dA to dI | dA to dG |
| (e.g. ADAR) | | | |
| DNA glycosylases | Purines | Abasic (AP) site | Random insertion of |
| (e.g. alkA, alkB, | | | nucleotide across the |
| MAG1, AAG, | | | abasic site. |
| R.pabI) | | | |
| ROS generators (e.g., | All nucleotides | Oxidized bases | Random mutations |
| miniSOG, Killer | | | |
| Red, Killer Orange) | | | |

### DNA-Based Ordered Memory and Iteration Network Operating System—DOMINO

Building robust and scalable computation and memory platforms in living cells is one of the main goals of synthetic biology and is important for building sophisticated gene circuits for bioengineering and biomedical applications, for example. Provided herein, in some embodiments, is a highly transformative platform for building compact and scalable logic and memory operations in living cells. The platform enables, for example, dynamic and highly-efficient unidirectional manipulations of DNA with single-nucleotide resolution in living cells. The order and combination of these DNA writing events can be programmed and controlled by external or internal cellular cues, thus enabling the execution different combinatorial and sequential logic and memory operations in vivo. Furthermore, the platform can be readily interfaced with cellular regulatory circuits to control cellular phenotype at different genetic, epigenetic and transcriptional levels.

The DOMINO (DNA-based Ordered Memory and Iteration Network Operating system as provided herein uses highly efficient and precise DNA writing to manipulate DNA dynamically and efficiently with single-nucleotide resolution in living cells. The order and combinations of these DNA writing events can be easily programmed by changing gRNA sequences, which in turn can be controlled by internal and external (e.g. small molecule) inputs, allowing the execution various combinatorial and sequential logic and memory operations in vivo. These unidirectional and sequential DNA writing events will enable highly compact and scalable logic and memory operators. These operators, in some embodiments, can be layered to build more sophisticated gene circuits and can be interfaced with the synthetic or natural regulatory circuits. In some embodiments, the DOMINO platform can be combined with the established CRISPR-based gene regulation platforms such as CRISPR interference (CRISPRi) and CRISPR activator (CRISPRa), which have been shown to be functional across various organisms, to achieve a versatile and generalizable technology for endowing cells with synthetic logic and memory and programming cellular phenotypes.

Exemplary features of DOMINO include, but are not limited to:
dynamic in vivo information processing based on DOMINOS logic, including unidirectional and cascade-based DNA memory and computation operators;
realization of both combinatorial and sequential logic;
propagation delay and multi-inputs can be readily incorporated into gene circuits;
interfacing in trans with other circuits (e.g., with the host regulatory circuits) without the need for specific modifications (such as recombinase sites) in the host genome;
greater resistance to noise, using cumulative DNA writing, rather than transcriptional modulation to control the memory states;
CRISPR-based, which renders DOMINO functional across different organisms, unlike current in vivo diversity generation technologies that are bound to a few organisms;
DNA based, using only one protein component (Cas9-cytidine deaminase), in some embodiments;
lower metabolic load;
higher complexity resulting from the additional of functional domains such as transcriptional (i.e., activation and repression) and epigenetic modulators to the DNA writer protein, in some embodiments; and
compact circuits that can be built on plasmids and the output recorded in DNA and characterized in high-throughput using next-generation sequencing, for example.

### RNA Guided Nucleases

A “RNA-guided endonuclease” refers to a nucleases with DNA binding specificity mediated by a guide nucleotide sequence (e.g., a gRNA). RNA-guided endonucleases may be catalytically active (e.g., Cas9) or catalytically inactive (e.g., dCas9).

Non-limiting examples of RNA-guided endonucleases include Clustered regularly interspaced short palindromic repeats (CRISPR) associated protein 9 (Cas9) nucleases, e.g., Cas9 from Streptococcus pyogenes (e.g., as described in Jinek et al., Science 337:816-821(2012), incorporated herein by reference), and Cas9 from Prevotella and Francisella 1 (e.g., as described in Zetsche et al., Cell, 163, 759-771, 2015, incorporated herein by reference).

Cas9 nuclease sequences and structures are well known to those of skill in the art (see, e.g., Ferretti et al., Proc. Natl. Acad. Sci. 98:4658-4663(2001); Deltcheva E. et al., Nature 471:602-607(2011); and Jinek et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737, incorporated herein by reference.

In some embodiments, the RNA-guided endonuclease used herein is a Cas9 nuclease from Streptococcus pyogenes (Uniprot Reference Sequence: Q99ZW2) (SEQ ID NO: 18).

In some embodiments, Cas9 refers to a Cas9 from, without limitation: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1).

In some embodiments, the RNA-guided nuclease is a Clustered Regularly Interspaced Short Palindromic Repeats from Prevotella and Francisella 1 (Cpf1). Similar to Cas9, Cpf1 is also a class 2 CRISPR effector. It has been shown that Cpf1mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

In some embodiments, the present disclosure contemplates the use of a catalytically-inactive RNA-guided endonuclease as RNA-guided DNA binding domain, which is guided by the guide RNA to specific target sequences. The RNA-guided DNA binding domains may be fused to various DNA modifying enzymes (e.g., nucleases, deaminases, or epigenetic modifiers) for targeted modification of a target sequence. In some embodiments, the RNA-guided DNA binding domain is a catalytically-inactive Cas9 (dCas9). The DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science 337:816-821(2012); Qi et al., Cell 28; 152(5):1173-83 (2013). In some embodiments, a partially inactive Cas9 (e.g., a Cas9 with one inactive DNA cleavage domain and one active DNA cleavage domain) is used as the RNA-guided DNA binding domain of the present disclosure. A partially inactive Cas9 cleaves one of the two DNA strands in the target sequence and is referred to herein as a “Cas9 nickase (nCas9).” In some embodiments, the nCas9 comprises an inactive RuvC domain. In some embodiments, the nCas9 comprises a D10A mutation that inactivates the RuvC domain. Non-limiting, exemplary dCas9 and nCas9 sequences are provided herein.

In some embodiments, the RNA-guided DNA binding domain is a catalytically inactive Clustered Regularly Interspaced Short Palindromic Repeats from Prevotella and Francisella 1 (dCpf1). The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 19) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 19. It is to be understood that any mutations, e.g., substitution mutations, deletions, or insertions that inactivates the RuvC domain of Cpf1 may be used in accordance with the present disclosure. Exemplary RNA-guided nuclease sequences are provided in Table 3.

### Guide RNA.

A RNA-guide nuclease is guided by a guide RNA (gRNA) to its target sequence. A native gRNA is comprised of a 20 nucleotide (nt) Specificity Determining Sequence (SDS), which specifies the DNA sequence to be targeted, and is immediately followed by a 80 nt scaffold sequence, which associates the sgRNA with Cas9. In addition to sequence homology with the SDS, targeted DNA sequences possess a Protospacer Adjacent Motif (PAM) (5′-NGG-3′) immediately adjacent to their 3′-end in order to be bound by the Cas9-sgRNA complex and cleaved. When a double-stranded break is introduced in the target DNA locus in the genome, the break is repaired by either homologous recombination (when a repair template is provided) or error-prone non-homologous end joining (NHEJ) DNA repair mechanisms, resulting in mutagenesis of targeted locus. Even though the normal DNA locus encoding the sgRNA sequence is perfectly homologous to the sgRNA, it is not targeted by the standard Cas9-sgRNA complex because it does not contain a PAM.

Unlike the wild-type CRISPR/Cas9 system, wherein a gRNA is specific for a single target, the molecular recorders of the present disclosure, in some embodiments, comprise a guide RNA with iterative self-targeting capability such that it directs a Cas9 nuclease (or other RNA-guided nuclease) to cleave the DNA that encodes the guide RNA, leading to generation of indels in the DNA that encodes the guide RNA, when the double-strand break is repaired (e.g., by NHEJ). The “self-targeting” activity of the gRNA can be achieved by introducing a PAM sequence into its own coding sequence, adjacent to an SDS sequence, e.g., as described in Perli, S D et al., Science. 2016 Sep. 9; 353(6304) and International Publication No. WO 2016/183438, each of which is incorporated herein by reference in its entirety). Introduction of a PAM sequence (e.g., “NGG”) into the template DNA leads to a modified gRNA that complexes with Cas9 (or other RNA-guided nuclease) and cleaves the DNA sequence encoding the gRNA, resulting in generation of indels (deletions or insertions) in the DNA sequence encoding the gRNA, while the PAM sequence is preserved in most cases. The gRNA that is modified to have self-targeting activity is referred to herein as a self-targeting guide RNA. The stgRNA can direct the Cas9 nuclease (or other RNA-guided nuclease) repeatedly to the DNA encoding the stgRNA, creating additional indels.

Thus, some aspects of the present disclosure are directed to an engineered nucleic acid comprising a promoter operably linked to a nucleotide sequence encoding a guide ribonucleic acid (gRNA) that comprises a specificity determining sequence (SDS) and a protospacer adjacent motif (PAM).

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a 20 nucleotide (nt) Specificity Determining Sequence (SDS), which specifies the DNA sequence to be targeted, and is immediately followed by a 80 nt scaffold sequence, which associates the gRNA with Cas9. In some embodiments, an SDS of the present disclosure has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS is 20 nucleotides long. For example, the SDS may be 15, 16, 17, 18, 19, 20, 21, 22, 23, 24, or 25 nucleotides long. At least a portion of the target DNA sequence is complementary to the SDS of the gRNA. For Cas9 to successfully bind to the DNA target sequence, a region of the target sequence is complementary to the SDS of the gRNA sequence and is immediately followed by the correct protospacer adjacent motif (PAM) sequence (e.g., NGG for Cas9 and TTN, TTTN, or YTN for Cpf1). In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. In some embodiments, the SDS of template DNA or target DNA may differ from a complementary region of a gRNA by 1, 2, 3, 4 or 5 nucleotides.

In addition to the SDS, the gRNA comprises a scaffold sequence (corresponding to the tracrRNA in the native CRISPR/Cas system) that is required for its association with Cas9 (referred to herein as the “gRNA handle”). In some embodiments, the gRNA comprises a structure 5′-[SDS]-[gRNA handle]-3′. In some embodiments, the scaffold sequence comprises the nucleotide sequence of 5′-guuuuagagcuagaaauagcaaguuaaaauaaaggcuaguc cguuaucaacuugaaaaaguggcaccgagucggugcuuuuu-3′ (SEQ ID NO: 1). Other non-limiting, suitable gRNA handle sequences that may be used in accordance with the present disclosure are listed in Table 2.

In some embodiments, the guide RNA is about 15-120 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97, 98, 99, 100, 101, 102, 103, 104, 105, 106, 107, 108, 109, 110, 111, 112, 113, 114, 115, 116, 117, 118, 119, or 120 nucleotides long. In some embodiments, the guide RNA comprises a sequence of 10, 11, 12, 13, 14, 15, 16, 17, 18, 19, 20 or more contiguous nucleotides that is complementary to a target sequence. Sequence complementarity refers to distinct interactions between adenine and thymine (DNA) or uracil (RNA), and between guanine and cytosine.

A “protospacer adjacent motif” (PAM) is typically a sequence of nucleotides located adjacent to (e.g., within 10, 9, 8, 7, 6, 5, 4, 3, 3, or 1 nucleotide(s) of a target sequence). A PAM sequence is “immediately adjacent to” a target sequence if the PAM sequence is contiguous with the target sequence (that is, if there are no nucleotides located between the PAM sequence and the target sequence). In some embodiments, a PAM sequence is a wild-type PAM sequence. Examples of PAM sequences include, without limitation, NGG, NGR, NNGRR(T/N), NNNNGATT, NNAGAAW, NGGAG, and NAAAAC, AWG, CC. In some embodiments, a PAM sequence is obtained from Streptococcus pyogenes (e.g., NGG or NGR). In some embodiments, a PAM sequence is obtained from Staphylococcus aureus (e.g., NNGRR(T/N)). In some embodiments, a PAM sequence is obtained from Neisseria meningitidis (e.g., NNNNGATT). In some embodiments, a PAM sequence is obtained from Streptococcus thermophilus (e.g., NNAGAAW or NGGAG). In some embodiments, a PAM sequence is obtained from Treponema denticola NGGAG (e.g., NAAAAC). In some embodiments, a PAM sequence is obtained from Escherichia coli (e.g., AWG). In some embodiments, a PAM sequence is obtained from Pseudomonas auruginosa (e.g., CC). Other PAM sequences are contemplated. A PAM sequence is typically located downstream (i.e., 3′) from the target sequence, although in some embodiments a PAM sequence may be located upstream (i.e., 5′) from the target sequence.

In some embodiments, a gRNA is a self-targeting stgRNA. A “stgRNA” is a gRNA that complexes with Cas9 and guides the stgRNA/Cas9 complex to the DNA sequence encoding itself. To obtain a stgRNA, a PAM sequence is introduced into the gRNA as such that the gRNA/Cas9 complex would recognize the gRNA-encoding DNA as a target sequence. In some embodiments, the PAM is introduced adjacent to (e.g., within 10, 9, 8, 7, 6, 5, 4, 3, 3, or 1 nucleotide(s) of the SDS). In some embodiments, the PAM is introduced “immediately adjacent to” the SDS (i.e., continuous with the SDS). In some embodiments, the PAM is introduced by mutating the nucleotides in the gRNA handle that is adjacent to the SDS. For example, for a gRNA handle from S. pyogenes (5′-GUUUAAGAGCUAUGCUG GAAAGCCACGGUGAAAAAGUUCAACUAUUGCCUGAUCGGAAUAAAUUUGAAC GAUACGACAGUCGGUGC-3′ (SEQ ID NO: 16)), the first 3 nucleotides (underlined) may be modified (e.g., GUU change to GGG) to create a PAM sequence that is recognized by the S. pyogenes Cas9. In some embodiments, to maintain the overall structure and activity of the stgRNA, more nucleotides in the gRNA handle may be modified. In some embodiments, the gRNA handle of a stgRNA comprises the nucleotide sequence of GGGTTAGAGCTAGAAATAGCAAGTTAACCTAAGGCTAGTCCGTTATCAACTTGA AAAAGTGGCACCGAGTCGGTGCTTTT (SEQ ID NO: 17, mutations compared to the wild-type gRNA handle are underlined). The examples provided herein are not meant to be limiting. Any PAM sequences may be introduced (e.g., via mutating the gRNA handle sequence or via insertion) adjacent to the SDS of the gRNA to create a stgRNA.

A “target site” or “target sequence” refers to a sequence within a nucleic acid molecule (e.g., a DNA molecule) that is cleaved or modified by the methods described herein. In some embodiments, the target sequence is a polynucleotide (e.g., a DNA), wherein the polynucleotide comprises a coding strand (a nucleic acid strand that codes for a product) and a complementary strand (a nucleic acid strand that is complementary to the coding strand). In some embodiments, the target sequence is a sequence in the genome of a prokaryotic cell (e.g., a bacterial cell). In some embodiments, the target sequence is a sequence in the genome of an eukaryotic cell. In some embodiments, the target sequence is a sequence in the genome of a mammal. In some embodiments, the target sequence is a sequence in the genome of a human. In some embodiments, the target sequence is a sequence in the genome of a non-human animal. When a stgRNA is used, the target site may refer to the stgRNA locus, or other target sites that the stgRNA is able to target.

The molecular recorder systems of the present disclosure comprises an enzyme (e.g., a DNA modifying enzyme) that introduces mutations to the target site. Different enzymes may be used to introduce different types of mutations. Also provided herein are different molecular recorder systems, their unique features, and their use in recording cellular memory.

### Engineered Nucleic Acids

A “nucleic acid” is at least two nucleotides covalently linked together, and in some instances, may contain phosphodiester bonds (e.g., a phosphodiester “backbone”). An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Engineered nucleic acids of the present disclosure may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA, a protein and/or an RNA interference molecule). Examples of genetic elements of the present disclosure include, without limitation, promoters, nucleotide sequences that encode gRNAs and proteins, SDSs, PAMs and terminators.

Engineered nucleic acids of the present disclosure may be produced using standard molecular biology methods (see, e.g., Green and Sambrook, Molecular Cloning, A Laboratory Manual, 2012, Cold Spring Harbor Press).

In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Also provided herein are vectors comprising engineered nucleic acids. A “vector” is a nucleic acid (e.g., DNA) used as a vehicle to artificially carry genetic material (e.g., an engineered nucleic acid) into another cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

### Promoters

Engineered nucleic acids of the present disclosure may comprise promoters operably linked to a nucleotide sequence encoding, for example, a gRNA. A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. Nos. 4,683,202 and 5,928,906).

Contemplated herein, in some embodiments, are RNA pol II and RNA pol III promoters. Promoters that direct accurate initiation of transcription by an RNA polymerase II are referred to as RNA pol II promoters. Examples of RNA pol II promoters for use in accordance with the present disclosure include, without limitation, human cytomegalovirus promoters, human ubiquitin promoters, human histone H2A1 promoters and human inflammatory chemokine CXCL 1 promoters. Other RNA pol II promoters are also contemplated herein. Promoters that direct accurate initiation of transcription by an RNA polymerase III are referred to as RNA pol III promoters. Examples of RNA pol III promoters for use in accordance with the present disclosure include, without limitation, a U6 promoter, a H1 promoter and promoters of transfer RNAs, 5S ribosomal RNA (rRNA), and the signal recognition particle 7SL RNA.

Promoters of an engineered nucleic acids may be “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Examples of cytokines include, but are not limited to, eotaxin-2, MPIF-2, eotaxin-3, MIP-4-alpha, Fas Fas/TNFRSF6/Apo-1/CD95, FGF-4, FGF-6, FGF-7, FGF-9, Flt-3 Ligand fms-like tyrosine kinase-3, FKN or FK, GCP-2, GCSF, GENE Glial, GITR, GITR, GM-CSF, GRO, GRO-α, HCC-4, hematopoietic growth factor, hepatocyte growth factor, 1-309, ICAM-1, ICAM-3, IFN-γ, IGFBP-1, IGFBP-2, IGFBP-3, IGFBP-4, IGFBP-6, IGF-I, IGF-I SR, IL-1α, IL-10, IL-1, IL-1 R4, ST2, IL-3, IL-4, IL-5, IL-6, IL-8, IL-10, IL-11, IL-12 p40, IL-12p′70, IL-13, IL-16, IL-17, I-TAC, alpha chemoattractant, lymphotactin, MCP-1, MCP-2, MCP-3, MCP-4, M-CSF, MDC, MIF, MIG, MIP-1α, MIP-1β, MIP-1δ, MIP-3α, MIP-3β, MSP-a, NAP-2, NT-3, NT-4, osteoprotegerin, oncostatin M, PARC, PDGF, PlGF, RANTES, SCF, SDF-1, soluble glycoprotein 130, soluble TNF receptor I, soluble TNF receptor II, TARC, TECK, TGF-beta 1, TGF-beta 3, TIMP-1, TIMP-2, TNF-α, TNF-β, thrombopoietin, TRAIL R3, TRAIL R4, uPAR, VEGF and VEGF-D.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Other inducible promoter systems are known in the art and may be used in accordance with the present disclosure.

In some embodiments, inducible promoters of the present disclosure function in prokaryotic cells (e.g., bacterial cells). Examples of inducible promoters for use prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), σS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, Lad, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), GS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and GB promoters. Other inducible microbial promoters may be used in accordance with the present disclosure.

In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

### Cells and Cell Expression

Engineered nucleic acids of the present disclosure may be expressed in a broad range of host cell types. In some embodiments, engineered nucleic acids are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, engineered nucleic acid constructs are expressed in mammalian cells. For example, in some embodiments, engineered nucleic acid constructs are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSYSY human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature (e.g., an engineered nucleic acid encoding a gRNA). In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis (e.g., gRNA/Cas9-mediated mutagenesis). In some embodiments, a cell is modified by introducing an engineered nucleic acid into the cell in order to produce a genetic change of interest (e.g., via insertion or homologous recombination).

In some embodiments, an engineered nucleic acid construct may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acid constructs of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Some aspects of the present disclosure provide cells that comprises 1 to 10 engineered nucleic acids (e.g., engineered nucleic acids encoding gRNAs). In some embodiments, a cell comprises 1, 2, 3, 4, 5, 6, 7, 8, 9, 10 or more engineered nucleic acids. It should be understood that a cell that “comprises an engineered nucleic acid” is a cell that comprises copies (more than one) of an engineered nucleic acid. Thus, a cell that “comprises at least two engineered nucleic acids” is a cell that comprises copies of a first engineered nucleic acid and copies of an engineered second nucleic acid, wherein the first engineered nucleic acid is different from the second engineered nucleic acid. Two engineered nucleic acids may differ from each other with respect to, for example, sequence composition (e.g., type, number and arrangement of nucleotides), length, or a combination of sequence composition and length. For example, the SDS sequences of two engineered nucleic acids in the same cells may differ from each other.

Some aspects of the present disclosure provide cells that comprises 1 to 10 episomal vectors, or more, each vector comprising, for example, an engineered nucleic acids (e.g., engineered nucleic acids encoding gRNAs). In some embodiments, a cell comprises 1, 2, 3, 4, 5, 6, 7, 8, 9, 10 or more vectors.

Also provided herein, in some aspects, are methods that comprise introducing into a cell an (e.g., at least one, at least two, at least three, or more) engineered nucleic acid or an episomal vector (e.g., comprising an engineered nucleic acid). As discussed elsewhere herein, an engineered nucleic acid may be introduced into a cell by conventional methods, such as, for example, electroporation, chemical (e.g., calcium phosphate or lipid) transfection, fusion with bacterial protoplasts containing recombinant plasmids, transduction, conjugation, or microinjection of purified DNA directly into the nucleus of the cell.

### Methods

Further provided herein are methods of generating different types of random additive barcodes in a target site (e.g., the stgRNA locus or other genomic loci) in a cell. The methods comprise maintaining the cells described herein under conditions suitable for the introduction of the different types of barcodes (e.g., suitable for enzymatic cleavage and addition of random nucleotides).

In some embodiments, cells comprising the ramSCRIBE system are maintained under conditions that result in the addition of random nucleotides to the SDS. In some embodiments, cells comprising the ENGRAM or ENGRAmSCRIBE system are maintained under conditions that result in targeted mutations in the target site (e.g., the array of repetitive dC-rich DNA sequence at the dC positions, or the C-rich SDS region of an stgRNA). In some embodiments, cells comprising the epiSCRIBE system are maintained under conditions that result in an accumulation of targeted epigenetic changes in the vicinity of the target sequence.

In some embodiments, the promoter that is operably linked to the nucleotide sequence encoding the gRNA or stgRNA is an inducible promoter. As such, the expression of the stgRNA may be coupled with an inducer signal, e.g., a signal produced by a cellular event. The expression of the stgRNA triggers the cleavage of a target site (e.g., the SDS of the stgRNA), including the stgRNA locus itself, following by the addition of random nucleotides by TdT during NHEJ. Repeated signals trigger multiple rounds of Cas9 cleavage of the target site and sequential addition (i.e., lengthening) of the target site (e.g., the SDS of the stgRNA). The additional sequence added by the process at the target site may be referred to as “barcodes,” which may be detected via any known techniques for nucleotide sequence determination (e.g., next-generation sequencing). The presence of the “barcodes” indicate the occurrence of the cellular event. Further, the sequential addition of the “barcodes” enable cellular lineage tracing. The modification generated to the target in the previous round is not obscured by the modifications generated in the next round, allowing unambiguous tracing of the “barcodes.”

In some embodiments, the “barcodes” are traced via sequencing of the target site. In some embodiments, the sequence is next-generation sequencing. In the case of epiSCRIBE, methods of detecting epigenetic modifications are used. In some embodiments, epigenetic modifications are detected by in vitro reporter assays or in vivo function assays. For example, if a reporter (e.g. GFP) is placed under control of the regulatory element (e.g. promoter), the activity of the promoter can be monitored over time.

In some embodiments, the molecular recorders described herein may be coupled with downstream synthetic circuits. For example, if a site specific recombinase is placed under the control of the regulatory element being targeted by an epiSCRIBE system, once the epigenetic memory accumulates to a certain threshold, it activates expression of the downstream recombinase which in turn could flip a downstream target flanked by recombinase target site. As such, the epigenetic memory can be converted into some form of permanent memory. Similar forms of interfacing biological memory and synthetic gene circuits are also contemplated herein.

### Exemplary Applications

The molecular recorders described herein, in some embodiments, are long-term, compact, scalable, and minimally disruptive DNA writers and can be used in a broad set of applications and communities. The molecular recorders described herein enable unprecedented ability to study spatiotemporal molecular events in their natural environmental contexts. For example, the molecular recorders may be used in developmental biology to perform long-term and high-resolution lineage tracking experiments in mammals, which has been impossible to date due to the lack of scalable and long-term methodologies.

As another example, the molecular recorders described herein may be used in neuroscience to map neural activity by driving the activity of DNA writers with regulators that respond to neural activity. Neuronal connectivity may also be mapped by using viruses that can cross between synapses and leave a record of pre-synaptic and post-synaptic neuronal barcodes in DNA.

Further, the molecular recorders described herein may be used in cancer biology to study the development of tumors from cancer stem cells to gain deeper insight into the cellular and environmental cues that are involved in tumor heterogeneity.

The molecular recorders described herein may also be used to encode arbitrary information into the DNA of living cells for DNA storage applications, to build sensors within the body or in the environment that sense and later report pathogens, toxins, or other signals of interest.

Additional non-limiting examples of applications in which the molecular recorders may be used are provided below.

### Lineage Tracing

The ENGRAmSCRIBE platform can be used to produce a high-resolution lineage map of Caenorhabditis elegans (C. elegans), a worm with only 959 cells in its entire body that has been used extensively as a model organism for developmental studies. The recorder can be genetically encoded into C. elegans embryos and lineage trajectories can be tracked by single-cell sequencing. The obtained results can then be validated by comparing them with the published cellular lineage map of C. elegans or independent imaging-based lineage tracing techniques. The approach can be extended to higher eukaryotes, where tracing of the developmental history of every cell in the human body is desired.

Alternatively, the recorder components (stgRNA and/or the d/nCas-cytidine deaminase fusion) can be placed under the control of lineage specific promoters to produce a lineage history of specific tissue/cell type. For example, they can be placed under the control of neural specific promoters to study development of different neural lineages and cell-types.

### Neural Activity Recording

The ENGRAmSCRIBE recorders can be used to record neural activity and map neural circuitry in the brain of live animals. The ENGRAmSCRIBE stgRNA can be linked to neural activity by placing it under the control of neuronal immediate early gene promoters (e.g. c-fos promoter) that are rapidly induced by neuronal activity. The neural activity-inducible stgRNAs can then be genomically encoded in the brain and be used as memory registers to record neural activity. Mutation accumulation of a known neural stimuli/promoter pair can be used to calibrate the recorder activity and as a reference to measure unknown neural activities.

Alternatively, the DNA recording can be combined with single-cell sequencing to map the neural circuitry that respond to a specific stimulus by identifying neurons that have accumulated mutations in their stgRNA memory register.

The ENGRAmSCRIBE recorders may be used in an animal model. For example, they can be used to study and map neural circuitry in Caenorhabditis elegans (C. elegans), a worm with only 302 neurons that has been used extensively as a well-established model to study neural circuitry. For example, the worm harboring genetically encoded neuronal activity inducible ENGRAmSCRIBE recorders can be exposed to different olfactory stimuli, allowing recording of the activities of individual neurons that are activated in response to a given stimuli in the stgRNA DNA memory registers, which can be later retrieved by single-cell sequencing. Combining the data with the identity of the activated neurons will reveal the neural circuitry that is activated in response to a given stimulus. The results can then be further validated independently by neural activity imaging techniques, and compared with the known neural circuitry map of given stimuli. The strategy can be extended to more complex neural circuits in the higher eukaryotes and human brain.

Instead of neural activity responsive promoters, other promoters and regulatory elements can also be used to record corresponding biological signals. The recorders can be combined and multiplexed to record multiple signals concurrently, or perform concurrent lineage tracing and signal dynamics recording.

Synthetic Lamarckian Evolution.

The hypermutagensis enabled by ENGRAM and ENGRAmSCRIBE systems can be used to increase the mutation rate of specific genomic segments connected to a phenotype of interest without increasing the global mutation rate. Synthetic circuits can be designed to link the activity of the recorders to cellular fitness, thus enabling building of organisms and synthetic gene circuits that could continuously and autonomously undergo Lamarckian evolution in response to signals of interest.

### Continuous In Vivo Evolution

In Vivo Diversity Generation and Biomolecule Scaffold Engineering.

Evolutionary engineering by continuous diversification of protein scaffolds and selection of desired variants is a powerful strategy to improve natural biomolecules scaffolds and to evolve new ones. For example, DRIVE may be used to evolve therapeutic biomolecules to target pathogens or cancer cells, to develop new protein-binding molecules, RNA and DNA-enzymes and aptamers, to change bacteriophage host range, among many other applications. As describe above, DRIVE platform offers a modular, tunable and easily programmable strategy for in vivo diversity generation that overcomes many limitations associated with in vitro diversity generation methods. The technology enables to introduce targeted mutations to genetically-encoded biomolecule scaffolds without increase the global mutation rate.

The DRIVE methods provided herein may be used to produce variant libraries that are more diverse than current in vitro diversity generation methods, which are limited by a transformation step. In some embodiments, in vitro diversity generation may be combined with in vivo diversity generation (e.g., start with a synthesized library, and diversify it further in vivo by DRIVE platform) to further increase diversity.

The DRIVE technology provided herein may also be used to diversify a single epitope. In vivo diversity generation can be multiplexed and can target multiple loci (e.g., multiple epitopes of antibody) for library generation, thus resulting much larger and diverse libraries that possible using in vitro mutagenesis.

Additionally, since the in vivo diversity generation achieved by DRIVE is mediated by CRISPR-Cas9, which has been shown to be functional in mammalian cells, it can be applied to mammalian cells. Extending evolutionary engineering techniques to mammalian cells, which have been limited before due to limited transformation efficiency of these cells, is another advantage of the DRIVE technology, opening up new avenues for performing biomolecule evolution in mammalian cell cultures, in a continuous and readily iterative manner.

Another advantage of DRIVE technology is that it transforms library generation into a streamlined and continuous process, in some embodiments, enabling iteration of many rounds of diversity generation and screening with minimal handling. In some embodiments, every step following the initial introduction of the scaffold of interest is conducted within cells; thus, there is no need for separate diversity generation and screening steps, and these steps can be iterated many times without in vitro DNA manipulations. Furthermore, unlike the current technologies, which are limited to species with high transformation efficiency such as yeast and E. coli, DRIVE technology can be applied to evolve proteins in non-traditional and less-transformable species. As Cas9-based systems have been shown to be functional in various organisms, the scaffolds can be engineered in their native contexts, or in orthogonal model organisms with well-established genetic tools.

Therefore, the elimination of the many transformation steps required to test an array of proteins represents a significant advancement. With this DRIVE technology, it is possible to continuously generate a huge amount of diversity in vivo, much larger than possible with in vitro methods, and without the need for in vitro DNA synthesis and passing through transformation bottlenecks. As the genetically-encoded moieties are diversified, cells can be screened for the particular phenotype of interest. A continuous cycle of biomolecule diversification and functional screening can be set in motion, for example, eliminating the cumbersome process of in vitro library generation and testing protein variations in discrete steps.

Engineering and Broadening Phage Host Range.

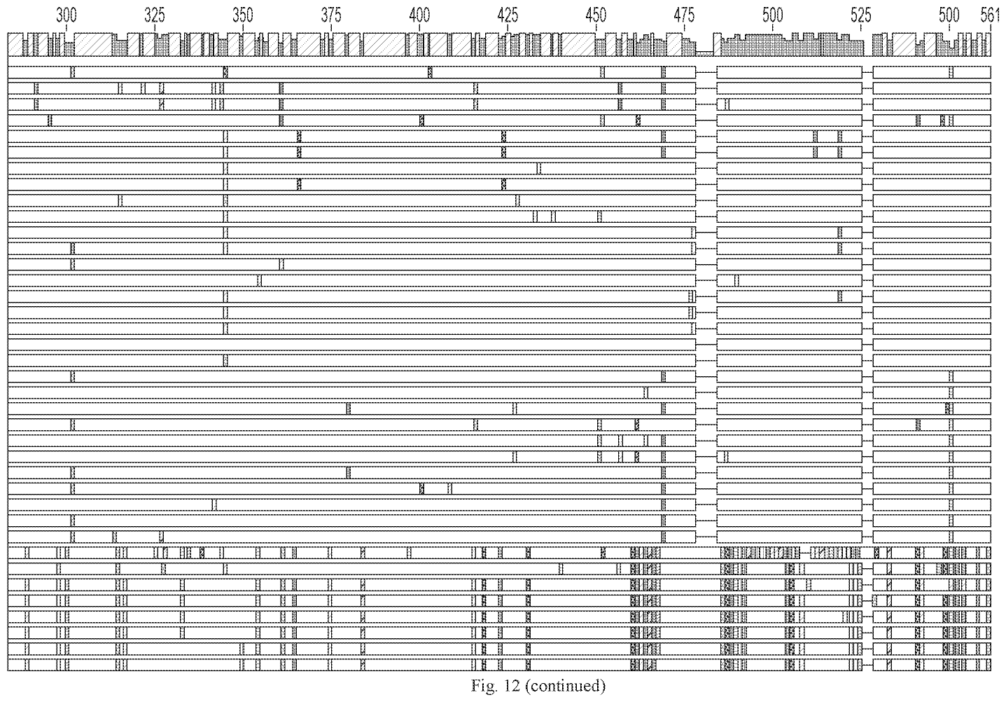

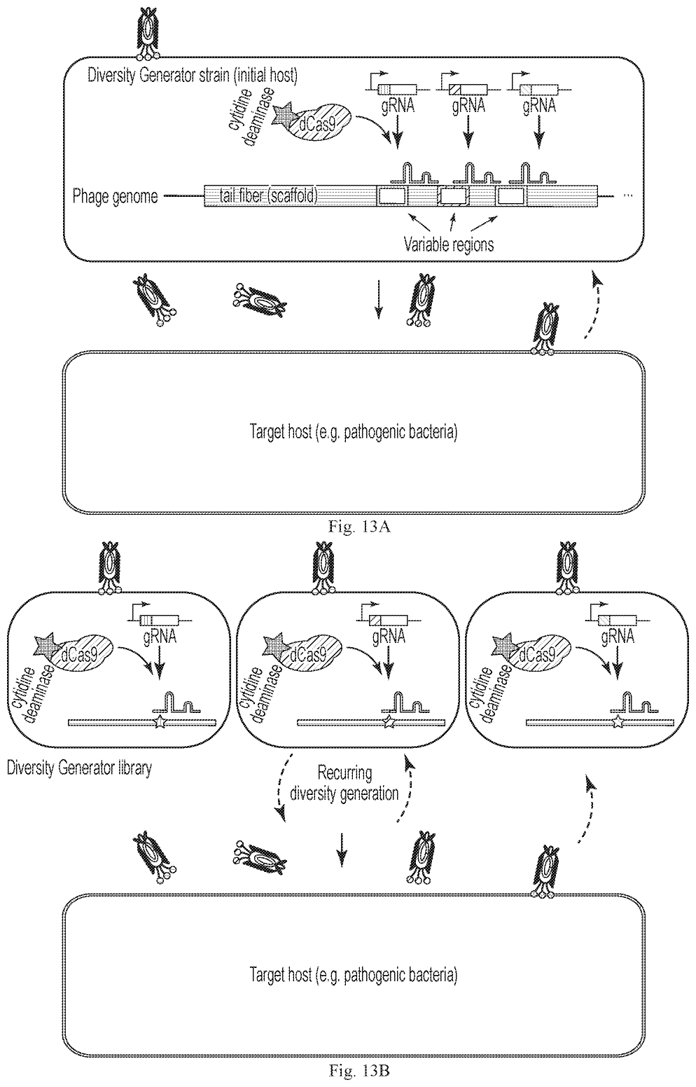

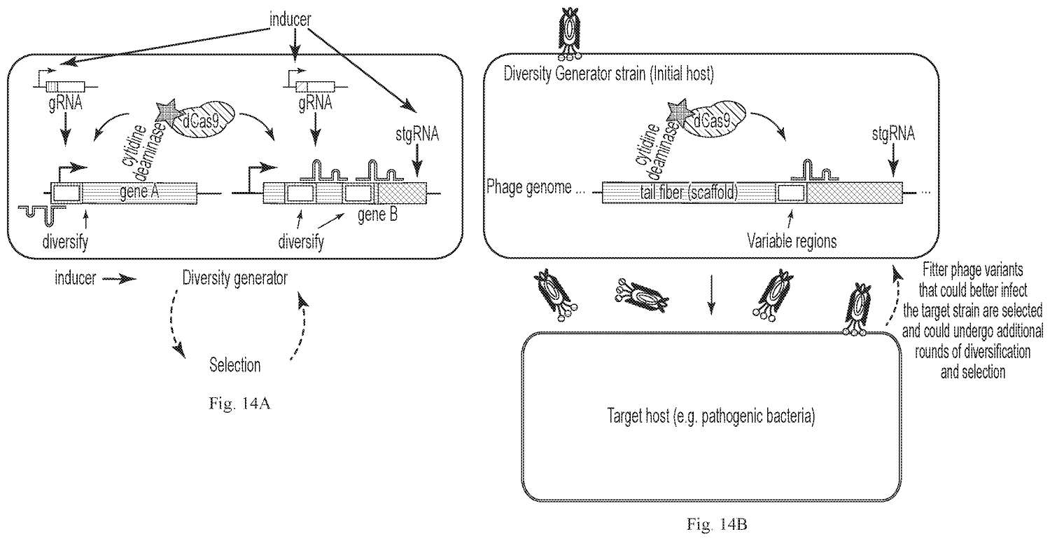

DRIVE technology can be applied, in some embodiments, for engineering and broadening phages (bacteriophage) host range in a continuous fashion for biomedical and biotechnological applications (e.g. to kill pathogenic bacterial), providing a potential treatment for antibiotic-resistant bacterial infections due to the rise of multi-drug resistant tuberculosis or methicillin-resistant Staphylococcus aureus (MRSA). One of the major determinant of bacteriophages host range is the specificity of their tail fiber, by which the bacteriophage interact with their host. Tail fiber proteins are an example of scaffold protein that shows conservation across many different types of phages, with certain variable positions (e.g., in the C-terminus) (FIG. 12). The variable regions are often involved in host specificity. Altering variable regions in tail fibers, and other host-range determinant sequences can change the phage host range (FIGS. 13A-13B).

Synthetic Lamarckian Evolution on Demand.

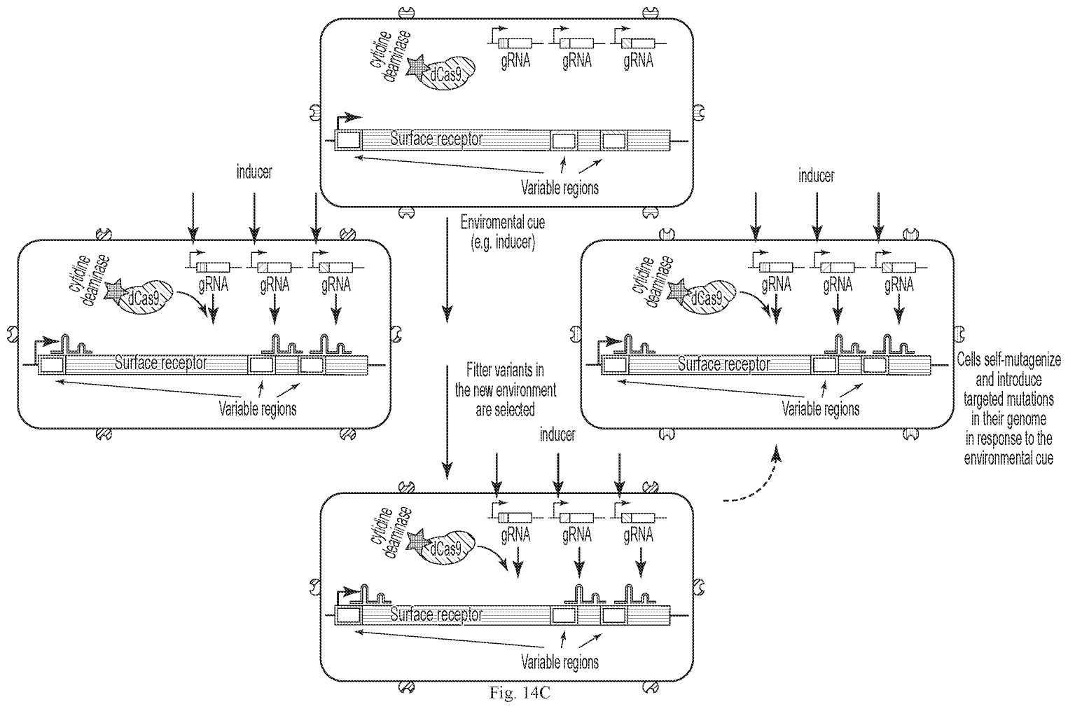

The DRIVE platform components, e.g., the mutator protein and gRNA, in some embodiments, can be placed under the control of inducible promoters and linked to internal and external cues. As such, cells can be endowed with the ability to diversify their genome on demand (e.g., environmental signals, such as small molecules) and at very specific sites. Under a selective pressure, these variants compete with each other and undergo accelerated evolution, similar to Lamarckian evolution. Cells and organisms that are endowed with a Lamarckian evolution mechanism can adapt to new environments much faster than those that adopt solely based on Darwinian evolution. As such, synthetic gene circuits and cells can be engineered to elevate their evolution rate when needed (when adapting to a new environment) and to taper down this process when adapted to the environment. For example, phage harboring DRIVE mutator circuits can be designed so that they can elevate mutation rate of their tail fiber autonomously and site-specifically when adapting to infect a new host (see, e.g., FIGS. 14A-14C). Once adapted, because mutagenesis is no longer needed and may be deleterious to phage infection, the circuit can then turn down the mutagenesis process, enabling phage to replicate efficiently in the new host. As another example, bacteria may be designed to mutagenize their surface receptors (or other genetic components connected to their fitness in the new environment) when exposed to a new environments (e.g., gastrointestinal tract), to allow them to adapt faster to new environment.

Functional Screening.

Functional screening is a powerful strategy to decipher molecular architecture and underlying mechanisms of cellular phenotypes. The DRIVE platform enables large-scale functional screening, e.g., in prokaryotes and eukaryotes. This is particularly advantageous for use in eukaryotes where many perturbations cannot be made by knockout or transcriptional regulations. For example, single nucleotide mutation or a few mutations in the regulatory elements of a gene using DRIVE result in expression patterns that is different from complete gene knockout or strong up- or down-regulations. DRIVE platform offers a high level of control on the type of perturbation in gene expression (i.e., knockout, and various degrees of up- and down regulation mutations can be readily produced). Because perturbations generated by DRIVE platform are in form of permanent mutations, the perturbations can be applied iteratively, without necessarily keeping the gRNAs in the cells, increasing the perturbation scale. As such, the DRIVE method can be easily scaled and multiplexed to many genes and tracked by high-throughput sequencing.

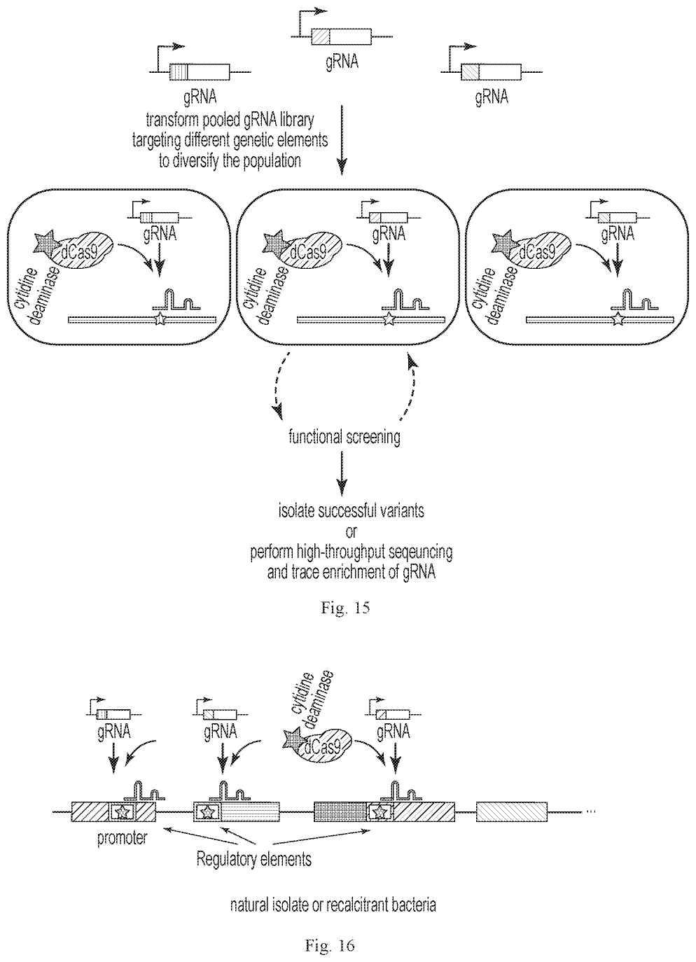

By targeting the DNA mutator proteins to ORFs and regulatory elements (e.g. promoters, ribosome binding sites, repressor and activator operator sites, etc.), for example, one can general knockouts, or downregulate and/or upregulate gene expression (FIG. 15). For example, cytidine deaminase-d/nCas9 writers can be used to mutate CAG codons to TAG to knockout the corresponding gene. Alternatively, cytidine deaminase-d/nCas9 writers can be targeted to promoter regulatory elements (e.g. −10 and −35 boxes), transcription operator sites or RBS to up-regulate or down-regulate gene expression. gRNA pooled libraries can be designed, in some embodiments, to generate the perturbations and produce libraries of variants in vivo. These libraries may then be subjected to functional screening and analyzed by high-throughput screening using gRNAs as barcodes, for example. Unlike transcriptional perturbations, the perturbations introduced by DRIVE platform are permanent mutations, thus multiple rounds of perturbations can be performed to increase the diversity of the libraries.

Activating Cryptic Gene Clusters in Recalcitrant Bacteria.

Metagenomics data has revealed the presence of a plethora of gene clusters in nature, especially in metabolically active environments such as soil and gastrointestinal tracts. Many of these gene cluster are known to produce high-value molecules, while the product of many of these clusters are still unknown. On the other hand, many of these (cryptic) clusters are silent in most conditions and are activated under very specific (and in most cases unknown) conditions that is not attainable in laboratory. For example, many bacteria encode cryptic gene cluster that produce valuable secondary metabolite (e.g. antibiotic and other small molecules). Because the production of these products are often very costly to cells, their expression is tightly regulated and limit to very certain conditions that is not known or achievable in laboratory conditions. The ability to activate these gene clusters would be highly desirable for many biotechnological applications and productions of high-value compounds.

The DRIVE platform provided herein enables efficient genetic modifications in recalcitrant and natural isolates of bacteria, without the requirement for efficient homologous recombination. For example, silent gene cluster in these organisms can be activated by mutating the regulatory elements (e.g. promoter, RBS and activator/repressors and their operator sites) using the DNA mutators and gRNAs targeting these regulatory elements (FIG. 16).

### Scalable Platform for Computing and Memory in Living Cells

Engineering highly efficient DNA writers. A platform that enables the manipulation of genomic DNA in vivo with single-nucleotide resolution provides powerful strategies for programming living cells and engineering cellular phenotypes. To build highly efficient DNA writers in living cells, mutated Cas9 variants was fused to a cytidine deaminase protein as DNA-writer module. The DNA writer was then directed and localized to desired target sites by expressing complementary guide RNAs (gRNAs). DNA writing events can be linked to internal or external (e.g. small molecules) inputs by placing the gRNA expression under the control of inducible promoters, for example.

For the DNA-writing module, dCas9 (or nCas9) has been fused to enzymes that can mutate specific nucleotides, such as cytidine deaminases. These modules can introduce mutations into dC positions, resulting in a DNA lesion that is preferentially repaired as dT. Using these DNA writers, depending on the DNA strand being targeted by the gRNA, targeted dC to dT or dG to dA mutations are introduced to the target site, resulting permanent records in the DNA. Introducing nicks into the DNA strand opposite to the deaminated base of DNA can enhance the incorporation of mutations into the sites of the deaminated bases. Thus, in some embodiments, nCas9 fused to cytidine deaminases can be used instead of dCas9 to enhance DNA writing efficiency. In some embodiments, the editing efficiency of cytidine deaminases can be improved by fusing the uracil DNA glycosylase inhibitor (ugi) protein to the d/nCas9-cytidine deaminase fusion. As alternatives to cytidine deaminases, other types of base editors, such as adenosine deaminases (ADA), DNA glycosylases (e.g., MAGI (3-methyladenine DNA glycosylase)) or other types of mutator domains may be used.

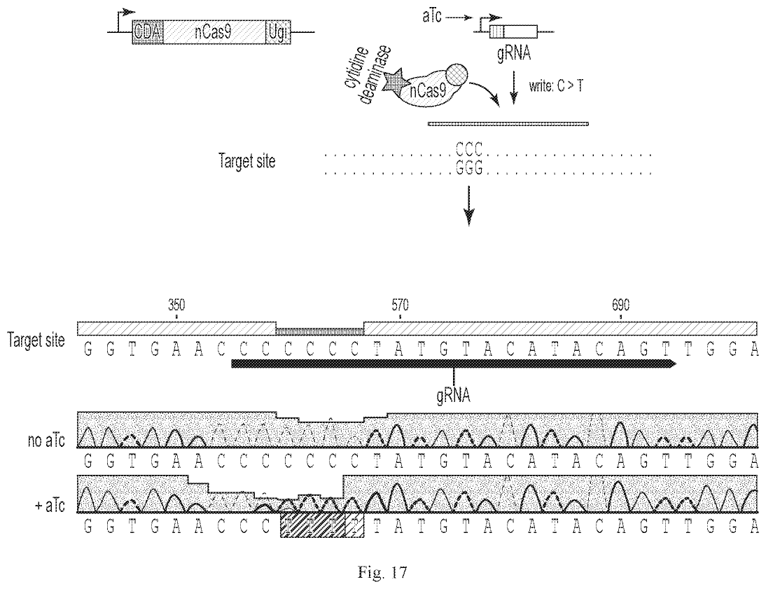

Provided herein is a highly efficient DNA writing system (e.g., in E. coli), which is used for designing robust DOMINO circuits. This platform allows highly efficient and precise modification of genomic DNA and high-copy number plasmids, such as colE1, under the control of cellular cues (e.g. small molecules) (FIG. 17).

Building Logic and Memory Operators in Living Cells Using DOMINOS.

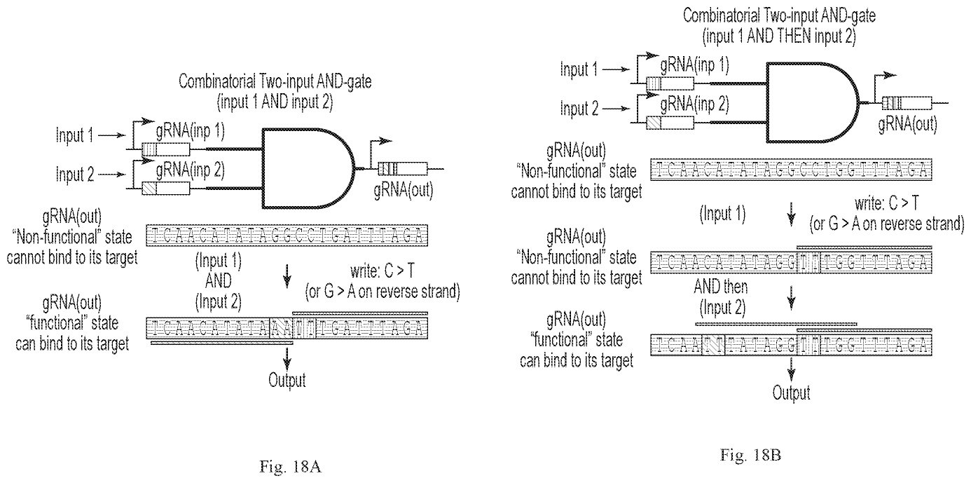

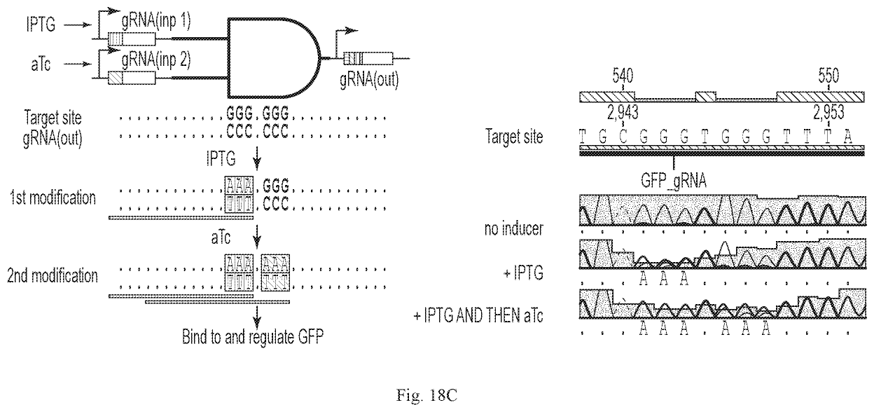

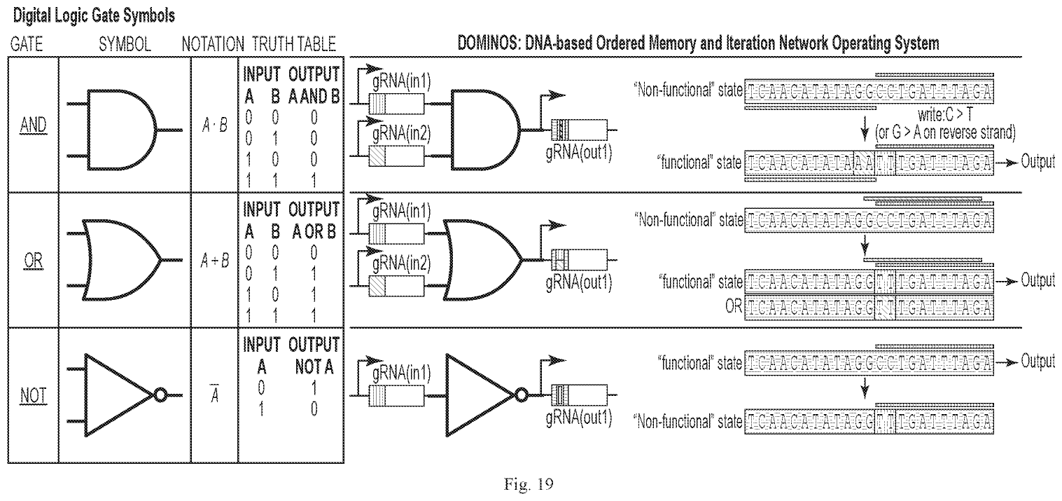

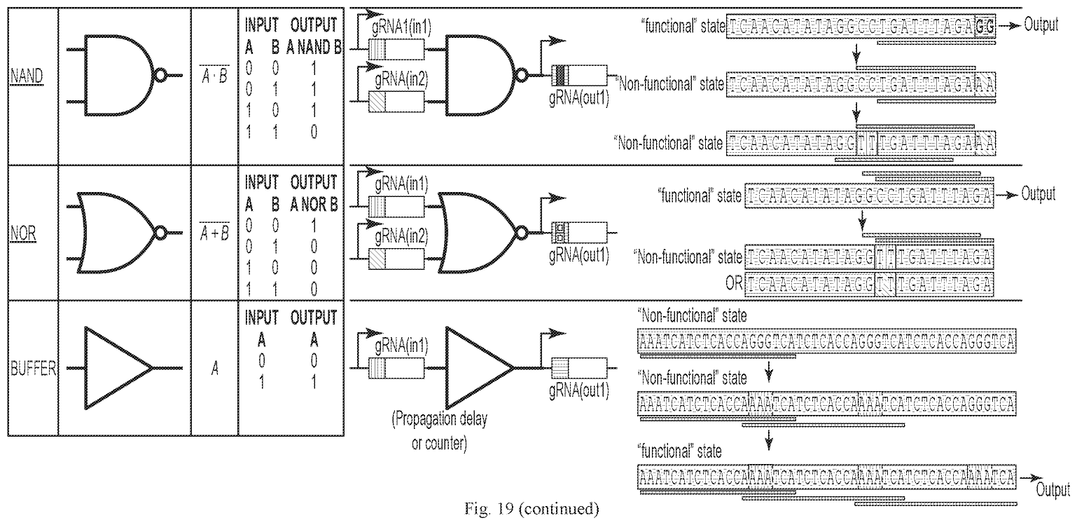

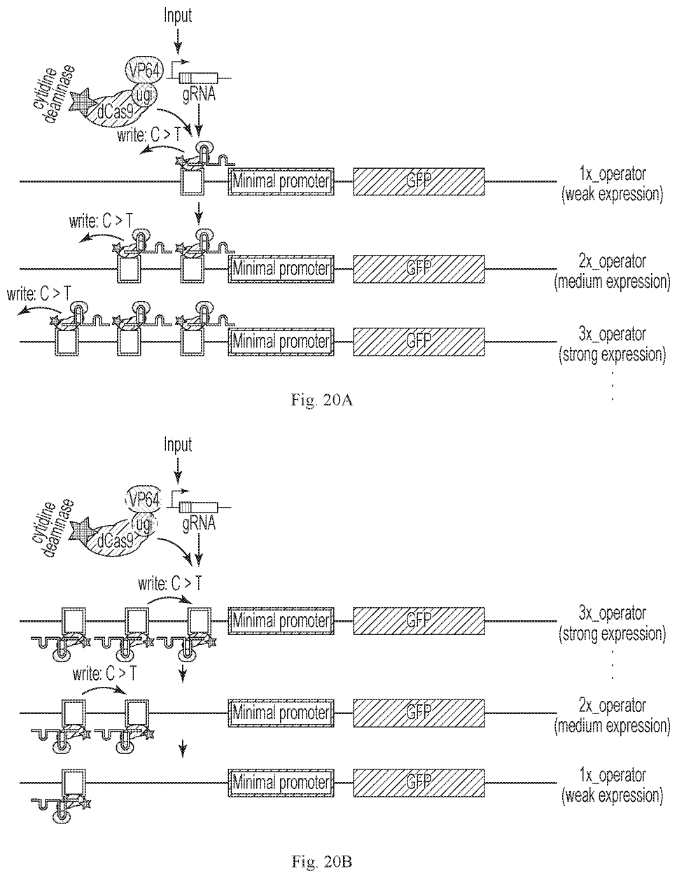

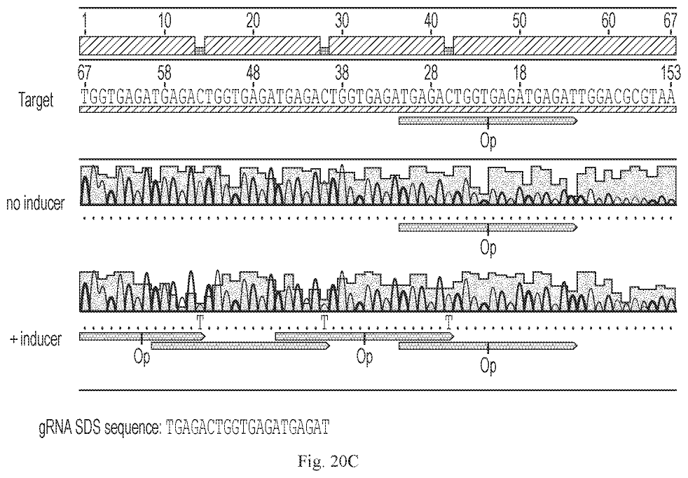

Logic and memory operators are the building blocks of biological circuits. The DOMINO platform enables to build robust, compact and scalable logic and memory operators in living cells by executing order and combinations of DNA writing events in a controlled fashion. By carefully positioning the mutable residues in the gRNA SDS, the frequency and occurrence of DNA writing events can be controlled. The DNA writer can then be directed to desired target sites by expressing complementary gRNAs. gRNA expression can be controlled, in some embodiments, by inducible promoters to couple DNA writing events to external (transcriptional) inputs. For example, two input AND logic operators can be built by layering two gRNAs placed under the control of inducible promoters that edit a third gRNA in response to their cognate gRNAs (FIGS. 18A-18C). Once both edits are applied to the third gRNA, it can activate a reporter gene, thus realizing the AND logic. Other logic operators can be made by changing the sequence of the guide RNAs (FIG. 19). While complex digital logics and circuits can be built by cascading these simple logic operators, more efficient design could be achieved, in some embodiments, by interconnecting DNA writing events and carefully designing sequence of DNA writing events that do not necessarily follow a cascade pattern.

Various orthogonal operators can be built, for example, by simply changing the sequence of the gRNAs, thus making the system highly scalable. Because the system mainly relies on small gRNAs and only one protein moiety, cellular resources are conserved (consuming too much of the limited cellular resources is one of the main limiting factors in scaling existing computation and memory technologies such as site-specific recombinases).

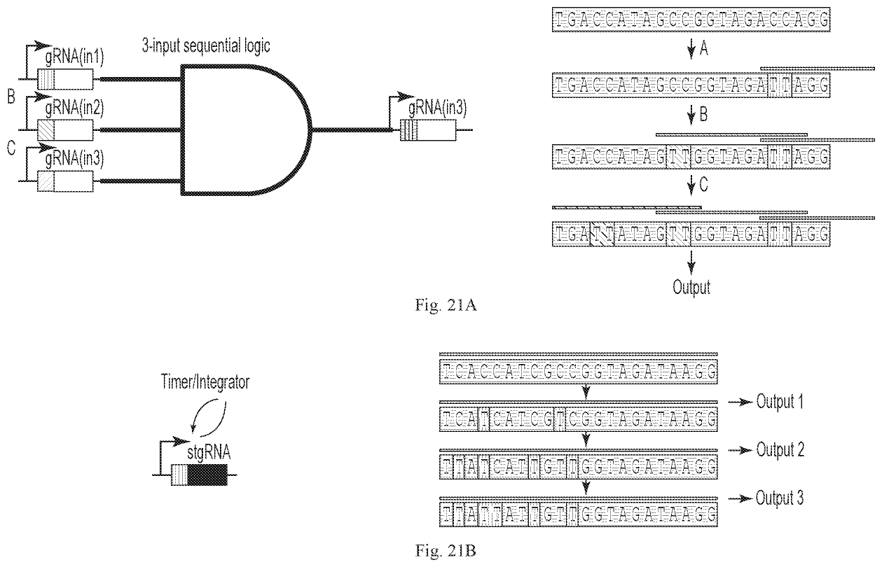

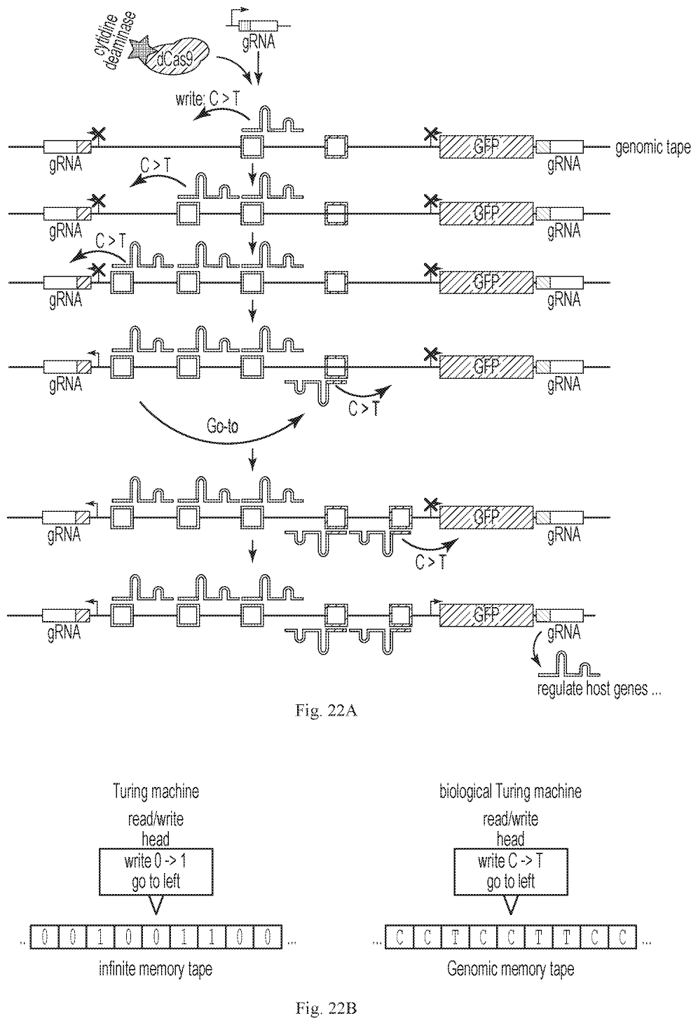

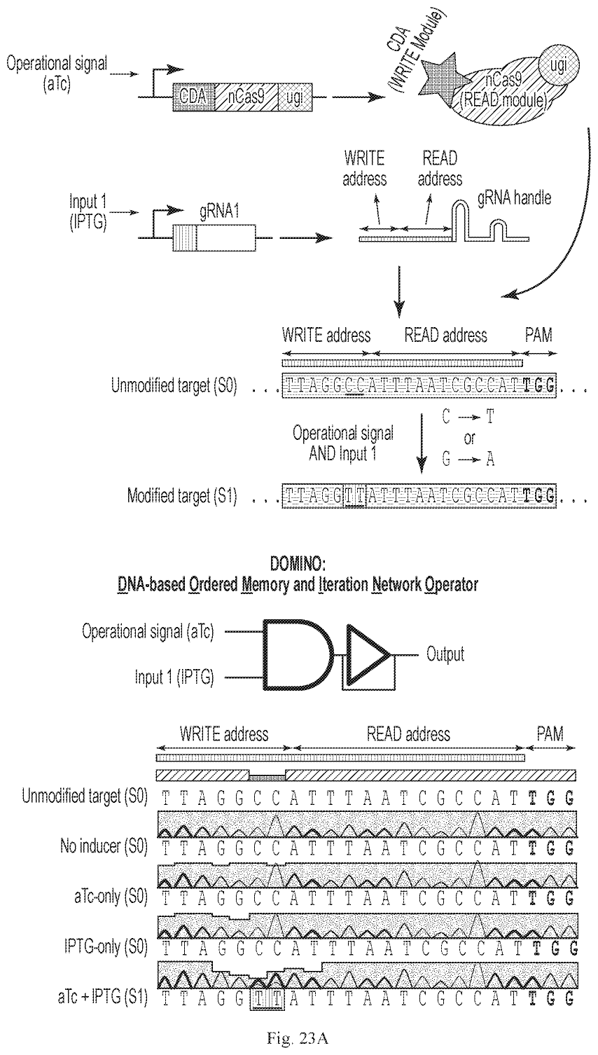

The DNA writer proteins can be further functionalized, in some embodiments, with additional effector domains (such as transcriptional activators and repressors) to achieve combined DNA writing and transcription regulation. As such, the platform offers capacity to perform both genetic and epigenetic modulation of synthetic and natural gene circuits. The DOMINO platform may be used to build advanced gene circuits with the capacity to learn, remember and undergo associative learning. For example, synthetic gene circuits for which a given output can be reinforced (or weaken) in the presence of a given stimulus may be devised (FIGS. 20A-20B). The DOMINOS platform may also be used as a foundation for building more complex and dynamic cellular programs (FIGS. 21A-21B), such as biological state machines and Turing machines (FIGS. 22A-22B).

Thus, the DOMINOS platform offers a highly scalable and modular strategy for dynamic programming of molecular events and incorporating memory and logic operations into living cells. The ability to perform cascades of DNA writing events lays the foundation for building robust and sophisticated synthetic gene circuits and programming cells for numerous biotechnological and biomedical applications. The platform is impactful across many different disciplines including developmental studies, stem cell differentiation, cancer, brain mapping, and many other areas. For example, these platforms can be used to design and program the progression of developmental stages within living animals, or to perform long-term and high-resolution lineage tracking experiments in mammals, which has been challenging to date due to the lack of scalable and long-term methodologies. The DNA writers could be adapted to map neural activity by driving the activity of DNA writers with regulators that respond to neural activity. The systems can be used to study the order and temporal nature of signaling events in their native contexts and robustly control cellular differentiation cascades ex vivo and in vivo. The DNA writers could be programmed to investigate tumor development and unveil the cellular and environmental cues involved in tumor heterogeneity. Arbitrary information could be programmed into the DNA of living cells for DNA storage applications. Finally, living sensors could be designed to sense pathogens, toxins, or other signals within the body or in the environment and then later report on this information in detail.

### Kits

Further provided herein are kits comprising components of the molecular recorders described herein. In some embodiments, a kit comprises: (a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM); (b) an RNA-guided endonuclease or an engineered nucleic acid encoding an RNA-guided endonuclease; and (c) an enzyme that adds random nucleotides to a dsDNA break (e.g., TdT) or an engineered nucleic acid encoding such an enzyme.

In some embodiments, a kit comprises (a) an engineered nucleic acid comprising an array of repetitive deoxycytosine nucleotides (dC)-rich DNA sequences; (b) an engineered nucleic acid comprising a promoter operably linked to a nucleotide sequence encoding a guide ribonucleic acid (gRNA) that targets the array of repetitive dC-rich DNA sequences; and (c) a fusion protein comprising a RNA-guided DNA binding domain (e.g., catalytically-inactive Cas9) fused to cytidine deaminase, or a nucleic acid encoding such a fusion protein.

In some embodiments, a kit comprises (a) an engineered nucleic acid comprising a promoter operably linked to a nucleotide sequence encoding a self-targeting guide ribonucleic acid (stgRNA) that comprises a C-rich specificity determining sequence (SDS) having and a protospacer adjacent motif (PAM); and (b) a fusion protein comprising a RNA-guided DNA binding domain (e.g., catalytically-inactive Cas9) fused to a cytidine deaminase.

The kit described herein may include one or more containers housing components for performing the methods described herein and optionally instructions of uses. Kits for research purposes may contain the components in appropriate concentrations or quantities for running various experiments. Any of the kits described herein may further comprise components needed for performing the methods. For example, it may contain components for use in detecting a signal directly or indirectly. In some examples, the detection step of the assay methods involves enzyme reaction, the kit may further contain the enzyme and a suitable substrate.

Each components of the kits, where applicable, may be provided in liquid form (e.g., in solution), or in solid form, (e.g., a dry powder). In certain cases, some of the components may be lyophilized, reconstituted, or processed (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which can also reflects approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention. Additionally, the kits may include other components depending on the specific application, as described herein.

The kits may contain any one or more of the components described herein in one or more containers. The components may be prepared sterilely, packaged in syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

### Additional Embodiments

Additional embodiments of the present disclosure are encompassed by the following numbered paragraphs:

1. A cell comprising:

(a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM);

(b) a RNA-guided endonuclease; and

(c) an enzyme that catalyzes the addition of nucleotides to the 3′ end of a nucleic acid.

2. The cell of paragraph 1, wherein the engineered nucleic acid is integrated into a locus of the genome of the cell.

3. The cell of paragraph 1 or 2, wherein the RNA-guided endonuclease is Cas9 or Cpf1.

4. The cell of any one of paragraphs 1-3, wherein the PAM is a wild-type PAM.
5. The cell of any one of paragraphs 1-4, wherein the PAM is downstream (3′) from the SDS.
6. The cell of any one of paragraphs 1-5, wherein the PAM is adjacent to the SDS.
7. The cell of any one of paragraphs 1-6, wherein the nucleotide sequence of the PAM is selected from the group consisting of NGG, NNGRR(T/N), NNNNGATT, NNAGAAW, and NAAAAC.
8. The cell of any one of paragraphs 1-7, wherein the length of the SDS is 15 to 75 nucleotides.
9. The cell of any one of paragraphs 1-8, wherein the promoter is an inducible promoter.
9.1. The cell of any one of paragraphs 1-9, wherein the enzyme of (c) is member of the X family of DNA polymerases.
9.2. The cell of paragraph 9.1, wherein the enzyme of (c) is a terminal deoxynucleotidyl transferase (TdT).
10. A method comprising:

maintaining a cell that comprises (a) a RNA-guided endonuclease, (b) an enzyme that catalyzes the addition of nucleotides to the 3′ end of a nucleic acid, and (c) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM), under conditions that result in the addition of random nucleotides to the SDS.

11. The method of paragraph 10, wherein the engineered nucleic acid is integrated into a locus of the genome of the cell.
12. The method of paragraph 10 or 11, wherein the RNA-guided endonuclease is Cas9 or Cpf1.
13. The method of any one of paragraphs 10-12, wherein the PAM is a wild-type PAM.
14. The method of any one of paragraphs 10-13, wherein the PAM is downstream (3′) from the SDS.
15. The method of any one of paragraphs 10-14, wherein the PAM is adjacent to the SDS.
16. The method of any one of paragraphs 10-15, wherein the nucleotide sequence of the PAM is selected from the group consisting of NGG, NNGRR(T/N), NNNNGATT, NNAGAAW, and NAAAAC.
17. The method of any one of paragraphs 10-16, wherein the length of the SDS is 15 to 75 nucleotides.
18. The method of any one of paragraphs 10-17, wherein the promoter is an inducible promoter.
18.1. The method of any one of paragraphs 10-18, wherein the enzyme of (c) is member of the X family of DNA polymerases.
18.2. The method of paragraph 18.1, wherein the enzyme of (b) is a terminal deoxynucleotidyl transferase (TdT).
19. The method of any one of paragraphs 10-18 further comprising introducing into the cell the engineered nucleic acid.
20. The method of any one of paragraphs 10-19 further comprising introducing into the cell the RNA-guided endonuclease or a nucleic acid encoding the RNA-guided endonuclease.
21. The method of any one of paragraphs 10-20 further comprising introducing into the cell the TdT or a nucleic acid encoding the TdT.
22. The method of any one of paragraphs 11-21 further comprising sequencing the locus of the cell into which the engineered nucleic acid is integrated to identify the composition and length of the stgRNA.
23. A kit comprising:

(a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM);

(b) an RNA-guided endonuclease or an engineered nucleic acid encoding an RNA-guided endonuclease; and

(c) a terminal deoxynucleotidyl transferase (TdT) or an engineered nucleic acid encoding a TdT.

24. The kit of paragraph 23, wherein the RNA-guided endonuclease is Cas9 or Cpf1.
25. The kit of paragraph 23 or 24, wherein the PAM is a wild-type PAM.
26. The kit of any one of paragraphs 23-25, wherein the PAM is downstream (3′) from the SDS.
27. The kit of any one of paragraphs 23-26, wherein the PAM is adjacent to the SDS.
28. The kit of any one of paragraphs 23-27, wherein the nucleotide sequence of the PAM is selected from the group consisting of NGG, NNGRR(T/N), NNNNGATT, NNAGAAW, and NAAAAC.
29. The kit of any one of paragraphs 23-28, wherein the length of the SDS is 15 to 75 nucleotides.
30. The kit of any one of paragraphs 23-29, wherein the promoter is an inducible promoter.
31. A cell engineered to include an array of repetitive deoxycytosine nucleotides (dC)-rich (dC-rich) DNA sequences that include deoxycytosine nucleotides integrated into a locus of the genome of the cell and comprising:

(a) an engineered nucleic acid comprising a promoter operably linked to a nucleotide sequence encoding a guide ribonucleic acid (gRNA) that targets the array of repetitive dC-rich DNA sequences; and

(b) a fusion protein comprising a catalytically-inactive Cas9 fused to cytidine deaminase.

32. The cell of paragraph 31, wherein the promoter is an inducible promoter.
33. The cell of paragraph 31 or 32, wherein the length of the SDS is 15 to 75 nucleotides.
34. The cell of any one of paragraphs 31-33, wherein the at least 10% of the nucleotides in the SDS are cytosine nucleotides.
35. A method comprising maintaining a cell engineered to include an array of repetitive deoxycytosine nucleotides (dC)-rich DNA sequences that include deoxycytosine nucleotides (dC) integrated into a locus of the genome of the cell and comprising (a) an engineered nucleic acid comprising a promoter operably linked to a nucleotide sequence encoding a guide ribonucleic acid (gRNA) targets the array of repetitive dC-rich DNA sequences, and (b) a fusion protein comprising a catalytically-inactive Cas9 fused to cytidine deaminase, under conditions that result in targeted mutations in the array of repetitive DNA sequences at dC positions.
36. The method of paragraph 35, wherein the promoter is an inducible promoter.
37. The method of paragraph 35 or 36, wherein the length of the SDS is 15 to 75 nucleotides.
38. The method of any one of paragraphs 35-37, wherein at least 10% of the nucleotides in the target are cytosine nucleotides.
39. The method of any one of paragraphs 35-38 further comprising introducing into the cell the engineered nucleic acid.
40. The method of any one of paragraphs 35-39 further comprising introducing into the cell the fusion protein or a nucleic acid encoding the fusion protein.
41. The method of any one of paragraphs 35-40 further comprising sequencing the locus of the cell to identify targeted mutations in the array of repetitive DNA sequences.
42. A kit comprising:

(a) an engineered nucleic acid comprising an array of repetitive deoxycytosine nucleotides (dC)-rich DNA sequences;

(b) an engineered nucleic acid comprising a promoter operably linked to a nucleotide sequence encoding a guide ribonucleic acid (gRNA) that targets the array of repetitive dC-rich DNA sequences; and

(c) a fusion protein comprising a catalytically-inactive Cas9 fused to cytidine deaminase, or a nucleic acid encoding a fusion protein comprising a catalytically-inactive Cas9 fused to cytidine deaminase.

43. The kit of paragraph 42, wherein the promoter is an inducible promoter.
44. The kit of paragraph 42 or 43, wherein the length of the SDS is 15 to 75 nucleotides.
45. The kit of any one of paragraphs 42-44, wherein at least 10% of the nucleotides in the SDS are cytosine nucleotides.
46. A cell comprising:

(a) an engineered nucleic acid comprising a promoter operably linked to a nucleotide sequence encoding a self-targeting guide ribonucleic acid (stgRNA) that comprises a C-rich specificity determining sequence (SDS) and a protospacer adjacent motif (PAM); and

(b) a fusion protein comprising a catalytically-inactive Cas9 fused to cytidine deaminase.

47. The cell of paragraph 46, wherein the engineered nucleic acid is integrated into a locus of the genome of the cell.
48. The cell of paragraph 46 or 47, wherein the PAM is a wild-type PAM.
49. The cell of any one of paragraphs 46-48, wherein the PAM is downstream (3′) from the SDS.
50. The cell of any one of paragraphs 46-49, wherein the PAM is adjacent to the SDS.
51. The cell of any one of paragraphs 46-50, wherein the nucleotide sequence of the PAM is selected from the group consisting of NGG, NNGRR(T/N), NNNNGATT, NNAGAAW, and NAAAAC.
52. The cell of any one of paragraphs 46-51, wherein the length of the SDS is 15 to 75 nucleotides.
53. The cell of any one of paragraphs 46-52, wherein at least 10% of the nucleotides in the SDS are cytosine nucleotides.
54. The cell of any one of paragraphs 46-53, wherein the promoter is an inducible promoter.
55. A method comprising:

maintaining a cell that comprises (a) an engineered nucleic acid comprising a promoter operably linked to a nucleotide sequence encoding a self-targeting guide ribonucleic acid (stgRNA) that comprises a C-rich specificity determining sequence (SDS) and a protospacer adjacent motif (PAM), and (b) a fusion protein comprising a catalytically-inactive Cas9 fused to cytidine deaminase, under conditions that result in targeted mutations in the stgRNA.

56. The method of paragraph 55, wherein the engineered nucleic acid is integrated into a locus of the genome of the cell.
57. The method of paragraph 55 or 56, wherein the PAM is a wild-type PAM.
58. The method of any one of paragraphs 55-57, wherein the PAM is downstream (3′) from the SDS.
59. The method of any one of paragraphs 55-58, wherein the PAM is adjacent to the SDS.
60. The method of any one of paragraphs 55-59, wherein the nucleotide sequence of the PAM is selected from the group consisting of NGG, NNGRR(T/N), NNNNGATT, NNAGAAW, and NAAAAC.
61. The method of any one of paragraphs 55-60, wherein the length of the SDS is 15 to 75 nucleotides.
62. The method of any one of paragraphs 55-61, wherein at least 10% of the nucleotides in the SDS are cytosine nucleotides.
63. The method of any one of paragraphs 55-62, wherein the promoter is an inducible promoter.
64. The method of any one of paragraphs 55-63 further comprising introducing into the cell the engineered nucleic acid.
65. The method of any one of paragraphs 55-64 further comprising introducing into the cell the fusion protein or a nucleic acid encoding the fusion protein.
66. The method of any one of paragraphs 56-65 further comprising sequencing the locus of the cell into which the engineered nucleic acid is integrated to determine the composition and length of the gRNA.
67. A kit comprising:

(a) an engineered nucleic acid comprising a promoter operably linked to a nucleotide sequence encoding a self-targeting guide ribonucleic acid (stgRNA) that comprises a C-rich specificity determining sequence (SDS) having and a protospacer adjacent motif (PAM); and

(b) a fusion protein comprising a catalytically-inactive Cas9 fused to cytidine deaminase.

68. The kit of paragraph 67, wherein the PAM is a wild-type PAM.
69. The kit of paragraph 67 or 68, wherein the PAM is downstream (3′) from the SDS.
70. The kit of any one of paragraphs 67-69, wherein the PAM is adjacent to the SDS.
71. The kit of any one of paragraphs 67-70, wherein the nucleotide sequence of the PAM is selected from the group consisting of NGG, NNGRR(T/N), NNNNGATT, NNAGAAW, and NAAAAC.
72. The kit of any one of paragraphs 67-71, wherein the length of the SDS is 15 to 75 nucleotides.
73. The kit of any one of paragraphs 67-72, wherein at least 10% of the nucleotides in the SDS are cytosine nucleotides.
74. The kit of any one of paragraphs 67-73, wherein the promoter is an inducible promoter.
75. A method comprising:

maintaining a cell that comprises (a) a nucleic acid comprising a regulatory element operably linked to a target sequence, (b) an engineered nucleic acid comprising an inducible promoter operably linked to a nucleotide sequence encoding a guide ribonucleic acid (gRNA) that comprises a specificity determining sequence (SDS) that targets the regulatory sequence, and (c) a fusion protein comprising a catalytically-inactive Cas9 fused to an epigenetic effector, under conditions that result in an accumulation of targeted epigenetic changes in the vicinity of the target sequence.

76. The method of paragraph 75, wherein the regulatory element is a promoter or an enhancer.
77. The method of paragraph 76, wherein the regulator element is a synthetic regulatory element.
78. The method of any one of paragraphs 75-77, wherein the accumulation of targeted epigenetic changes results in activation or repression of the target sequence.
79. The method of any one of paragraphs 75-78 further comprising performing a functional assay on an extract of the cell to identify expression of the target sequence.
80. The method of paragraph 79, wherein the functional assay is an in vivo functional assay.
81. The method of paragraph 79, wherein a nucleic acid encoding a reporter molecule is operably linked to the regulatory element.
82. The method of paragraph 79, wherein a nucleic acid encoding a recombinase is operably linked to the regulatory element.
83. The method of paragraph 79, wherein the functional assay is a Western blot or an immunoassay.
84. An in vivo diversification method, comprising:

(a) introducing into a cell (i) an engineered nucleic acid encoding a biomolecule that has at least one variable region, (ii) an engineered nucleic acid encoding a guide ribonucleic acid (gRNA) that targets the at least one variable region, and (iii) an engineered nucleic acid encoding a fusion protein comprising a catalytically-inactive Cas9 fused to a mutator domain or a Cas9 nickase fused to a mutator domain; and

(b) maintaining the cell under conditions that results in diversification of the at least one variable region to produce diversified biomolecules.

85. The method of paragraph 84, wherein the mutator domain is selected from cytidine deaminases, adenine deaminases, DNA glycosylases, and ROS generators.
85.1. The method of paragraph 85, wherein the mutator domain is a cytidine deaminase.
85.2. The method of paragraph 85.1, wherein the at least one variable regions comprises an initial variable codon in the form of CCN, where N is any nucleotide.
85.3. The method of any one of paragraphs 84-85.2, wherein the fusion protein further comprises a uracil glycosylase inhibitor (UGI) domain.
85.4. The method of any one of paragraphs 84-85.3, wherein the gRNA is a stgRNA.
86. The method of any one of paragraphs 84-85.4, wherein the cell is a prokaryotic cell.
87. The method of paragraph 86, wherein the prokaryotic cell is an Escherichia coli cell.
88. The method of paragraph 84 or 85, wherein the cell is a eukaryotic cell.
89. The method of paragraph 88, wherein the eukaryotic cell is a yeast cell.
89. The method of paragraph 88, wherein the eukaryotic cell is a mammalian cell.
90 The method of any one of paragraphs 84-89, wherein the biomolecule is a therapeutic protein.
91. The method of any one of paragraphs 84-90, wherein the biomolecule is selected from proteins, RNA-enzymes, DNA-enzymes, and aptamers.
92. The method of paragraph 90 or 91, wherein the biomolecule is selected from antibodies, nanobodies, affibodies, and antibody mimetic proteins.
93. The method of paragraph 92, wherein the biomolecule is an antibody.
94. The method of paragraph 93, wherein the variable region is an epitope.
95. The method of any one of paragraphs 84-94, wherein the engineered nucleic acid of (i), (ii) and/or (iii) is operably linked to a promoter.
96. The method of paragraph 95, wherein the promoter is an inducible promoter.
97. The method of any one of paragraphs 84-96, wherein biomolecule has at least two variable regions targeted by a gRNA.
98. The method paragraph 97, wherein biomolecule has at least three variable regions targeted by a gRNA.
99. The method of any one of paragraphs 84-89, wherein the biomolecule is a bacteriophage tail fiber.
100. The method of any one of paragraph 84-89, wherein the biomolecule comprises a protein-binding domain that binds to a protein of interest, and the gRNA is a stgRNA encoded downstream from the sequence encoding the protein binding domain.
101. The method of any one of paragraphs 84-100 further comprising isolating from the cell nucleic acids encoding the diversified biomolecules.
102. The method of paragraph 101 further comprising inserting the nucleic acids encoding the diversified biomolecules into genes encoding bacteriophage coat proteins, and delivering to the bacteriophage the genes encoding bacteriophage coat proteins.
103. The method of paragraph 102 further comprising assessing the bacteriophage for binding to the protein of interest.
104. A cell comprising (i) an engineered nucleic acid encoding a bacteriophage tail fiber that has at least one variable region, (ii) an engineered nucleic acid encoding a guide ribonucleic acid (gRNA) that targets the at least one variable region, and (iii) an engineered nucleic acid encoding a fusion protein comprising a catalytically-inactive Cas9 fused to a mutator domain or a Cas9 nickase fused to a mutator domain.
105. A bacteriophage comprising the cell of paragraph 104.
106. A cell comprising:

(a) a first inducible promoter operably linked to a nucleic acid encoding a first input gRNA that targets a first SDS region of an output gRNA;

(b) a second inducible promoter operably linked to a nucleic acid encoding a second input gRNA that targets a second SDS region of the output gRNA;

(c) a third promoter operably linked to a nucleic acid encoding the output gRNA;

(d) a fourth promoter operably linked to a nucleic acid encoding a fusion protein comprising a catalytically-inactive Cas9 fused to a mutator domain or a Cas9 nickase fused to a mutator domain; and

(e) a target nucleic acid,

wherein the output gRNA targets the target nucleic only following transcription of the first and second input gRNAs and binding of the first and second input gRNAs to the output gRNA.

107. The cell of paragraph 106, wherein the output gRNA comprises the following nucleotide sequence in the 5′ to 3′ direction: X_{N}GGCCY_{N}, where X is any nucleotide, Y is any nucleotide, and N is any integer greater than 0.
108. The cell of paragraph 107,

wherein the first input gRNA comprises the following nucleotide sequence in the 5′ to 3′ direction: Y′_{N}GG-, and Y′_{N} comprises a nucleotide sequence complementary to Y_{N}; and

wherein the second input gRNA comprises the following nucleotide sequence in the 5′ to 3′ direction: CCX′_{N}, and X′_{N} comprises a nucleotide sequence complementary to X_{N}.

109. The cell of paragraph 106, wherein the output gRNA comprises the following nucleotide sequence in the 5′ to 3′ direction: X_{N}CCY_{N}CCZ_{N}, where X is any nucleotide, Y is any nucleotide, Z is any nucleotide, and N is any integer greater than 0.
110. The cell of paragraph 109,

wherein the first input gRNA comprises the following nucleotide sequence in the 5′ to 3′ direction: Z′_{N}GGY′_{N}, and Z′_{N} comprises a nucleotide sequence complementary to Z_{N}, and Y′_{N} comprises a nucleotide sequence complementary to Y_{N}; and

wherein the second input gRNA comprises the following nucleotide sequence in the 5′ to 3′ direction: AAY′_{N}GG, and Y′_{N} comprises a nucleotide sequence complementary to Y_{N}.

111. A cell engineered to include an array of repetitive deoxycytosine nucleotides (dC)-rich (dC-rich) DNA sequences that include deoxycytosine nucleotides integrated into a locus of the genome of the cell and comprising:

(a) an engineered nucleic acid comprising a promoter operably linked to a nucleotide sequence encoding a guide ribonucleic acid (gRNA) that targets the array of repetitive dC-rich DNA sequences; and

(b) an engineered nucleic acid comprising a promoter operably linked to a nucleotide sequence encoding a fusion protein comprising a catalytically-inactive Cas9 fused to a cytidine deaminase.

112. The cell of paragraph 111, wherein the promoter of (a) is an inducible promoter.
113. The cell of paragraph 111 or paragraph 112, wherein the promoter of (b) is an inducible promoter.
114. The cell of any one of paragraphs 111-113, wherein the length of the SDS is 15 to 75 nucleotides.
115. The cell of any one of paragraphs 111-114, wherein the at least 10% of the nucleotides in the SDS are cytosine nucleotides.
116. The cell of any one of paragraphs 111-115, wherein the fusion protein of (b) further comprises a uracil glycosylase inhibitor (UGI) domain.
117. A cell comprising:

(a) an engineered nucleic acid comprising a promoter operably linked to a nucleotide sequence encoding a self-targeting guide ribonucleic acid (stgRNA) that comprises a deoxycytosine nucleotides (dC)-rich (dC-rich) specificity determining sequence (SDS) and a protospacer adjacent motif (PAM); and

(b) an engineered nucleic acid comprising a promoter operably linked to a nucleotide sequence encoding a fusion protein comprising a catalytically-inactive Cas9 fused to cytidine deaminase.

118. The cell of paragraph 118, wherein the engineered nucleic acid is integrated into a locus of the genome of the cell.
119. The cell of paragraph 117 or 118, wherein the PAM is a wild-type PAM.
120. The cell of any one of paragraphs 117-119, wherein the PAM is downstream (3′) from the SDS.
121. The cell of any one of paragraphs 117-120, wherein the PAM is adjacent to the SDS.
122. The cell of any one of paragraphs 117-121, wherein the nucleotide sequence of the PAM is selected from the group consisting of NGG, NNGRR(T/N), NNNNGATT, NNAGAAW, and NAAAAC.
123. The cell of any one of paragraphs 117-122, wherein the length of the SDS is 15 to 75 nucleotides.
124. The cell of any one of paragraphs 117-123, wherein at least 10% of the nucleotides in the SDS are cytosine nucleotides.
125. The cell of any one of paragraphs 117-124, wherein the promoter of (a) is an inducible promoter.
126. The cell of any one of paragraphs 117-125, wherein the promoter of (b) is an inducible promoter.
127. The cell of any one of paragraphs 117-126, wherein the promoter of (a) is different from the promoter of (b).
128. The cell of any one of paragraphs 117-127, wherein the fusion protein further comprises a uracil glycosylase inhibitor (UGI) domain.
129. A cell comprising:

(a) an engineered nucleic acid comprising a first inducible promoter operably linked to a nucleotide sequence encoding a first input guide RNA (gRNA) that targets a first target sequence;

(b) an engineered nucleic acid comprising a second inducible promoter operably linked to a nucleotide sequence encoding a second input gRNA that targets a second target sequence; and

(c) an engineered nucleic acid comprising a third inducible promoter operably linked to a nucleotide sequence encoding a fusion protein comprising a catalytically-inactive Cas9 fused to a cytidine deaminase;

wherein the first target sequence and second target sequence are in a nucleotide sequence encoding an output molecule, and wherein the output molecule is expressed only following transcription of the first and second input gRNAs and binding of the first and second input gRNAs to the first and second target sequences.

130. The cell of paragraph 129, wherein the first inducible promoter is different from the second inducible promoter.
131. The cell of paragraph 129 or paragraph 130, wherein the second input gRNA targets the second target sequence only following the binding of the first input gRNA to the first target sequence.
132. The cell of any one of paragraphs 129-131, wherein the fusion protein further comprises a uracil glycosylase inhibitor (UGI) domain.
133. A cell comprising:

(a) an engineered nucleic acid comprising a first inducible promoter operably linked to a nucleotide sequence encoding a first input guide RNA (gRNA) that targets a first target sequence;

(b) an engineered nucleic acid comprising a second inducible promoter operably linked to a nucleotide sequence encoding a second input gRNA that targets a second target sequence; and

(c) an engineered nucleic acid comprising a third inducible promoter operably linked to a nucleotide sequence encoding a fusion protein comprising a catalytically-inactive Cas9 fused to a cytidine deaminase;

wherein the first target sequence and second target sequence are in a nucleotide sequence encoding an output molecule, and wherein the output molecule is expressed only following transcription the first input gRNAs and binding of the first input gRNA to the first or target sequence, or following transcription the second input gRNAs and binding of the second input gRNA to the second or target sequence, but not both.

134. The cell of paragraph 133, wherein the first inducible promoter, the second inducible promoter, and the third inducible promoter are each different promoters.
135. The cell of any one of paragraph 133 or paragraph 134, wherein the fusion protein further comprises a uracil glycosylase inhibitor (UGI) domain.
136. A cell comprising:
(a) a nucleotide sequence encoding a biomolecule that has at least one variable region;
(b) an engineered nucleic acid comprising a promoter operably linked to a nucleotide sequence encoding a guide ribonucleic acid (gRNA) that targets the at least one variable region; and
(c) an engineered nucleic acid comprising a promoter operably linked to a nucleotide acid encoding a fusion protein comprising a catalytically-inactive Cas9 fused to a cytidine deaminase domain.
137. The cell of paragraph 136, wherein the fusion protein further comprises a uracil glycosylase inhibitor (UGI) domain.
138. The cell of paragraph 136 or paragraph 137, wherein the biomolecule is a therapeutic protein.
139. The cell of any one of paragraphs 136-138, wherein the biomolecule is selected from proteins, RNA-enzymes, DNA-enzymes, and aptamers.
140. The cell of any one of paragraphs 136-139, wherein the biomolecule is selected from antibodies, nanobodies, affibodies, and antibody mimetic proteins.
141. The cell of paragraph 140, wherein the biomolecule is an antibody.
142. The cell of paragraph 141, wherein the variable region is an epitope.
143. The cell of paragraph 136 or paragraph 137, wherein the biomolecule is a bacteriophage tail fiber.
144. The cell of paragraph 136 or paragraph 137, wherein the biomolecule is a cell surface receptor.
145. The cell of any one of paragraphs 136-144, wherein the inducible promoter of (a) and/or (b) is an inducible promoter.
146. The cell of any one of paragraphs 136-145, wherein the nucleotide sequence of (a) has at least two variable regions.
147. The cell of any one of paragraphs 136-146, wherein the nucleotide sequence of (a) has at least three variable regions.
148. The cell of any one of paragraphs 129-147, wherein the output molecule is a detectable molecule.
149. The cell of paragraph 148, wherein detectable molecule is a fluorescent protein.
150. The cell of any one of paragraphs 111-149, wherein the cell is a prokaryotic cell.
151. The cell of paragraph 150, wherein the prokaryotic cell is an Escherichia coli cell.
152. The cell of any one of paragraphs 111-149, wherein the cell is a eukaryotic cell.
153. The cell of paragraph 152, wherein the eukaryotic cell is a yeast cell.
154. The cell of paragraph 152, wherein the eukaryotic cell is a mammalian cell.
155. A method, the method comprising maintaining the cell of any one of paragraphs 111-154.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

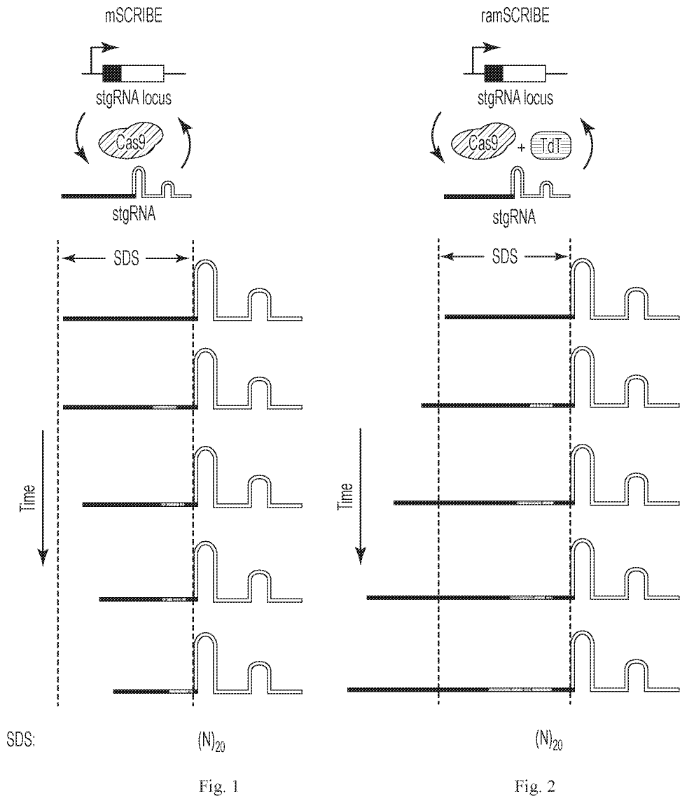

The molecular recorders of the present disclosure are composed of a self-contained memory device that enables the recording of molecular stimuli in the form of DNA modifications, and a DNA modifying protein that produces specific modifications that may be traced. The self-contained memory device (also termed “mSCRIBE,” FIG. 1) includes a self-targeting guide RNA (stgRNA) cassette that repeatedly directs Streptococcus pyogenes Cas9 nuclease towards the DNA that encodes the stgRNA, thereby enabling localized, continuous DNA modification as a function of stgRNA expression.

The mSCRIBE system relies on the continuous cleavage of the stgRNA locus in the presence of Cas9. The double-stranded DNA (dsDNA) breaks targeted to the stgRNA locus are repaired by the error-prone non-homologous end joining (NHEJ) repair mechanism, which result in mutated stgRNAs (indel formation) that could undergo additional rounds of cleavage and error-prone repair. The indels that are accumulate in the stgRNA locus can serve as barcodes to trace cells history.

As illustrated herein, by using different DNA modifying proteins in conjunction with the mSCRIBE system, traceable DNA modification that are genetic (e.g., addition of random nucleotides, or base change) or epigenetic (e.g., methylation, acetylation, or histone modification) may be generated and accumulated. Non-limiting examples of molecular recorder systems described herein and their specific features are summarized in Table 1.

TABLE 1
Molecular Recorder Systems

| Property | mSCRIBE | ramSCRIBE | ENGRAmSCRIBE | ENGRAM | epiSCRIBE |
|---|---|---|---|---|---|
| Continuous | Yes | Yes | Yes | Yes | Yes |
| recording | | | | | |
| dsDNA breaks | Yes | Yes | No | No | No |
| Preservation of | Yes | Yes | Yes | Yes | Yes |
| existing | | | | | |
| barcodes | | | | | |
| gRNA length | Yes | Yes | Constant | Constant | Constant |
| change | | | | | |
| Barcodes | No | Yes | No | No | No |
| recorded | | | | | |
| sequentially | | | | | |
| Memory type | genetic | genetic | genetic | genetic | epigenetic |
| SDS Sequence | NNNNNNNNN | NNNNNNNNN | CCCCCCCCDDDD | CCCCCCC | NNNNNNNNN |
| | NNNNNNNNN | NNNNNNNNN | DDDDDDDD | CCCCCCC | NNNNNNNNN |
| | NN | NN | | CCCCCC | NN |
| guide RNA | GGGTTAGAG | GGGTTAGAG | GTTTTAGAGCTA | GGGTTAG | GTTTTAGAG |
| handle sequence | CTAGAAATA | CTAGAAATA | GAAATAGCAAG | AGCTAGA | CTAGAAATA |
| | GCAAGTTAA | GCAAGTTAA | TTAAAATAAGGC | AATAGCA | GCAAGTTAA |
| | CCTAAGGCT | CCTAAGGCT | TAGTCCGTTATC | AGTTAAC | AATAAGGCT |
| | AGTCCGTTA | AGTCCGTTA | AACTTGAAAAA | CTAAGGC | AGTCCGTTA |
| | TCAACTTGA | TCAACTTGA | GTGGCACCGAGT | TAGTCCG | TCAACTTGA |
| | AAAAGTGGC | AAAAGTGGC | CGGTGCTTTT | TTATCAA | AAAAGTGGC |
| | ACCGAGTCG | ACCGAGTCG | (SEQ ID | CTTGAAA | ACCGAGTCG |
| | GTGCTTTT | GTGCTTTT | NO: 75) | AAGTGGC | GTGCTTTT |
| | (SEQ ID NO: | (SEQ ID NO: | | ACCGAGT | (SEQ ID NO: |
| | 73) | 74) | | CGGTGCT | 77) |
| | | | | TTT (SEQ | |
| | | | | ID NO: 76) | |

### Example 1. Random Additive Memory SCRIBE (ramSCRIBE)

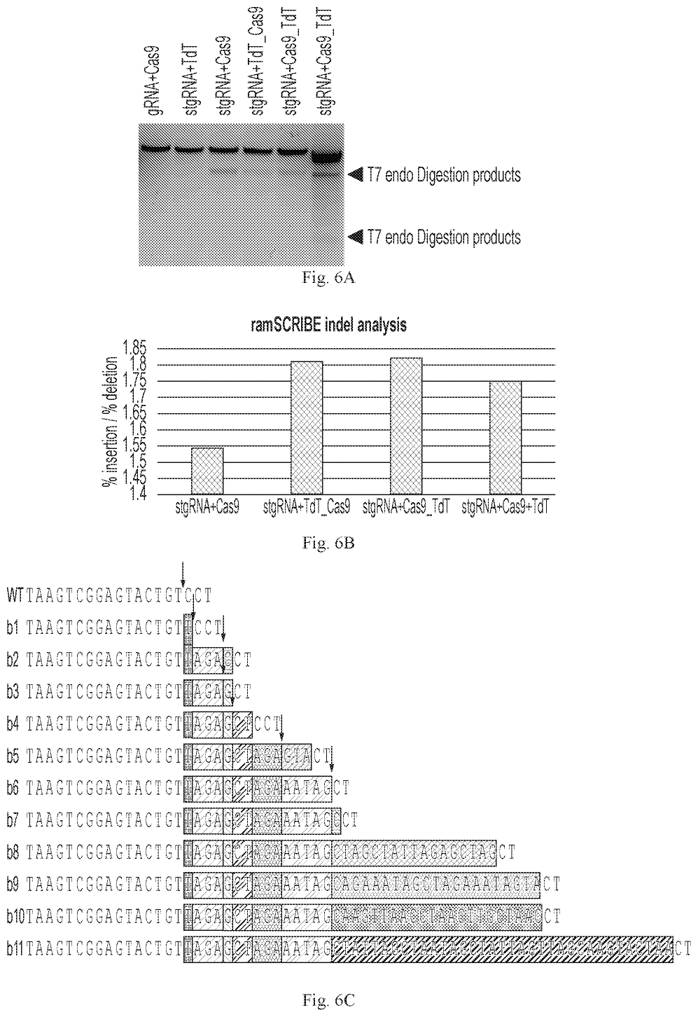

To demonstrate the addition of random bar codes at dsDNA breaks introduced by Cas9 in the stgRNA locus, HEK293 cells harboring integrated stgRNA locus was transfected with plasmids expressing TdT, Cas9, TdT_Cas9, or Cas9_TdT, or cotransfected with plasmids expressing TdT and Cas9. Transfected cells were grown for 48 hours, diluted 1:10 and grown for additional 48 hours. Cells were harvested and genomic DNA of the stgRNA locus was PCR amplified and analyzed by T7 Endonuclease assay (FIG. 6A) and high-throughput sequencing. Insertion are favored when TdT is expressed with Cas9 (FIG. 6B). A trace of random barcodes sequentially added to the stgRNA locus detected in cells expressing ramSCRIBE system is shown in FIG. 6C. Barcode calling and resolution of individual barcodes can be improved by increasing the sequencing depth.

### Example 2. ENGineered Random Accumulative Memory (ENGRAM) and ENGRAmSCRIBE

To demonstrate that the ENGRAM system introduces C to T mutations in an integrated genomic locus, yeast cells harboring integrated 2× a1 repeats and DOX-inducible a1_gRNA (or a non-specific (NS)_gRNA) as well as either pGAL1_dCas9, pGAL1_dCas9_PmCDA1 or PGAL1_nCas9_PmCDA1 were generated. Cells were induced (gal+DOX) for ˜10 generations and the genomic DNA were purified. The genomic locus containing the integrated a1 repeats was PCR amplified from the purified genomic DNA and analyzed by T7 Endonuclease assay (FIG. 7). Mutations were detected in cells expressing a1_gRNA and nCas9_PmCDA1, and to lesser extent in those expressing dCas9_PmCDA1 and a1_gRNA. No T7 endo cleavage products were detected in cells expressing NS_gRNA.

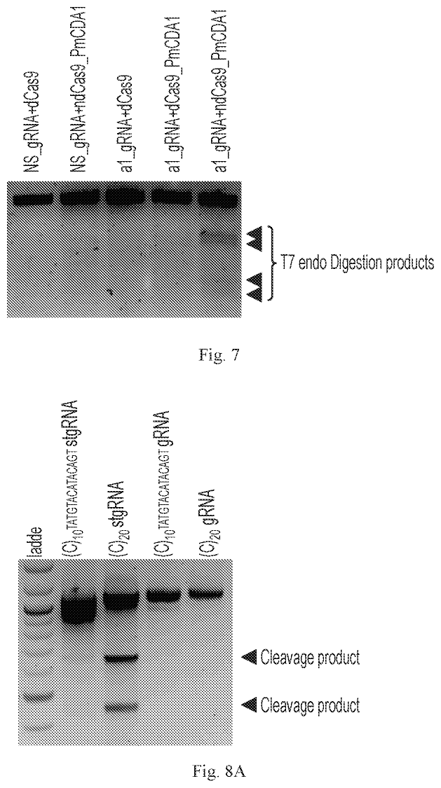

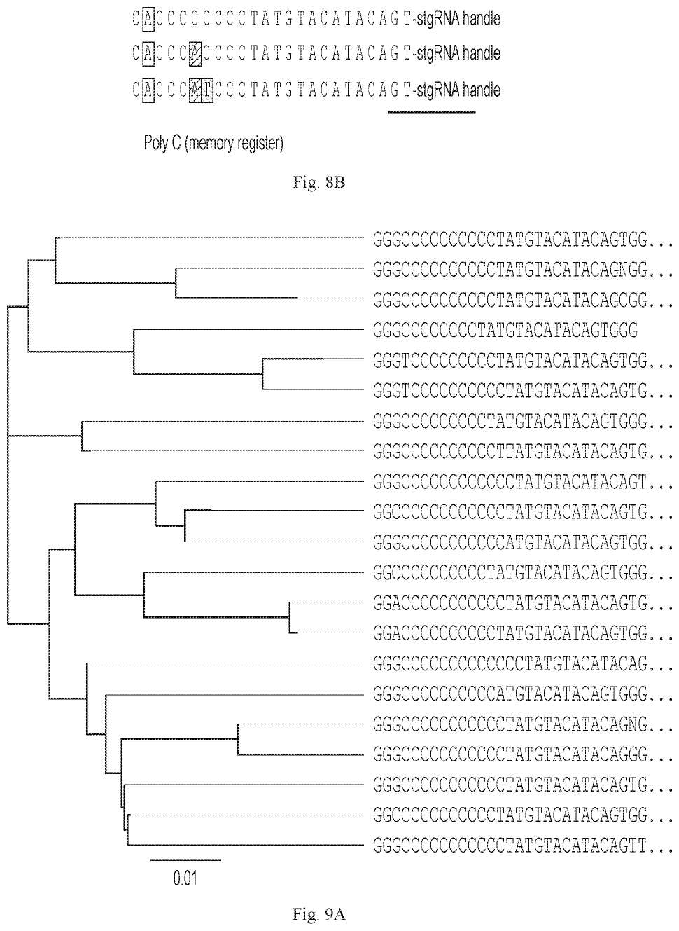

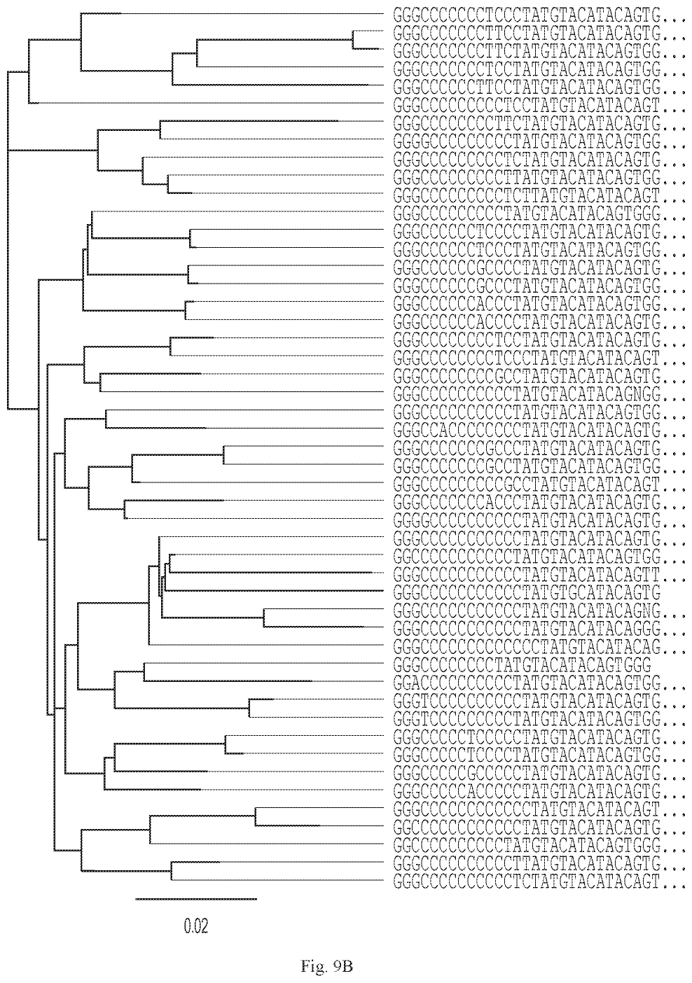

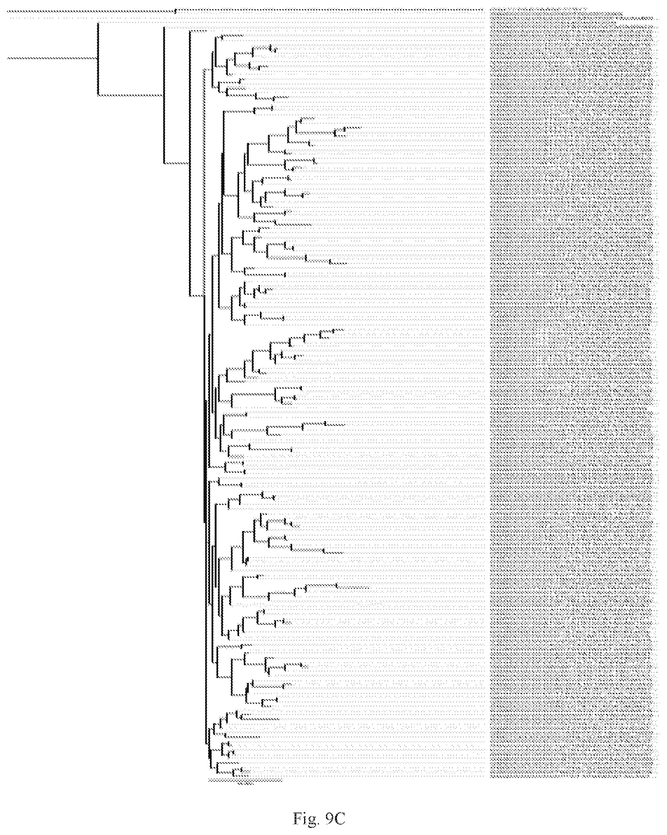

To demonstrate that continuous C to T mutations may be introduced into the stgRNA locus by the ENGRAmSCRIBE system, yeast cells harboring C-rich stgRNA or gRNAs were transformed with pGAL1_nCas9_PmCDA1. Cells were induced (gal+DOX) for ˜10 generations and the genomic DNA were purified. The genomic stgRNA (or gRNA) locus was PCR amplified from the purified genomic DNA and analyzed by T7 Endonuclease assay. Mutations were detected in cells expressing stgRNA and nCas9_PmCDA1. No T7 endo cleavage products were detected in cells expressing gRNA (FIG. 8A). A trace of random mutations that accumulated in the poly C region was detected in cells expressing (C)₁₀ TATGTACATACAGT stgRNA (SEQ ID NO: 78) (FIG. 8B)

### Example 3. Continuous In Vivo Evolution

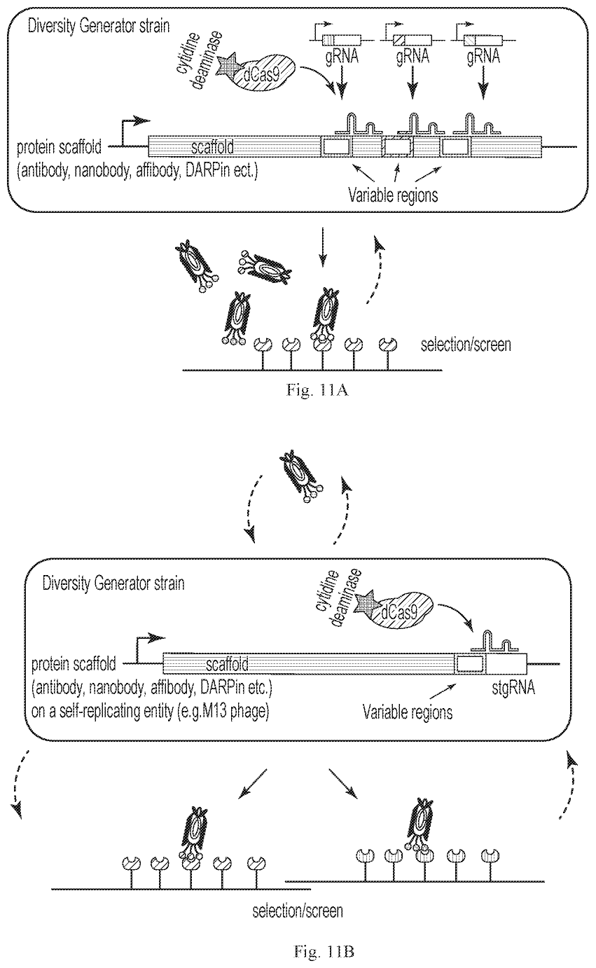

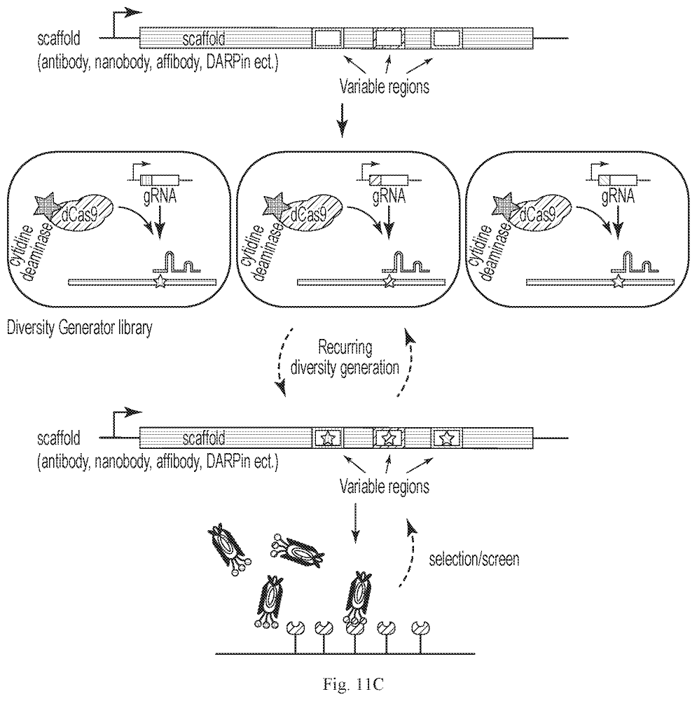

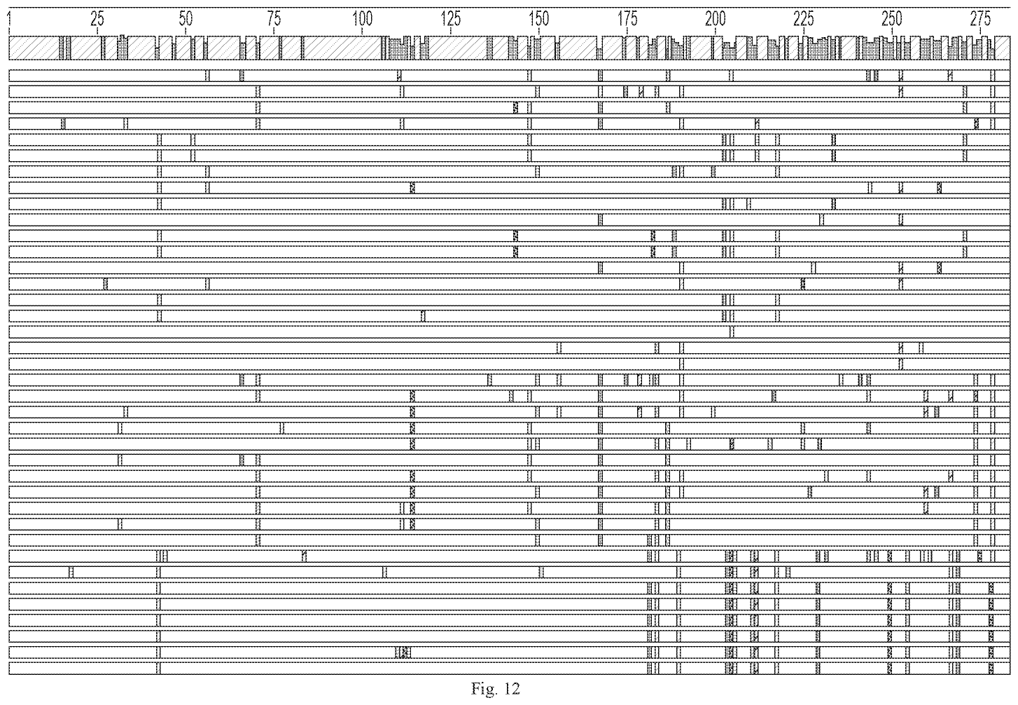

The analysis of natural variations in a protein can indicate the variable regions (mutation hotspots permissive for diversity generation) and the highly conserved regions. Here, as in antibody generation, mutations are localized to a region of permissible variability. After identification of variable regions, a recoded scaffold, with strategically placed PAM domains in the vicinity of targeted variable regions, is synthesized. When using a cytidine deaminase as mutator module, the initial scaffold contains dC residues in the variable codons and a PAM domain positioned in their vicinity. Cytidine deaminase activity is then be targeted to these codons to diversify these sequences. When using an adenine deaminase as mutator domain, the variable positions in the initial scaffold contain dA residues. The recoded scaffold is introduced to cells expressing a library of gRNA and diversity generator module to produce a library of variants. The library diversification step may be repeated multiple rounds to increase the diversity before subjecting variants to appropriate selection or screening step (FIGS. 11A-11C).

The DRIVE platform can be readily incorporated into the established protein engineering platform such as phage display and yeast display. It can be combined with (or replace) the in vitro diversity generating step in these techniques to produce a much larger and diverse libraries than currently possible.

The sequence subject to diversification may a functional DNA motif, or one that encodes a functional RNA (e.g., RNAzyme, RNA aptamer) or a protein scaffold. Various natural and synthetic protein scaffolds can be subjected to mutagenesis and screening for different purposes. These include evolving antigen binding protein scaffolds (e.g. antibody, nanobody, affibody, Obodies, DARPins and etc.) for therapeutic purposes, evolving phage tail fibers for engineering phage host range, or evolving RNA and DNA aptamers with novel functions in vivo. In general, DRIVE can be used to diversify any DNA-encoded biomolecule scaffold in vivo and replace the traditional, inefficient, labor- and time-intensive in vitro diversity generation procedures in techniques such as phage, bacterial or yeast display.

### Example 4. In Vivo Diversification of Biomolecules Scaffolds Using DRIVE

In this example, DRIVE-mediated in vivo diversity generation is combined with the well-established phage display technique. The diversity generator strain contains the mutator protein and gRNAs targeting desired sites on the protein scaffold. Upon introduction of the scaffold DNA, new variants containing mutations defined by the gRNAs are generated, which can then be screened or selected by established techniques. The variants can be reintroduced to the diversity generator host for additional rounds of diversifications and screening (FIG. 11A). A self-targeting stgRNA can be encoded downstream of a scaffold of interest to build a fast-evolvable system. For example, stgRNA is placed downstream of a protein binding domain, in the phage display system, and the produced phages are assessed for binding to desired antigen. The selected variants can be reintroduced in a bacterial host simply by infecting these cells with the selected phages for additional rounds of evolution. The diversity generation and selection can be performed continuously without minimal handling requirement (FIG. 11B). Individual gRNAs can be transformed into a population of bacteria, which can be then used a diversity generator population. The scaffold plasmids can be reintroduced to this population multiple times for multiplexed mutations and increasing the library diversity, before being subjected to screen or selection. After each round of screen, improved variants can be reintroduced to the diversity generator population for additional rounds of diversification and screening (FIG. 11C).

### Example 5. Continuous Phage Host Range Engineering Using DRIVE

In this example, targeted diversity is introduced into bacteriophage tail fiber (and/or other segments of a phage genome that are connected to its host specificity) by passaging a phage on a diversity generator strain containing the DRIVE system and a library of gRNAs targeting the tail fiber and other desired loci for mutagenesis (FIG. 13A). The diversified phages are then introduced to the target strain, and successful variants that have gained the ability to infect target bacteria are obtained. These variants can be reintroduced into the diversity generator host for additional rounds of diversification and screening to improve their specificity for the target host in a continuous faction (FIG. 13A). Instead of using a single-diversity generator host, individual gRNAs can be transformed into a population of bacteria which can then be used as a diversity generator population. Wild-type (or evolved phages obtained from previous rounds of diversification) can be propagated on this population (to various degree) to produce various spectrums of phage variants in the library diversity, before being subjected to screen or selection. After each round of screen, improved variants can be reintroduced to the diversity generator population for additional rounds of diversification followed by screening (FIG. 13B).

### Example 6. Lamarckian Evolution

In this example, DNA writing and diversity generation by Cas9-mutators coupled to external inputs are used to build organisms and gene networks with the ability to undergo Lamarckian evolution. These cells and organisms can mutate and diversify their genome in demand (e.g. in response to an external input or inducer) and at very specific sites (without increasing their global mutation rate) to increase their fitness in a new environment (FIG. 14A). Phages harboring a site specific mutator circuit can use the DRIVE system to increase the evolution of their tail fiber when adapting to new hose. In the presence of a defined signal, the phage will diversify its tail fiber. Once exposed to a new host, these variants can compete for replication on these new host. Over time, fit variants are selected and enrich the population, enabling the phage to adapt to a new host by Lamarckian evolution (FIG. 14B). Cas9-mutator and a gRNA (or a self-targeting gRNA (stgRNA)) targeting the (C-terminus of) the phage tail fiber can be engineered to in a phage genome, to enable to continuously mutagenize this region. As a result, these phages can site-specifically mutagenize their tail fiber and adapt to infect new hosts much faster than naturally possible (e.g., via Darwinian evolution). Cells can also be engineered to diversify key residues in their surface receptors (e.g. those are essential for binding to surfaces), and adapt to new niches much faster than is possible with Darwinian evolution. Bacteria may designed to increase the mutation of genes (e.g. surface receptor) connected to their fitness in a new environment (such as specific niche in the gastrointestinal tract). Once exposed to an environmental cue, these cells can activate the internal targeted mutagenesis process and undergo accelerated evolution to adapt to the new environment (FIG. 14C).

### Example 7. Functional Screening

A pooled gRNA library targeting ORFs and regulatory elements are transformed into cell populations, enabling the production of gene knockout, as well as up-regulation and down-regulation of gene expression. The in vivo-generated variants can then be screened for a desired phenotype (FIG. 15). The identified variants can be subjected to additional rounds of diversification if desired. The gRNA sequences can be used as barcodes to trace enrichment of successful variants by high-throughput sequencing, for example.

### Example 8. Activating Silent Gene Clusters in Natural Isolates or Recalcitrant Bacteria

Cis-regulatory and trans-regulatory elements of silent gene clusters can be targeted by DNA mutators, and the variants with up-regulated gene clusters be identified by functional screening cells for products of gene cluster (e.g. using HPLC) (FIG. 16).

### Example 9. DNA Writing System

This example tests a DNA writing system. The gRNA targeting a C-rich sequence on a plasmid harboring high-copy number colE1 plasmid was placed under the control of aTc-inducible promoter. The DNA writer module (cytidine deaminase(CDA)-nCas9-Uracil DNA glycosylase (Ugi) fusion) was placed under the control of a constitutive promoter. E. coli cells were co-transformed with both plasmids and transformants were grown at the presence or absence of aTc (FIG. 17, left panel). Sanger sequencing results for purified plasmids and the gRNA target in each sample are shown in FIG. 17, right panel. In cells induced with aTc, dC residues at the 5-end of the target were converted to dT, indicating successful inducible site-specific writing.

### Example 10. Combinatorial Two Input AND Gate Built by DOMINOS Logic

The input gRNAs (red and blue) are designed to modify a third (output) gRNA in response to their corresponding inducer (FIG. 18A). Once the output gRNA is modified by both input gRNAs, it becomes functional and activates a downstream reporter or a downstream gRNA. In this example, the order of editing events is not important, and each input gRNA can modify the target gRNA independent of the action of the other input gRNA, thus a combinatorial logic is realized. FIG. 18B shows an example of sequential two-input AND gate built by DOMINOS logic. The input gRNAs (red and blue) are designed to modify a third (output) gRNA in response to their corresponding inducer. Once the output gRNA is modified by both input gRNAs, it becomes functional and activates a downstream reporter or a downstream gRNA. In this example, the order of DNA editing events is important; binding of the second input gRNA (i.e. blue) depends on the action of the first (i.e. red) gRNA. Both modifications (i.e. activation of the output gRNA) only happen when first gRNA1 is expressed and then gRNA2, thus a sequential logic is realized. FIG. 18C shows an examples of sequential two-input DOMINO logic AND gate built in E. coli. Starting from a non-functional state, the output gRNA is modified by sequential addition of IPTG and aTc to media, thus changing the sequence of the output gRNA to a functional state that could bind to a predesigned sequence (in this case GFP).

### Example 11. Two-Input DOMINO Logic Gates

The input gRNAs (red and blue), which are expressed in response to their corresponding inducer, are designed to bind to and modify a third (output). Once initially non-functional output gRNA is modified by the input gRNA(s), its sequence is changed to a “functional” state which can now bind to and modulate a downstream gRNA or reporter (this is the case for AND and OR gates shown above) (FIG. 19). Alternative, an initially “functional” output gRNA can be modified by input gRNAs and turn into a “non-functional” state, enabling to realize another subset of logic gates (e.g., NOT, NOR and NAND logics).

### Example 12. Multifunctional DNA Writers

FIG. 20A shows a synthetic circuit with the capacity to associate the presence of a given input to the gene expression and reinforce expression of reporter in the presence of a desired input. The DNA writer fused to an activator domain (VP64) binds to an operator site (red box) upstream of a minimal promoter, resulting in a weak expression of the reporter gene. Once bound, the DNA writer can edit the neighboring site upstream of the first operator site, generating a new operator site which now the DNA editor can bind to. This result in stronger activation of the reporter gene. In the presence of a persistent signal, new operator sites are generated upstream of the existing operator site, resulting stronger and stronger activation of the reporter as a function of the input. If the input is removed, the gRNA expression is halted and reporter expression is stopped; however, if the cells are exposed to the input again, the response would be as strong as the response before the removal of the inducer (associative learning). FIG. 20B shows an example of a design where the circuit “forgets” an existing reinforced expression. In this case, at presence of an input, an operator array upstream of the reporter is gradually destroyed as a function of the DNA writer/gRNA expression, reducing the number of transactivator binding sites (i.e. operator sites), thus weakening of the reporter promoter. FIG. 20C shows the generation of gRNA operator arrays by stepwise editing of a DNA sequence in vivo using DNA writers. In response to the inducer (aTc), gRNA (with the given sequence) binds to the first operator (Op) site, and edits a dC residue in this region. This result in the generation of a new Op upstream of the original Op which in turn leads to new editing and Op sites.

### Example 13. Complex DOMINO Genetic Programs

FIG. 21A shows a three input sequential AND-gate. Ordered expression of the three input gRNAs (red, blue and brown, respectively) by their corresponding inducers lead to sequential change of the initially inactive output gRNA. Once all three modifications are made on the output gRNA, it is activated and can execute a function on a downstream gene (e.g. base editing, repression, or activation) or a gRNA. FIG. 21B shows an example of a timer/integrator device. A self-targeting gRNA (stgRNA) module is modified by the DNA writer in response to the incoming signal controlling the stgRNA promoter. As a result, mutations accumulate in the stgRNA region over time as a function of the magnitude and duration of the incoming signal. Different states of the specificity determining sequence (SDS) of the stgRNA can be linked to different outputs. As the mutations accumulate in the stgRNA locus, different outputs are sequentially executed.

### Example 14. Examples of DOMINO-Based State and Turing Machines

FIG. 22A shows an example of a complex sequential circuit that uses genomic DNA as a memory tape to achieve a state-dependent genetic program. In this circuit, in the presence of an input, the first (pink) gRNA initiates a cascades of DNA writing events. The pink gRNA binds to cognate target (pink box) and modifies the neighboring DNA bases so that a new target sites is produced, to which the first gRNA can bind. This in turn leads to a series of subsequent modifications and production of a new target sites for first gRNA which eventually leads to activation of the second (green) gRNA promoter (which is initially inactive). Once expressed, the second gRNA initiates another series of DNA writing events that eventually leads to activation of downstream reporter gene (GFP) and modulation of host regulatory genes. FIG. 22B, left panel, shows a schematic representative of a Turing machine, which is a hypothetical computing machine that can perform computation by modifying symbols on an infinite memory tape in using a read/write head, based on a predefined set of rules and input variables. In the simplest form, the symbols on the memory tapes are digital (e.g., 0s and 1s). A Turing machine that has conditional branching function (i.e., if and goto functions) is called Turing complete. FIG. 22B, right panel, shows that to build a biological Turing machine, the genomic DNA of living cells can be used as a form of memory tape, where A, C, G and T are the symbols on this tape. DNA writers can modify the symbols on this tape (cytidine deaminase writer module to encode C->T mutations (or G->A mutations on the reverse strand), and adenine deaminase writer module to encode A->G (or T->C mutations on the reverse strand). The Cas9 variant fused to these writer module can read the sequence of memory tape, and write new information based on a predefined set of rules (e.g., gRNA sequence “if” the sequence homology requirement between the gRNA and the target is met). The “goto” function can be encoded by gRNAs configured in a cascade (as shown in FIG. 21A). As such, the DOMINO platform and the described DNA writers can be used to build complete biological Turing machines.

### Example 15. Engineering an Efficient Read-Write Head for Genomic DNA

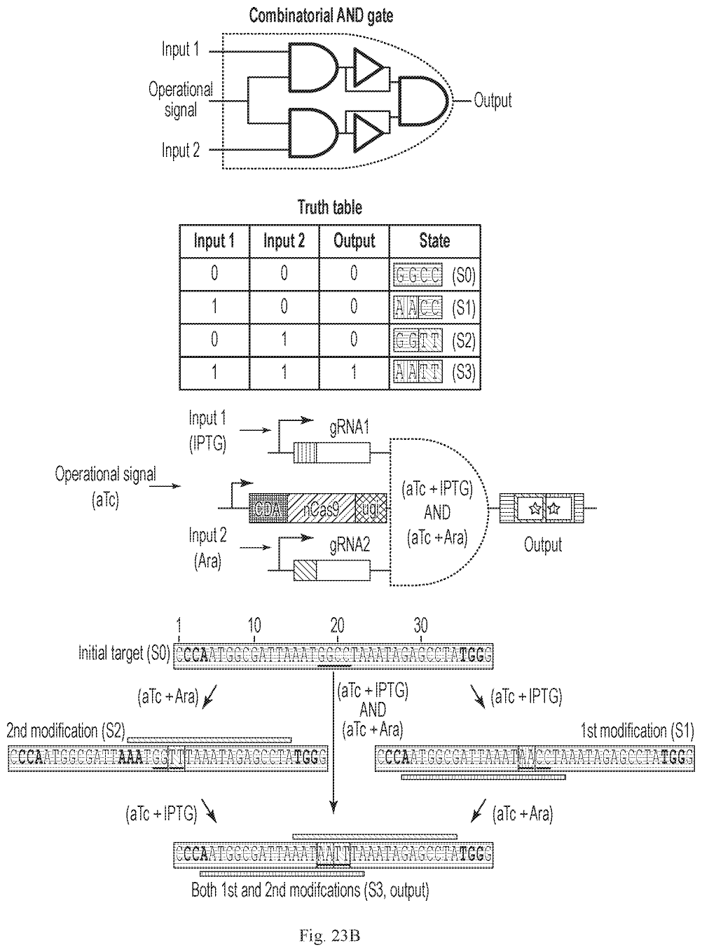

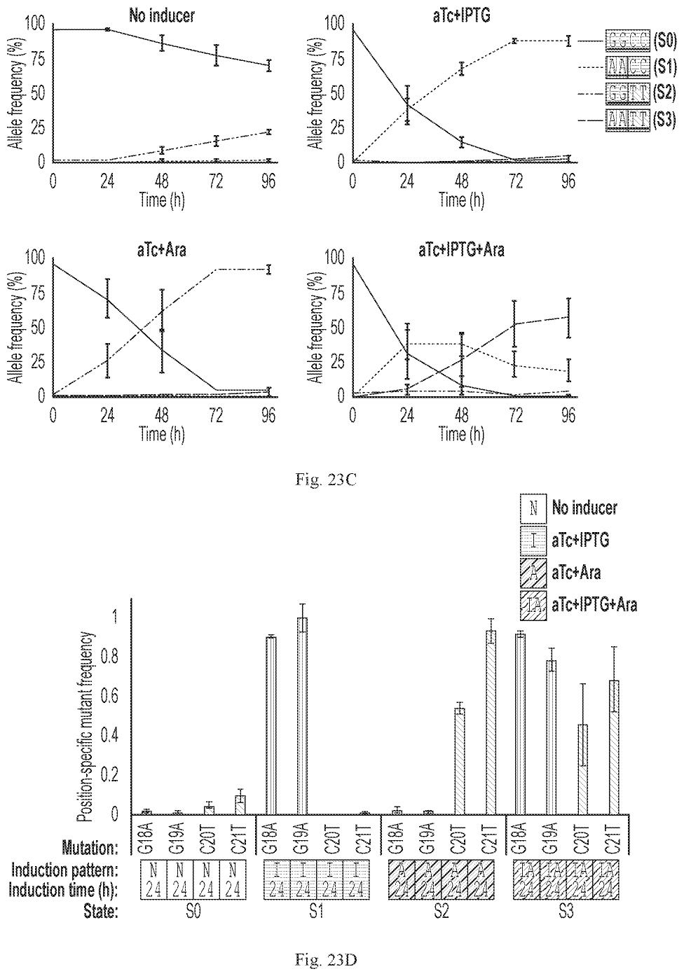

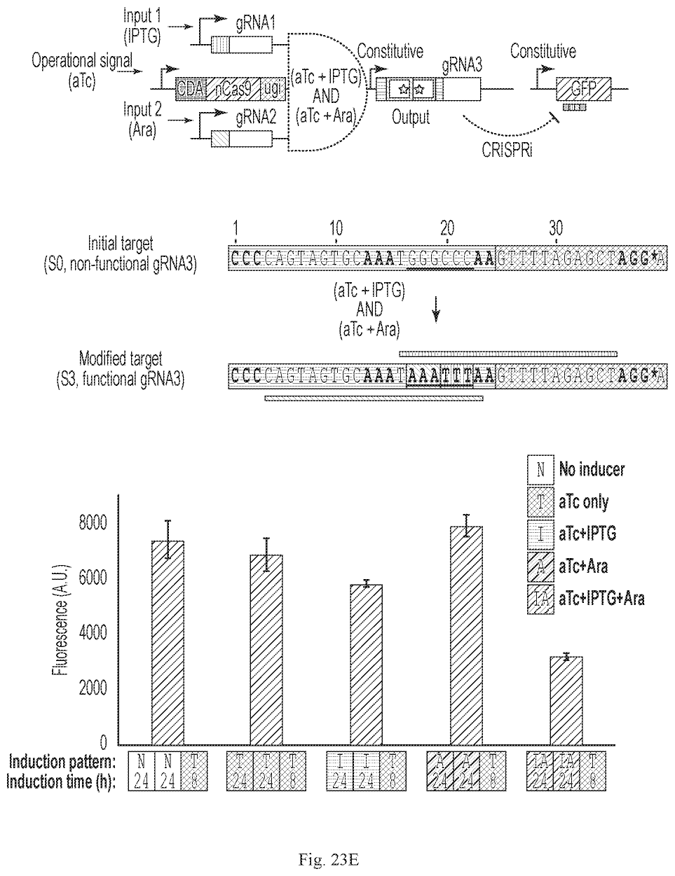

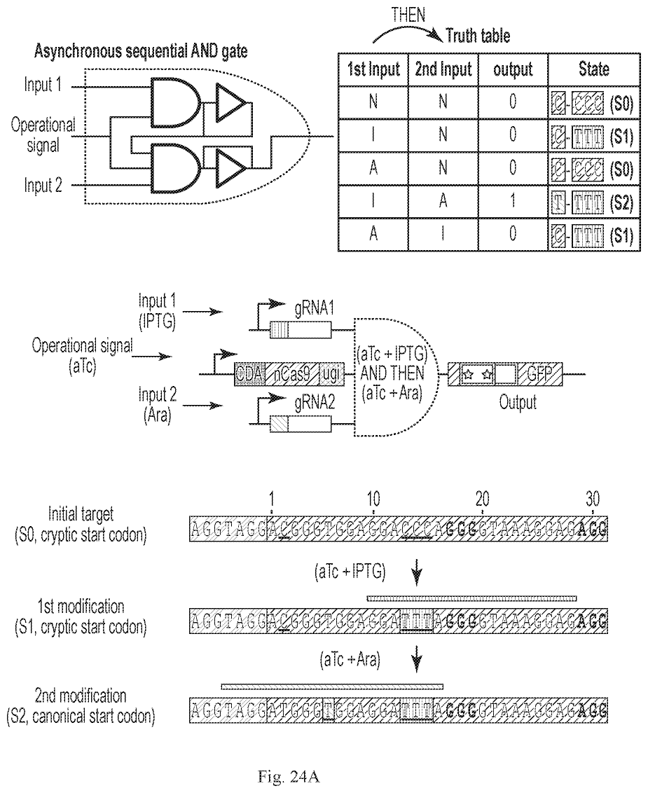

In order to efficiently manipulate genomic DNA in living cells, a single-nucleotide resolution “read-write head” was built for this medium. To this end, a Cas9 nickase (nCas9, an addressable DNA “reader” module that is directed by gRNA to bind to specific DNA targets and nicks them) was fused to cytidine deaminase (CDA, a DNA “writer” module that edits the DNA) and uracil DNA glycosylase inhibitor (ugi, a peptide which has been shown to improve the DNA writing efficiency by blocking cellular repair machinery) to create CDA-nCas9-ugi (7). Once localized to the target based on the 12 bp gRNA seed sequence (“READ” address), the writer module can deaminate dC positions in the vicinity of 5′-end of the target (“WRITE” address), thus resulting in DNA lesions that are preferentially repaired as dT (7, 8). Using cytidine deaminase as the DNA writer module enables dC to dT mutations (or dG to dA mutations if the reverse complement strand is targeted) to be introduced to the WRITE address, resulting in permanent records in DNA. In this memory scheme, an individual mutation or a group of mutations in a target site can be designated as a unique memory state for the corresponding memory register, and mutations introduced by DNA writing events can be considered as transitions between DNA memory states (FIG. 23A). DNA writing events can be controlled by internal or external inputs by placing both the gRNA expression and CDA-nCas9-ugi under regulation by inducible promoters.

This approach enables highly efficient, robust and scalable DNA writing in E. coli. First CDA-nCas9-ugi was placed under the control of anhydrotetracycline (aTc)-inducible promoter. Using an Isopropyl β-D-1-thiogalactopyranoside (IPTG)-inducible gRNA as an input, efficient and inducible DNA writing (dC to dT mutations) was demonstrated at desired target sites in the presence of aTc and IPTG induction (FIG. 23A). In this design, which forms the basis of DOMINO operators, the signal controlling the expression of CDA-nCas9-ugi (aTc) that is required for the overall circuit to function can be considered as the “operational signal”, while the signals controlling expression of individual gRNAs can be considered as independently controllable “inputs”.

### Example 16. Combinatorial DOMINO Logic

DOMINO operators can be arrayed and interconnected in a highly scalable fashion to build robust and complex forms of computing and memory circuits that execute a series of combinatorial and/or sequential unidirectional DNA writing events. The frequency and order of these DNA writing events can be controlled by internal and external cues, as well as by carefully selecting the position of mutable residues within the target. For example, a two-input combinatorial AND logic gate was built by layering two DOMINO operators (FIG. 23B). In this design, two distinct gRNAs were placed under the control of IPTG- and Arabinose (Ara)-inducible promoters, respectively. In the presence of its corresponding inducer, each gRNA is expressed and directs the DNA read-write module (which itself is expressed in the presence of the operational signal, aTc) to its cognate target site, resulting in precise dC to dT mutations (or dG to dA mutations in cases where the gRNA targets the reverse-complement strand) within the WRITE address.

To assess the performance of the combinatorial DOMINO AND gate, cells harboring this circuit were induced with different combinations of the inducers for multiple days and analyzed dynamics of allele frequencies at the target locus by high-throughput sequencing (HTS) over multiple time points. As shown in FIG. 23C, in the presence of the operational signal (aTc) and each of the two inputs (IPTG or Ara), mutations were accumulated in the target sites of the induced gRNA in a linear fashion within the population and comprised ˜100% of the population after 72 hours of induction. This corresponds to transitions from the unmodified state (state S0) to either of the two singly modified states (state S1 or S2). The time required for transitioning between the two states can be considered as the “propagation delay” of the corresponding DOMINO operator. On the other hand, when cells were induced with both inputs (IPTG AND Ara), the target sites for both gRNAs were edited, resulting in the accumulation of doubly edited sites (state S3) in the target locus. States S0, 51, and S2 were defined as the OFF states and S3 as the ON state, which means that this system implements AND logic. In this experiment, low levels of a singly mutated allele (state S2) accumulated in the absence of any induction, likely due to leakiness of the Ara-inducible promoter (pBAD) in these cells and/or high binding efficiency of its corresponding gRNA. The ideal performance of the circuit can be improved by lowering this basal activity, for example by overexpressing pBAD repressor (araC) or using tighter promoters, or alternatively, by lowering copy numbers of DOMINO operators. Nevertheless, the doubly edited allele (state S3) only accumulated in the presence of both IPTG and Ara.

Notably, these results show that in DOMINO operators, the accumulation of the singly mutated alleles in the presence of the operational signal and individual inducer inputs follows a linear trend over the course of few days. About 3 days were required for the unmodified allele to be fully converted into the modified allele(s), thus indicating the propagation delays of the corresponding operators. This feature enables one to use DOMINO to implement both analog and digital computing, since continuous changes that occur within the propagation delay window can be used to implement analog computation, while fully converted states can be considered as transitions between digital states and thus used for digital computation.

The states designated in the AND gate logic described in this example are arbitrary defined; for example, the doubly mutated allele (state 3) was defined as the ON state. The same circuit can be defined, for example, as a NAND gate if the unmodified state (state 0) is designated as ON (“1”) output and states S1 through S3 are designated as OFF (“0”) outputs. Alternatively, each of the four different states can be defined as distinct outputs, in which case the circuit can be considered as a 2-input/4-output demultiplexer system.

In this experiment, two mutable residues within the editing window of each gRNA were used, and the memory states were defined so that mutations in both of these residues were required to be considered as a state transition. One could call mutations in only one of the two nucleotides available for editing as intermediate states, or if desired, discrete transient memory states. The number of memory states as well as the response dynamics (e.g., propagation delay) for each DOMINO operator can be tuned by using different numbers of mutable residues (dC or dG) within the WRITE window, or adjusting the position of these residues within this window.

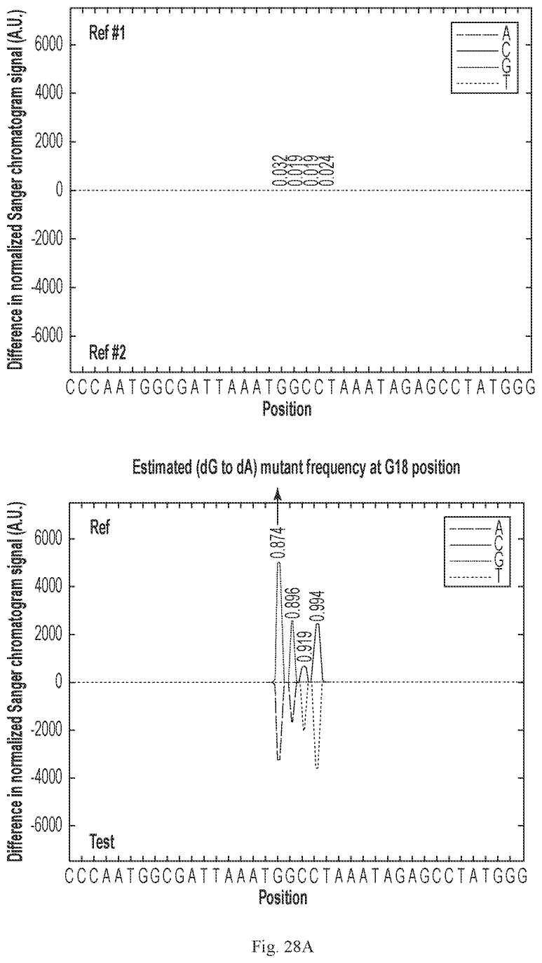

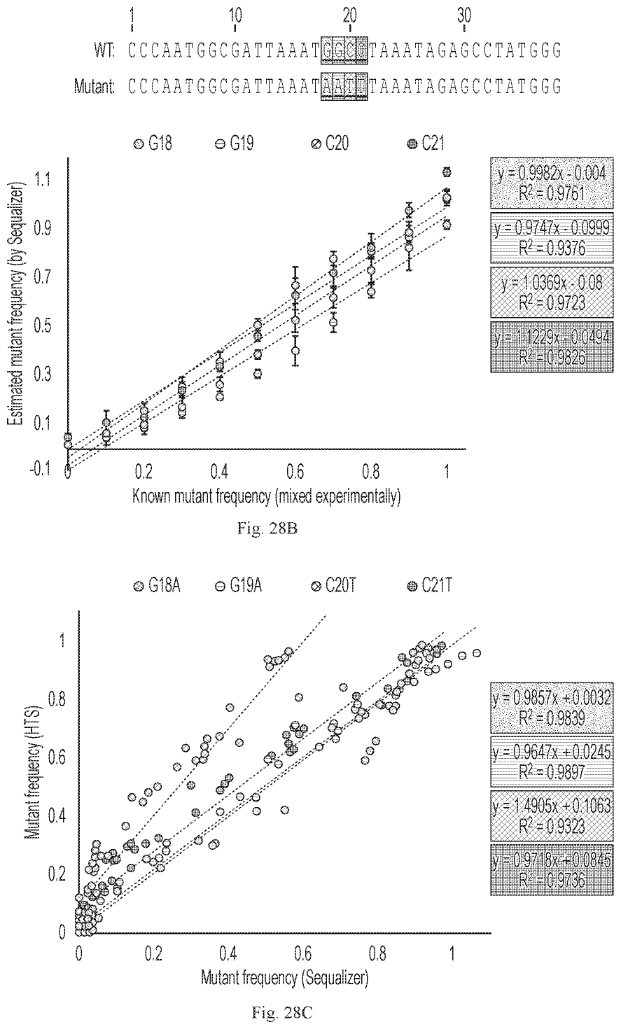

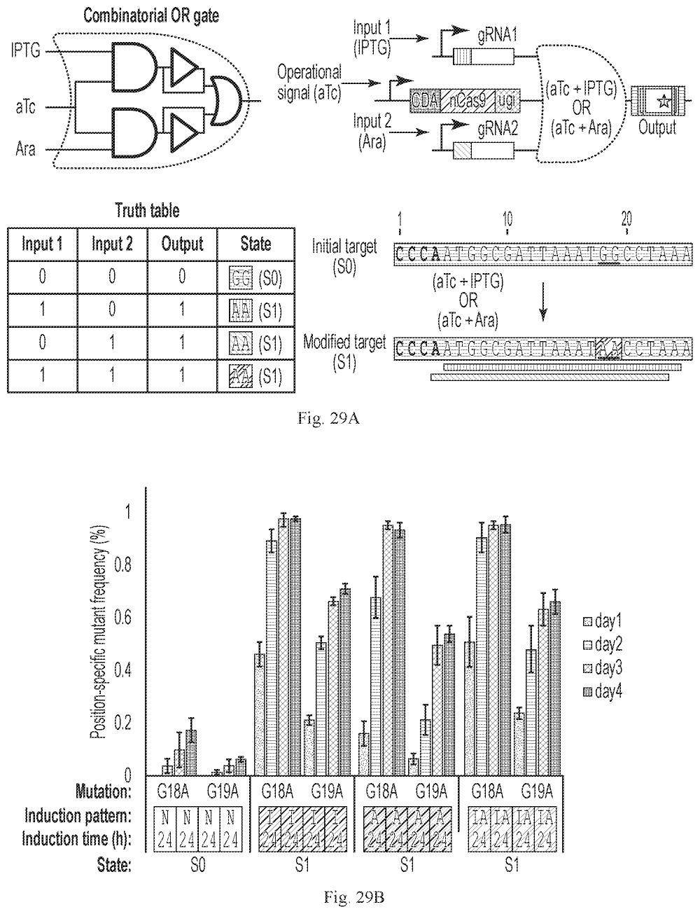

While HTS offers a powerful way to quantify the outcome of DOMINO circuits, its relatively high cost led to the development of a strategy for using Sanger sequencing chromatograms to quantify position-specific mutant frequencies within a mixture of DNA species. This algorithm, named Sequalizer (for Sequence equalizer), normalizes Sanger chromatogram signals and calculates the difference between the normalized signals from a test sample and an unmodified reference to identify position-specific mutations. It then uses this calculated difference to estimate position-specific mutant frequencies at any given target position. The accuracy of this method was validated by constructing a standard curve based on known ratios of mutant sequences, and comparing the Sequalizer results with next-generation sequencing (see Example 21 and FIGS. 28A-28C). The Sequalizer output, which is based on population-averaged Sanger sequencing results, provides an estimate of position-specific mutant frequencies in an entire population. However, unlike HTS, it does not provide insights into the identities and frequencies of individual alleles in the population. Given the high specificity of the DNA writers and predefined target sites for DNA writing, however, this approach can be used as a low-cost alternative to HTS to assess performance of DOMINO and other precise genome-editing platforms.

In addition to HTS, the samples obtained from the experiment shown in FIG. 23B were analyzed by Sanger sequencing and Sequalizer. As shown in FIG. 23D and FIG. 28C, the Sequalizer results were consistent with and could estimate position-specific mutant frequencies obtained by HTS. Specifically, in samples induced with either of the two inputs, the frequencies of mutants in positions corresponding to the cognate target sites of the induced gRNA increased in the population. In addition, in samples that were induced with both gRNAs, the mutation frequencies in the target sites of both gRNAs were increased (state S3).

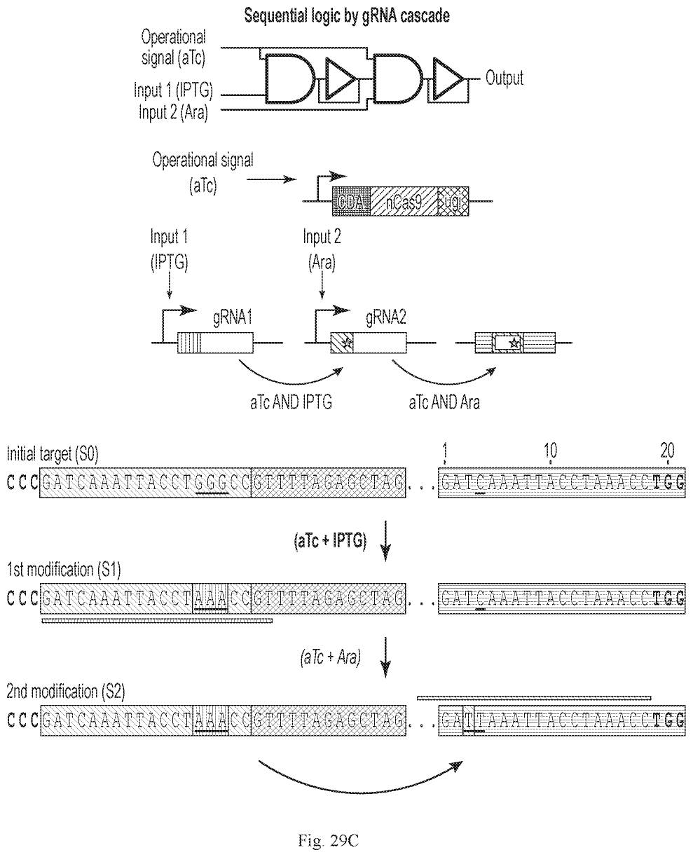

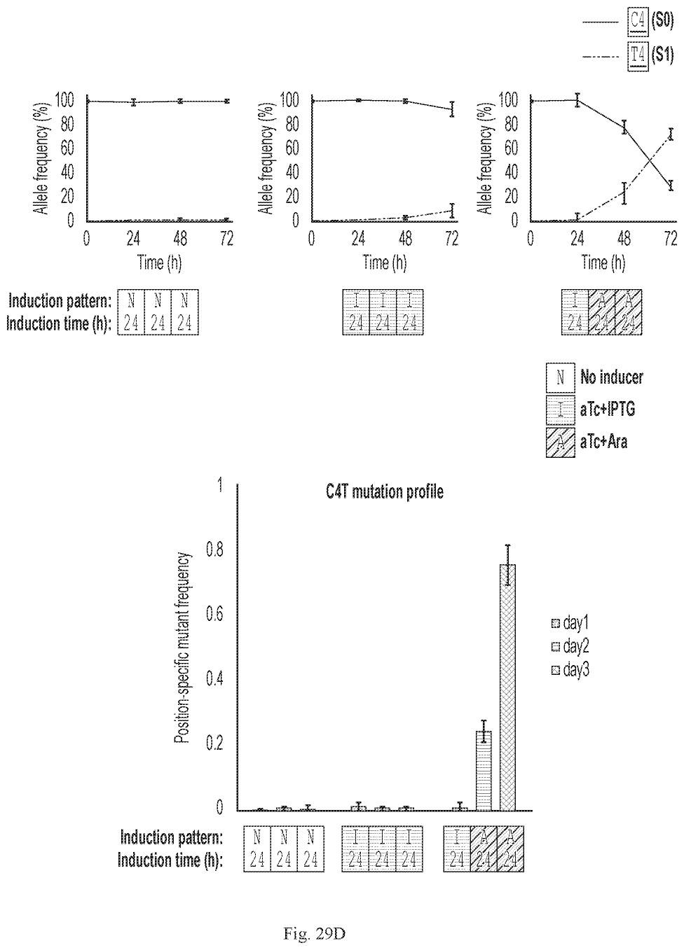

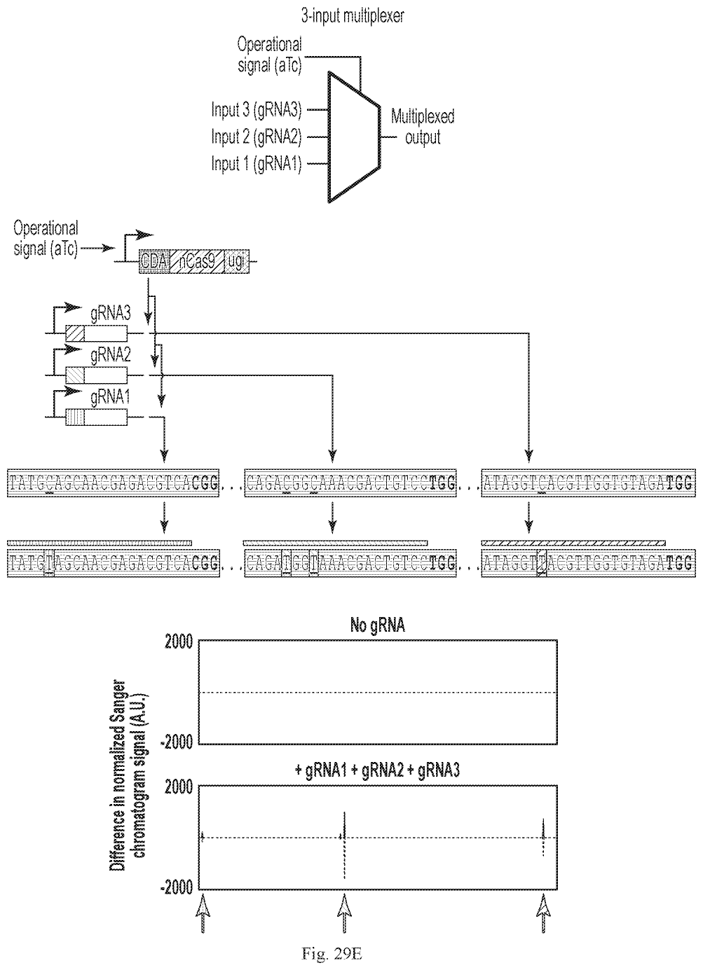

In addition to AND gate, other logic can be readily implemented by carefully positioning mutable residues on the targets, as well as designing the combinations and order of DNA writing events. Furthermore, additional input gRNAs can be incorporated to achieve operators with more than two inputs, thus demonstrating scalability of this approach (FIG. 29).

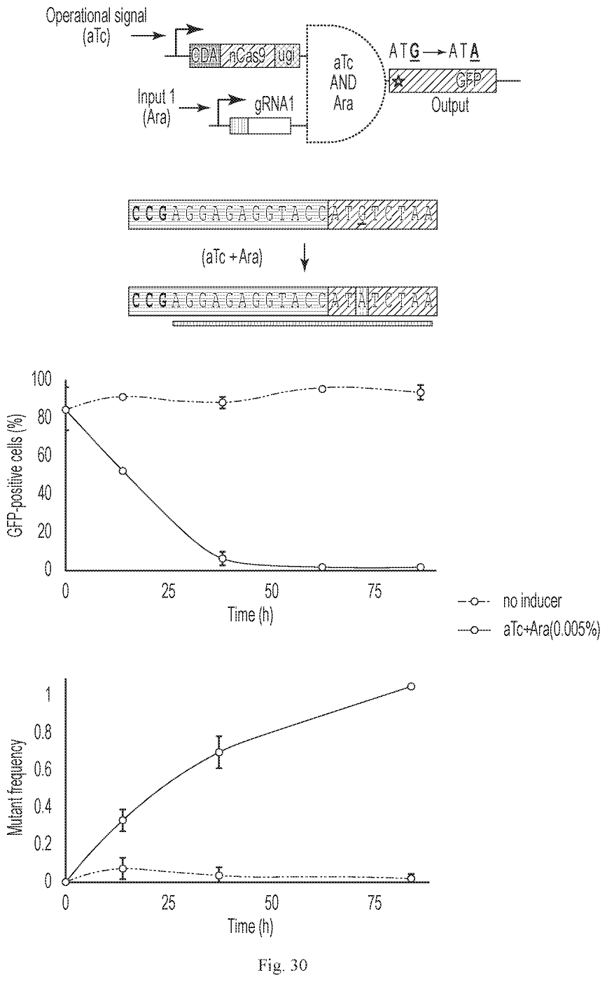

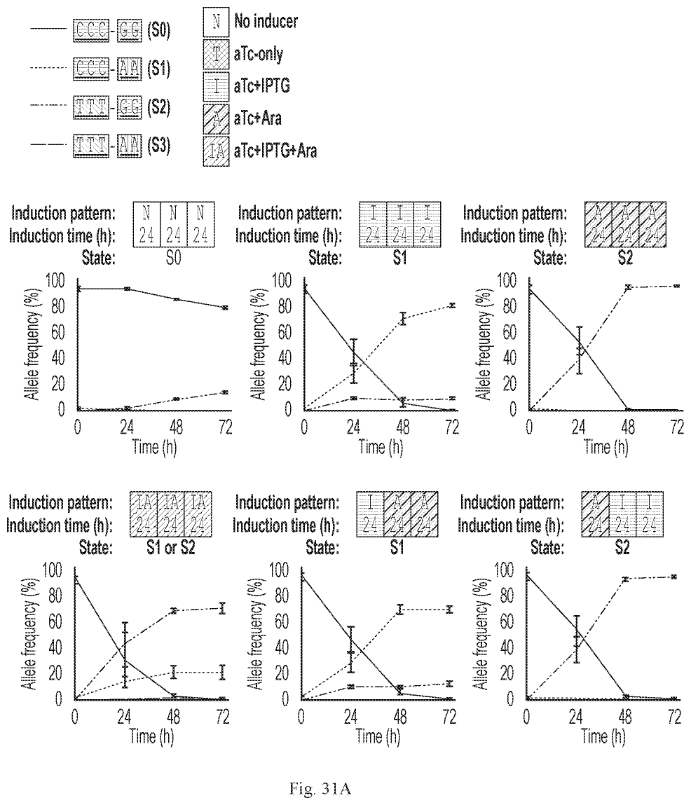

The output of DOMINO operators takes the form of DNA mutations that accumulate at a target site. One can flank this target site with a desired promoter and a gRNA handle to convert the output of a given DOMINO operator into downstream gRNA expression. The output gRNA can then be interconnected with other DOMINO operators to build more complex circuits. In addition, it can be combined with CRISPR-based gene regulation platforms such as CRISPRi and CRISPRa to dynamically regulate cellular phenotypes. To demonstrate this, an AND operator was engineered by layering two DOMINO operators under the control of inducible promoters to edit a third gRNA as the output (FIG. 23E). The input gRNAs were controlled by IPTG- and Ara-inducible promoters, respectively. In the presence of both inducers, the output gRNA was modified by both input gRNAs such that it could then bind to and repress a downstream reporter gene (GFP) (FIG. 23E, aTc+IPTG+Ara co-induction for two 8-hour periods followed by aTc-induction for 8 hours ([IA][IA][T] induction pattern)). When targeting gRNA as an output, both the Specificity Determining Sequence (SDS) of the output gRNA as well as its constant region (handle) can be modified. Mutating the SDS is useful when the creation of a unique gRNA is the desired output. On the other hand, mutating the gRNA handle enables one to activate/deactivate an entire set of gRNAs. Furthermore, one can also target gene regulatory and functional elements, such as promoters, ribosome binding sites, start/stop codons, as well as active sites within proteins to tune the expression or activity of downstream components as shown in FIG. 30.

### Example 17. Sequential DOMINO Logic

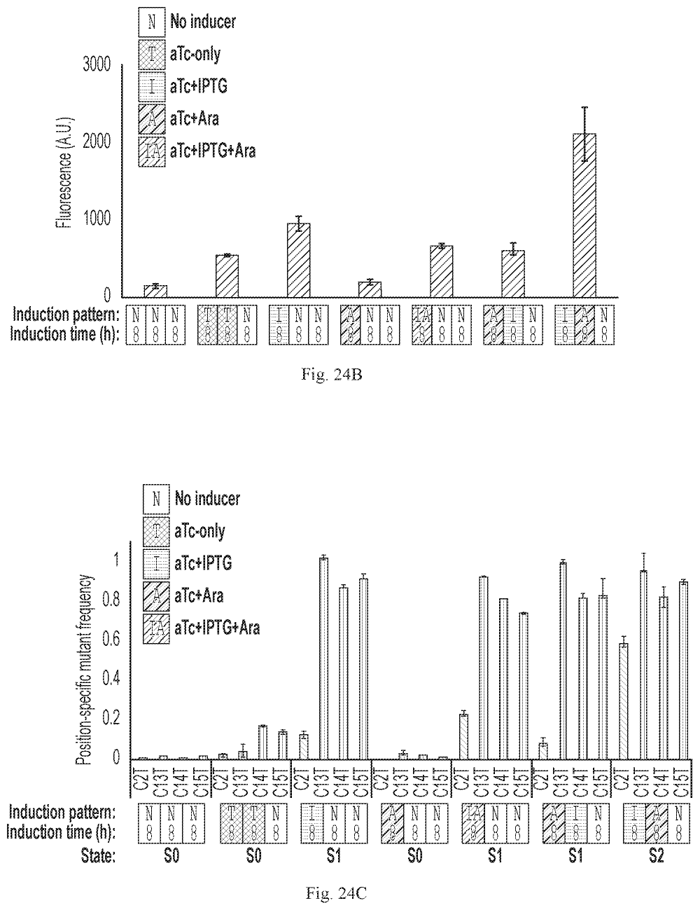

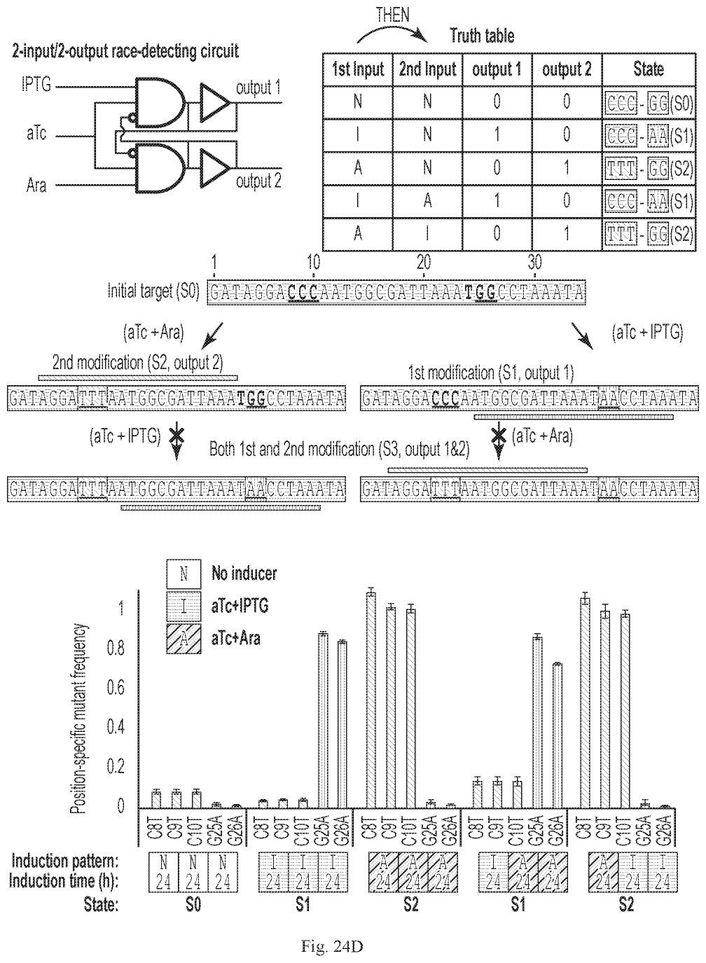

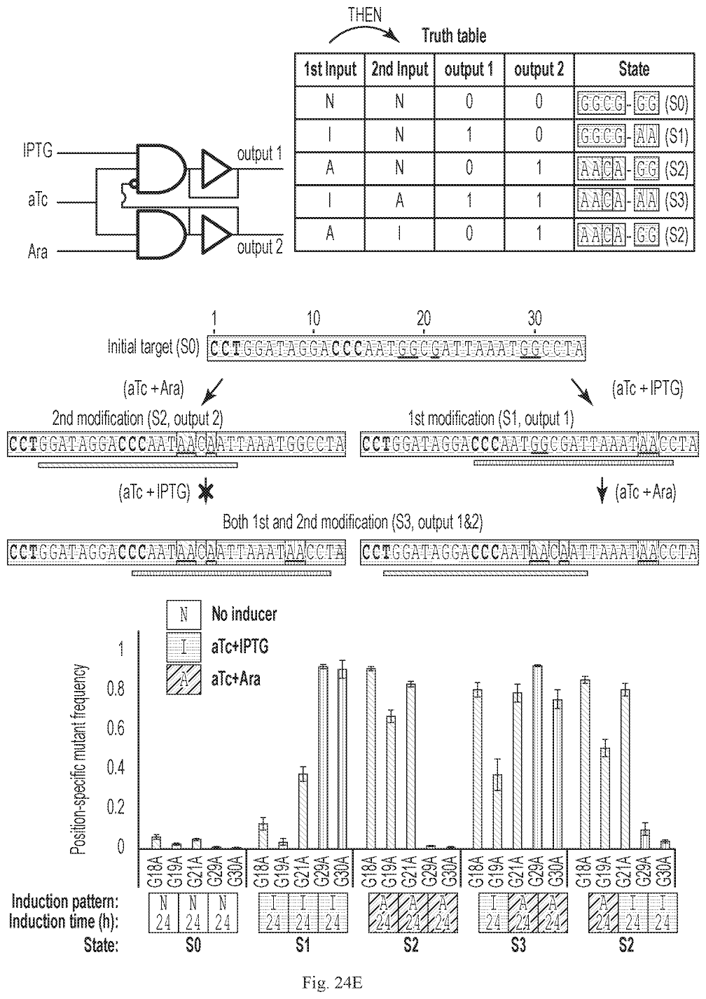

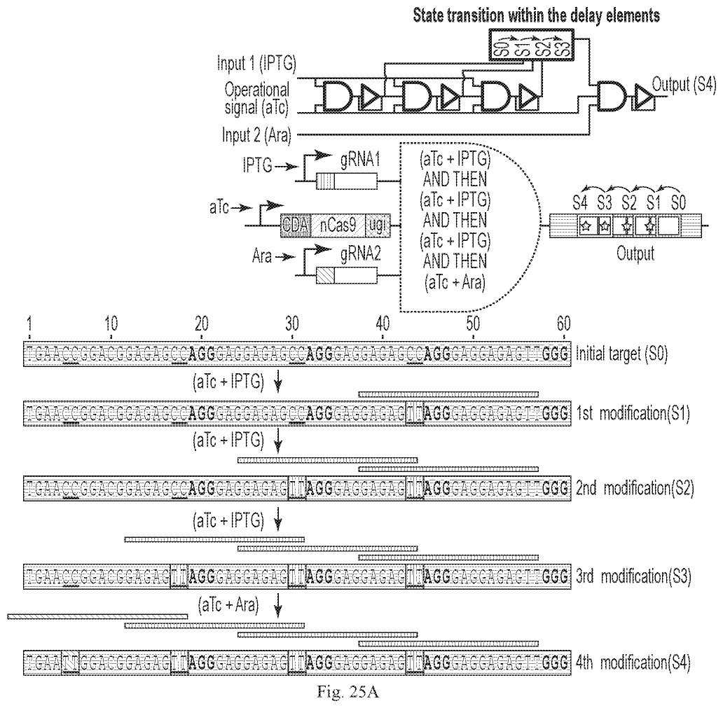

In addition to realizing combinatorial logic, one can carefully control the sequence and timing of DNA writing events executed by DOMINO operators to achieve sequential logic, where desired outputs are generated only when the correct order of inducers is added. To achieve this, for example, one can design the gRNA output of one operator to be used as the input for a downstream operator (FIG. 29C). This design can be used to functionally connect DOMINO operators that are not physically co-located, and offers control over the individual DOMINO operators. Alternatively, sequential logic can be achieved by overlapping mutable residues in the WRITE address of one operator with the READ address of a downstream operator (FIGS. 24A-24E). This design uses DNA mutations rather than cascades of gRNAs as a way to interconnect cis-encoded DOMINO operators, thus offering a highly compact and scalable strategy for encoding sequential logic.

To demonstrate the latter strategy, an asynchronous sequential AND gate was first constructed, where sequential addition of the two inputs in the correct order (IPTG AND THEN Ara) leads to mutation of a cryptic start codon (ACG) into the canonical (and more efficient) start codon (ATG) in the GFP ORF, thus increasing the GFP signal (FIGS. 24A and 24B). Slight increases in GFP signal was observed in cells that had been induced with the first inducer (i.e., IPTG) or those that had been co-induced with both inducers (FIG. 24B). The former was likely caused by the leakiness of the second (Ara-inducible) promoter while the latter was likely due to the simultaneous presence of both inducers in the media, which could result in the execution of sequential DNA mutations in the correct order to some extent. Nevertheless, the GFP signal was significantly higher when cells were exposed to the correct order of the inducers. These results were further confirmed by analyzing Sanger sequencing chromatograms by Sequalizer (FIG. 24C). Consistent with flow cytometry data, samples induced with the correct order of the inputs showed the highest level of the dC to dT mutation in the position corresponding to the cryptic start codon (FIG. 24C), indicating the execution of a cascade of DNA writing events that lead to execution of sequential AND logic.

As another example, an asynchronous 2-input/2-output race-detecting circuit was built, where the output of the circuit is determined by the inducer added first and not the other inducer added second (FIG. 24D). In this design, the PAM domain for each gRNA is placed within the WRITE window of the other, in a way that editing mediated by one gRNA destroys the PAM domain for the other gRNA, thus preventing binding and subsequent editing by that gRNA. As shown in FIG. 24D, Sequalizer analysis of cells induced with different combinations of inducers showed that the output of the circuit depends on the identity of the first inducer. Specifically, cells that were first induced with IPTG were converted to state 51, independent of addition of the second inducer (Ara) at a later stage, and those cells that were first induced with Ara were converted to state S2 independent of IPTG induction.

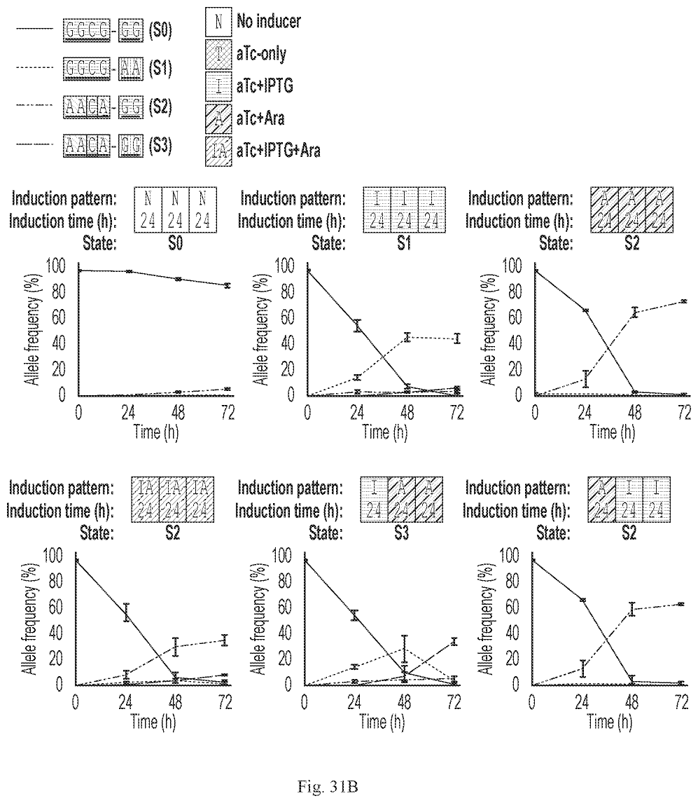

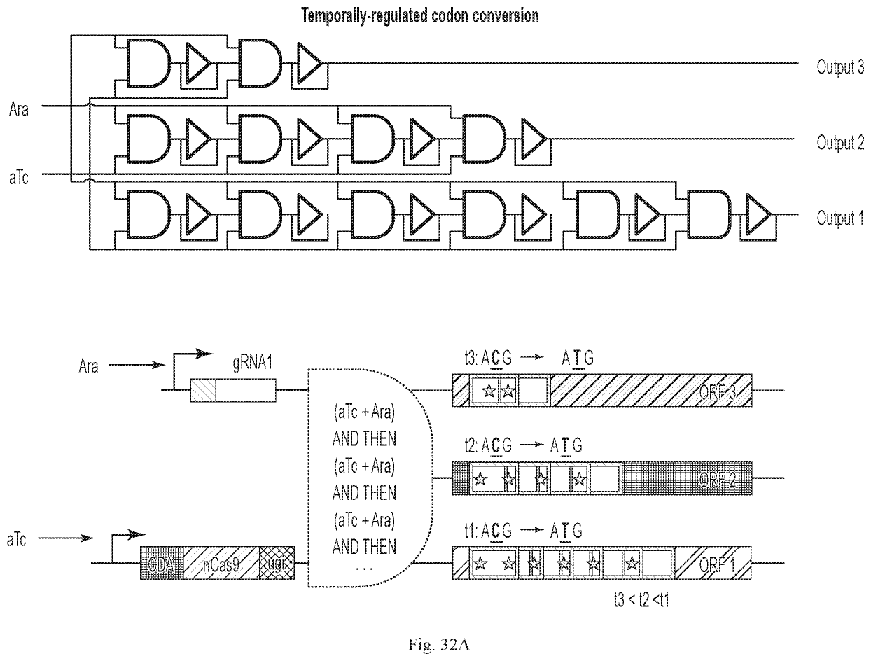

When cells were induced with IPTG AND THEN Ara (FIG. 24D, IPTG induction for one day AND THEN Ara induction for two days ([I][A][A] induction pattern)), a slight increase in the mutant frequency was observed in the positions corresponding to targets of the Ara-inducible gRNA. It was suspected that this was due to leakiness of the Ara-inducible promoter during IPTG induction period (i.e., before ending the propagation delay of the first operator), which would lead to expression of gRNA2 and aberrant transition of a small subpopulation of cells to state S2. Nevertheless, since editing by one gRNA should destroy the PAM domain for the second gRNA, the race-detecting logic should still hold within each single DNA molecule. High-throughput sequencing of these samples revealed that indeed this was the case since doubly edited allele (i.e., state S3, corresponding to editing events by both gRNAs) were extremely rare (FIG. 31A).

This experiment indicates that the ratio between edited alleles in a population can be tuned by controlling the induction time of each of the inputs, while ensuring that the desired logic is applied at the level of each individual DNA molecule. Alternatively, if conversion of the whole population to a final state is desired, one can perform each induction step for periods longer than operator's propagation delay (i.e., multiple days) to allow the full conversion of cells to a given state before moving to the next induction step. This control over the degree of commitment of cells to different states could be useful for dividing biological tasks between different subpopulations in a community. For example, one subpopulation of cells could be edited to activate metabolic pathway 1 and the other subpopulation of cells could be edited activate metabolic pathway 2; the relative ratio of activation could be tuned using the DOMINO circuits to control the overall population performance.

Finally, a 2-input/2-output sequential logic circuit was constructed, where induction with IPTG AND THEN Ara results in step-wise transition between two modified states (a sequential AND gate) while induction in the opposite direction (i.e., Ara AND THEN IPTG) results in transition to a different state. In this circuit, editing mediated by one gRNA destroys the binding site of the other gRNA, while editing mediated by the second gRNA does not interfere with the binding or editing of the first gRNA. As shown in FIG. 24E, this circuit is an intermediate circuit between the sequential AND gate (FIG. 24A) and the race-detecting circuit (FIG. 24D). Induction of this circuit with IPTG resulted in the transition of the target register from the initial unmodified state (state S1) to the first modified state (state S1). Subsequent induction of these cells with the second inducer (Ara) led to transition of these cells to the doubly mutated state (state S3). On the other hand, when cells were first induced with Ara, they were converted to an alternative singly modified state (state S2). However, subsequent induction of these cells with IPTG did not result in a transition, thus realizing the expected behavior. Using high-throughput sequencing, it was confirmed that expected transitions between the states, and thus the circuit logic, held at the single-molecule level (FIG. 31B).

### Example 18. Temporal DOMINO Logic

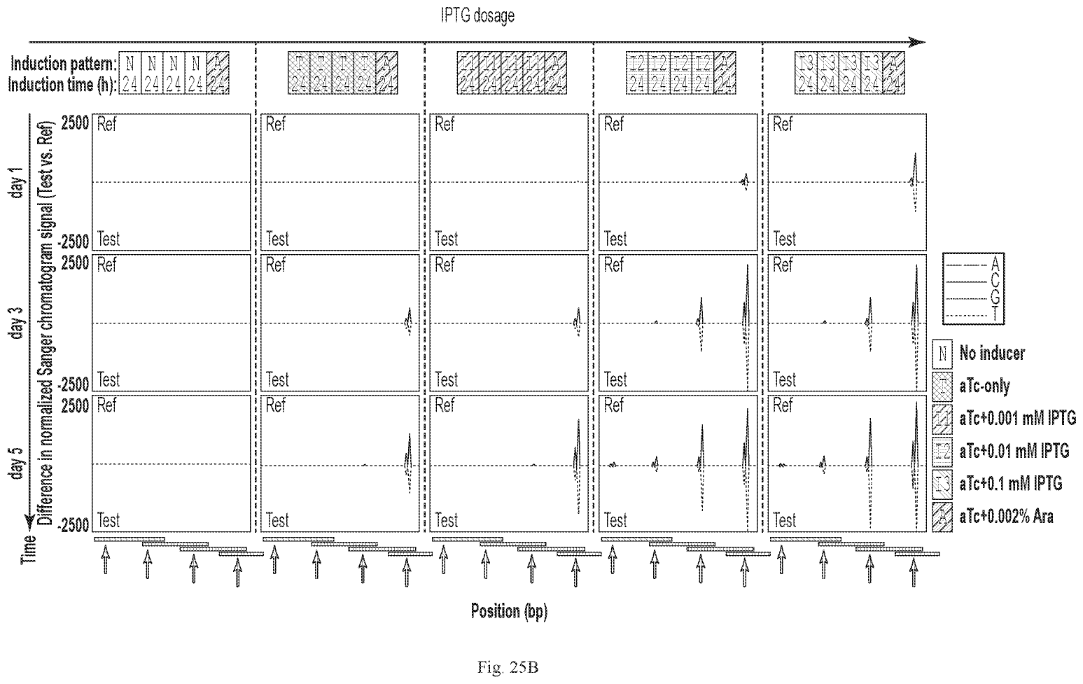

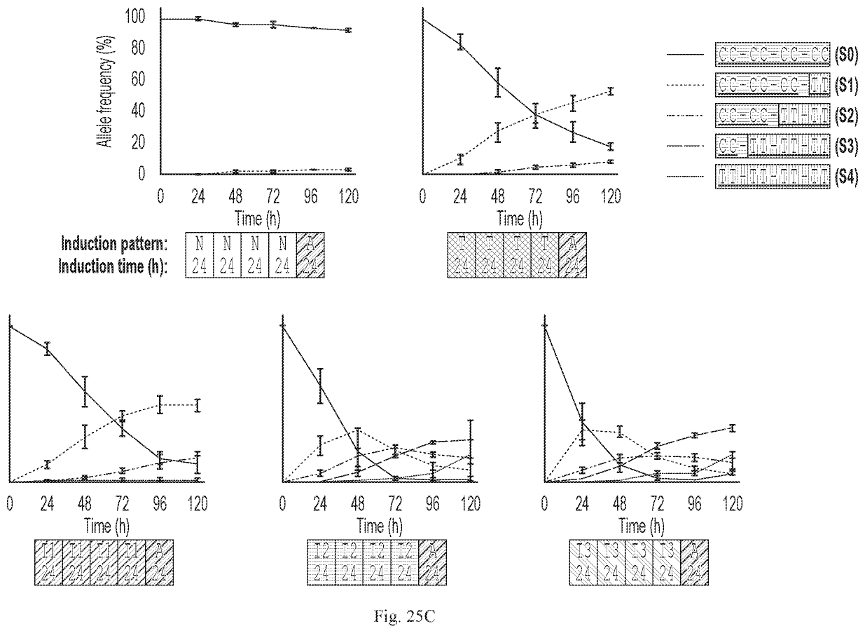

The above examples demonstrate that the sequence and timing of DNA writing events mediated by DOMINO operators can be controlled by external cues. In addition to building sequential logic, where the execution of events in a specified order leads to a desired output, the propagation delay in DOMINO operators can be exploited to incorporate temporal logic into circuits, where a desired output is produced only after a certain period of time has passed. In a simple form, DOMINO delay operators can be built by constructing a series of overlapping repeats to act as target sites for a desired gRNA (FIG. 25A). This repeat configuration allows one to overlap the READ address of each gRNA operator site with the WRITE address of the previous gRNA. Initially, the gRNA can bind to the first (i.e., 3′-end) repeat, but not to the upstream copies of the repeat that harbor dC residues (instead of dT) in the sequence corresponding to the gRNA READ address (i.e., the gRNA seed sequence). Upon binding to the first repeat, the gRNA can mutate the dC residues in the repeat immediately upstream of its binding site (i.e., the second repeat), thus converting that repeat to a new binding site for another copy of the same gRNA. This process is sequentially repeated to generate new binding sites for the gRNA. Much like an array of physical domino pieces that fall down one by one, each genome-editing event is initiated only after editing in the previous repeat has occurred, thus ensuring a sequential cascade of DNA writing events. The total delay can be tuned by changing the number of the repeats, modifying the overlapping distance between the repeats, or adjusting the distance of mutable residues from their corresponding PAM sequences.

In addition, the output of the delay elements can be combined with additional logic operators and internal or external cues to create more complex forms of temporal logic. To demonstrate this concept, three DOMINO delay elements were placed into an array and linked the output of the array to a second DOMINO operator that implements sequential AND logic (FIG. 25A). This design achieves temporal and sequential AND logic since the first (IPTG-inducible) gRNA has to execute three consecutive DNA writing events before the Ara-inducible gRNA corresponding to the last operator can bind to and edit its target. Cells harboring this circuit were induced with different IPTG concentrations for 4 consecutive days followed by a final day of induction with Ara. Using Sanger sequencing on the population and Sequalizer analysis, a time- and IPTG-dosage-dependent accumulation of mutations in the target sites within repeats was observed, corresponding to propagation of the signal through the repeat array (FIG. 25B). The rate of propagation of the mutation cascade through the delay elements correlated with both the concentration and duration of exposure to IPTG. By the end of the experiment, mutations in the position corresponding to the target site of the second gRNA (shown by the blue arrow in FIG. 25B) were detected only in conditions in which mutations had accumulated through the entire cascade, corresponding to the samples that had been induced with the highest IPTG concentrations.

These results were further confirmed by analyzing these samples with HTS. This analysis also showed time- and IPTG dosage-dependent mutation accumulation within the repeats (FIG. 25C). Furthermore, the mutation corresponding to the target of the Ara-inducible gRNA only accumulated in the later time points and only in cultures induced with high concentrations of IPTG. Upon induction of the samples by Ara, the frequency of the allele corresponding to final output of the circuit (i.e., state S4) only increased significantly in samples that had been previously induced with high (i.e., 0.01 mM and 0.1 mM) IPTG concentration. These results further demonstrates that, in addition to enacting delays in gene circuits, an array of DOMINO delay elements can be used as a multi-state memory register that undergoes transitions between different discrete states (i.e., sequential mutations) in a time- and dosage-dependent fashion. In this design, the number of memory states can be tuned by changing the number of repeats. Moreover, the timing and probability of transitions between repeats can be adjusted by changing the position of mutable residues within the repeat overlaps, or tuned dynamically by external cues.

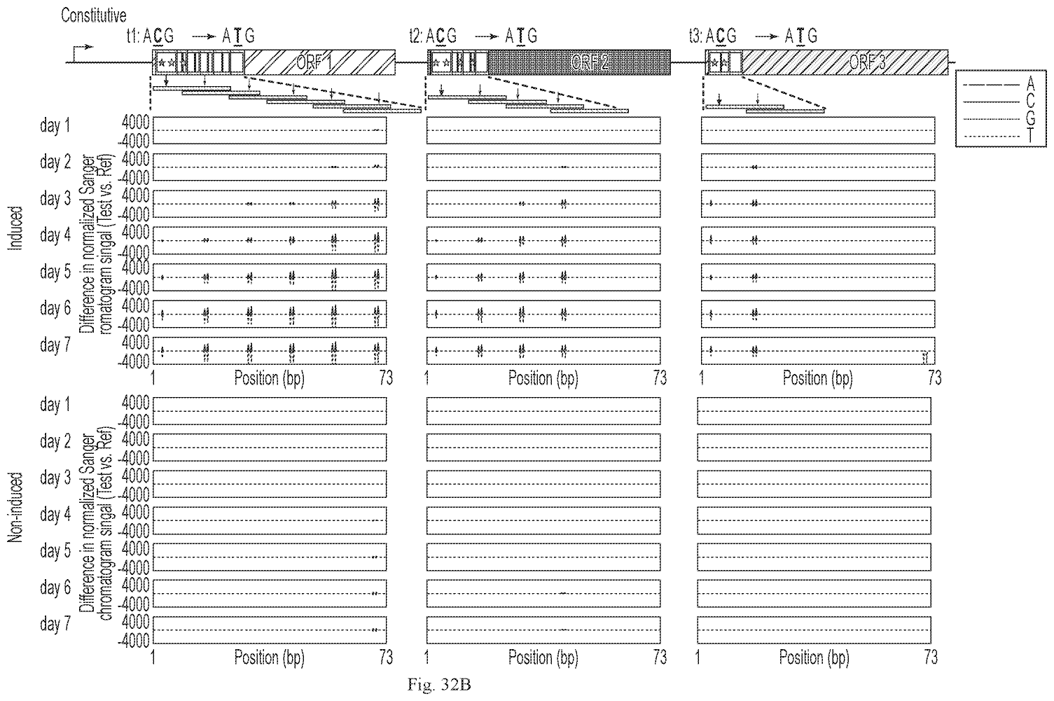

Finally, to demonstrate the power of the technique, DOMINO delay elements were used to build a gene expression program in which the conversion of cryptic ACG start codons into canonical ATG start codons in three different ORFs was temporally controlled by a single input (FIGS. 32A-32B). It is envisioned that more complex versions of temporal logic, such as counters, can be constructed by integrating delay elements into multiple-input DOMINO operators.

### Example 19. Associative Learning Circuits and Online DNA-State Reporters

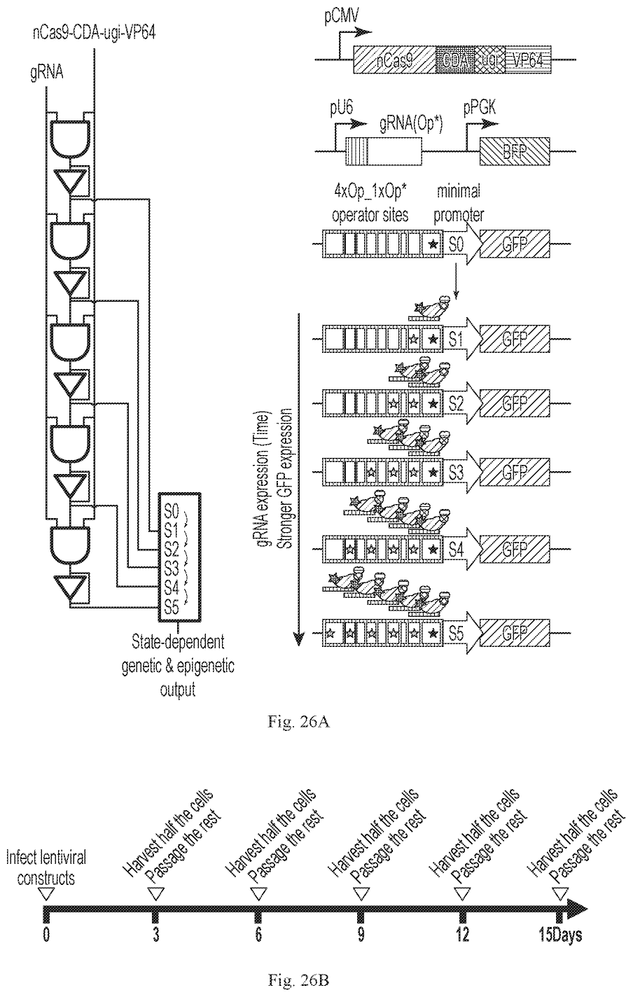

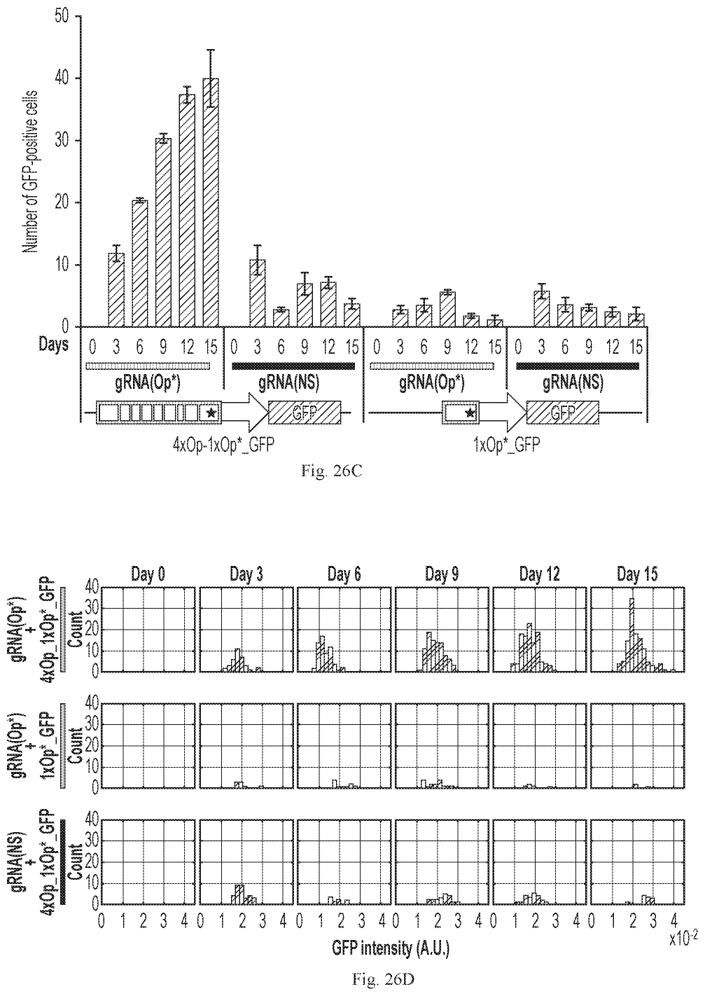

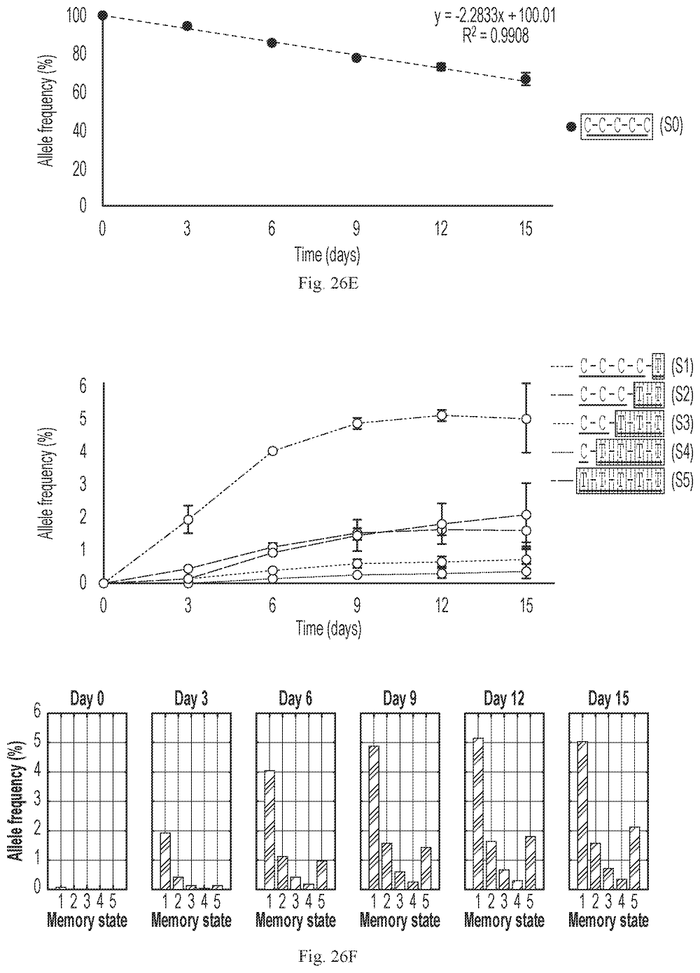

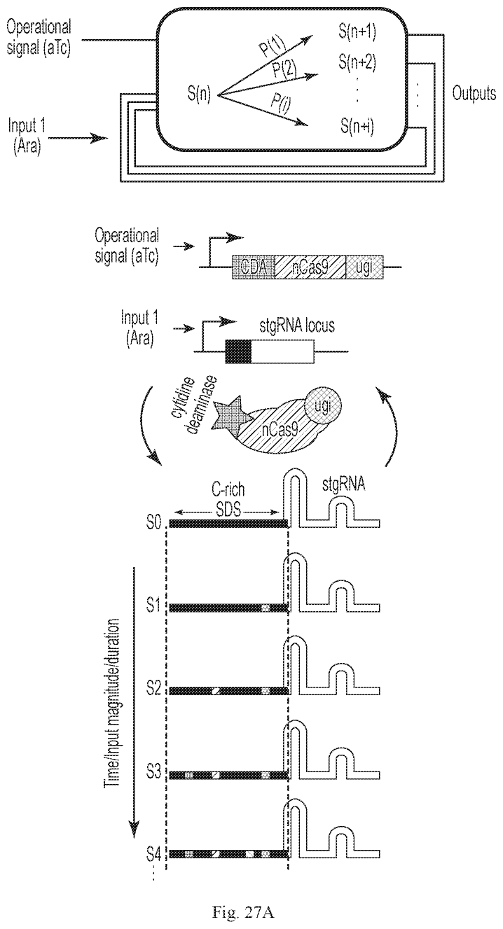

A unique feature of DOMINO operators compared to other memory platforms is that the DOMINO DNA read-write head can be further functionalized with additional effector domains, such as transcriptional activators and repressors, to achieve combined DNA writing and transcriptional regulation. This offers the unprecedented capacity to perform both genetic and epigenetic modulation and thus combine DNA memory states with functional outcomes. For example, this feature enables the construction of circuits that can learn and remember. Specifically, a synthetic gene circuit was devised that undergoes associative learning (15-18) such that its gene expression output is reinforced by a given stimulus (FIG. 26A). While transcriptional positive feedback loop can also be used to implement synthetic self-reinforcing circuits, the state of such circuits can fluctuate due to their reliance on continuous transcription for state maintenance. In contrast, an associative learning circuit that uses genetically encoded memory to gradually reinforce a response remains intact and stable even after the initial stimuli is removed.

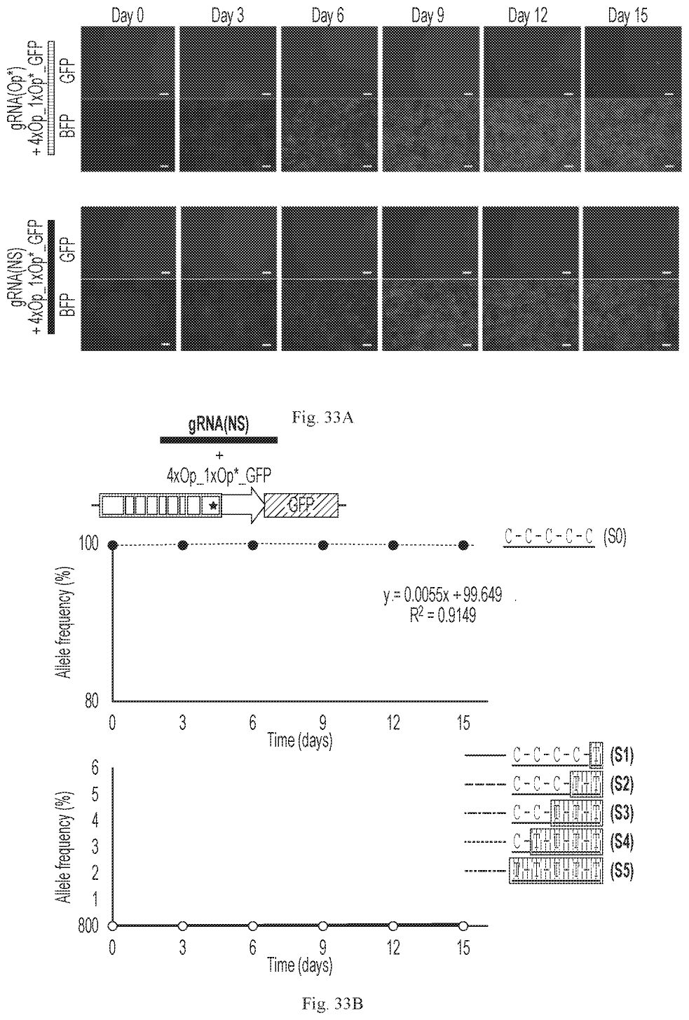

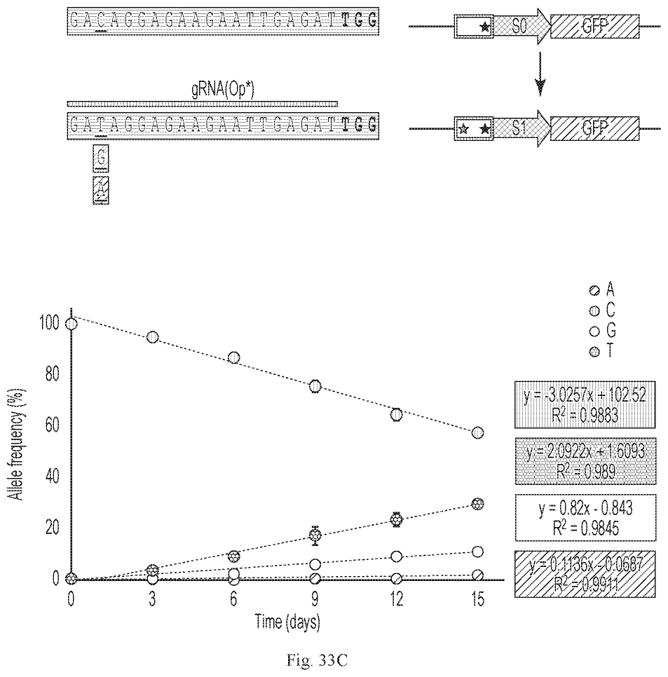

To demonstrate this concept, an array of overlapping repeats (operators) was made, composed of four WT repeats (4xOp) and a downstream mutant repeat (1xOp*) which harbored a dC to dT mutation. This repeat array was then placed upstream of a minimal promoter driving GFP to build 4xOp_1xOp*_GFP reporter construct. Additionally, a second reporter (1xOp*_GFP) was built by placing a single Op* repeat upstream of the minimal promoter driving GFP. The DNA read-write head (nCas9-CDA-ugi) was also functionalized with a transcriptional activator domain (VP64) and the nCas9-CDA-ugi-VP64 fusion construct was cloned along with either of the two reporter constructs into lentiviral vectors which were subsequently introduced into the human HEK 293T cell line. A second lentiviral vector encoding a Op*-specific gRNA (gRNA(Op*)) (or a non-specific gRNA (gRNA(NS)) as negative control) was then delivered to these cells. Upon binding, gRNA(Op*) could bind to Op* repeat and mutate the critical dC residue in the WT Op repeat immediately upstream of its binding site, thus converting Op repeat to a new Op* sequence that could serve as a new binding site for the same gRNA; this strategy enables sequential rounds of mutations (i.e., Op to Op* conversion) and gRNA binding events (FIG. 26A). Cells harboring these circuits were sequentially passaged every three days for fifteen days (FIG. 26B) and GFP expression and the genotype of the cells were observed by microscopy (FIGS. 26C-26D and 33A) and HTS (FIGS. 26E-26F), respectively. As shown in FIG. 26C, the frequency of GFP-positive cells in cultures harboring the 4xOp_1xOp*_GFP reporter and gRNA(Op*) increased over time, indicating the gradual activation of the reporter in the population. On the other hand, the frequency of GFP-positive cells did not change significantly in cultures that were transfected with gRNA(NS), or those that contained the 1xOp*_GFP reporter.

In addition to observing an increased frequency of GFP-positive cells, it was observed that the intensity of the GFP signal in GFP-positive cells increased in cultures that harbored the 4xOp_1xOp*_GFP reporter and gRNA(Op*) over time (FIG. 26D). This data suggests that the number of bound transactivators, and thus, the number of activated (i.e., Op*) repeats that can serve as operator sites for the chimeric read-write-transactivator protein increased in these cells. On the other hand, no significant increase was observed in negative controls that harbored gRNA(NS) or those that that contained the 1xOp*_GFP reporter.

These results were further confirmed by analysis of the allele frequencies throughout the experiment by HTS. As shown in FIG. 26E, the frequency of the WT allele (state S0) in cells containing the repeat array and gRNA(Op*) decreased linearly with time over the course of the experiment. On the other hand, the frequency of intermediate states (51 through S4) gradually increased and reached a plateau towards the end of the experiment, suggesting that these intermediate states reached steady state (FIG. 26F). The allele frequency of the final state (S5) gradually increased over the course of the experiment. No significant change in allele frequency was observed in cells that were transduced with a non-specific gRNA (FIG. 33B). Together with the microscopy data, these results show that the analog properties of a signal, such as the duration of exposure to gRNA(Op*), can be faithfully and permanently recorded within the distribution of memory states of the DNA recorder within the population. On the other hand, at the single cell level, each repeat forms a multi-bit digital recorder that associates longer or higher intensity of exposures to an incoming signal with transitions to higher memory states in the form of more accumulated mutations. The permanently recorded mutations are preserved even after the input gRNA is removed, and thus “learned”. If the cells are re-exposed to the same signal, the response is similar to the state when the signal was initially removed and different from the beginning of the initial exposure (state S0).

In samples harboring the gRNA(Op*) and either of 1xOp*_GFP or 4xOp_1xOP* GFP reporters, in addition to dC to dT mutations, dC to dG and dC to dA mutations were also observed, albeit with lower frequencies (FIG. 33C). This is consistent with previous results reported in mammalian cell lines (7, 8), and reflects the promiscuous outcome of repair of deaminated dC (dU) lesions in these cells. Notably, in samples containing the 1xOp*_GFP reporter, the frequency of the WT allele (state S0) decreased and the frequency of the mutant alleles increased linearly over time (FIG. 33C). Thus, even without having a repeat array, the accumulation of mutations in a specific target site can be used as an analog readout of an incoming signal.

Besides serving as a proof of concept for associative learning, the synthetic genetic circuit described in this experiment can be used as an online functional reporter for DNA memory states. Unlike existing DNA-based molecular recording technologies that rely on DNA sequencing to be read, the precise and sequential DNA writing achieved by DOMINO enables one to correlate the DNA memory state (i.e., the number of edited repeats) with the intensity of a fluorescence reporter signal that can be monitored in living cells without disrupting the cells (FIG. 26A-26F). This feature makes DOMINO recorders especially useful for studying biological events in living cells in an online fashion.

In this experiment, VP64 was used as an activator domain. However, the activation level and dynamic range of the reporter output can be tuned by using stronger activator domains such as VPR (20). Alternatively, other effector domains (such as repressors (19), DNA methyl transferases (21), acetyl transferases (22), or other types of hi stone modification domains) could be used to implement more sophisticated forms of gene regulation programs.

### Example 20. Concurrent Recording of Analog Information and Chronicle of Molecular Events into DNA

DOMINO circuits that rely on deterministic DNA modifications are useful when transitions between a handful of memory states are desired. The autonomous and continuous nature of these DNA writers are especially useful for building long-term DNA recorders to study signaling dynamics and event histories in their native contexts. However, for some applications, such as lineage tracing, the number of memory states needed to record event histories with high resolution could be orders of magnitude higher than what can be practically achieved by deterministic DNA mutations. Although the memory capacity of DOMINO circuits can be increased by incorporating multiple gRNAs or by increasing the number of repeats in DOMINO arrays, these designs are still not as compact as they could be and may require encoding large numbers of memory registers using dozens of gRNAs and/or hundreds and thousands of bps of DNA.

Existing Cas9-based recording technologies (5, 4) rely on stochastic DNA memory states resulting from indels generated by double-strand DNA breaks. These recorders lose their recording capacity after one or a few recording events due to deletions and loss of gRNA target sites and are therefore not ideal for long-term recording of event histories and generating high-resolution cellular lineages. To address some of these problems, the previously described mSCRIBE system (6) engineered a self-targeting gRNA (stgRNA) that could recruit Cas9 to its own encoding locus and execute cycles of double-strand break generation and successive indel formation by the Non-Homologous End Joining (NHEJ) pathway. However, due to prevalence of deletions as a product of NHEJ, these recorders could exhaust their recording capacity due to deletions in the stgRNA handle. Furthermore, new mutations could destroy the previous mutations (i.e., overwrite the previous memory states), which makes deducing lineage histories from these stochastically generated memory states challenging.

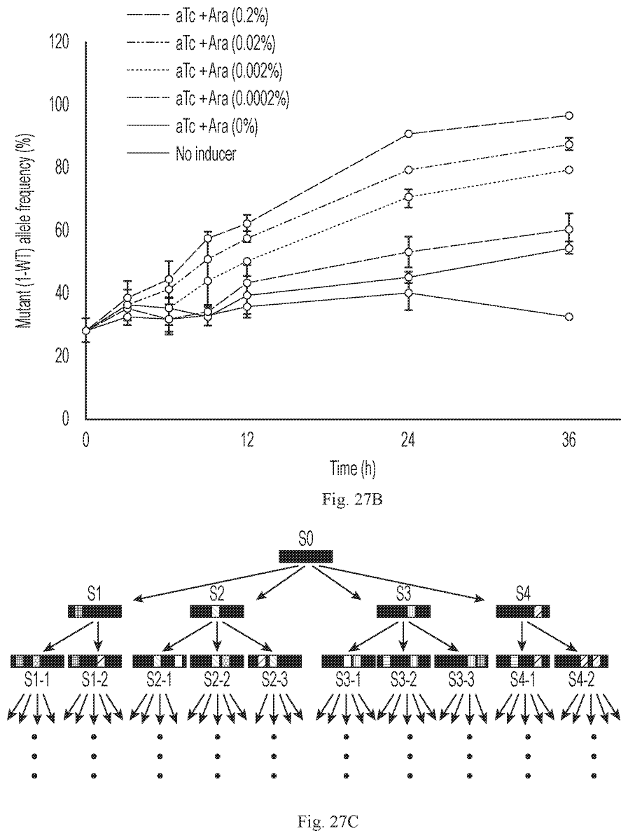

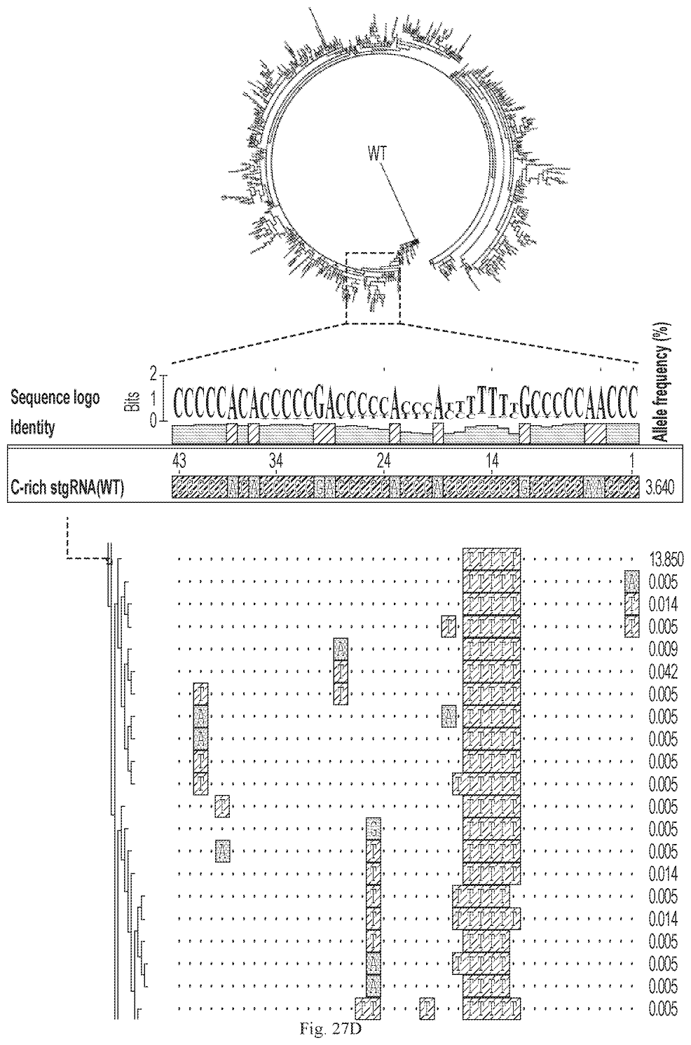

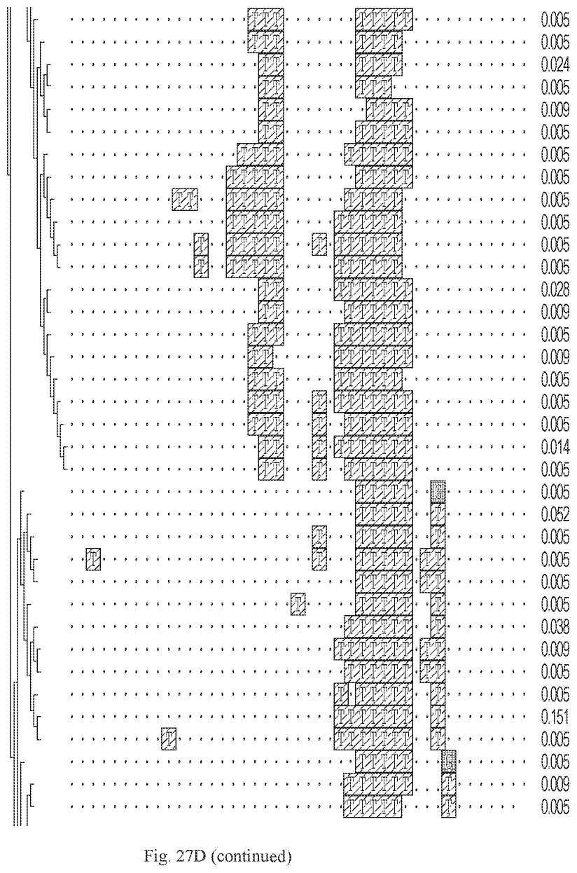

To address these limitations, a sequential mutation accumulation strategy was developed that can be used to build long-term, autonomous, and minimally disruptive molecular recorders in a compact, and high-capacity memory register. In this strategy, the CDA-nCas9-ugi read-write head continuously incorporates pseudo-random mutations into a (C-rich) stgRNA locus as a function of time and duration of stgRNA expression (FIG. 27A). Mutation accumulation in the stgRNA memory register can be coupled to signals of interest by placing stgRNA expression under the control of the corresponding signal. The degree to which mutations accumulate in this memory register can then be read out by HTS and used to deduce signaling dynamics of the original signal.

To demonstrate this concept, a C-rich stgRNA (43 bp SDS with 34 dC residues) was placed under the control of an Ara-inducible promoter (FIG. 27A) and this construct was transformed into E. coli cells harboring an aTc-inducible CDA-nCas9-ugi plasmid. The transformants were then grown in the presence or absence of aTc and different concentrations of Ara for multiple cycles with serial dilutions. Mutation accumulation in the stgRNA locus was monitored over the course of the experiment. As shown in FIG. 27B, the frequency of mutant alleles in the populations increased in a time- and Ara-dosage-dependent manner, indicating that these recorders are capable of recording analog information in a continuous fashion.

The unidirectional and minimally disruptive nature of CDA-mediated mutations generated by these recorders ensures that previous mutations (i.e., memory states) are preserved after each editing step (FIG. 27C). The pseudo-random yet position-specific mutations in locations corresponding to dC residues of the stgRNA memory register can be considered as discrete memory states of the register. Accumulation of mutations in the stgRNA locus can be thus considered as transitions between memory states. The memory capacity of these recorders is basically the number of memory states, which can be exponentially increased by increasing the number of dC residues within the stgRNA locus. These features make the mutation profiles generated by these recorders especially useful for investigating cellular event histories and lineages in an autonomous and high-resolution fashion. FIG. 27D shows an example of a lineage map generated for one of the samples (36 hours induction with aTc+Ara (0.2%)) in the experiment described in FIG. 27B. More than 1000 discrete memory states (unique mutations) could be detected in the 43 bps stgRNA memory register.

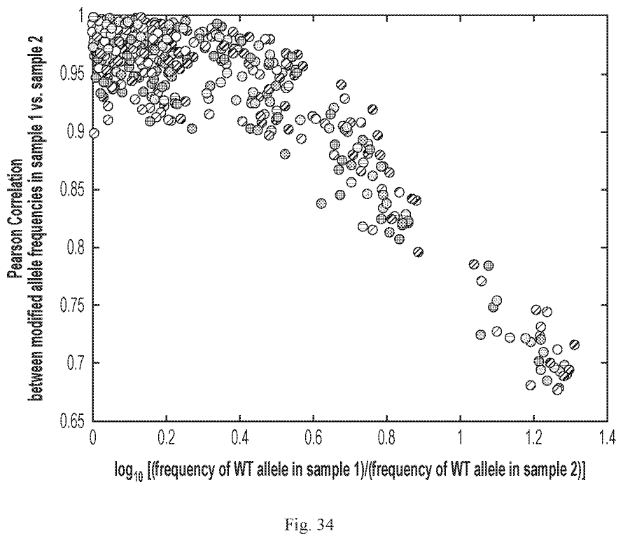

Further analysis of these samples revealed that samples with similar fractions of non-mutated stgRNA (state S0), often had a similar distribution of mutated alleles (states >S0) (FIG. 34). This suggests that the average rate of transitions between memory states depends on the allele frequencies in the current state, and not the input history. In other words, if a sample that has been induced with a high concentration of the input for a short time and a sample that has been induced with a low concentration of the input for a long time have similar frequencies of the unmutated allele (S0), they are very likely to have similar distributions of mutant allele frequencies. This suggests that while at the single-molecule level any transitions may occur randomly from a lower memory state (less mutation) to a higher memory state (more mutations) with some non-zero probability, at the population level, these transitions are more deterministic and are defined by the frequency of each memory state within the population.

This memory scheme (termed herein as “ENGRAmSCRIBE”), that operates in a distinct probabilistic fashion that distinguishes them from the deterministic DOMINO operators. While the memory states and orders of state transitions can be accurately designed and predicted in DOMINO-based memory registers, the exact transitions between memory states in ENGRAM registers are unpredictable and probabilistic. In ENGRAmSCRIBE registers, at the single molecule level each possible transition (i.e., from a lower memory state to a higher memory state) is likely to happen with some probability, however, at the population level, transitions are likely to be statistically predictable (FIG. 34) and are thus pseudo-random.

Overall, ENGRAmSCRIBE offers a compact, high-capacity, and long-term molecular recorder that can record the analog properties of a desired signal as well as the chronicle of events (lineages) produced by that signal over many generations. Combining these recorders with single-cell sequencing and more advanced barcoding schemes, as well as future development of this recording technology in mammalian cells, could pave the way to high-resolution maps of cellular lineages and other applications that require high-density memory storage capacities in living cells.

### Materials and Methods for Examples 15-20

### Estimating Position-Specific Mutant Frequencies by Sequalizer

A MATLAB program, dubbed Sequalizer (for Sequence equalizer), was developed to calculate the frequency of base-pair substitutions in specific positions in a mixture of DNA species from Sanger sequencing chromatograms. Analyzing Sanger chromatograms by Sequalizer offers a low-cost strategy to HTS for assessing and quantifying frequency of precise mutations (i.e. nucleotide substitutions) that are generated by base-editing and other targeted genome engineering platforms.

Sequalizer uses a previously described algorithm (SeqDoC (23)) to normalize and compute difference between Sanger chromatogram of a reference (unmodified) sequence and a test sample (which is expected to contain a mixture of DNA species containing mutations in specific positions). It then overlays the computed difference for all the four nucleotides (A, C, G, and T) on a single plot for the reference (top) and test sample (inverted, bottom) as a function of nucleotide position (x-axis) (FIG. 28A). A peak in this plot, indicates a difference in the normalized chromatogram signal between the reference and the test sample, and thus a mutation (i.e. base substitution) in that specific mutation. Sequalizer then estimates the frequency of mutants in each specific (targeted) position in the test sample using the difference between the heights of peaks corresponding to the reference and test samples in that position and reports that frequency as a number on top of the corresponding peaks. A test sample that has the same position-specific mutant frequency as the reference would result in no peaks in the Sequalizer plots (FIG. 28A, top panel). On the other hand, base-substitutions in the test sample compared to the reference sample can be detected as a peak in the Sequalizer plots (FIG. 28A, bottom panel). If a pure WT sample is used as the reference sample, the number printed on top of the peak estimates the frequency of molecules with mutation in that specific position in the test sample.

Since there is a high degree of variation between height of peaks between different positions along a Sanger chromatogram, for each position Sequalizer normalizes the computed difference to the height of the peak for the reference chromatogram in that specific position. However, the height of the Sanger chromatogram containing 100% mutant alleles in a position could be different from the reference in that position, which could result in under- or over-estimation of mutant frequencies by Sequalizer. Since the Sanger chromatogram, and thus the height of peaks for samples with the 100% mutant alleles are not always known, Sequalizer uses an experimentally determined parameter to account for the difference in height of peaks of Sanger chromatogram in each position. This parameter was calculated by mixing pure WT and pure mutant samples with different ratios, sequencing the mixtures, and using the Sequalizer output of the corresponding chromatograms to calculate a standard curve. As shown in FIG. 28B, the Sequalizer algorithm is able to compute frequencies of mutants at different positions solely based on Sanger chromatogram data, which correlates well with the mutant ratios in the mixtures.

Sequalizer was further verified by measuring position-specific mutant frequencies and comparing the output with the HTS for samples obtained from the combinatorial AND gate circuit for the experiment described in FIG. 23B. As shown in FIG. 28C, high correlation (R² values) was observed between mutant frequencies measured by both methods in all the targeted positions, indicating that Sequalizer output can be used as a low-cost alternative to HTS. Deviation of the regression slope from unity (e.g., for C20 position) could be partially due to variations in the height of peaks of Sanger chromatograms between pure WT and pure mutant at different positions. As mentioned above, Sequalizer algorithm tries to minimize the effect of such variations by normalizing the differences to the height of the WT peak in corresponding positions. However, since the heights of Sanger chromatograms for a pure mutant species also could affect the Sequalizer and this value is often unknown, it could cause the Sequalizer to underestimate or overestimate mutant frequencies compared to those measured by HTS. Nevertheless, the high correlation between Sequalizer outputs and HTS results indicate that changes in Sequalizer output can be used as a quantitative measure of changes in allele frequencies in a given position, even if they are not used for absolute measurements.

### Strains and Plasmids

Standard molecular biology and cloning techniques, including ligation, Gibson assembly (24) and Golden Gate assembly (25) were used to construct the plasmids. Chemically competent E. coli DH5a F′ lacI^{q} (NEB) and E. cloni 10G (Lucigen) were used for cloning. MG1655 PRO strain (MG1655 strain that harbors PRO cassette (pZS4Int-lacI/tetR, Expressys) and expresses lacI and tetR at high levels) (26) was used for all the bacterial experiments. HEK 293T cells (ATCC CRL-11268) were purchased from and authenticated by ATCC and were used for mammalian cell experiments. Lists of plasmids, synthetic parts and sequencing primers used are provided in Tables 7, 8, and 9, respectively. Plasmids and their corresponding maps will be available on Addgene.

### Antibiotics and Inducers

Antibiotics were used at the following concentrations: Carbenicillin (Carb, 50 μg/mL), and Chloramphenicol (Cam, 25-30 μg/mL).

For the experiments shown in FIGS. 23E, 24D, 24E, 29C, and 31A-31B different combinations of 200 ng/ml anhydrotetracycline (aTc), 0.1 mM Isopropyl β-D-1-thiogalactopyranoside (IPTG) and 0.2% Arabinose (Ara) were used to induce the corresponding circuits. For the experiments shown in FIGS. 30 and 32A-32B, 250 ng/ml aTc and 0.005% Ara were used. For the experiment shown in FIG. 24A, 150 ng/ml aTc and 0.1 mM IPTG were used. For all the other experiments, unless otherwise noted, 250 ng/ml aTc, 1 mM IPTG and 0.2% Ara were used. All concentrations are final concentrations.

### Bacterial Cell Experiments

Different plasmids expressing gRNAs and targets (listed in Table 7) were transformed into the reporter cells (MG1655 PRO) harboring aTc-inducible CDA-nCas9-ugi (for bacterial experiments, APOBEC1 CDA (7) was used as the writing module). Single transformant colonies were grown in LB+Carb+Cam for 6-8 hours to obtain seed cultures. Seed cultures were diluted (1:100) in fresh media containing different combinations of inducers and grown in 96-well plates for multiple days with serial dilution as indicated in induction patterns in corresponding figures. Samples for various analyses including HTS, Sequalizer, and flow cytometry were taken at indicated time points.

### Cell Cultures and Mammalian Cell Experiments

Cell culture and transfections were performed as described previously (6). HEK 293T cells were grown in DMEM supplemented with 10% fetal bovine serum (FBS) and 1% penicillin-streptomycin. Lentiviruses were packaged using the FUGW backbone (Addgene #25870) and psPAX2 and pVSV-G helper plasmids in HEK 293T cells. Filtered lentiviruses were used to infect respective cell lines in the presence of polybrene (8 μg/mL). Successful lentiviral integration was confirmed by using lentiviral plasmid constructs constitutively expressing fluorescent proteins or antibiotic resistance genes to serve as infection markers.

A lentiviral plasmid construct was made by placing the nCas9-CDA-ugi-VP64 fusion protein with nuclear localization signals linked to the Puromycin resistance gene with the P2A sequence under the control of constitutive CMV promoter (for mammalian experiments, PmCDA (8) was used as the writing module). In addition, repeat arrays (4xOp_1xOp* or 1xOp*) were placed upstream of the minimal pMLV promoter driving EGFP and the resultant reporter constructs were cloned into the same lentiviral construct. The clonal cell lines harboring the two transcriptional units were constructed by infecting early passage HEK 293T cells with high titer lentiviral particles, selecting for pooled populations grown in the presence of Puromycin (7 μg/mL) and picking up clonal populations after seeding pooled population with the density of 0.5 cells per well in a 96-well plate.

On day 0, 440,000 clonal reporter cells were infected with high titer lentiviral particles encoding the sgRNAs driven by the U6 promoter in a 6-well plate with triplicates. Infection efficiency was more than 90% in every sample. The cells were harvested every 3 days until day 15 after the infection. Half of the harvested cells were seeded in a 6-well plate for further culture and a quarter of cells were collected for next-generation sequencing. Microscopic images were obtained just before the harvests.

### Microscopy Image Analysis

Fluorescence microscopy images of cells in tissue culture plates were obtained by using the ZEISS ZEN microscope software. For each sample, total number of EGFP-positive cells and signal intensities were measured from microscopic images of 5 random fields using CellProfiler image analysis software by using the ‘ColorToGray’, ‘IdentifyPrimaryObjects’, MeasureObjectIntensity′ and ‘ExportToSpreadsheet’ modules.

### Flow Cytometry

An LSR Fortessa II flow cytometer (Becton Dickinson, N.J.) was used for all the experiments. GFP expression was measured using 488/FITC laser/filter set. All samples were uniformly gated and flow cytometry data were analyzed by FACSDiva and FlowJo (Becton Dickinson, N.J.). For each gated sample, the mean fluorescence and percent of GFP-positive cells were calculated.

### High-Throughput Sequencing

For each sample, 5 μl of culture was resuspended in 15 μl of QuickExtract DNA Extraction Solution (Epicentre, Wis.) and lysed by a two-step protocol (15 minutes incubation at 65° C. followed by 2 minutes incubation at 98° C.). Target sites were PCR amplified using 2 μl of lysed cultures as template and the appropriate primers listed in Table 9. The obtained amplicons were directly used as templates in a second round of PCR to add Illumina barcodes and adaptors. The amplicons were then multiplexed and analyzed by Illumina MiSeq. The obtained sequencing reads were demultiplexed and allele frequencies were calculated using a custom MATLAB script.

### Sanger Sequencing and Sequalizer Analysis

For each sample, target sites were PCR amplified by target-specific primers and Sanger sequenced by Quintara Biosciences. The obtained Sanger chromatograms were then analyzed by Sequalizer using seed cultures as reference as described above.

### Example 21. Directed and Recurring In Vivo Evolution

In addition to rational implementation of logic and memory, in an approach called DRIVE (for Directed and Recurring In Vivo Evolution), it was demonstrated that this in vivo DNA writing platform can be used to endow cells with the ability to autonomously target and mutagenize their genome and undergo synthetic Lamarckian evolution under suitable selective pressure. This less-explored but powerful approach that converts genetic DNA into a targetable substrate for evolution in the laboratory, could open up new avenues to study and engineer biological systems.

### Synthetic Lamarckian Evolution

Genomic DNA is the ultimate storage medium for life. The information stored in this medium is mainly written, rewritten and scoured by Darwinian evolution forces over evolutionary timescales. However, in certain cases, where the rate of Darwinian evolution is not enough to adapt and cope with treat of ever-changing an environment, living cells have evolved mechanisms to selectively elevate mutation rate in specific segments of their genome, to evolve faster than possible by natural Darwinian evolution. The immune system in higher eukaryotes and their counterpart in prokaryotes, CRISPR spacer acquisition system, as well as diversity generating retroelements and phase variation mechanisms are natural examples of such active DNA writing mechanisms. These mechanisms can be all considered as examples of natural Lamarckian evolution that act at the molecular level.

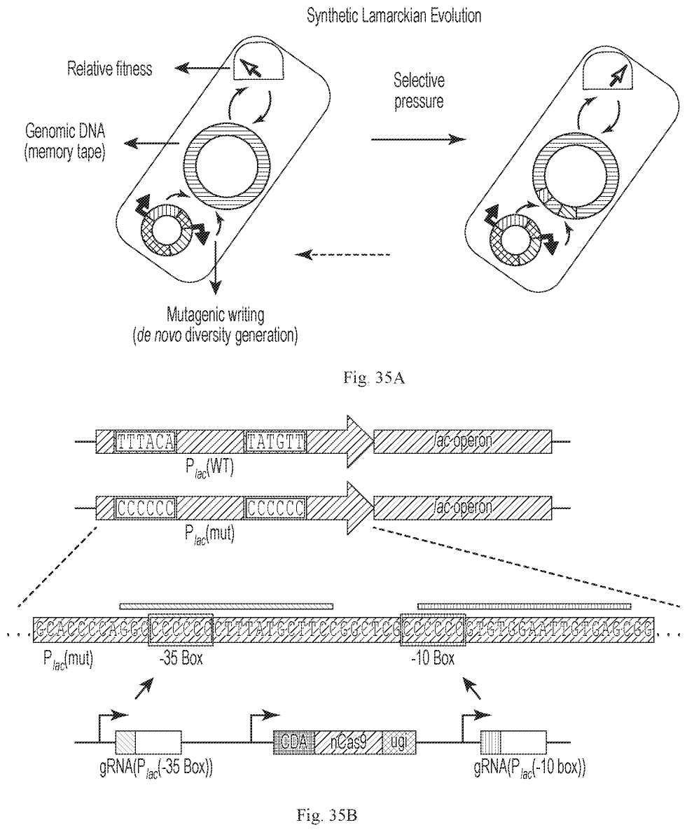

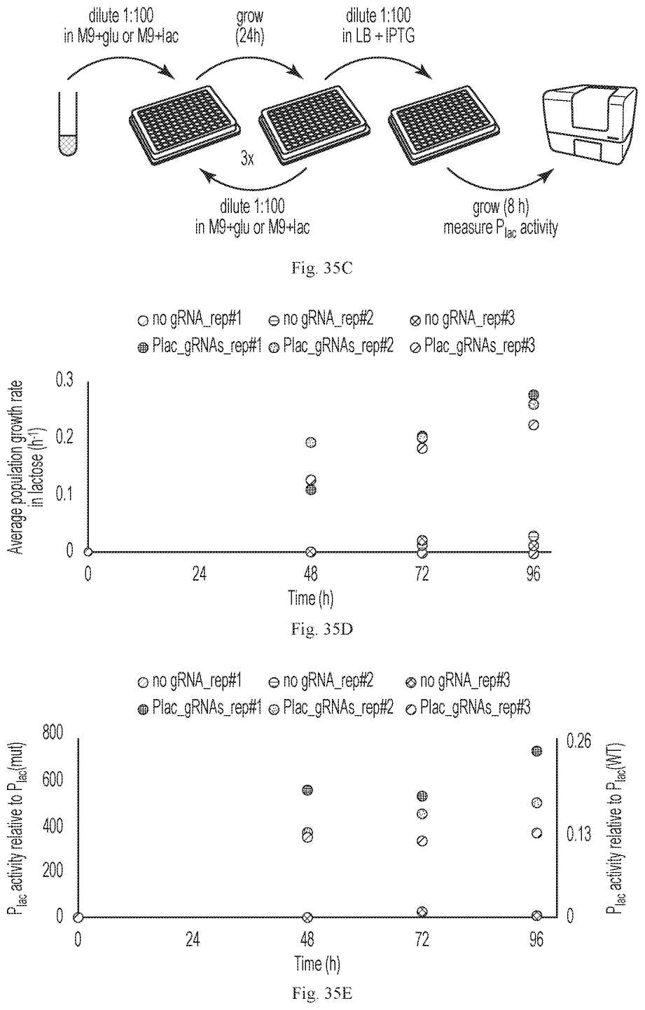

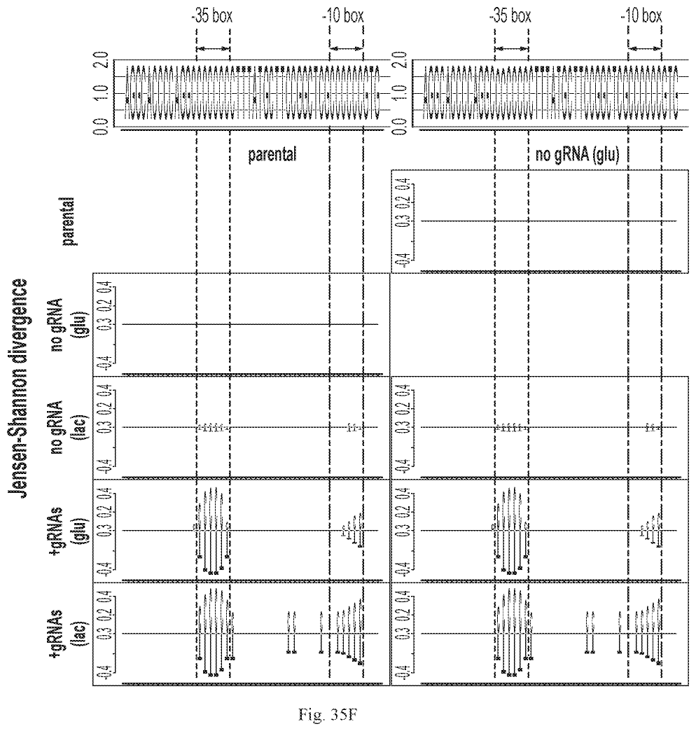

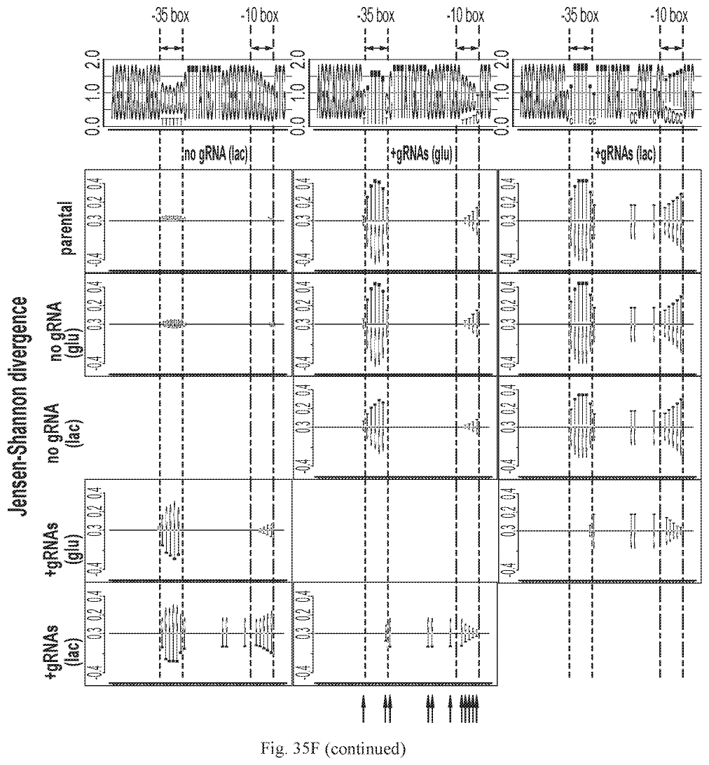

Endowing living cells with a synthetic ability to undergo Lamarckian evolution could have a great potential for studying and evolutionary engineering of these systems. However, the abovementioned strategies are not currently amenable to be redirected to desired targets. The CDA-nCas9 DNA writing platform, however, can be easily redirected to desired genomic segments connected to phenotype of interest to introduce de novo targeted diversity to that segment. Under a selective pressure, this could result in an increase in fitness and evolution much faster than possible by natural Darwinian evolution (FIG. 35A). Thus, this type of continuous de novo targeted diversity generation and adaptation at the presence of a selective pressure can be considered as a form of synthetic molecular Lamarckian evolution, which could be especially useful in tuning evolvability of living cells and evolutionary engineering of cellular phenotypes.

The concept was demonstrated by coupling targeted diversity generation achieved by DOMINO with a selective pressure, in a technique referred to as DRIVE (for Directed and Recurring In Vivo Evolution). Using this technique, it was shown that E. coli cells with an initially weak lac operon promoter (P_{lac}) can be engineered to evolve a stronger promoter at the presence of lactose as the sole carbon source, with a rate much faster than possible by natural evolution. Lactose utilization in E. coli relies on the activity of lac operon, and at the presence of lactose as the sole carbon source, cells fitness (i.e. growth rate) correlates with their ability to metabolize lactose (i.e. P operon activity). In order to increase the fitness range, the wild-type P_{lac} (P_{lac}(WT)) was weakened by replacing the −35 and −10 boxes of this promoter with dC residues. This mutant promoter (P_{lac}(mut)) has a very low activity and cells harboring this promoter (which hereafter are referred to as parental cells) grow very poorly at the presence of lactose (see the first time point in FIGS. 35D and 35E). The CDA-nCas9-ugi writer was then introduced with or without two gRNAs targeting the −35 and −10 boxes of the P_{lac}(mut) into these cells and grew the cells at the presence of glucose (glu) and lactose (lac) for multiple days (FIGS. 35B and 35C). The lac operon in E. coli is repressed at the presence of glucose, thus, glucose-containing media acts as a non-selective media for these cells. However, in media containing lactose as the sole carbon source, the diversified P_{lac} alleles would compete for consumption of lactose, and those with higher P_{lac} activity are expected to enrich the population over time.

The growth rate and P_{lac} activity of cultures were monitored throughout this experiment. As shown in FIG. 35D, the growth rate (in lactose) of cultures that did not express gRNAs only slightly increased toward the end of the experiment (after 72 hours). On the other hand, the growth rate (in lactose) of cultures harboring the P_{lac} containing promoters significantly increased over time, indicating a significant increase in the fitness and that these cells had evolved the ability to metabolize much faster than cells that did not express the gRNAs. These results were further confirmed by measuring the P_{lac} activity, where a significant increase in the activity of P_{lac} was observed in cultures that express P_{lac} targeting gRNAs, while the activity of P_{lac} in cells that did not express the gRNAs did not increase overtime.

To investigate the evolution of P_{lac} alleles at the molecular level, the P_{lac} locus was PCR amplified and the amplicons were sequenced by high-throughput sequencing. As shown in FIG. 35F, dC to dT mutations accumulated in the vicinity of the P_{lac} promoter in gRNA expressing cells, indicating targeted de novo diversity generation in this locus. Analysis of the enriched variants between gRNA-expressing cells grown in and glucose reveled a series of positions (marked by red arrows in FIG. 35F) in which mutations were more strongly enriched in the selective medium (lac) than non-selective medium (glu). The differential enrichment of mutation in these positions suggests that these positions were under positive selection and thus their corresponding mutations can be considered as adaptive mutations.

Some level of mutations was also observed in cells with no gRNA that were grown in lactose, but these mutations were only detectable in the later time-points and were significantly lower than level of mutations in cells expressing the gRNAs. These mutations were likely generated non-specifically as a result of increase in global mutation rate due to overexpression of the cytidine deaminase, which is further supported by that fact that these mutations only enriched in cells that were under selection (grown in lactose) and not those that were grown in non-selective media (glucose).

These results demonstrate that de novo targeted diversity generation achieved by an addressable DNA writer can be combined with suitable selective pressure to engineer cells that can autonomously increase the mutation rate of specific segments of their genomes and undergo (synthetic Lamarckian) evolution with a rate much faster than possible by Darwinain evolution. The outcome of the DRIVE platform is a remnant of natural diversity generation mechanism by the DGR system in phages and bacteria, but instead of dA residues in the DGR system, here dC residues are targeted for mutation, and the system can be easily retargeted to desired sequences. This less explored evolutionary engineering strategy, could have could have broad applicability in studying and evolutionary engineering of living systems, from engineering smart, fast-adaptable cells that can tune their response and find new solution in response to internal or external cues, to engineering adaptable therapeutics and biomolecules to devising continuous in vivo evolution strategies, to optimizing cellular traits and metabolic pathways, to engineering bacteriophages that can autonomously mutagenize their tail fiber and expand their host-range with a rate much faster than possible by natural evolution under specific user-specified condition.

### Example 22. Nucleotide Sequences and Amino Acid Sequences

Provided herein are exemplary guide RNA handle sequence (Table 2), exemplary RNA-guided nuclease sequences (Table 3), exemplary DNA polymerase sequences (Table 4), exemplary cytidine deaminase sequences (Table 5), exemplary primers (Table 7), exemplary synthetic parts and their corresponding sequences (Table 8), and exemplary HTS primers and their corresponding sequences (Table 9).

TABLE 2
Exemplary Guide RNA Handle Sequences

| Organism | gRNA handle sequence | SEQ ID NO |
|---|---|---|
| S. pyogenes | GUUUAAGAGCUAUGCUGGAAAGCCACGGUGA | 2 |
| | AAAAGUUCAACUAUUGCCUGAUCGGAAUAAA | |
| | UUUGAACGAUACGACAGUCGGUGCUUUUUUU | |
| S. pyogenes | GUUUAAGAGCUAGAAAUAGCAAGUUUAAAUA | 3 |
| | AGGCUAGUCCGUUAUCAACUUGAAAAAGUGG | |
| | CACCGAGUCGGUGCUTJTJTJUU | |
| S. thermophilus | GUUUUUGUACUCUCAAGAUUCAAUAAUCUUG | 4 |
| CRISPR1 | CAGAAGCUACAAAGAUAAGGCUUCAUGCCGAA | |
| | AUCAACACCCUGUCAUUUUAUGGCAGGGUGUU | |
| | UU | |
| S. thermophilus | GUUUUAGAGCUGUGUUGUUUGUUAAAACAAC | 5 |
| CRISPR3 | ACAGCGAGUUAAAAUAAGGCUUAGUCCGUAC | |
| | UCAACUUGAAAAGGUGGCACCGAUUCGGUGU | |
| | UUUU | |
| C. jejuni | AAGAAAUUUAAAAAGGGACUAAAAUAAAGAG | 6 |
| | UUUGCGGGACUCUGCGGGGUUACAAUCCCCUA | |
| | AAACCGCUUUU | |
| F. novicida | AUCUAAAAUUAUAAAUGUACCAAAUAAUUAA | 7 |
| | UGCUCUGUAAUCAUUUAAAAGUAUUUUGAAC | |
| | GGACCUCUGUUUGACACGUCUGAAUAACUAAA | |
| | A | |
| S. | UGUAAGGGACGCCUUACACAGUUACUUAAAUC | 8 |
| thermophilus2 | UUGCAGAAGCUACAAAGAUAAGGCUUCAUGCC | |
| | GAAAUCAACACCCUGUCAUUUUAUGGCAGGGU | |
| | GUUUUCGUUAUUU | |
| M. mobile | UGUAUUUCGAAAUACAGAUGUACAGUUAAGA | 9 |
| | AUACAUAAGAAUGAUACAUCACUAAAAAAAG | |
| | GCUUUAUGCCGUAACUACUACUUAUUUUCAAA | |
| | AUAAGUAGUUUUUUUU | |
| L. innocua | AUUGUUAGUAUUCAAAAUAACAUAGCAAGUU | 10 |
| | AAAAUAAGGCUUUGUCCGUUAUCAACUUUUA | |
| | AUUAAGUAGCGCUGUUUCGGCGCUUUUUUU | |
| S. pyogenes | GUUGGAACCAUUCAAAACAGCAUAGCAAGUU | 11 |
| | AAAAUAAGGCUAGUCCGUUAUCAACUUGAAA | |
| | AAGUGGCACCGAGUCGGUGCUUUUUUU | |
| S. nutans | GUUGGAAUCAUUCGAAACAACACAGCAAGUU | 12 |
| | AAAAUAAGGCAGUGAUUUUUAAUCCAGUCCG | |
| | UACACAACUUGAAAAAGUGCGCACCGAUUCGG | |
| | UGCUUUUUUAUUU | |
| S. thermophilus | UUGUGGUUUGAAACCAUUCGAAACAACACAGC | 13 |
| | GAGUUAAAAUAAGGCUUAGUCCGUACUCAAC | |
| | UUGAAAAGGUGGCACCGAUUCGGUGUUUUUU | |
| | UU | |
| N. meningitidis | ACAUAUUGUCGCACUGCGAAAUGAGAACCGUU | 14 |
| | GCUACAAUAAGGCCGUCUGAAAAGAUGUGCCG | |
| | CAACGCUCUGCCCCUUAAAGCUUCUGCUUUAA | |
| | GGGGCA | |
| P. multocida | GCAUAUUGUUGCACUGCGAAAUGAGAGACGU | 15 |
| | UGCUACAAUAAGGCUUCUGAAAAGAAUGACC | |
| | GUAACGCUCUGCCCCUUGUGAUUCUUAAUUGC | |
| | AAGGGGCAUCGUUUUU | |

TABLE 3
Exemplary RNA-guided Nuclease Sequences

| | | SEQ ID |
|---|---|---|
| Name | Sequence | NO: |
| S. pyogenes | MDKKYSIGLDIGTNSVGWAVITDEYKVPSKKFKVLGNTDRHSIKKNLIGALL | 18 |
| Cas9 | FDSGETAEATRLKRTARRRYTRRKNRICYLQEIFSNEMAKVDDSFFHRLEESF | |
| | LVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAI | |
| | LSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKEL | |
| | GSQILKEHPVENTQLQNEKLYLYYLQNGRDMYVDQELDINRLSDYDVDHIVP | |
| | QSFLKDDSIDNKVLTRSDKNRGKSDNVPSEEVVKKMKNYWRQLLNAKLITQ | |
| | RKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKS | |
| | KKLKSVKELLGITIMERSSFEKNPIDFLEAKGYKEVKKDLIIKLPKYSLFELEN | |
| | GRKRMLASAGELQKGNELALPSKYVNFLYLASHYEKLKGSPEDNEQKQLFV | |
| | EQHKHYLDEIIEQISEFSKRVILADANLDKVLSAYNKHRDKPIREQAENIIHLF | |
| | TLTNLGAPAAFKYFDTTIDRKRYTSTKEVLDATLIHQSITGLYETRIDLSQLGG | |
| | D | |
| Francisella | MSIYQEFVNKYSLSKTLRFELIPQGKTLENIKARGLILDDEKRAKDYKKAKQII | 19 |
| novicida | DKYHQFFIEEILSSVCISEDLLQNYSDVYFKLKKSDDDNLQKDFKSAKDTIKK | |
| Cpf1 | QISEYIKDSEKFKNLFNQNLIDAKKGQESDLILWLKQSKDNGIELFKANSDITD | |
| (Uniport | IDEALEIIKSFKGWTTYFKGFHENRKNVYSSNDIPTSIIYRIVDDNLPKFLENKA | |
| Reference | KYESLKDKAPEAINYEQIKKDLAEELTFDIDYKTSEVNQRVFSLDEVFEIANF | |
| Sequence: | NNYLNQSGITKFNTIIGGKFVNGENTKRKGINEYINLYSQQINDKTLKKYKMS | |
| A0Q7Q2): | VLFKQILSDTESKSFVIDKLEDDSDVVTTMQSFYEQIAAFKTVEEKSIKETLSL | |
| | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIDRGERHLAYYTLVDGKGNIIKQDTFNIIGNDRMKTNYHDKLA | |
| | AIEKDRDSARKDWKKINNIKEMKEGYLSQVVHEIAKLVIEYNAIVVFEDLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDADANGAYHIGLKGLMLL | |
| | GRIKNNQEGKKLNLVIKNEEYFEFVQNRNN | |
| S. pyogenes | MDKKYSIGLAIGTNSVGWAVITDEYKVPSKKFKVLGNTDRHSIKKNLIGALL | 20 |
| dCas9 | FDSGETAEATRLKRTARRRYTRRKNRICYLQEIFSNEMAKVDDSFFHRLEESF | |
| (D10A and | LVEEDKKHERHPIFGNIVDEVAYHEKYPTIYHLRKKLVDSTDKADLRLIYLAL | |
| H840A, | AHMIKFRGHFLIEGDLNPDNSDVDKLFIQLVQTYNQLFEENPINASGVDAKAI | |
| mutated | LSARLSKSRRLENLIAQLPGEKKNGLFGNLIALSLGLTPNFKSNFDLAEDAKL | |
| residues are | QLSKDTYDDDLDNLLAQIGDQYADLFLAAKNLSDAILLSDILRVNTEITKAPL | |
| underlined) | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKEL | |
| | GSQILKEHPVENTQLQNEKLYLYYLQNGRDMYVDQELDINRLSDYDVDAIVP | |
| | QSFLKDDSIDNKVLTRSDKNRGKSDNVPSEEVVKKMKNYWRQLLNAKLITQ | |
| | RKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKS | |
| | KKLKSVKELLGITIMERSSFEKNPIDFLEAKGYKEVKKDLIIKLPKYSLFELEN | |
| | GRKRMLASAGELQKGNELALPSKYVNFLYLASHYEKLKGSPEDNEQKQLFV | |
| | EQHKHYLDEIIEQISEFSKRVILADANLDKVLSAYNKHRDKPIREQAENIIHLF | |
| | TLTNLGAPAAFKYFDTTIDRKRYTSTKEVLDATLIHQSITGLYETRIDLSQLGG | |
| | D | |
| S. pyogenes | MDKKYSIGLAIGTNSVGWAVITDEYKVPSKKFKVLGNTDRHSIKKNLIGALL | 21 |
| Cas9 | FDSGETAEATRLKRTARRRYTRRKNRICYLQEIFSNEMAKVDDSFFHRLEESF | |
| Nickase | LVEEDKKHERHPIFGNIVDEVAYHEKYPTIYHLRKKLVDSTDKADLRLIYLAL | |
| (D10A, | AHMIKFRGHFLIEGDLNPDNSDVDKLFIQLVQTYNQLFEENPINASGVDAKAI | |
| mutation is | LSARLSKSRRLENLIAQLPGEKKNGLFGNLIALSLGLTPNFKSNFDLAEDAKL | |
| underlined | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKEL | |
| | GSQILKEHPVENTQLQNEKLYLYYLQNGRDMYVDQELDINRLSDYDVDHIVP | |
| | QSFLKDDSIDNKVLTRSDKNRGKSDNVPSEEVVKKMKNYWRQLLNAKLITQ | |
| | RKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKS | |
| | KKLKSVKELLGITIMERSSFEKNPIDFLEAKGYKEVKKDLIIKLPKYSLFELEN | |
| | GRKRMLASAGELQKGNELALPSKYVNFLYLASHYEKLKGSPEDNEQKQLFV | |
| | EQHKHYLDEIIEQISEFSKRVILADANLDKVLSAYNKHRDKPIREQAENIIHLF | |
| | TLTNLGAPAAFKYFDTTIDRKRYTSTKEVLDATLIHQSITGLYETRIDLSQLGG | |
| | D | |
| Francisella | MSIYQEFVNKYSLSKTLRFELIPQGKTLENIKARGLILDDEKRAKDYKKAKQII | 22 |
| novicida | DKYHQFFIEEILSSVCISEDLLQNYSDVYFKLKKSDDDNLQKDFKSAKDTIKK | |
| dCpf1 | QISEYIKDSEKFKNLFNQNLIDAKKGQESDLILWLKQSKDNGIELFKANSDITD | |
| (D917A, | IDEALEIIKSFKGWTTYFKGFHENRKNVYSSNDIPTSIIYRIVDDNLPKFLENKA | |
| mutation is | KYESLKDKAPEAINYEQIKKDLAEELTFDIDYKTSEVNQRVFSLDEVFEIANF | |
| underlined) | NNYLNQSGITKFNTIIGGKFVNGENTKRKGINEYINLYSQQINDKTLKKYKMS | |
| | VLFKQILSDTESKSFVIDKLEDDSDVVTTMQSFYEQIAAFKTVEEKSIKETLSL | |
| | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIARGERHLAYYTLVDGKGNIIKQDTFNIIGNDRMKTNYHDKLA | |
| | AIEKDRDSARKDWKKINNIKEMKEGYLSQVVHEIAKLVIEYNAIVVFEDLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDADANGAYHIGLKGLMLL | |
| | GRIKNNQEGKKLNLVIKNEEYFEFVQNRNN | |
| Francisella | MSIYQEFVNKYSLSKTLRFELIPQGKTLENIKARGLILDDEKRAKDYKKAKQII | 23 |
| novicida | DKYHQFFIEEILSSVCISEDLLQNYSDVYFKLKKSDDDNLQKDFKSAKDTIKK | |
| dCpf1 | QISEYIKDSEKFKNLFNQNLIDAKKGQESDLILWLKQSKDNGIELFKANSDITD | |
| (E1006A, | IDEALEIIKSFKGWTTYFKGFHENRKNVYSSNDIPTSIIYRIVDDNLPKFLENKA | |
| mutation is | KYESLKDKAPEAINYEQIKKDLAEELTFDIDYKTSEVNQRVFSLDEVFEIANF | |
| underlined) | NNYLNQSGITKFNTIIGGKFVNGENTKRKGINEYINLYSQQINDKTLKKYKMS | |
| | VLFKQILSDTESKSFVIDKLEDDSDVVTTMQSFYEQIAAFKTVEEKSIKETLSL | |
| | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIDRGERHLAYYTLVDGKGNIIKQDTFNIIGNDRMKTNYHDKLA | |
| | AIEKDRDSARKDWKKINNIKEMKEGYLSQVVHEIAKLVIEYNAIVVFADLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDADANGAYHIGLKGLMLL | |
| | GRIKNNQEGKKLNLVIKNEEYFEFVQNRNN | |
| Francisella | MSIYQEFVNKYSLSKTLRFELIPQGKTLENIKARGLILDDEKRAKDYKKAKQII | 25 |
| novicida | DKYHQFFIEEILSSVCISEDLLQNYSDVYFKLKKSDDDNLQKDFKSAKDTIKK | |
| dCpf1 | QISEYIKDSEKFKNLFNQNLIDAKKGQESDLILWLKQSKDNGIELFKANSDITD | |
| (D1255A, | IDEALEIIKSFKGWTTYFKGFHENRKNVYSSNDIPTSIIYRIVDDNLPKFLENKA | |
| mutation is | KYESLKDKAPEAINYEQIKKDLAEELTFDIDYKTSEVNQRVFSLDEVFEIANF | |
| underlined) | NNYLNQSGITKFNTIIGGKFVNGENTKRKGINEYINLYSQQINDKTLKKYKMS | |
| | VLFKQILSDTESKSFVIDKLEDDSDVVTTMQSFYEQIAAFKTVEEKSIKETLSL | |
| | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIDRGERHLAYYTLVDGKGNIIKQDTFNIIGNDRMKTNYHDKLA | |
| | AIEKDRDSARKDWKKINNIKEMKEGYLSQVVHEIAKLVIEYNAIVVFEDLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDAAANGAYHIGLKGLMLL | |
| | GRIKNNQEGKKLNLVIKNEEYFEFVQNRNN | |
| Francisella | MSIYQEFVNKYSLSKTLRFELIPQGKTLENIKARGLILDDEKRAKDYKKAKQII | 26 |
| novicida | DKYHQFFIEEILSSVCISEDLLQNYSDVYFKLKKSDDDNLQKDFKSAKDTIKK | |
| dCpf1 | QISEYIKDSEKFKNLFNQNLIDAKKGQESDLILWLKQSKDNGIELFKANSDITD | |
| (D917A/ | IDEALEIIKSFKGWTTYFKGFHENRKNVYSSNDIPTSIIYRIVDDNLPKFLENKA | |
| D1255A, | KYESLKDKAPEAINYEQIKKDLAEELTFDIDYKTSEVNQRVFSLDEVFEIANF | |
| mutations | NNYLNQSGITKFNTIIGGKFVNGENTKRKGINEYINLYSQQINDKTLKKYKMS | |
| are | VLFKQILSDTESKSFVIDKLEDDSDVVTTMQSFYEQIAAFKTVEEKSIKETLSL | |
| underlined) | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIARGERHLAYYTLVDGKGNIIKQDTFNIIGNDRMKTNYHDKLA | |
| | AIEKDRDSARKDWKKINNIKEMKEGYLSQVVHEIAKLVIEYNAIVVFEDLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDAAANGAYHIGLKGLMLL | |
| | GRIKNNQEGKKLNLVIKNEEYFEFVQNRNN | |
| Francisella | MSIYQEFVNKYSLSKTLRFELIPQGKTLENIKARGLILDDEKRAKDYKKAKQII | 27 |
| novicida | DKYHQFFIEEILSSVCISEDLLQNYSDVYFKLKKSDDDNLQKDFKSAKDTIKK | |
| dCpf1 | QISEYIKDSEKFKNLFNQNLIDAKKGQESDLILWLKQSKDNGIELFKANSDITD | |
| (E1006A/ | IDEALEIIKSFKGWTTYFKGFHENRKNVYSSNDIPTSIIYRIVDDNLPKFLENKA | |
| D1255A, | KYESLKDKAPEAINYEQIKKDLAEELTFDIDYKTSEVNQRVFSLDEVFEIANF | |
| mutations | NNYLNQSGITKFNTIIGGKFVNGENTKRKGINEYINLYSQQINDKTLKKYKMS | |
| are | VLFKQILSDTESKSFVIDKLEDDSDVVTTMQSFYEQIAAFKTVEEKSIKETLSL | |
| underlined) | LFDDLKAQKLDLSKIYFKNDKSLTDLSQQVFDDYSVIGTAVLEYITQQIAPKN | |
| | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIDRGERHLAYYTLVDGKGNIIKQDTFNIIGNDRMKTNYHDKLA | |
| | AIEKDRDSARKDWKKINNIKEMKEGYLSQVVHEIAKLVIEYNAIVVFADLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDAAANGAYHIGLKGLMLL | |
| | GRIKNNQEGKKLNLVIKNEEYFEFVQNRNN | |
| Francisella | MSIYQEFVNKYSLSKTLRFELIPQGKTLENIKARGLILDDEKRAKDYKKAKQII | 28 |
| novicida | DKYHQFFIEEILSSVCISEDLLQNYSDVYFKLKKSDDDNLQKDFKSAKDTIKK | |
| Cpfl | QISEYIKDSEKFKNLFNQNLIDAKKGQESDLILWLKQSKDNGIELFKANSDITD | |
| (D917A/ | IDEALEIIKSFKGWTTYFKGFHENRKNVYSSNDIPTSIIYRIVDDNLPKFLENKA | |
| E1006A/ | KYESLKDKAPEAINYEQIKKDLAEELTFDIDYKTSEVNQRVFSLDEVFEIANF | |
| D1255A, | NNYLNQSGITKFNTIIGGKFVNGENTKRKGINEYINLYSQQINDKTLKKYKMS | |
| mutations | VLFKQILSDTESKSFVIDKLEDDSDVVTTMQSFYEQIAAFKTVEEKSIKETLSL | |
| are | LFDDLKAQKLDLSKIYFKNDKSLTDLSQQVFDDYSVIGTAVLEYITQQIAPKN | |
| underlined) | LDNPSKKEQELIAKKTEKAKYLSLETIKLALEEFNKHRDIDKQCRFEEILANFA | |
| | AIPMIFDEIAQNKDNLAQISIKYQNQGKKDLLQASAEDDVKAIKDLLDQTNNL | |
| | LHKLKIFHISQSEDKANILDKDEHFYLVFEECYFELANIVPLYNKIRNYITQKP | |
| | YSDEKFKLNFENSTLANGWDKNKEPDNTAILFIKDDKYYLGVMNKKNNKIF | |
| | DDKAIKENKGEGYKKIVYKLLPGANKMLPKVFFSAKSIKFYNPSEDILRIRNH | |
| | STHTKNGSPQKGYEKFEFNIEDCRKFIDFYKQSISKHPEWKDFGFRFSDTQRY | |
| | NSIDEFYREVENQGYKLTFENISESYIDSVVNQGKLYLFQIYNKDFSAYSKGR | |
| | PNLHTLYWKALFDERNLQDVVYKLNGEAELFYRKQSIPKKITHPAKEAIANK | |
| | NKDNPKKESVFEYDLIKDKRFTEDKFFFHCPITINFKSSGANKFNDEINLLLKE | |
| | KANDVHILSIARGERHLAYYTLVDGKGNIIKQDTFNIIGNDRMKTNYHDKLA | |
| | AIEKDRDSARKDWKKINNIKEMKEGYLSQVVHEIAKLVIEYNAIVVFADLNF | |
| | GFKRGRFKVEKQVYQKLEKMLIEKLNYLVFKDNEFDKTGGVLRAYQLTAPF | |
| | ETFKKMGKQTGIIYYVPAGFTSKICPVTGFVNQLYPKYESVSKSQEFFSKFDKI | |
| | CYNLDKGYFEFSFDYKNFGDKAAKGKWTIASFGSRLINFRNSDKNHNWDTR | |
| | EVYPTKELEKLLKDYSIEYGHGECIKAAICGESDKKFFAKLTSVLNTILQMRN | |
| | SKTGIELDYLISPVADVNGNFFDSRQAPKNMPQDAAANGAYHIGLKGLMLL | |
| | GRIKNNQEGKKLNLVIKNEEYFEFVQNRNN | |

TABLE 4
Exemplary DNA Polymerases in ramSCRIBE

| | | SEQ |
|---|---|---|
| Name | Sequence | ID NO |
| Human terminal | MDPPRASHLSPRKKRPRQTGALMASSPQDIKFQDLVVFILEKKMGTT | 29 |
| deoxynucleotidyl | RRAFLMELARRKGFRVENELSDSVTHIVAENNSGSDVLEWLQAQKV | |
| transferase | QVSSQPELLDVSWLIECIRAGKPVEMTGKHQLVVRRDYSDSTNPGPP | |
| | KTPPIAVQKISQYACQRRTTLNNCNQIFTDAFDILAENCEFRENEDSC | |
| | VTFMRAASVLKSLPFTIISMKDTEGIPCLGSKVKGIIEEIIEDGESSEVK | |
| | AVLNDERYQSFKLFTSVFGVGLKTSEKWFRMGFRTLSKVRSDKSLKF | |
| | TRMQKAGFLYYEDLVSCVTRAEAEAVSVLVKEAVWAFLPDAFVTM | |
| | TGGFRRGKKMGHDVDFLITSPGSTEDEEQLLQKVMNLWEKKGLLLY | |
| | YDLVESTFEKLRLPSRKVDALDHFQKCFLIFKLPRQRVDSDQSSWQE | |
| | GKTWKAIRVDLVLCPYERRAFALLGWTGSRQFERDLRRYATHERK | |
| | MILDNHALYDKTKRIFLKAESEEEIFAHLGLDYIEPWERNA | |
| Human DNA | MDPRGILKAFPKRQKIHADASSKVLAKIPRREEGEEAEEWLSSLRAH | 30 |
| polymerase | VVRTGIGRARAELFEKQIVQHGGQLCPAQGPGVTHIVVDEGMDYER | |
| lambda | ALRLLRLPQLPPGAQLVKSAWLSLCLQERRLVDVAGFSIFIPSRYLDH | |
| | PQPSKAEQDASIPPGTHEALLQTALSPPPPPTRPVSPPQKAKEAPNTQA | |
| | QPISDDEASDGEETQVSAADLEALISGHYPTSLEGDCEPSPAPAVLDK | |
| | WVCAQPSSQKATNHNLHITEKLEVLAKAYSVQGDKWRALGYAKAI | |
| | NALKSFHKPVTSYQEACSIPGIGKRMAEKIIEILESGHLRKLDHISESVP | |
| | VLELFSNIWGAGTKTAQMWYQQGFRSLEDIRSQASLTTQQAIGLKH | |
| | YSDFLERMPREEATEIEQTVQKAAQAFNSGLLCVACGSYRRGKATC | |
| | GDVDVLITHPDGRSHRGIFSRLLDSLRQEGFLTDDLVSQEENGQQQK | |
| | YLGVCRLPGPGRRHRRLDIIVVPYSEFACALLYFTGSAHFNRSMRAL | |
| | AKTKGMSLSEHALSTAVVRNTHGCKVGPGRVLPTPTEKDVFRLLGL | |
| | PYREPAERDW | |

TABLE 5
Exemplary Cytidine deaminases

| | | SEQ ID |
|---|---|---|
| Name | Sequence | NO |
| Human AID | MDSLLMNRRKFLYQFKNVRWAKGRRETYLCYVVKRRDSATSFSLDFGYL | 49 |
| | RNKNGCHVELLFLRYISDWDLDPGRCYRVTWFTSWSPCYDCARHVADFL | |
| | RGNPNLSLRIFTARLYFCEDRKAEPEGLRRLHRAGVQIAIMTFKDYFYCWN | |
| | TFVENHERTFKAWEGLHENSVRLSRQLRRILLPLYEVDDLRDAFRTLGL | |
| Mouse AID | MDSLLMKQKKFLYHFKNVRWAKGRHETYLCYVVKRRDSATSCSLDFGHL | 50 |
| | RNKSGCHVELLFLRYISDWDLDPGRCYRVTWFTSWSPCYDCARHVAEFLR | |
| | WNPNLSLRIFTARLYFCEDRKAEPEGLRRLHRAGVQIGIMTFKDYFYCWNT | |
| | FVENRERTFKAWEGLHENSVRLTRQLRRILLPLYEVDDLRDAFRMLGF | |
| Dog AID | MDSLLMKQRKFLYHFKNVRWAKGRHETYLCYVVKRRDSATSFSLDFGHL | 51 |
| | RNKSGCHVELLFLRYISDWDLDPGRCYRVTWFTSWSPCYDCARHVADFLR | |
| | GYPNLSLRIFAARLYFCEDRKAEPEGLRRLHRAGVQIAIMTFKDYFYCWNT | |
| | FVENREKTFKAWEGLHENSVRLSRQLRRILLPLYEVDDLRDAFRTLGL | |
| Bovine AID | MDSLLKKQRQFLYQFKNVRWAKGRHETYLCYVVKRRDSPTSFSLDFGHL | 52 |
| | RNKAGCHVELLFLRYISDWDLDPGRCYRVTWFTSWSPCYDCARHVADFL | |
| | RGYPNLSLRIFTARLYFCDKERKAEPEGLRRLHRAGVQIAIMTFKDYFYCW | |
| | NTFVENHERTFKAWEGLHENSVRLSRQLRRILLPLYEVDDLRDAFRTLGL | |
| Mouse | MGPFCLGCSHRKCYSPIRNLISQETFKFHFKNLGYAKGRKDTFLCYEVTRK | 53 |
| APOBEC-3 | DCDSPVSLHHGVFKNKDNIHAEICFLYWFHDKVLKVLSPREEFKITWYMS | |
| | WSPCFECAEQIVRFLATHHNLSLDIFSSRLYNVQDPETQQNLCRLVQEGAQ | |
| | VAAMDLYEFKKCWKKFVDNGGRRFRPWKRLLTNFRYQDSKLQEILRPCYI | |
| | PVPSSSSSTLSNICLTKGLPETRFCVEGRRMDPLSEEEFYSQFYNQRVKHLC | |
| | YYHRMKPYLCYQLEQFNGQAPLKGCLLSEKGKQHAEILFLDKIRSMELSQ | |
| | VTITCYLTWSPCPNCAWQLAAFKRDRPDLILHIYTSRLYFHWKRPFQKGLC | |
| | SLWQSGILVDVMDLPQFTDCWTNFVNPKRPFWPWKGLEIISRRTQRRLRRI | |
| | KESWGLQDLVNDFGNLQLGPPMS | |
| Rat APOBEC- | MGPFCLGCSHRKCYSPIRNLISQETFKFHFKNLRYAIDRKDTFLCYEVTRKD | 54 |
| 3 | CDSPVSLHHGVFKNKDNIHAEICFLYWFHDKVLKVLSPREEFKITWYMSW | |
| | SPCFECAEQVLRFLATHHNLSLDIFSSRLYNIRDPENQQNLCRLVQEGAQVA | |
| | AMDLYEFKKCWKKFVDNGGRRFRPWKKLLTNFRYQDSKLQEILRPCYIPV | |
| | PSSSSSTLSNICLTKGLPETRFCVERRRVHLLSEEEFYSQFYNQRVKHLCYY | |
| | HGVKPYLCYQLEQFNGQAPLKGCLLSEKGKQHAEILFLDKIRSMELSQVIIT | |
| | CYLTWSPCPNCAWQLAAFKRDRPDLILHIYTSRLYFHWKRPFQKGLCSLW | |
| | QSGILVDVMDLPQFTDCWTNFVNPKRPFWPWKGLEIISRRTQRRLHRIKES | |
| | WGLQDLVNDFGNLQLGPPMS | |
| Rhesus | MVEPMDPRTFVSNFNNRPILSGLNTVWLCCEVKTKDPSGPPLDAKIFQGKV | 55 |
| macaque | YSKAKYHPEMRFLRWFHKWRQLHHDQEYKVTWYVSWSPCTRCANSVAT | |
| APOBEC-3G | FLAKDPKVTLTIFVARLYYFWKPDYQQALRILCQKRGGPHATMKIMNYNE | |
| | FQDCWNKFVDGRGKPFKPRNNLPKHYTLLQATLGELLRHLMDPGTFTSNF | |
| | NNKPWVSGQHETYLCYKVERLHNDTWVPLNQHRGFLRNQAPNIHGFPKG | |
| | RHAELCFLDLIPFWKLDGQQYRVTCFTSWSPCFSCAQEMAKFISNNEHVSL | |
| | CIFAARIYDDQGRYQEGLRALHRDGAKIAMMNYSEFEYCWDTFVDRQGRP | |
| | FQPWDGLDEHSQALSGRLRAI | |
| Chimpanzee | MKPHFRNPVERMYQDTFSDNFYNRPILSHRNTVWLCYEVKTKGPSRPPLD | 56 |
| APOBEC-3G | AKIFRGQVYSKLKYHPEMRFFHWFSKWRKLHRDQEYEVTWYISWSPCTK | |
| | CTRDVATFLAEDPKVTLTIFVARLYYFWDPDYQEALRSLCQKRDGPRATM | |
| | KIMNYDEFQHCWSKFVYSQRELFEPWNNLPKYYILLHIMLGEILRHSMDPP | |
| | TFTSNFNNELWVRGRHETYLCYEVERLHNDTWVLLNQRRGFLCNQAPHK | |
| | HGFLEGRHAELCFLDVIPFWKLDLHQDYRVTCFTSWSPCFSCAQEMAKFIS | |
| | NNKHVSLCIFAARIYDDQGRCQEGLRTLAKAGAKISIMTYSEFKHCWDTFV | |
| | DHQGCPFQPWDGLEEHSQALSGRLRAILQNQGN | |
| Green monkey | MNPQIRNMVEQMEPDIFVYYFNNRPILSGRNTVWLCYEVKTKDPSGPPLD | 57 |
| APOBEC-3G | ANIFQGKLYPEAKDHPEMKFLHWFRKWRQLHRDQEYEVTWYVSWSPCTR | |
| | CANSVATFLAEDPKVTLTIFVARLYYFWKPDYQQALRILCQERGGPHATM | |
| | KIMNYNEFQHCWNEFVDGQGKPFKPRKNLPKHYTLLHATLGELLRHVMD | |
| | PGTFTSNFNNKPWVSGQRETYLCYKVERSHNDTWVLLNQHRGFLRNQAP | |
| | DRHGFPKGRHAELCFLDLIPFWKLDDQQYRVTCFTSWSPCFSCAQKMAKFI | |
| | SNNKHVSLCIFAARIYDDQGRCQEGLRTLHRDGAKIAVMNYSEFEYCWDT | |
| | FVDRQGRPFQPWDGLDEHSQALSGRLRAI | |
| Human | MKPHFRNTVERMYRDTFSYNFYNRPILSRRNTVWLCYEVKTKGPSRPPLD | 58 |
| APOBEC-3G | AKIFRGQVYSELKYHPEMRFFHWFSKWRKLHRDQEYEVTWYISWSPCTKC | |
| | TRDMATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPPT | |
| | FTFNFNNEPWVRGRHETYLCYEVERMFINDTWVLLNQRRGFLCNQAPHKH | |
| | GFLEGRHAELCFLDVIPFWKLDLDQDYRVTCFTSWSPCFSCAQEMAKFISK | |
| | NKHVSLCIFTARIYDDQGRCQEGLRTLAEAGAKISIMTYSEFKHCWDTFVD | |
| | HQGCPFQPWDGLDEHSQDLSGRLRAILQNQEN | |
| Human | MKPHFRNTVERMYRDTFSYNFYNRPILSRRNTVWLCYEVKTKGPSRPRLD | 59 |
| APOBEC-3F | AKIFRGQVYSQPEHHAEMCFLSWFCGNQLPAYKCFQITWFVSWTPCPDCV | |
| | AKLAEFLAEHPNVTLTISAARLYYYWERDYRRALCRLSQAGARVKIMDDE | |
| | EFAYCWENFVYSEGQPFMPWYKFDDNYAFLHRTLKEILRNPMEAMYPHIF | |
| | YFHFKNLRKAYGRNESWLCFTMEVVKHHSPVSWKRGVFRNQVDPETHCH | |
| | AERCFLSWFCDDILSPNTNYEVTWYTSWSPCPECAGEVAEFLARHSNVNLT | |
| | IFTARLYYFWDTDYQEGLRSLSQEGASVEIMGYKDFKYCWENFVYNDDEP | |
| | FKPWKGLKYNFLFLDSKLQEILE | |
| Human | MNPQIRNPMERMYRDTFYDNFENEPILYGRSYTWLCYEVKIKRGRSNLLW | 60 |
| APOBEC-3B | DTGVFRGQVYFKPQYHAEMCFLSWFCGNQLPAYKCFQITWFVSWTPCPDC | |
| | VAKLAEFLSEHPNVTLTISAARLYYYWERDYRRALCRLSQAGARVTIMDY | |
| | EEFAYCWENFVYNEGQQFMPWYKFDENYAFLHRTLKEILRYLMDPDTFTF | |
| | NFNNDPLVLRRRQTYLCYEVERLDNGTWVLMDQHMGFLCNEAKNLLCGF | |
| | YGRHAELRFLDLVPSLQLDPAQIYRVTWFISWSPCFSWGCAGEVRAFLQEN | |
| | THVRLRIFAARIYDYDPLYKEALQMLRDAGAQVSIMTYDEFEYCWDTFVY | |
| | RQGCPFQPWDGLEEHSQALSGRLRAILQNQGN | |
| Human | MNPQIRNPMKAMYPGTFYFQFKNLWEANDRNETWLCFTVEGIKRRSVVS | 61 |
| APOBEC-3C | WKTGVFRNQVDSETHCHAERCFLSWFCDDILSPNTKYQVTWYTSWSPCPD | |
| | CAGEVAEFLARHSNVNLTIFTARLYYFQYPCYQEGLRSLSQEGVAVEIMDY | |
| | EDFKYCWENFVYNDNEPFKPWKGLKTNFRLLKRRLRESLQ | |
| Human | MEASPASGPRHLMDPHIFTSNFNNGIGRHKTYLCYEVERLDNGTSVKMDQ | 62 |
| APOBEC-3A | HRGFLHNQAKNLLCGFYGRHAELRFLDLVPSLQLDPAQIYRVTWFISWSPC | |
| | FSWGCAGEVRAFLQENTHVRLRIFAARIYDYDPLYKEALQMLRDAGAQVS | |
| | IMTYDEFKHCWDTFVDHQGCPFQPWDGLDEHSQALGRLRAILQNQGN | |
| Human | MALLTAETFRLQFNNKRRLRRPYYPRKALLCYQLTPQNGSTPTRGYFENK | 63 |
| APOBEC-3H | KKCHAEICFINEIKSMGLDETQCYQVTCYLTWSPCSSCAWELVDFIKAHDH | |
| | LNLGIFASRLYYHWCKPQQKGLRLLCGSQVPVEVMGFPKFADCWENFVD | |
| | HEKPLSFNPYKMLEELDKNSRAIKRRLERIKIPGVRAQGRYMDILCDAEV | |
| Human | MNPQIRNPMERMYRDTFYDNFENEPILYGRSYTWLCYEVKIKRGRSNLLW | 64 |
| APOBEC-3D | DTGVFRGPVLPKRQSNHRQEVYFRFENHAEMCFLSWFCGNRLPANRRFQI | |
| | TWFVSWNPCLPCVVKVTKFLAEHPNVTLTISAARLYYYRDRDWRWVLLR | |
| | LHKAGARVKIMDYEDFAYCWENFVCNEGQPFMPWYKFDDNYASLHRTL | |
| | KEILRNPMEAMYPHIFYFHFKNLLKACGRNESWLCFTMEVTKHHSAVFRK | |
| | RGVFRNQVDPETHCHAERCFLSWFCDDILSPNTNYEVTWYTSWSPCPECA | |
| | GEVAEFLARHSNVNLTIFTARLCYFWDTDYQEGLCSLSQEGASVKIMGYK | |
| | DFVSCWKNFVYSDDEPFKPWKGLQTNFRLLKRRLREILQ | |
| Human | MTSEKGPSTGDPTLRRRIEPWEFDVFYDPRELRKEACLLYEIKWGMSRKIW | 65 |
| APOBEC-1 | RSSGKNTTNHVEVNFIKKFTSERDFHPSMSCSITWFLSWSPCWECSQAIREF | |
| | LSRHPGVTLVIYVARLFWHMDQQNRQGLRDLVNSGVTIQIMRASEYYHC | |
| | WRNFVNYPPGDEAHWPQYPPLWMMLYALELHCIILSLPPCLKISRRWQNH | |
| | LTFFRLHLQNCHYQTIPPHILLATGLIHPSVAWR | |
| Mouse | MSSETGPVAVDPTLRRRIEPHEFEVFFDPRELRKETCLLYEINWGGRHSVW | 66 |
| APOBEC-1 | RHTSQNTSNHVEVNFLEKFTTERYFRPNTRCSITWFLSWSPCGECSRAITEF | |
| | LSRHPYVTLFIYIARLYHHTDQRNRQGLRDLISSGVTIQIMTEQEYCYCWRN | |
| | FVNYPPSNEAYWPRYPHLWVKLYVLELYCIILGLPPCLKILRRKQPQLTFFT | |
| | ITLQTCHYQRIPPHLLWATGLK | |
| Rat APOBEC- | MSSETGPVAVDPTLRRRIEPHEFEVFFDPRELRKETCLLYEINWGGRHSIWR | 67 |
| 1 | HTSQNTNKHVEVNFIEKFTTERYFCPNTRCSITWFLSWSPCGECSRAITEFLS | |
| | RYPHVTLFIYIARLYHHADPRNRQGLRDLISSGVTIQIMTEQESGYCWRNFV | |
| | NYSPSNEAHWPRYPHLWVRLYVLELYCIILGLPPCLNILRRKQPQLTFFTIA | |
| | LQSCHYQRLPPHILWATGLK | |
| Petromyzon | MTDAEYVRIHEKLDIYTFKKQFFNNKKSVSHRCYVLFELKRRGERRACFW | 68 |
| marinus CDA1 | GYAVNKPQSGTERGIHAEIFSIRKVEEYLRDNPGQFTINWYSSWSPCADCA | |
| (pmCDA1) | EKILEWYNQELRGNGHTLKIWACKLYYEKNARNQIGLWNLRDNGVGLNV | |
| | MVSEHYQCCRKIFIQSSHNQLNENRWLEKTLKRAEKRRSELSIMIQVKILHT | |
| | TKSPAV | |
| Human | MKPHFRNTVERMYRDTFSYNFYNRPILSRRNTVWLCYEVKTKGPSRPPLD | 69 |
| APOBEC3G | AKIFRGQVYSELKYHPEMRFFHWFSKWRKLHRDQEYEVTWYISWSPCTKC | |
| D316R_D317R | TRDMATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPPT | |
| | FTFNFNNEPWVRGRHETYLCYEVERMTINDTWVLLNQRRGFLCNQAPHKH | |
| | GFLEGRHAELCFLDVIPFWKLDLDQDYRVTCFTSWSPCFSCAQEMAKFISK | |
| | NKHVSLCIFTARIYRRQGRCQEGLRTLAEAGAKISIMTYSEFKHCWDTFVD | |
| | HQGCPFQPWDGLDEHSQDLSGRLRAILQNQEN | |
| Human | MDPPTFTFNFNNEPWVRGRHETYLCYEVERMTINDTWVLLNQRRGFLCNQ | 70 |
| APOBEC3G | APHKHGFLEGRHAELCFLDVIPFWKLDLDQDYRVTCFTSWSPCFSCAQEM | |
| chain A | AKFISKNKHVSLCIFTARIYDDQGRCQEGLRTLAEAGAKISIMTYSEFKHCW | |
| | DTFVDHQGCPFQPWDGLDEHSQDLSGRLRAILQ | |
| Human | MDPPTFTFNFNNEPWVRGRHETYLCYEVERMTINDTWVLLNQRRGFLCNQ | 71 |
| APOBEC3G | APHKHGFLEGRHAELCFLDVIPFWKLDLDQDYRVTCFTSWSPCFSCAQEM | |
| chain A | AKFISKNKHVSLCIFTARIYRRQGRCQEGLRTLAEAGAKISIMTYSEFKHCW | |
| D120R_D121R | DTFVDHQGCPFQPWDGLDEHSQDLSGRLRAILQ | |

TABLE 7
Exemplary plasmids

| Name | Plasmid Code | Marker | Used in |
|---|---|---|---|
| P_{tetO}_{_{—}}CDA-nCas9-ugi | pFF1454 | Cam | FIGS. 23A-E, |
| | | | 24A-24E, 25A- |
| | | | 25C & 27A-27D |
| | | | FIGS. 28A-28C, |
| | | | 29A-29E, 30, |
| | | | 31A-31B, 32A- |
| | | | 32B, & 34 |
| Comb_AND_gate | pFF1581 | Carb | FIG. 23B-23D |
| Comb_AND_gatc_gRNA_output | pFF1590 | Carb | FIG. 23E |
| Seq_AND_gate | pFF1610 | Carb | FIG. 24A-24C |
| Race_detecting | pFF1684 | Cam | FIG. 24D |
| | | | FIG. 31A |
| Mixed_seq_logic | pFF1685 | Carb | FIG. 24E |
| | | | FIG. 3 IB |
| 3x_propagation_delay_seq_AND | pFF1588 | Carb | FIG. 25A-25C |
| gRNA(Op*) | pYH383 | Carb | FIG. 26A-26F |
| | | Hygro | FIG. 33A-33C |
| gRNA(NS) | pYH384 | Carb | FIG. 26A-26F |
| | | Hygro | FIG. 33A-33C |
| 4xOp*_1xOp_GFP_pCMV_nCas9_CDA_ugi_VP64 | pYH396 | Carb | FIG. 26A-26F |
| | | Puro | FIG. 33A-33C |
| 1xOp*_GFP_pCMV_nCas9_CDA_ugi_VP64 | pYH404 | Carb | FIG. 26A-26F |
| | | Puro | FIG. 33A-33C |
| Ara_inducible_C-rich_stgRNA | pFF1531 | Carb | FIG. 27A-27D |
| | | | FIG. 34 |
| OR_gate | pFF1583 | Carb | FIG. S29A-29B |
| gRNA_cascade | pFF1586 | Carb | FIG. 29C-29D |
| Multiplexer | pFF1572 | Carb | FIG. 29E |
| Temporal_start_codon_conversion | pFF1573 | Carb | FIG. 32A-32B |
| ATG_conversion | PFF1604 | Carb | FIG. 30 |

TABLE 8
Exemplary synthetic parts and their corresponding sequences

| Part name | Type | Sequence | Source | SEQ ID NO: |
|---|---|---|---|---|
| P_{lacO} (P_{LlacO-1}) | IPTG- | AATTGTGAGCGGATAACAATTGACATTGTGAGCGGATAACAAG | (26) | 72 |
| | inducible | ATACTGAGCACATCAGCAGGACGCACTGACC | | |
| | promoter | | | |
| P_{tetO} | aTc-inducible | TCCCTATCAGTGATAGAGAAAAGAATTCAAAAGATCTAAAGAG | (26) | 73 |
| | promoter | GAGAAAGGATCT | | |
| pBAD | Ara-inducible | ACATTGATTATTTGCACGGCGTCACACTTTGCTATGCCATAGCA | E. coli | 74 |
| | promoter | TTTTTATCCATAAGATTAGCGGATCCTACCTGACGCTTTTTATCG | genome | |
| | | CAACTCTCTACTGTTTCTCCATA | | |
| 4xOp_1xOp* | 4xOp_1xOp* | GACAGGAGAAGAATTGAGACAGGAGAAGAATTGAGACAGGAG | This work | 75 |
| | array | AAGAATTGAGACAGGAGAAGAATTGAGACAGGAGAAGAATTG | | |
| | upstream of | AGATTGGTGGGGGGCTATAAAAGGGGGTGGGGGCGTTCGTCCT | | |
| | minimal MLP | CACTCTAGATCTGCGATCTAAGTAAGCTTGGCATTCCGGTACTG | | |
| | promoter | TTGGTAAAGCCACCATGGC | | |
| 1xOp* | 1xOp* | GACAGGAGAAGAATTGAGATTGGTGGGGGGCTATAAAAGGGG | This work | 76 |
| | upstream of | GTGGGGGCGTTCGTCCTCACTCTAGATCTGCGATCTAAGTAAGC | | |
| | minimal MLP | TTGGCATTCCGGTACTGTTGGTAAAGCCACCATGGC | | |
| | promoter | | | |
| pU6 | Constitutive | TGTACAAAAAAGCAGGCTTTAAAGGAACCAATTCAGTCGACTG | | 77 |
| | RNA Pol III | GATCCGGTACCAAGGTCGGGCAGGAAGAGGGCCTATTTCCCAT | | |
| | promoter | GATTCCTTCATATTTGCATATACGATACAAGGCTGTTAGAGAGA | | |
| | | TAATTAGAATTAATTTGACTGTAAACACAAAGATATTAGTACAA | | |
| | | AATACGTGACGTAGAAAGTAATAATTTCTTGGGTAGTTTGCAGT | | |
| | | TTTAAAATTATGTTTTAAAATGGACTATCATATGCTTACCGTAA | | |
| | | CTTGAAAGTATTTCGATTTCTTGGCTTTATATATCTTGTGGAAAG | | |
| | | GACGAAACACC | | |
| CDA-nCas9- | read-write | ATGAGCTCAGAGACTGGCCCAGTGGCTGTGGACCCCACATTGA | (7) | 78 |
| ugi | head ORF | GACGGCGGATCGAGCCCCATGAGTTTGAGGTATTCTTCGATCCG | | |
| For use in | | AGAGAGCTCCGCAAGGAGACCTGCCTGCTTTACGAAATTAATT | | |
| bacterial | | GGGGGGGCCGGCACTCCATTTGGCGACATACATCACAGAACAC | | |
| experiments. | | TAACAAGCACGTCGAAGTCAACTTCATCGAGAAGTTCACGACA | | |
| The | | GAAAGATATTTCTGTCCGAACACAAGGTGCAGCATTACCTGGTT | | |
| APOBEC1 | | TCTCAGCTGGAGCCCATGCGGCGAATGTAGTAGGGCCATCACT | | |
| CDA protein | | GAATTCCTGTCAAGGTATCCCCACGTCACTCTGTTTATTTACATC | | |
| used as the | | GCAAGGCTGTACCACCACGCTGACCCCCGCAATCGACAAGGCC | | |
| writing | | TGCGGGATTTGATCTCTTCAGGTGTGACTATCCAAATTATGACT | | |
| module. | | GAGCAGGAGTCAGGATACTGCTGGAGAAACTTTGTGAATTATA | | |
| | | GCCCGAGTAATGAAGCCCACTGGCCTAGGTATCCCCATCTGTG | | |
| | | GGTACGACTGTACGTTCTTGAACTGTACTGCATCATACTGGGCC | | |
| | | TGCCTCCTTGTCTCAACATTCTGAGAAGGAAGCAGCCACAGCTG | | |
| | | ACATTCTTTACCATCGCTCTTCAGTCTTGTCATTACCAGCGACTG | | |
| | | CCCCCACACATTCTCTGGGCCACCGGGTTGAAAAGCGGCAGCG | | |
| | | AGACTCCCGGGACCTCAGAGTCCGCCACACCCGAAAGTGATAA | | |
| | | AAAGTATTCTATTGGTTTAGCCATCGGCACTAATTCCGTTGGAT | | |
| | | GGGCTGTCATAACCGATGAATACAAAGTACCTTCAAAGAAATT | | |
| | | TAAGGTGTTGGGGAACACAGACCGTCATTCGATTAAAAAGAAT | | |
| | | CTTATCGGTGCCCTCCTATTCGATAGTGGCGAAACGGCAGAGGC | | |
| | | GACTCGCCTGAAACGAACCGCTCGGAGAAGGTATACACGTCGC | | |
| | | AAGAACCGAATATGTTACTTACAAGAAATTTTTAGCAATGAGA | | |
| | | TGGCCAAAGTTGACGATTCTTTCTTTCACCGTTTGGAAGAGTCC | | |
| | | TTCCTTGTCGAAGAGGACAAGAAACATGAACGGCACCCCATCT | | |
| | | TTGGAAACATAGTAGATGAGGTGGCATATCATGAAAAGTACCC | | |
| | | AACGATTTATCACCTCAGAAAAAAGCTAGTTGACTCAACTGAT | | |
| | | AAAGCGGACCTGAGGTTAATCTACTTGGCTCTTGCCCATATGAT | | |
| | | AAAGTTCCGTGGGCACTTTCTCATTGAGGGTGATCTAAATCCGG | | |
| | | ACAACTCGGATGTCGACAAACTGTTCATCCAGTTAGTACAAACC | | |
| | | TATAATCAGTTGTTTGAAGAGAACCCTATAAATGCAAGTGGCGT | | |
| | | GGATGCGAAGGCTATTCTTAGCGCCCGCCTCTCTAAATCCCGAC | | |
| | | GGCTAGAAAACCTGATCGCACAATTACCCGGAGAGAAGAAAAA | | |
| | | TGGGTTGTTCGGTAACCTTATAGCGCTCTCACTAGGCCTGACAC | | |
| | | CAAATTTTAAGTCGAACTTCGACTTAGCTGAAGATGCCAAATTG | | |
| | | CAGCTTAGTAAGGACACGTACGATGACGATCTCGACAATCTAC | | |
| | | TGGCACAAATTGGAGATCAGTATGCGGACTTATTTTTGGCTGCC | | |
| | | AAAAACCTTAGCGATGCAATCCTCCTATCTGACATACTGAGAGT | | |
| | | TAATACTGAGATTACCAAGGCGCCGTTATCCGCTTCAATGATCA | | |
| | | AAAGGTACGATGAACATCACCAAGACTTGACACTTCTCAAGGC | | |
| | | CCTAGTCCGTCAGCAACTGCCTGAGAAATATAAGGAAATATTCT | | |
| | | TTGATCAGTCGAAAAACGGGTACGCAGGTTATATTGACGGCGG | | |
| | | AGCGAGTCAAGAGGAATTCTACAAGTTTATCAAACCCATATTA | | |
| | | GAGAAGATGGATGGGACGGAAGAGTTGCTTGTAAAACTCAATC | | |
| | | GCGAAGATCTACTGCGAAAGCAGCGGACTTTCGACAACGGTAG | | |
| | | CATTCCACATCAAATCCACTTAGGCGAATTGCATGCTATACTTA | | |
| | | GAAGGCAGGAGGATTTTTATCCGTTCCTCAAAGACAATCGTGA | | |
| | | AAAGATTGAGAAAATCCTAACCTTTCGCATACCTTACTATGTGG | | |
| | | GACCCCTGGCCCGAGGGAACTCTCGGTTCGCATGGATGACAAG | | |
| | | AAAGTCCGAAGAAACGATTACTCCATGGAATTTTGAGGAAGTT | | |
| | | GTCGATAAAGGTGCGTCAGCTCAATCGTTCATCGAGAGGATGA | | |
| | | CCAACTTTGACAAGAATTTACCGAACGAAAAAGTATTGCCTAA | | |
| | | GCACAGTTTACTTTACGAGTATTTCACAGTGTACAATGAACTCA | | |
| | | CGAAAGTTAAGTATGTCACTGAGGGCATGCGTAAACCCGCCTTT | | |
| | | CTAAGCGGAGAACAGAAGAAAGCAATAGTAGATCTGTTATTCA | | |
| | | AGACCAACCGCAAAGTGACAGTTAAGCAATTGAAAGAGGACTA | | |
| | | CTTTAAGAAAATTGAATGCTTCGATTCTGTCGAGATCTCCGGGG | | |
| | | TAGAAGATCGATTTAATGCGTCACTTGGTACGTATCATGACCTC | | |
| | | CTAAAGATAATTAAAGATAAGGACTTCCTGGATAACGAAGAGA | | |
| | | ATGAAGATATCTTAGAAGATATAGTGTTGACTCTTACCCTCTTT | | |
| | | GAAGATCGGGAAATGATTGAGGAAAGACTAAAAACATACGCTC | | |
| | | ACCTGTTCGACGATAAGGTTATGAAACAGTTAAAGAGGCGTCG | | |
| | | CTATACGGGCTGGGGACGATTGTCGCGGAAACTTATCAACGGG | | |
| | | ATAAGAGACAAGCAAAGTGGTAAAACTATTCTCGATTTTCTAA | | |
| | | AGAGCGACGGCTTCGCCAATAGGAACTTTATGCAGCTGATCCA | | |
| | | TGATGACTCTTTAACCTTCAAAGAGGATATACAAAAGGCACAG | | |
| | | GTTTCCGGACAAGGGGACTCATTGCACGAACATATTGCGAATCT | | |
| | | TGCTGGTTCGCCAGCCATCAAAAAGGGCATACTCCAGACAGTC | | |
| | | AAAGTAGTGGATGAGCTAGTTAAGGTCATGGGACGTCACAAAC | | |
| | | CGGAAAACATTGTAATCGAGATGGCACGCGAAAATCAAACGAC | | |
| | | TCAGAAGGGGCAAAAAAACAGTCGAGAGCGGATGAAGAGAAT | | |
| | | AGAAGAGGGTATTAAAGAACTGGGCAGCCAGATCTTAAAGGAG | | |
| | | CATCCTGTGGAAAATACCCAATTGCAGAACGAGAAACTTTACC | | |
| | | TCTATTACCTACAAAATGGAAGGGACATGTATGTTGATCAGGA | | |
| | | ACTGGACATAAACCGTTTATCTGATTACGACGTCGATCACATTG | | |
| | | TACCCCAATCCTTTTTGAAGGACGATTCAATCGACAATAAAGTG | | |
| | | CTTACACGCTCGGATAAGAACCGAGGGAAAAGTGACAATGTTC | | |
| | | CAAGCGAGGAAGTCGTAAAGAAAATGAAGAACTATTGGCGGC | | |
| | | AGCTCCTAAATGCGAAACTGATAACGCAAAGAAAGTTCGATAA | | |
| | | CTTAACTAAAGCTGAGAGGGGTGGCTTGTCTGAACTTGACAAG | | |
| | | GCCGGATTTATTAAACGTCAGCTCGTGGAAACCCGCCAAATCA | | |
| | | CAAAGCATGTTGCACAGATACTAGATTCCCGAATGAATACGAA | | |
| | | ATACGACGAGAACGATAAGCTGATTCGGGAAGTCAAAGTAATC | | |
| | | ACTTTAAAGTCAAAATTGGTGTCGGACTTCAGAAAGGATTTTCA | | |
| | | ATTCTATAAAGTTAGGGAGATAAATAACTACCACCATGCGCAC | | |
| | | GACGCTTATCTTAATGCCGTCGTAGGGACCGCACTCATTAAGAA | | |
| | | ATACCCGAAGCTAGAAAGTGAGTTTGTGTATGGTGATTACAAA | | |
| | | GTTTATGACGTCCGTAAGATGATCGCGAAAAGCGAACAGGAGA | | |
| | | TAGGCAAGGCTACAGCCAAATACTTCTTTTATTCTAACATTATG | | |
| | | AATTTCTTTAAGACGGAAATCACTCTGGCAAACGGAGAGATAC | | |
| | | GCAAACGACCTTTAATTGAAACCAATGGGGAGACAGGTGAAAT | | |
| | | CGTATGGGATAAGGGCCGGGACTTCGCGACGGTGAGAAAAGTT | | |
| | | TTGTCCATGCCCCAAGTCAACATAGTAAAGAAAACTGAGGTGC | | |
| | | AGACCGGAGGGTTTTCAAAGGAATCGATTCTTCCAAAAAGGAA | | |
| | | TAGTGATAAGCTCATCGCTCGTAAAAAGGACTGGGACCCGAAA | | |
| | | AAGTACGGTGGCTTCGATAGCCCTACAGTTGCCTATTCTGTCCT | | |
| | | AGTAGTGGCAAAAGTTGAGAAGGGAAAATCCAAGAAACTGAA | | |
| | | GTCAGTCAAAGAATTATTGGGGATAACGATTATGGAGCGCTCG | | |
| | | TCTTTTGAAAAGAACCCCATCGACTTCCTTGAGGCGAAAGGTTA | | |
| | | CAAGGAAGTAAAAAAGGATCTCATAATTAAACTACCAAAGTAT | | |
| | | AGTCTGTTTGAGTTAGAAAATGGCCGAAAACGGATGTTGGCTA | | |
| | | GCGCCGGAGAGCTTCAAAAGGGGAACGAACTCGCACTACCGTC | | |
| | | TAAATACGTGAATTTCCTGTATTTAGCGTCCCATTACGAGAAGT | | |
| | | TGAAAGGTTCACCTGAAGATAACGAACAGAAGCAACTTTTTGT | | |
| | | TGAGCAGCACAAACATTATCTCGACGAAATCATAGAGCAAATT | | |
| | | TCGGAATTCAGTAAGAGAGTCATCCTAGCTGATGCCAATCTGG | | |
| | | ACAAAGTATTAAGCGCATACAACAAGCACAGGGATAAACCCAT | | |
| | | ACGTGAGCAGGCGGAAAATATTATCCATTTGTTTACTCTTACCA | | |
| | | ACCTCGGCGCTCCAGCCGCATTCAAGTATTTTGACACAACGATA | | |
| | | GATCGCAAACGATACACTTCTACCAAGGAGGTGCTAGACGCGA | | |
| | | CACTGATTCACCAATCCATCACGGGATTATATGAAACTCGGATA | | |
| | | GATTTGTCACAGCTTGGGGGTGACTCTGGTGGTTCTACTAATCT | | |
| | | GTCAGATATTATTGAAAAGGAGACCGGTAAGCAACTGGTTATC | | |
| | | CAGGAATCCATCCTCATGCTCCCAGAGGAGGTGGAAGAAGTCA | | |
| | | TTGGGAACAAGCCGGAAAGCGATATACTCGTGCACACCGCCTA | | |
| | | CGACGAGAGCACCGACGAGAATGTCATGCTTCTGACTAGCGAC | | |
| | | GCCCCTGAATACAAGCCTTGGGCTCTGGTCATACAGGATAGCA | | |
| | | ACGGTGAGAACAAGATTAAGATGCTCTCTGGTGGTTCTCCCAA | | |
| | | GAAGAAGAGGAAAGTCTAA | | |
| nCas9-CDA- | read-write- | ATGGCACCGAAGAAGAAGCGTAAAGTCGGAATCCACGGAGTTC | This work | 79 |
| ugi-VP64 | transactivator | CTGCGGCAATGGACAAGAAGTACTCCATTGGGCTCGCTATCGG | | |
| For use in | ORF | CACAAACAGCGTCGGTTGGGCCGTCATTACGGACGAGTACAAG | | |
| mammalian | | GTGCCGAGCAAAAAATTCAAAGTTCTGGGCAATACCGATCGCC | | |
| cell | | ACAGCATAAAGAAGAACCTCATTGGCGCCCTCCTGTTCGACTCC | | |
| experiments. | | GGGGAGACGGCCGAAGCCACGCGGCTCAAAAGAACAGCACGG | | |
| PmCDA | | CGCAGATATACCCGCAGAAAGAATCGGATCTGCTACCTGCAGG | | |
| protein (8) | | AGATCTTTAGTAATGAGATGGCTAAGGTGGATGACTCTTTCTTC | | |
| and minimal | | CATAGGCTGGAGGAGTCCTTTTTGGTGGAGGAGGATAAAAAGC | | |
| VP64 (10) | | ACGAGCGCCACCCAATCTTTGGCAATATCGTGGACGAGGTGGC | | |
| domain were | | GTACCATGAAAAGTACCCAACCATATATCATCTGAGGAAGAAG | | |
| used as the | | CTTGTAGACAGTACTGATAAGGCTGACTTGCGGTTGATCTATCT | | |
| write and the | | CGCGCTGGCGCATATGATCAAATTTCGGGGACACTTCCTCATCG | | |
| trans- | | AGGGGGACCTGAACCCAGACAACAGCGATGTCGACAAACTCTT | | |
| activation | | TATCCAACTGGTTCAGACTTACAATCAGCTTTTCGAAGAGAACC | | |
| modules, | | CGATCAACGCATCCGGAGTTGACGCCAAAGCAATCCTGAGCGC | | |
| respectively. | | TAGGCTGTCCAAATCCCGGCGGCTCGAAAACCTCATCGCACAG | | |
| | | CTCCCTGGGGAGAAGAAGAACGGCCTGTTTGGTAATCTTATCGC | | |
| | | CCTGTCACTCGGGCTGACCCCCAACTTTAAATCTAACTTCGACC | | |
| | | TGGCCGAAGATGCCAAGCTTCAACTGAGCAAAGACACCTACGA | | |
| | | TGATGATCTCGACAATCTGCTGGCCCAGATCGGCGACCAGTAC | | |
| | | GCAGACCTTTTTTTGGCGGCAAAGAACCTGTCAGACGCCATTCT | | |
| | | GCTGAGTGATATTCTGCGAGTGAACACGGAGATCACCAAAGCT | | |
| | | CCGCTGAGCGCTAGTATGATCAAGCGCTATGATGAGCACCACC | | |
| | | AAGACTTGACTTTGCTGAAGGCCCTTGTCAGACAGCAACTGCCT | | |
| | | GAGAAGTACAAGGAAATTTTCTTCGATCAGTCTAAAAATGGCT | | |
| | | ACGCCGGATACATTGATGGCGGAGCAAGCCAGGAGGAATTTTA | | |
| | | CAAATTTATTAAGCCCATCTTGGAAAAAATGGACGGCACCGAG | | |
| | | GAGCTGCTGGTAAAGCTTAACAGAGAAGATCTGTTGCGCAAAC | | |
| | | AGCGCACTTTCGACAATGGAAGCATCCCCCACCAGATTCACCT | | |
| | | GGGCGAACTGCACGCTATCCTCAGGCGGCAAGAGGATTTCTAC | | |
| | | CCCTTTTTGAAAGATAACAGGGAAAAGATTGAGAAAATCCTCA | | |
| | | CATTTCGGATACCCTACTATGTAGGCCCCCTCGCCCGGGGAAAT | | |
| | | TCCAGATTCGCGTGGATGACTCGCAAATCAGAAGAGACCATCA | | |
| | | CTCCCTGGAACTTCGAGGAAGTCGTGGATAAGGGGGCCTCTGC | | |
| | | CCAGTCCTTCATCGAAAGGATGACTAACTTTGATAAAAATCTGC | | |
| | | CTAACGAAAAGGTGCTTCCTAAACACTCTCTGCTGTACGAGTAC | | |
| | | TTCACAGTTTATAACGAGCTCACCAAGGTCAAATACGTCACAG | | |
| | | AAGGGATGAGAAAGCCAGCATTCCTGTCTGGAGAGCAGAAGAA | | |
| | | AGCTATCGTGGACCTCCTCTTCAAGACGAACCGGAAAGTTACC | | |
| | | GTGAAACAGCTCAAAGAAGACTATTTCAAAAAGATTGAATGTT | | |
| | | TCGACTCTGTTGAAATCAGCGGAGTGGAGGATCGCTTCAACGC | | |
| | | ATCCCTGGGAACGTATCACGATCTCCTGAAAATCATTAAAGAC | | |
| | | AAGGACTTCCTGGACAATGAGGAGAACGAGGACATTCTTGAGG | | |
| | | ACATTGTCCTCACCCTTACGTTGTTTGAAGATAGGGAGATGATT | | |
| | | GAAGAACGCTTGAAAACTTACGCTCATCTCTTCGACGACAAAG | | |
| | | TCATGAAACAGCTCAAGAGGCGCCGATATACAGGATGGGGGCG | | |
| | | GCTGTCAAGAAAACTGATCAATGGGATCCGAGACAAGCAGAGT | | |
| | | GGAAAGACAATCCTGGATTTTCTTAAGTCCGATGGATTTGCCAA | | |
| | | CCGGAACTTCATGCAGTTGATCCATGATGACTCTCTCACCTTTA | | |
| | | AGGAGGACATCCAGAAAGCACAAGTTTCTGGCCAGGGGGACAG | | |
| | | TCTTCACGAGCACATCGCTAATCTTGCAGGTAGCCCAGCTATCA | | |
| | | AAAAGGGAATACTGCAGACCGTTAAGGTCGTGGATGAACTCGT | | |
| | | CAAAGTAATGGGAAGGCATAAGCCCGAGAATATCGTTATCGAG | | |
| | | ATGGCCCGAGAGAACCAAACTACCCAGAAGGGACAGAAGAAC | | |
| | | AGTAGGGAAAGGATGAAGAGGATTGAAGAGGGTATAAAAGAA | | |
| | | CTGGGGTCCCAAATCCTTAAGGAACACCCAGTTGAAAACACCC | | |
| | | AGCTTCAGAATGAGAAGCTCTACCTGTACTACCTGCAGAACGG | | |
| | | CAGGGACATGTACGTGGATCAGGAACTGGACATCAATCGGCTC | | |
| | | TCCGACTACGACGTGGATCATATCGTGCCCCAGTCTTTTCTCAA | | |
| | | AGATGATTCTATTGATAATAAAGTGTTGACAAGATCCGATAAA | | |
| | | AATAGAGGGAAGAGTGATAACGTCCCCTCAGAAGAAGTTGTCA | | |
| | | AGAAAATGAAAAATTATTGGCGGCAGCTGCTGAACGCCAAACT | | |
| | | GATCACACAACGGAAGTTCGATAATCTGACTAAGGCTGAACGA | | |
| | | GGTGGCCTGTCTGAGTTGGATAAAGCCGGCTTCATCAAAAGGC | | |
| | | AGCTTGTTGAGACACGCCAGATCACCAAGCACGTGGCCCAAAT | | |
| | | TCTCGATTCACGCATGAACACCAAGTACGATGAAAATGACAAA | | |
| | | CTGATTCGAGAGGTGAAAGTTATTACTCTGAAGTCTAAGCTGGT | | |
| | | CTCAGATTTCAGAAAGGACTTTCAGTTTTATAAGGTGAGAGAG | | |
| | | ATCAACAATTACCACCATGCGCATGATGCCTACCTGAATGCAGT | | |
| | | GGTAGGCACTGCACTTATCAAAAAATATCCCAAGCTTGAATCTG | | |
| | | AATTTGTTTACGGAGACTATAAAGTGTACGATGTTAGGAAAAT | | |
| | | GATCGCAAAGTCTGAGCAGGAAATAGGCAAGGCCACCGCTAAG | | |
| | | TACTTCTTTTACAGCAATATTATGAATTTTTTCAAGACCGAGATT | | |
| | | ACACTGGCCAATGGAGAGATTCGGAAGCGACCACTTATCGAAA | | |
| | | CAAACGGAGAAACAGGAGAAATCGTGTGGGACAAGGGTAGGG | | |
| | | ATTTCGCGACAGTCCGGAAGGTCCTGTCCATGCCGCAGGTGAA | | |
| | | CATCGTTAAAAAGACCGAAGTACAGACCGGAGGCTTCTCCAAG | | |
| | | GAAAGTATCCTCCCGAAAAGGAACAGCGACAAGCTGATCGCAC | | |
| | | GCAAAAAAGATTGGGACCCCAAGAAATACGGCGGATTCGATTC | | |
| | | TCCTACAGTCGCTTACAGTGTACTGGTTGTGGCCAAAGTGGAGA | | |
| | | AAGGGAAGTCTAAAAAACTCAAAAGCGTCAAGGAACTGCTGGG | | |
| | | CATCACAATCATGGAGCGATCAAGCTTCGAAAAAAACCCCATC | | |
| | | GACTTTCTCGAGGCGAAAGGATATAAAGAGGTCAAAAAAGACC | | |
| | | TCATCATTAAGCTTCCCAAGTACTCTCTCTTTGAGCTTGAAAAC | | |
| | | GGCCGGAAACGAATGCTCGCTAGTGCGGGCGAGCTGCAGAAAG | | |
| | | GTAACGAGCTGGCACTGCCCTCTAAATACGTTAATTTCTTGTAT | | |
| | | CTGGCCAGCCACTATGAAAAGCTCAAAGGGTCTCCCGAAGATA | | |
| | | ATGAGCAGAAGCAGCTGTTCGTGGAACAACACAAACACTACCT | | |
| | | TGATGAGATCATCGAGCAAATAAGCGAATTCTCCAAAAGAGTG | | |
| | | ATCCTCGCCGACGCTAACCTCGATAAGGTGCTTTCTGCTTACAA | | |
| | | TAAGCACAGGGATAAGCCCATCAGGGAGCAGGCAGAAAACATT | | |
| | | ATCCACTTGTTTACTCTGACCAACTTGGGCGCGCCTGCAGCCTT | | |
| | | CAAGTACTTCGACACCACCATAGACAGAAAGCGGTACACCTCT | | |
| | | ACAAAGGAGGTCCTGGACGCCACACTGATTCATCAGTCAATTA | | |
| | | CGGGGCTCTATGAAACAAGAATCGACCTCTCTCAGCTCGGTGG | | |
| | | AGACAGCAGGGCTGACCCCAAGAAGAAGAGGAAGGTGGGTGG | | |
| | | AGGAGGTACCGGCGGTGGAGGCTCAGCAGAATACGTACGAGCT | | |
| | | CTGTTTGACTTCAATGGGAATGACGAGGAGGATCTCCCCTTTAA | | |
| | | GAAGGGCGATATTCTCCGCATCAGAGATAAGCCCGAAGAACAA | | |
| | | TGGTGGAATGCCGAGGATAGCGAAGGGAAAAGGGGCATGATTC | | |
| | | TGGTGCCATATGTGGAGAAATATTCCGGTGACTACAAAGACCA | | |
| | | TGATGGGGATTACAAAGACCACGACATCGACTACAAAGACGAC | | |
| | | GACGATAAATCAGGGATGACAGACGCCGAGTACGTGCGCATTC | | |
| | | ATGAGAAACTGGATATTTACACCTTCAAGAAGCAGTTCTTCAAC | | |
| | | AACAAGAAATCTGTGTCACACCGCTGCTACGTGCTGTTTGAGTT | | |
| | | GAAGCGAAGGGGCGAAAGAAGGGCTTGCTTTTGGGGCTATGCC | | |
| | | GTCAACAAGCCCCAAAGTGGCACCGAGAGAGGAATACACGCTG | | |
| | | AGATATTCAGTATCCGAAAGGTGGAAGAGTATCTTCGGGATAA | | |
| | | TCCTGGGCAGTTTACGATCAACTGGTATTCCAGCTGGAGTCCTT | | |
| | | GCGCTGATTGTGCCGAGAAAATTCTGGAATGGTATAATCAGGA | | |
| | | ACTTCGGGGAAACGGGCACACATTGAAAATCTGGGCCTGCAAG | | |
| | | CTGTACTACGAGAAGAATGCCCGGAACCAGATAGGACTCTGGA | | |
| | | ATCTGAGGGACAATGGTGTAGGCCTGAACGTGATGGTTTCCGA | | |
| | | GCACTATCAGTGTTGTCGGAAGATTTTCATCCAAAGCTCTCATA | | |
| | | ACCAGCTCAATGAAAACCGCTGGTTGGAGAAAACACTGAAACG | | |
| | | TGCGGAGAAGTGGAGATCCGAGCTGAGCATCATGATCCAGGTC | | |
| | | AAGATTCTGCATACCACTAAGTCTCCAGCCGTTGGTCCCAAGAA | | |
| | | GAAAAGAAAAGTCGGTACCATGACCAACCTTTCCGACATCATA | | |
| | | GAGAAGGAAACAGGCAAACAGTTGGTCATCCAAGAGTCGATAC | | |
| | | TCATGCTTCCTGAAGAAGTTGAGGAGGTCATTGGGAATAAGCC | | |
| | | GGAAAGTGACATTCTCGTACACACTGCGTATGATGAGAGCACC | | |
| | | GATGAGAACGTGATGCTGCTCACGTCAGATGCCCCAGAGTACA | | |
| | | AACCCTGGGCTCTGGTGATTCAGGACTCTAATGGAGAGAACAA | | |
| | | GATCAAGATGCTATCTGGTGGTTCTCCCAAGAAGAAGAGGAAA | | |
| | | GTCGAGGATCCAAAGAAGAAAAGGAAGGTTGAAGACCCCAAG | | |
| | | AAAAAGAGGAAGGTGGATGGGATCGGCTCAGGCAGCAACGGC | | |
| | | GGTGGAGGTTCAGACGCTTTGGACGATTTCGATCTCGATATGCT | | |
| | | CGGTTCTGACGCCCTGGATGATTTCGATCTGGATATGCTCGGCA | | |
| | | GCGACGCTCTCGACGATTTCGACCTCGACATGCTCGGGTCAGAT | | |
| | | GCCTTGGATGATTTTGACCTGGATATGCTC | | |

TABLE 9
Exemplary HTS primers and their corresponding sequences

| name | Type | Sequence | Used in | SEQ ID NO: |
|---|---|---|---|---|
| FF_oligo_2525 | HTS_Primer_ | ACACTCTTTCCCTACACGACGCTCTTCC | FIGS. 23C, 25C, | 80 |
| | Forward | GATCTNNNNNTGCTGCCCGACAACCAC | 28C, 29D, 31A- | |
| | | TA | 31B | |
| FF_oligo_2526 | HTS_Primer_ | CGGCATTCCTGCTGAACCGCTCTTCCGA | FIGS. 23C, 25C, | 81 |
| | Reverse | TCTNNNNNTGAACAACCACCACTTCAA | 28C, 29D, 31A- | |
| | | GTGGG | 31B | |
| FF_oligo_2527 | HTS_Primer_ | CACTCTTTCCCTACACGACGCTCTTCCG | FIGS. 26A-26F | 82 |
| | Forward | ATCTNNNNNGGACAGCAGAGATCCAGT | & 33A-33C | |
| | | TTGGT | | |
| FF_oligo_2528 | HTS_Primer_ | GGCATTCCTGCTGAACCGCTCTTCCGAT | FIGS. 26A-26F | 83 |
| | Reverse | CTNNNNNTCGCAGATCTAGAGTGAGGA | & 33A-33C | |
| | | CGAAC | | |
| FF_oligo_2399 | HTS_Primer_ | ACACTCTTTCCCTACACGACGCTCTTCC | FIGS. 27A-27D | 84 |
| | Forward | GATCTNNNNNTTT TAT | & 34 | |
| | | CGCAACTCTCTACTGTTT | | |
| FF_oligo_2124 | HTS_Primer_ | GGCATTCCTGCTGAACCGCTCTTCCGAT | FIGS. 27A-27D | 85 |
| | Reverse | CTNNNNNTTCAAGTTGATAACGGACTA | & 34 | |
| | | GCCTT | | |

### REFERENCES

1. P. Siuti, J. Yazbek, T. K. Lu, Synthetic circuits integrating logic and memory in living cells. Nature Biotechnology 31, 448-452 (2013); published online EpubMay (10.1038/nbt.2510).
2. N. Roquet, A. P. Soleimany, A. C. Ferris, S. Aaronson, T. K. Lu, Synthetic recombinase-based state machines in living cells. Science 353, aad8559 (2016); published online EpubJul 22 (10.1126/science.aad8559).
3. F. Farzadfard, T. K. Lu, Genomically encoded analog memory with precise in vivo DNA writing in living cell populations. Science 346, 1256272 (2014); published online EpubNov 14 (10.1126/science.1256272).
4. A. McKenna, G. M. Findlay, J. A. Gagnon, M. S. Horwitz, A. F. Schier, J. Shendure, Whole-organism lineage tracing by combinatorial and cumulative genome editing. Science 353, aaf7907 (2016); published online EpubJul 29 (10.1126/science.aaf7907).
5. K. L. Frieda, J. M. Linton, S. Hormoz, J. Choi, K. K. Chow, Z. S. Singer, M. W. Budde, M. B. Elowitz, L. Cai, Synthetic recording and in situ readout of lineage information in single cells. Nature 541, 107-111 (2017); published online EpubJan 05 (10.1038/nature20777).
6. S. D. Perli, C. H. Cui, T. K. Lu, Continuous genetic recording with self-targeting CRISPR-Cas in human cells. Science 353, (2016); published online EpubSep 09 (10.1126/science.aag0511).
7. A. C. Komor, Y. B. Kim, M. S. Packer, J. A. Zuris, D. R. Liu, Programmable editing of a target base in genomic DNA without double-stranded DNA cleavage. Nature 533, 420-424 (2016); published online EpubMay 19 (10.1038/nature17946).
8. K. Nishida, T. Arazoe, N. Yachie, S. Banno, M. Kakimoto, M. Tabata, M. Mochizuki, A. Miyabe, M. Araki, K. Y. Hara, Z. Shimatani, A. Kondo, Targeted nucleotide editing using hybrid prokaryotic and vertebrate adaptive immune systems. Science 353, (2016); published online EpubSep 16 (10.1126/science.aaff729).
9. B. J. Glassner, L. J. Rasmussen, M. T. Najarian, L. M. Posnick, L. D. Samson, Generation of a strong mutator phenotype in yeast by imbalanced base excision repair. Proceedings of the National Academy of Sciences of the United States of America 95, 9997-10002 (1998); published online EpubAug 18.
10. S. B. Rubin-Pitel, H. Zhao, Recent advances in biocatalysis by directed enzyme evolution. Comb Chem High Throughput Screen 9, 247-257 (2006); published online EpubMay.
11. N. J. Turner, Directed evolution drives the next generation of biocatalysts. Nat Chem Biol 5, 567-573 (2009); published online EpubAug (nchembio.203 [pii] 10.1038/nchembio.203).
12. A. Kumar, S. Singh, Directed evolution: tailoring biocatalysts for industrial applications. Crit Rev Biotechnol, (2012); published online EpubSep 18 (10.3109/07388551.2012.716810).
13. H. H. Wang, F. J. Isaacs, P. A. Carr, Z. Z. Sun, G. Xu, C. R. Forest, G. M. Church, Programming cells by multiplex genome engineering and accelerated evolution. Nature 460, 894-898 (2009); published online EpubAug 13 (nature08187 [pii] 10.1038/nature08187).
14. K. M. Esvelt, J. C. Carlson, D. R. Liu, A system for the continuous directed evolution of biomolecules. Nature 472, 499-503 (2011); published online EpubApr 28 (nature09929 [pii] 10.1038/nature09929).
15. D. N. Nesbeth, A. Zaikin, Y. Saka, M. C. Romano, C. V. Giuraniuc, O. Kanakov, T. Laptyeva, Synthetic biology routes to bio-artificial intelligence. Essays in biochemistry 60, 381-391 (2016); published online EpubNov 30 (10.1042/EBC20160014).
16. N. Gandhi, G. Ashkenasy, E. Tannenbaum, Associative learning in biochemical networks. Journal of theoretical biology 249, 58-66 (2007); published online EpubNov 07 (10.1016/j.jtbi.2007.07.004).
17. D. Bray, Molecular networks: the top-down view. Science 301, 1864-1865 (2003); published online EpubSep 26 (10.1126/science.1089118).
18. I. Tagkopoulos, Y. C. Liu, S. Tavazoie, Predictive behavior within microbial genetic networks. Science 320, 1313-1317 (2008); published online EpubJun 06 (10.1126/science.1154456).
19. F. Farzadfard, S. D. Perli, T. K. Lu, Tunable and multifunctional eukaryotic transcription factors based on CRISPR/Cas. ACS synthetic biology 2, 604-613 (2013); published online EpubOct 18 (10.1021/sb400081r).
20. A. Chavez, J. Scheiman, S. Vora, B. W. Pruitt, M. Tuttle, P. R. I. E, S. Lin, S. Kiani, C. D. Guzman, D. J. Wiegand, D. Ter-Ovanesyan, J. L. Braff, N. Davidsohn, B. E. Housden, N. Perrimon, R. Weiss, J. Aach, J. J. Collins, G. M. Church, Highly efficient Cas9-mediated transcriptional programming. Nature methods 12, 326-328 (2015); published online EpubApr (10.1038/nmeth.3312).
21. X. S. Liu, H. Wu, X. Ji, Y. Stelzer, X. Wu, S. Czauderna, J. Shu, D. Dadon, R. A. Young, R. Jaenisch, Editing DNA Methylation in the Mammalian Genome. Cell 167, 233-247 e217 (2016); published online EpubSep 22 (10.1016/j.cell.2016.08.056).
22. I. B. Hilton, A. M. D′Ippolito, C. M. Vockley, P. I. Thakore, G. E. Crawford, T. E. Reddy, C. A. Gersbach, Epigenome editing by a CRISPR-Cas9-based acetyltransferase activates genes from promoters and enhancers. Nature biotechnology 33, 510-517 (2015); published online EpubMay (10.1038/nbt.3199).
23. M. L. Crowe, SeqDoC: rapid SNP and mutation detection by direct comparison of DNA sequence chromatograms. BMC bioinformatics 6, 133 (2005); published online EpubMay 31 (10.1186/1471-2105-6-133).
24. D. G. Gibson, Enzymatic assembly of overlapping DNA fragments. Methods in enzymology 498, 349-361 (2011)10.1016/B978-0-12-385120-8.00015-2).
25. C. Engler, S. Marillonnet, Golden Gate cloning. Methods in molecular biology 1116, 119-131 (2014)10.1007/978-1-62703-764-8_9).
26. R. Lutz, H. Bujard, Independent and tight regulation of transcriptional units in Escherichia coli via the LacR/O, the TetR/O and AraC/I1-I2 regulatory elements. Nucleic Acids Res 25, 1203-1210 (1997); published online EpubMar 15 (gka167 [pii]).
27. A. E. Briner, P. D. Donohoue, A. A. Gomaa, K. Selle, E. M. Slorach, C. H. Nye, R. E. Haurwitz, C. L. Beisel, A. P. May, R. Barrangou, Guide RNA functional modules direct Cas9 activity and orthogonality. Molecular cell 56, 333-339 (2014); published online EpubOct 23 (10.1016/j.molcel.2014.09.019).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United

States Patent Office Manual of Patent Examining Procedures, Section 2111.03.

What is claimed is:

1. A method for encoding memory in a cell, comprising:
(a) delivering to the cell
(i) a nucleic acid comprising a first inducible promoter operably linked to a nucleotide sequence encoding a fusion protein comprising a catalytically-inactive Cas9 (dCas9) or a Cas9 nickase (nCas9), and a base editor enzyme, and
(ii) a nucleic acid comprising a second inducible promoter operably linked to a nucleotide sequence encoding a first guide RNA (gRNA) comprising a specificity determining sequence (SDS) complementary to a first target sequence in the cell, wherein the first target sequence comprises at least one nucleotide base targeted by the base editor enzyme and the second inducible promoter differs from the first inducible promoter, and
(iii) a nucleic acid comprising a third inducible promoter operably linked to a nucleotide sequence encoding at least one other gRNA comprising a SDS complementary to at least one additional target sequence or a modified version of the first target sequence in the cell, wherein the modified version of the first target sequence comprises at least one nucleotide base mutation, and the third inducible promoter, optionally differs from the second inducible promoter;
(b) delivering to the cell first inducer signal that activates transcription from the first inducible promoter, a second inducer signal that activates transcription from the second inducible promoter, and optionally a third inducer signal that activates transcription from the third inducible promoter; and
(c) producing a cell that comprises a nucleotide base mutation in the first target sequence and optionally in the at least one additional target sequence.

2. The method of claim 1, wherein the fusion protein comprises nCas9.

3. The method of claim 1 or 2, wherein the fusion protein further comprises uracil DNA glycosylase inhibitor (ugi).

4. The method of any one of claims 1-3, wherein the base editor enzyme is cytidine deaminase, the at least one nucleotide base targeted by the base editor enzyme is cytidine, and the at least one nucleotide base mutation is a cytidine to thymine mutation.

5. The method of any one of claims 1-3, wherein the base editor enzyme is adenosine deaminase, the at least one nucleotide base targeted by the base editor enzyme is adenosine, and the at least one nucleotide base mutation is an adenosine to inosine mutation.

6. The method of any one of claims 1-5, wherein the target sequence is a genomic sequence.

7. The method of any one of claims 1-6, wherein the third inducible promoter differs from the second inducible promoter, and the method comprises delivering to the cell a third inducer signal that activates transcription from the third inducible promoter.

8. The method of any one of claims 1-7, wherein at least one nucleotide base mutation is produced in the first target sequence and in the at least one additional target sequence.

9. The method of any one of claims 1-8, wherein the at least one additional gRNA comprises a SDS complementary to a region spanning a modified region of the first target sequence and a second target sequence in the cell.

10. The method of any one of claims 1-9, wherein the first, second, and/or third inducer signals are delivered simultaneously or sequentially.

11. The method of any one of claims 1-10, wherein the cell is a bacterial cell.

12. The method of any one of claims 1-10, wherein the cell is a mammalian cell, and optionally wherein the mammalian cell is a human cell.

13. The method of any one of claims 1-12, wherein the first, second, and/or third inducible promoter is selected from isopropyl β-D-1-thiogalactopyranoside (IPTG)-inducible promoters, arabinose (Ara)-inducible promoters, and anhydrotetracycline (aTc)-inducible promoters.

14. A cell comprising
(a) a nucleic acid comprising a first inducible promoter operably linked to a nucleotide sequence encoding a fusion protein comprising a catalytically-inactive Cas9 (dCas9) or a Cas9 nickase (nCas9), and a base editor enzyme, and
(b) a nucleic acid comprising a second inducible promoter operably linked to a nucleotide sequence encoding a first guide RNA (gRNA) comprising a specificity determining sequence (SDS) complementary to a first target sequence in the cell, wherein the first target sequence comprises at least one nucleotide base targeted by the base editor enzyme and the second inducible promoter differs from the first inducible promoter, and
(c) a nucleic acid comprising a third inducible promoter operably linked to a nucleotide sequence encoding at least one other gRNA comprising a specificity determining sequence (SDS) complementary to at least one additional target sequence or a modified version of the first target sequence in the cell, wherein the modified version of the first target sequence comprises at least one nucleotide base mutation, and the third inducible promoter, optionally differs from the second inducible promoter.

15. A cell comprising:
(a) an engineered nucleic acid comprising a promoter operably linked to a nucleotide sequence encoding a self-targeting guide ribonucleic acid (stgRNA) that comprises a specificity determining sequence (SDS) and a protospacer adjacent motif (PAM); and
(b) a fusion protein comprising a catalytically-inactive Cas9 fused to cytidine deaminase.

16. The cell of claim 15, wherein the RNA-guided endonuclease is Cas9 or Cpf1.

17. The cell of claim 15 or 16, wherein the promoter is an inducible promoter.

18. The cell of any one of claims 1-17, wherein at least 20% of the nucleotides of the SDS comprises cytosine bases.

19. An in vivo diversification method, comprising:
(a) introducing into a cell (i) a nucleic acid encoding a biomolecule that has at least one variable region, (ii) a nucleic acid encoding a guide ribonucleic acid (gRNA) that targets the at least one variable region, and (iii) a nucleic acid encoding a fusion protein comprising a catalytically-inactive Cas9 fused (dCas9) to a base editor enzyme or a Cas9 nickase (nCas9) fused to a base editor enzyme; and
(b) producing diversified biomolecules comprising at least one diversified variable region.

20. The method of claim, wherein the base editor enzyme is selected from cytidine deaminases, adenine deaminases, DNA glycosylases, and ROS generators.
